(19) United States
(12) Patent Application Publication
(10) Pub. No.: US 2015/0065343 A1
(43) Pub. Date: Mar. 5, 2015

(54) Acrylamide compounds for combating invertebrate pests

(71) Applicant: BASF SE, Ludwigshafen (DE)

(72) Inventors: Pascal Bindschaedler, Roemerberg (DE); Wolfgang Von Deyn, Neustadt (DE); Karsten Koerber, Eppelheim (DE); Florian Kaiser, Mannheim (DE); Ralph Paulini, Bad Duerkheim (DE); Deborah L. Culbertson, Fuquay Varina, NC (US); Paul Neese, Apex, NC (US); Franz Josef Braun, Durham, NC (US)

(21) Appl. No.: 14/389,120

(22) PCT Filed: Mar. 28, 2013
(86) PCT No.: PCT/EP2013/056718

§ 371 Date: Sep. 29, 2014

Related U.S. Application Data
(60) Provisional application No. 61/618,867, filed on Apr. 2, 2012.
(60) Provisional application No. 61/706,135, filed on Sep. 27, 2012.

(51) Int. Cl. C07D 333/24; A01N 43/78; A01N 43/40; C07D 417/12; A01N 43/10; C07D 409/12

(52) U.S. Cl. CPC C07D 333/24; A01N 43/10; C07D 409/12; A01N 43/40; C07D 417/12; A01N 43/78
USPC 504/100; 549/77; 514/438; 549/60; 514/444; 549/59; 546/280.4; 514/336; 548/204; 514/365

(57) ABSTRACT

The present invention relates to acrylamide compounds which are useful for combating or controlling invertebrate pests, in particular arthropod pests and nematodes, and to a method for producing them. The invention also relates to a method for controlling invertebrate pests by using these compounds and to plant propagation material and to an agricultural and a veterinary composition comprising said compounds.

The present invention relates to acrylamide compounds which are useful for combating or controlling invertebrate pests, in particular arthropod pests and nematodes, and to a method for producing them. The invention also relates to a method for controlling invertebrate pests by using these compounds and to plant propagation material and to an agricultural and a veterinary composition comprising said compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating invertebrate pests, in particular insects, arachnids and nematodes.

Related insecticidal acrylamide compounds are described in WO 2011/054436. However, this document does not describe compounds having the characteristic substituents as claimed in the present invention.

It is an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by acrylamide compounds of the formula I below, by their stereoisomers and by their salts, in particular their agriculturally or veterinarily acceptable salts.

Therefore, in a first aspect, the invention relates to acrylamide compounds of formula I

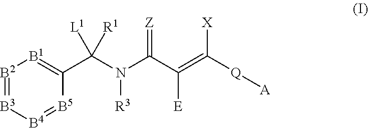

wherein
B¹, B², B³, B⁴ and B⁵ are each independently selected from the group consisting of N and CR², with the proviso that at most two of B¹, B², B³, B⁴ and B⁵ are N;
Z is selected from O and S;
Q is a 5-membered saturated, partially unsaturated or aromatic heteromonocyclic ring containing 1, 2, or 3 heteroatoms or heteroatom groups selected from N, NR^{4a}, O, S, NO, SO and SO₂ as ring members and optionally containing also 1 or 2 groups selected from C═O and C═S as ring members, where the heteromonocyclic ring is optionally substituted with 1, 2 or 3 substituents R⁴;
A is a group A¹, A², A³ or A⁴,
   wherein
   A¹ is selected from the group consisting of —C(═NR⁶)R⁸, —S(O)ₙR⁹ and —N(R⁵)R⁶;
   A² is a group of following formula:

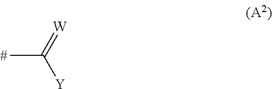

wherein
# denotes the bond to Q;
W is selected from O and S;
Y is selected from hydrogen, —N(R⁵)R⁶ and —OR⁹;
A³ is a group of following formula:

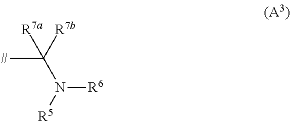

wherein
# denotes the bond to Q;
A⁴ is a 3-, 4-, 5-, 6- or 7-membered saturated, partially unsaturated or maximally unsaturated heteromonocyclic ring containing 1, 2, 3 or 4 heteroatoms or heteroatom groups selected from N, O, S, NO, SO and SO₂ as ring members, or is a 8-, 9- or 10-membered saturated, partially unsaturated or maximally unsaturated heterobicyclic ring containing 1, 2, 3 or 4 heteroatoms or heteroatom groups selected from N, O, S, NO, SO and SO₂ as ring members, where the heteromonocyclic or heterobicyclic ring is optionally substituted with one or more substituents R¹¹;
L¹ is selected from the group consisting of hydrogen, halogen, cyano, hydroxy, amino, C₁-C₆-alkyl, C₃-C₈-cycloalkyl, C₂-C₆-alkenyl, C₂-C₆-alkynyl, C₁-C₆-alkoxy, wherein the aliphatic and cycloaliphatic moieties in the five last-mentioned radicals may be partially or fully halogenated and/or may be substituted by one or more radicals R⁸,
   phenyl which may be substituted by 1, 2, 3, 4 or 5 radicals R¹¹, and a 3-, 4-, 5-, 6- 7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂ as ring members, where the heteromono- or heterobicyclic ring may be substituted by one or more radicals R¹¹;
   or
   L¹ and a radical R² bound in the position of B¹ or B⁵, together with the carbon atoms they are bound to, form a 5-, 6-, or 7-membered saturated, partially unsaturated or maximally unsaturated ring, where the ring may contain 1 or 2 heteroatoms or heteroatom-containing groups selected from O, S, N, SO, SO₂, C═O and C═S as ring members, wherein the ring may be substituted with 1, 2, 3, 4 or 5 substituents independently selected from the group consisting of halogen, C₁-C₆-alkyl and C₁-C₆-haloalkyl;
E is selected from the group consisting of hydrogen, halogen, C₁-C₆-alkyl which may be partially or fully halogenated and/or may be substituted by one or more radicals selected from C₁-C₆-alkoxy, and C₃-C₆-cycloalkyl which may be partially or fully halogenated and/or may be substituted by one or more radicals selected from the group consisting of C₁-C₆-alkyl and C₁-C₆-alkoxy;
   or
   E and a radical R⁴ bound on the heteromonocyclic ring Q, together with the atoms to which they are bound, form a 5-, 6-, or 7-membered saturated, partially unsaturated or maximally unsaturated ring, where the ring may contain 1 or 2 heteroatoms or heteroatom-containing groups selected from O, S, N, SO, SO₂, C═O and C═S as ring members, wherein the ring may be substituted with 1, 2, 3, 4 or 5 substituents independently selected from the group consisting of halogen, C₁-C₆-alkyl, C₁-C₆-haloalkyl and C₁-C₆-alkoxy; or
   or E and a radical R^{4a} bound on the heteromonocyclic ring Q, together with the atoms to which they are bound, form a 5-, 6-, or 7-membered saturated, partially unsaturated or maximally unsaturated heterocyclic ring, where the heterocyclic ring may contain 1 or 2 further heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, C₁-C₆-alkyl, C₁-C₆-haloalkyl and C₁-C₆-alkoxy;
X is selected from the group consisting of hydrogen, halogen, C₁-C₆-alkyl, C₃-C₈-cycloalkyl, C₁-C₆-alkoxy, C₃-C₈-cycloalkyl-C₁-C₆-alkyl- and C₁-C₆-alkoxy-C₁-C₆-alkyl-, wherein the aliphatic and cycloaliphatic moieties in the five last-mentioned radicals may be partially or fully halogenated;
R¹ is selected from the group consisting of C₁-C₄-alkyl, C₂-C₄-alkenyl, C₂-C₄-alkynyl, C₃-C₆-cycloalkyl, C₁-C₄-alkylsulfonyl and C₁-C₄-alkoxycarbonyl, wherein the aliphatic and cycloaliphatic moieties in the six last-mentioned radicals may be partially or fully halogenated and/or may be substituted by one or more radicals selected from the group consisting of hydroxy, cyano, C₁-C₄-alkoxy, C₁-C₄-haloalkoxy, C₁-C₄-alkoxycarbonyl, C₁-C₄-alkylaminocarbonyl and C₁-C₄-dialkylaminocarbonyl;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last-mentioned aliphatic and cycloaliphatic radicals may be partially or fully halogenated and/or may be substituted by one or more radicals R⁸,
   —Si(R¹²)₃, —OR⁹, —S(O)ₙR⁹, —N(R^{10a})R^{10b},
   phenyl which may be substituted by 1, 2, 3, 4 or 5 radicals R¹¹, and a 3-, 4-, 5-, 6- 7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂ as ring members, where the heteromono- or heterobicyclic ring may be substituted by one or more radicals R¹¹;
R³ is selected from the group consisting of hydrogen, C₁-C₆-alkyl, C₃-C₆-cycloalkyl, C₂-C₆-alkenyl, C₂-C₆-alkynyl and C₁-C₆-alkylsulfonyl, wherein the aliphatic and cycloaliphatic moieties in the five last-mentioned radicals may be partially or fully halogenated and/or may be substituted with one or more substituents R⁸;
each R⁴ is independently selected from the group consisting of halogen, cyano, azido, nitro, —SCN, —SF₅, C₁-C₅-alkyl, C₃-C₈-cycloalkyl, C₂-C₆-alkenyl, C₂-C₆-alkynyl, wherein the four last-mentioned aliphatic and cycloaliphatic radicals may be partially or fully halogenated and/or may be substituted by one or more radicals R⁸, —Si(R¹²)₃, —OR⁹, —S(O)ₙR⁹, —N(R^{10a})R^{10b},
   phenyl which may be substituted by 1, 2, 3, 4 or 5 radicals R¹¹, and a 3-, 4-, 5-, 6- 7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂ as ring members, where the heteromonocyclic or heterobicyclic ring may be substituted by one or more radicals R¹¹;
   or
   two radicals R⁴ bound on neighboring carbon atoms form together with the atoms to which they are bound a 5-, 6-, or 7-membered saturated, partially unsaturated or maximally unsaturated ring, where the ring may contain 1 or 2 heteroatoms or heteroatom-containing groups selected from O, S, N, SO, SO₂, C═O and C═S as ring members, wherein the ring may be substituted with 1, 2, 3, 4 or 5 substituents independently selected from the group consisting of halogen, C₁-C₆-alkyl, C₁-C₆-haloalkyl and C₁-C₆-alkoxy;
R^{4a} is selected from the group consisting of hydrogen, C₁-C₆-alkyl, C₃-C₆-cycloalkyl, C₂-C₆-alkenyl and C₂-C₆-alkynyl, wherein the four last-mentioned aliphatic and cycloaliphatic radicals may be partially or fully halogenated and/or may be substituted with one or more substituents R⁸, and —S(O)ₙR⁹;
   or R⁴ and R^{4a}, together with the atoms to which they are bound, form a 5-, 6-, or 7-membered saturated, partially unsaturated or maximally unsaturated heterocyclic ring, where the heterocyclic ring may contain 1 or 2 further heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, C₁-C₆-alkyl, C₁-C₆-haloalkyl and C₁-C₆-alkoxy;
each R⁵ is independently selected from the group consisting of hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more substituents R⁸, and
   —S(O)ₙR⁹,
   or
   R⁵ and a radical R⁴ or R⁵ and a radical R^{4a} bound on the heteromonocyclic ring Q, together with the atoms to which they are bound, form a 5-, 6-, or 7-membered saturated, partially unsaturated or maximally unsaturated heterocyclic ring, where the ring may contain 1 or 2 further heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, C₁-C₆-alkyl, C₁-C₆-haloalkyl and C₁-C₆-alkoxy;
each R⁶ is independently selected from the group consisting of hydrogen, cyano, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted by one or more substituents R⁸, —OR⁹, —N(R^{10a})R^{10b}, —S(O)ₙR⁹, —C(═O)N(R^{10a})N(R^{10a})R^{10b}, —Si(R¹²)₃, —C(═O)R⁸, —P(═O)(OR⁹)₂, —P(═S)(OR⁹)₂,
   phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂ as ring members, where the heteromonocyclic or heterobicyclic ring may be substituted with one or more substituents R¹¹;
   or R⁵ and R⁶, together with the nitrogen atom to which they are bound, form a 3-, 4-, 5-, 6-, 7- or 8-membered saturated, partially unsaturated or maximally unsaturated heteromonocyclic ring, where the ring may further contain 1, 2, 3 or 4 heteroatoms or heteroatom-containing groups selected from O, S, N, SO, SO₂, C═O and C═S as ring members, or form a 7-, 8-, 9- or 10-membered saturated, partially unsaturated or maximally unsaturated heterobicyclic ring, where the ring may further contain 1, 2, 3 or 4 heteroatoms or heteroatom groups selected from N, O, S, NO, SO, SO₂, C═O and C═S, wherein the heteromonocyclic or heterobicyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
   or R⁵ and R⁶ together form a group ═C(R⁸)₂, ═S(O)ₘ(R⁹)₂, ═NR^{10a} or ═NOR⁹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, where the cycloaliphatic moieties in the two last-mentioned radicals may be substituted by one or more radicals R¹³;
   —Si(R¹²)₃, —OR⁹, —OSO₂R⁹, —S(O)ₙR⁹, —N(R^{10a})R^{10b}, —C(═O)R¹³, —C(═O)N(R^{10a})R^{10b}, —C(═S)N(R^{10a})R^{10b}, —C(═O)OR⁹,
   phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁶,
   or
   two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
   or
   two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, where the heterocyclic ring comprises 1, 2, 3 or 4 heteroatoms or heteroatom groups independently selected from N, O, S, NO, SO and SO₂ as ring members, and where the carbocyclic or heterocyclic ring is optionally substituted with one or more substituents R¹⁶; and
   R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
   R⁸ in the groups —C(═NR⁶)R⁸, —C(═O)R⁸ and ═C(R⁸)₂ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³,
   —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵,
   phenyl, optionally substituted with one or more substituents R¹⁶; and
   a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} are selected independently from one another from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the eight last-mentioned radicals may be substituted by one or more radicals R¹³;
   —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, C₁-C₆-alkoxy, C₁-C₆-haloalkoxy, C₁-C₆-alkylthio, C₁-C₆-haloalkylthio,
   S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³;
   phenyl, optionally substituted with 1, 2, 3 or 4, substituents R¹⁶; and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R¹⁶;
   or
   R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂ as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6,- or 7-membered saturated, partially unsaturated or maximally unsaturated heterocyclic ring comprising 1, 2 or 3 heteroatoms or heteroatom groups selected from N, O, S, NO, SO and SO₂ as ring members, where the heterocyclic ring optionally carries one or more substituents R¹⁶;
   or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
each R¹¹ is independently selected from the group consisting of halogen, cyano, azido, nitro, —SCN, —SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸,
   —OR⁹, —N(R^{10a})R^{10b}, —S(O)ₙR⁹, —Si(R¹²)₃;
   phenyl, optionally substituted with 1, 2, 3, 4, or 5 substituents selected independently from R¹⁶; and
   a 3-, 4-, 5-, 6- or 7-membered saturated, partially unsaturated or maximally unsaturated aromatic heterocyclic ring comprising 1, 2, 3 or 4 heteroatoms or heteroatom groups selected from N, O, S, NO, SO and SO₂ as ring members, where the heterocyclic ring is optionally substituted with one or more substituents selected independently from R¹⁶;
   or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
   or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂ as ring members, where the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl-, C₁-C₆-haloalkoxy-C₁-C₆-alkyl-, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₆-haloalkylsulfonyl, trimethylsilyl, triethylsilyl, tert-butyldimethylsilyl, C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy and oxo; phenyl, benzyl, phenoxy, where the phenyl moiety in the last three radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂ as ring members, where the heterocyclic ring may be substituted by 1, 2 or 3 substituents R¹⁶;
   or
   two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl);
   and
   R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from CN, C₃-C₄-cycloalkyl, C₁-C₄-alkoxy, C₁-C₄-haloalkoxy and oxo;
   and
   R¹³ in the groups ═C(R¹³)₂, —N═C(R¹³)₂, —C(═O)R¹³, —C(═S)R¹³ and
   —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl,
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from CN, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, where the cycloalkyl moiety in the two last-mentioned radicals may be substituted by 1 or 2 substituents selected from halogen and cyano; and oxo;
   phenyl, benzyl, pyridyl, phenoxy, wherein the cyclic moieties in the four last-mentioned radicals may be unsubstituted and/or carry 1, 2 or 3 substituents selected from halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from N, O, S, NO, SO and SO₂ as ring members, where the heterocyclic ring is optionally substituted with one or more substituents R¹⁶;
R^{14a} and R^{14b}, independently of each other, have one of the meanings given for R¹⁴;
   or
   R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from N, O, S, NO, SO and SO₂ as ring members, where the heterocyclic ring optionally carries one or more substituents selected from halogen, C₁-C₄-alkyl, C₁-C₄-haloalkyl, C₁-C₄-alkoxy and C₁-C₄-haloalkoxy;
   or
   R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from N, O, S, NO, SO and SO₂ as ring members, where the heterocyclic ring optionally carries one or more substituents selected from halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl,
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo;
   phenyl, benzyl, pyridyl and phenoxy, wherein the four last-mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl;
   C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy and oxo;
   C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy and oxo;
   phenyl, benzyl, pyridyl and phenoxy, wherein the four last-mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
   or
   two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
   or
   two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, S, NO, SO and SO₂ as ring members, and wherein the ring optionally carries one or more substituents selected from halogen, C₁-C₄-haloalkyl, C₁-C₄-alkoxy and C₁-C₄-haloalkoxy;
each n is independently 0, 1 or 2; and
each m is independently 0 or 1;
   and the N-oxides, stereoisomers and agriculturally or veterinarily acceptable salts thereof.

The present invention also provides an agricultural composition comprising at least one compound of the formula I as defined herein and/or an agriculturally acceptable salt thereof and at least one liquid or solid carrier.

The present invention also provides a veterinary composition comprising at least one compound of the formula I as defined herein and/or a veterinarily acceptable salt thereof and at least one liquid or solid carrier.

The present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein. This method does not encompass the therapeutic treatment of human beings or animals.

The present invention also relates to plant propagation material, in particular seed, comprising at least one compound of formula I and/or an agriculturally acceptable salt thereof as defined herein.

The present invention further relates to a method for treating or protecting an animal from infestation or infection by parasites which comprises bringing the animal in contact with a parasiticidally effective amount of a compound of the formula I or a veterinarily acceptable salt thereof as defined herein. Bringing the animal in contact with the compound I, its salt or the veterinary composition of the invention means applying or administering it to the animal. The invention also relates to the use of a compound of the formula I or a veterinarily acceptable salt thereof as defined herein for preparing a medicament for treating or protecting an animal from infestation or infection by parasites, and to a compound of the formula I or a veterinarily acceptable salt thereof as defined herein for preparing a medicament for treating or protecting an animal from infestation or infection by parasites.

The term “steroisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. One center of chirality is the carbon atom carrying radicals R¹ and L¹. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO).

The compounds of the present invention may be amorphous or may exist in one ore more different crystalline states (polymorphs) which may have a different macroscopic properties such as stability or show different biological properties such as activities. The present invention includes both amorphous and crystalline compounds of the formula I, mixtures of different crystalline states of the respective compound I, as well as amorphous or crystalline salts thereof.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. The plant propagation materials may be treated prophylactically with a plant protection compound either at or before planting or transplanting. Said young plants may also be protected before transplantation by a total or partial treatment by immersion or pouring.

The term “plants” comprises any types of plants including “non-cultivated plants” and in particular “cultivated plants”.

The term “non-cultivated plants” refers to any wild type species or related species or related genera of a cultivated plant.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxyl-phenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the CryIAb toxin), YieldGard® Plus (corn cultivars producing CryIAb and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the CryIAc toxin), Bollgard® I (cotton cultivars producing the CryIAc toxin), Bollgard® II (cotton cultivars producing CryIAc and Cry2Ab2 toxins); VIPCOT® (cotton cultivars producing a VIP-toxin); NewLeaf® (potato cultivars producing the Cry3A toxin); BtXtra®, NatureGard®, KnockOut®, BiteGard®, Protecta®, Bt11 (e. g. Agrisure® CB) and Bt176 from Syngenta Seeds SAS, France, (corn cultivars producing the CryIAb toxin and PAT enyzme), MIR⁶⁰⁴ from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the CryIAc toxin) and 1507 from Pioneer Overseas Corporation, Belgium (corn cultivars producing the CryIF toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₃-Alkyl is additionally propyl and isopropyl. C₁-C₄-Alkyl is additionally butyl, 1-methylpropyl(sec-butyl), 2-methylpropyl(isobutyl) or 1,1-dimethylethyl(tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like. Examples for C₁-C₆-haloalkyl are, apart those mentioned for C₁-C₄-haloalkyl, 5-chloropentyl, 6-chlorohexyl and the like.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”), 2 to 6 (“C₂-C₆-alkenyl”), 2 to 8 (“C₂-C₈-alkenyl”) or 2 to 10 (“C₂-C₁₀-alkenyl”) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “haloalkenyl” as used herein, which is also expressed as “alkenyl which is partially or fully halogenated”, refers to unsaturated straight-chain or branched hydrocarbon radicals having 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “alkynyl” as used herein refers to straight-chain or branched hydrocarbon groups having 2 to 3 (“C₂-C₃-alkynyl”), 2 to 4 (“C₂-C₄-alkynyl”), 2 to 6 (“C₂-C₆-alkynyl”), 2 to 8 (“C₂-C₈-alkynyl”), or 2 to 10 (“C₂-C₁₀-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “haloalkynyl” as used herein, which is also expressed as “alkynyl which is partially or fully halogenated”, refers to unsaturated straight-chain or branched hydrocarbon radicals having 2 to 4 (“C₂-C₄-haloalkynyl”), 3 to 4 (“C₃-C₄-haloalkynyl”), 2 to 6 (“C₂-C₆-haloalkynyl”), 2 to 8 (“C₂-C₈-haloalkynyl”) or 2 to 10 (“C₂-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine;

The term “cycloalkyl” as used herein refers to mono- or bi- or polycyclic saturated hydrocarbon radicals having 3 to 8 (“C₃-C₈-cycloalkyl”), in particular 3 to 6 carbon atoms (“C₃-C₆-cycloalkyl”) or 3 or 4 carbon atoms (“C₃-C₄-cycloalkyl”). Preferably, C₃-C₄-cycloalkyl and C₃-C₄-cycloalkyl are monocyclic. Examples for C₃-C₄-cycloalkyl are cyclopropyl and cyclobutyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “halocycloalkyl” as used herein, which is also expressed as “cycloalkyl which is partially or fully halogenated”, refers to mono- or bi- or polycyclic saturated hydrocarbon groups having 3 to 8 (“C₃-C₈-halocycloalkyl”) or preferably 3 to 6 (“C₃-C₆-halocycloalkyl”) carbon ring members (as mentioned above) in which some or all of the hydrogen atoms are replaced by halogen atoms as mentioned above, in particular fluorine, chlorine and bromine.

The term “cycloalkyl-C₁-C₄-alkyl” refers to a C₃-C₈-cycloalkyl group (“C₃-C₈-cycloalkyl-C₁-C₄-alkyl”), preferably a C₃-C₆-cycloalkyl group (“C₃-C₆-cycloalkyl-C₁-C₄-alkyl”), more preferably a C₃-C₄-cycloalkyl group (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl. Examples for C₃-C₈-cycloalkyl-C₁-C₄-alkyl, apart those mentioned for C₃-C₆-cycloalkyl-C₁-C₄-alkyl, are cycloheptylmethyl, cycloheptylethyl, cyclooctylmethyl and the like. The term “C₃-C₈-cycloalkyl-C₁-C₆-alkyl” refers to a C₃-C₈-cycloalkyl group as defined above (preferably a monocyclic cycloalkyl group) which is bound to the remainder of the molecule via a C₁-C₆-alkyl group, as defined above. Examples for C₃-C₈-cycloalkyl-C₁-C₆-alkyl, apart those mentioned for C₃-C₈-cycloalkyl-C₁-C₄-alkyl, are cyclopropylpentyl, cyclopropylhexyl, cyclobutylpentyl, cyclobutylhexyl, cyclopentylpenty, cyclopentylhexyl and the like.

The term “C₃-C₈-halocycloalkyl-C₁-C₄-alkyl” refers to a C₃-C₈-halocycloalkyl group as defined above which is bound to the remainder of the molecule via a C₁-C₄-alkyl group, as defined above.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (secbutoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Haloalkoxy is, for example, OCH₂F, OCHF₂, OCF₃, OCH₂Cl, OCHCl2, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₄-alkoxy-C₁-C₄-alkyl” as used herein, refers to a straight-chain or branched alkyl group having 1 to 4 carbon atoms, as defined above, where one hydrogen atom is replaced by a C₁-C₄-alkoxy group, as defined above. The term “C₁-C₆-alkoxy-C₁-C₆-alkyl” as used herein, refers to a straight-chain or branched alkyl group having 1 to 6 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “C₁-C₆-alkoxy-methyl” as used herein, refers to methyl in which one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, pentyloxymethyl, hexyloxymethyl and the like.

C₁-C₆-Haloalkoxy-C₁-C₆-alkyl is a straight-chain or branched alkyl group having from 1 to 6, especially 1 to 4 carbon atoms (═C₁-C₆-haloalkoxy-C₁-C₄-alkyl), wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by halogen atoms. C₁-C₄-Haloalkoxy-C₁-C₄-alkyl is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by halogen atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoro-methoxy-methyl (CH₃OCF₂), 1,1-difluoro-2-methoxyethyl, 2,2-difluoro-2-methoxyethyl and the like.

The term “C₁-C₂-alkylthio” is a C₁-C₂-alkyl group, as defined above, attached via a sulfur atom. The term “C₁-C₃-alkylthio” is a C₁-C₃-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₃-Alkylthio is additionally, for example, n-propylthio or 1-methylethylthio(isopropylthio). C₁-C₄-Alkylthio is additionally, for example, butylthio, 1-methylpropylthio(sec-butylthio), 2-methylpropylthio(isobutylthio) or 1,1-dimethylethylthio(tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₃-haloalkylthio” is a C₁-C₃-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₃-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio or 1-(CH₂Br)-2-bromoethylthio. C₁-C₄-Haloalkylthio is additionally, for example, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl(isopropylsulfinyl), butylsulfinyl, 1-methylpropylsulfinyl(sec-butylsulfinyl), 2-methylpropylsulfinyl(isobutylsulfinyl) or 1,1-dimethylethylsulfinyl(tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₃-alkylsulfonyl” is a C₁-C₃-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₃-Alkylsulfonyl is additionally, for example, n-propylsulfonyl or 1-methylethylsulfonyl(isopropylsulfonyl). C₁-C₄-Alkylsulfonyl is additionally, for example, butylsulfonyl, 1-methylpropylsulfonyl(sec-butylsulfonyl), 2-methylpropylsulfonyl(isobutylsulfonyl) or 1,1-dimethylethylsulfonyl(tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₃-haloalkylsulfonyl” is a C₁-C₃-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₃-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonylor 1-(CH₂Br)-2-bromoethylsulfonyl. C₁-C₄-Haloalkylsulfonyl is additionally, for example, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The substituent “oxo” replaces a CH₂ group by a C(═O) group.

The term “alkylcarbonyl” is a C₁-C₆-alkyl (“C₁-C₆-alkylcarbonyl”), preferably a C₁-C₄-alkyl (“C₁-C₄-alkylcarbonyl”) group, as defined above, attached via a carbonyl [C(═O)] group. Examples are acetyl(methylcarbonyl), propionyl(ethylcarbonyl), propylcarbonyl, isopropylcarbonyl, n-butylcarbonyl and the like.

The term “haloalkylcarbonyl” is a C₁-C₆-haloalkyl (“C₁-C₄-haloalkylcarbonyl”), preferably a C₁-C₄-haloalkyl (“C₁-C₄-haloalkylcarbonyl”) group, as defined above, attached via a carbonyl [C(═O)] group. Examples are trifluoromethylcarbonyl, 2,2,2-trifluoroethylcarbonyl and the like.

The term “alkoxycarbonyl” is a C₁-C₆-alkoxy (“C₁-C₆-alkoxycarbonyl”), preferably a C₁-C₄-alkoxy (“C₁-C₄-alkoxycarbonyl”) group, as defined above, attached via a carbonyl [C(═O)] group. Examples are methoxycarbonyl), ethoxycarbonyl, propoxycarbonyl, isopropoxycarbonyl, n-butoxycarbonyl and the like.

The term “haloalkoxycarbonyl” is a C₁-C₆-haloalkoxy (“C₁-C₆-haloalkoxycarbonyl”), preferably a C₁-C₄-haloalkoxy (“C₁-C₄-haloalkoxycarbonyl”) group, as defined above, attached via a carbonyl [C(═O)] group. Examples are trifluoromethoxycarbonyl, 2,2,2-trifluoroethoxycarbonyl and the like.

The term “C₁-C₆-alkylamino” is a group —N(H)C₁-C₆-alkyl. Examples are methylamino, ethylamino, propylamino, isopropylamino, butylamino and the like.

The term “di-(C₁-C₆-alkyl)amino” is a group —N(C₁-C₆-alkyl)₂. Examples are dimethylamino, diethylamino, ethylmethylamino, dipropylamino, diisopropylamino, methylpropylamino, methylisopropylamino, ethylpropylamino, ethylisopropylamino, dibutylamino and the like.

The term “3-, 4-, 5-, 6-, 7-, 8-, 9- or 10-membered saturated, partially unsaturated or maximally unsaturated heterocyclic ring (or heteromonocyclic or heterobicyclic ring) containing 1, 2 or 3 (or 4) heteroatoms or heteroatom groups selected from N, O, S, NO, SO and SO₂, as ring members” denotes a 3-, 4-, 5-, 6-, 7-, 8-, 9- or 10-membered, preferably a 3-, 4-, 5-, 6- or 7-membered saturated, partially unsaturated or maximum unsaturated heteromonocyclic ring or a 7-, 8-, 9- or 10-membered saturated, partially unsaturated or maximally unsaturated heterobicyclic ring containing 1, 2 or 3 (or 4) heteroatoms or heteroatom groups selected from N, O, S, NO, SO and SO₂, as ring members.

Unsaturated rings contain at least one C—C and/or C—N and/or N—N double bond(s). Maximally unsaturated rings contain as many conjugated C—C and/or C—N and/or N—N double bonds as allowed by the ring size. Maximally unsaturated 5- or 6-membered heterocyclic rings are aromatic. Partially unsaturated rings contain less than the maximum number of C—C and/or C—N and/or N—N double bond(s) allowed by the ring size. The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

The term “3-, 4-, 5-, 6- or 7-membered saturated, partially unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein denotes monocyclic radicals, the monocyclic radicals being saturated, partially unsaturated or maximum unsaturated (including aromatic). The term “3-, 4-, 5-, 6-, 7- or 8-membered saturated, partially unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein further also encompasses 8-membered heteromonocyclic radicals containing 1, 2 or 3 (or 4) heteroatoms or heteroatom groups selected from N, O, S, NO, SO and SO₂, as ring members, the monocyclic radicals being saturated, partially unsaturated or maximum unsaturated (including aromatic). Unsaturated rings contain at least one C—C and/or C—N and/or N—N double bond(s). Maximum unsaturated rings contain as many conjugated C—C and/or C—N and/or N—N double bonds as allowed by the ring size. Maximum unsaturated 5- or 6-membered heterocyclic rings are aromatic. 7- and 8-membered rings cannot be aromatic. They are homoaromatic (7-membered ring, 3 double bonds) or have 4 double bonds (8-membered ring). The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

Examples of a 3-, 4-, 5-, 6- or 7-membered saturated heterocyclic ring include: Oxiranyl, thiiranyl, aziridinyl, oxetanyl, thietanyl, azetidinyl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, 4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

Examples for a 3-, 4-, 5-, 6- or 7-membered maximally unsaturated (including aromatic) heterocyclic ring are 5- or 6-membered heteroaromatic rings, such as 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl, and also homoaromatic radicals, such as 1H-azepine, 1H-[1,3]-diazepine and 1H-[1,4]-diazepine.

In the present invention, the “heterobicyclic rings” contain two rings which have at least one ring atom in common. At least one of the two rings contains a heteroatom or heteroatom group selected from N, O, S, NO, SO and SO₂ as ring member. The term comprises condensed (fused) ring systems, in which the two rings have two neighboring ring atoms in common, as well as spiro systems, in which the rings have only one ring atom in common, and bridged systems with at least three ring atoms in common.

Examples for fused systems:

Examples for a 7-, 8-, 9- or 10-membered saturated heterobicyclic ring containing 1, 2 or 3 (or 4) heteroatoms or heteroatom groups selected from N, O, S, NO, SO and SO₂, as ring members are:

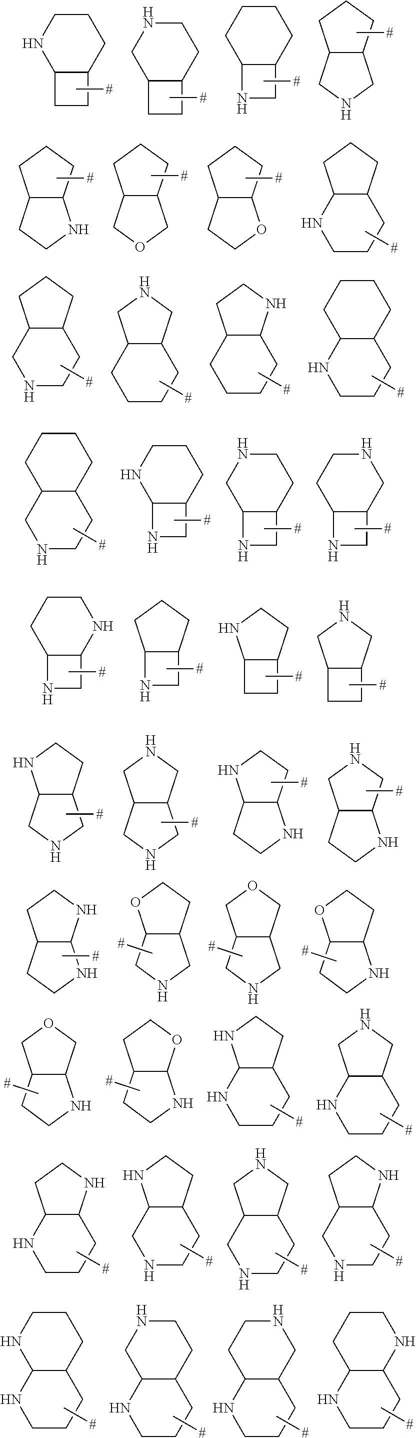

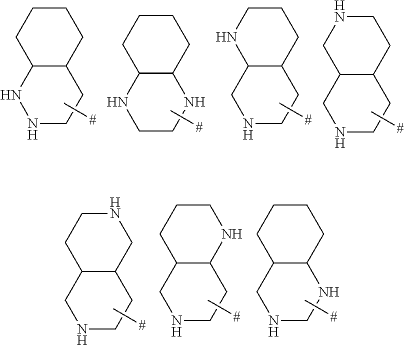

Examples for a 8-, 9- or 10-membered partially unsaturated heterobicyclic ring containing 1, 2 or 3 (or 4) heteroatoms or heteroatom groups selected from N, O, S, NO, SO and SO₂, as ring members are:

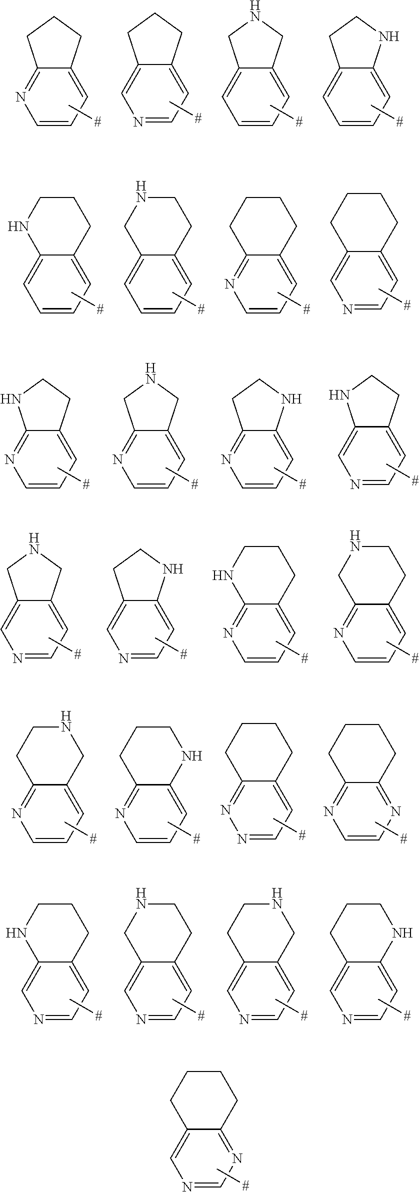

Examples for a 8-, 9- or 10-membered maximally unsaturated heterobicyclic ring containing 1, 2 or 3 (or 4) heteroatoms or heteroatom groups selected from N, O, S, NO, SO and SO₂, as ring members are:

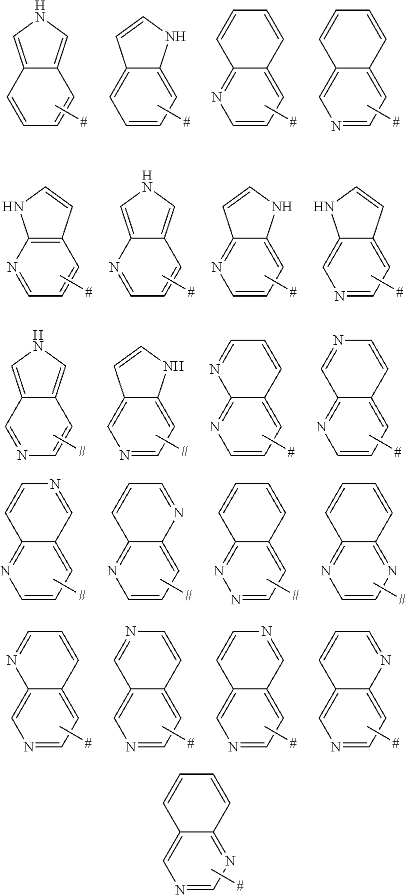

Examples for spiro-bound 7-, 8-, 9- or 10-membered heterobicyclic rings containing 1, 2 or 3 (or 4) heteroatoms or heteroatom groups selected from N, O, S, NO, SO and SO₂, as ring members are

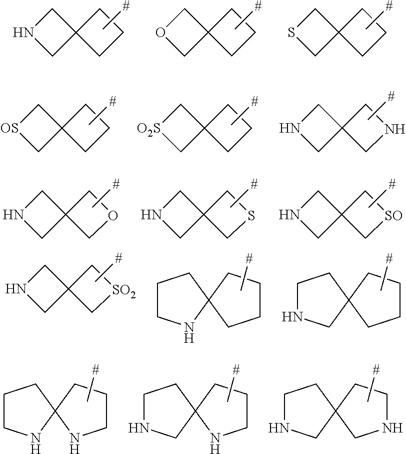

Examples for bridged 7-, 8-, 9- or 10-membered heterobicyclic rings containing 1, 2 or 3 (or 4) heteroatoms or heteroatom groups selected from N, O, S, NO, SO and SO₂, as ring members are

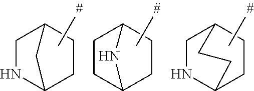

and the like.

In the above structures # denotes the attachment point to the remainder of the molecule. The attachment point is not restricted to the ring on which is shown, but can be on either of the fused rings, and may be on a carbon or on a nitrogen ring atom. If the rings carry one or more substituents, these may be bound to carbon and/or to nitrogen ring atoms (if the latter are not part of a double bond).

A saturated 3-, 4-, 5-, 6-, 7-, 8- or 9-membered ring, wherein the ring may contain 1 or 2 heteroatoms or heteroatom groups selected from O, S, N, NR¹⁴, NO, SO and SO₂ and/or 1 or 2 groups selected from C═O, C═S and C═NR¹⁴ as ring members is either carbocyclic or heterocyclic. Examples are, in addition to the saturated heteromonocyclic rings mentioned above, carbocyclic rings, such as cyclopropyl, cyclopropanonyl, cyclobutyl, cyclobutanonyl, cyclopentyl, cyclopentanonyl, cyclohexyl, cyclohexanonyl, cyclohexadienonyl, cycloheptyl, cycloheptanonyl, cyclooctyl, cyclooctanonyl, furan-2-onyl, pyrrolidine-2-onyl, pyrrolidine-2,5-dionyl, piperidine-2-only, piperidine-2,6-dionyl and the like.

The 5-membered saturated, partially unsaturated or aromatic heteromonocyclic ring Q containing 1, 2, or 3 heteroatoms or heteroatom groups selected from N, NR^{4a}, O, S, NO, SO and SO₂ as ring members and optionally containing also 1 or 2 groups selected from C═O and C═S as ring members is for example tetrahydrofuran-2-yl, tetrahydrofuran-3-yl, 2-oxo-tetrahydrofuran-3-yl, tetrahydrothien-2-yl, tetrahydrothien-3-yl, 1-oxo-tetrahydrothien-2-yl, 1-oxo-tetrahydrothien-3-yl, 1,1-dioxo-tetrahydrothien-2-yl, 1,1-dioxo-tetrahydrothien-3-yl, 2-oxo-tetrahydrothien-3-yl, pyrrolidin-1-yl, pyrrolidin-2-yl, pyrrolidin-3-yl, 2-oxo-pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, 2-oxoimidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, 1-oxo-thiazolidin-2-yl, 1-oxo-thiazolidin-3-yl, 1-oxo-thiazolidin-4-yl, 1-oxo-thiazolidin-5-yl, 1,1-dioxo-thiazolidin-2-yl, 1,1-dioxo-thiazolidin-3-yl, 1,1-dioxo-thiazolidin-4-yl, 1,1-dioxo-thiazolidin-5-yl, isothiazolidin-2-yl, isothiazolidin-3-yl, isothiazolidin-4-yl, isothiazolidin-5-yl, 1-oxo-isothiazolidin-2-yl, 1-oxo-isothiazolidin-3-yl, 1-oxo-isothiazolidin-4-yl, 1-oxo-isothiazolidin-5-yl, 1,1-dioxo-isothiazolidin-2-yl, 1,1-dioxo-isothiazolidin-3-yl, 1,1-dioxo-isothiazolidin-4-yl, 1,1-dioxo-isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-furyl, 3-furyl, 2-thienyl, 3-thienyl, 1-oxo-2-thienyl, 1-oxo-3-thienyl, 1,1-dioxo-2-thienyl, 1,1-dioxo-3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-oxo-2-thiazolyl, 1-oxo-4-thiazolyl, 1-oxo-5-thiazolyl, 1,1-dioxo-2-thiazolyl, 1,1-dioxo-4-thiazolyl, 1,1-dioxo-5-thiazolyl, 3-isothiazolyl, 4-isothiazolyl, 5-isothiazolyl, 1-oxo-3-isothiazolyl, 1-oxo-4-isothiazolyl, 1-oxo-5-isothiazolyl, 1,1-dioxo-3-isothiazolyl, 1,1-dioxo-4-isothiazolyl, 1,1-dioxo-5-isothiazolyl, 1-imidazolyl, 2-imidazolyl, 4-imidazolyl, 1,3,4-triazol-1-yl, 1,3,4-triazol-2-yl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 1-oxo-1,2,4-thiadiazol-3-yl, 1-oxo-1,2,4-thiadiazol-5-yl, 1-oxo-1,3,4-thiadiazol-2-yl, 1,1-dioxo-1,2,4-thiadiazol-3-yl, 1,1-dioxo-1,2,4-thiadiazol-5-yl, 1,1-dioxo-1,3,4-thiadiazol-2-yl, and the like.

The remarks made below concerning preferred embodiments of the variables of the compounds of formula I, especially with respect to their substituents A, A¹, A², A³, A⁴, B¹, B², B³, B⁴, B⁵, L¹, Z, E, X, Q, R¹, R², R³, R⁴, R^{4a}, R^{4b}, R^{4c}, R⁵, R⁶, R^{7a}, R^{7b}, R⁸, R⁹, R^{10a}, R^{10b}, R¹¹, R¹², R¹³, R¹⁴, R^{14a}, R^{14b}, R¹⁵, R¹⁶, m and n, the features of the use and method according to the invention and of the composition of the invention are valid both on their own and, in particular, in every possible combination with each other.

Preferably, Q is a 5-membered heteroaromatic ring containing 1, 2, or 3 heteroatoms or heteroatom groups selected from N, NR^{4a}, O, S, NO, SO and SO₂ as ring members, where the heteroaromatic ring is optionally substituted with 1 or 2 substituents R⁴.

In a preferred embodiment, R⁴ is selected from halogen, cyano, C₁-C₄-alkyl, C₁-C₄-haloalkyl, C₃-C₆-cycloalkyl, C₃-C₆-halocycloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₆-alkoxy-C₁-C₆-alkyl-, C₁-C₄-alkylthio and C₁-C₄-haloalkylthio. More preferably, R⁴ is selected from halogen, C₁-C₄-alkyl, C₁-C₄-haloalkyl, C₁-C₄-alkoxy, C₁-C₄-haloalkoxy, C₁-C₄-alkylthio and C₁-C₄-haloalkylthio, and in particular from C₁-C₄-alkyl. Specifically, R⁴ is methyl.

In a preferred embodiment, the moiety -Q-A is selected from the moieties Q-1 to Q-36

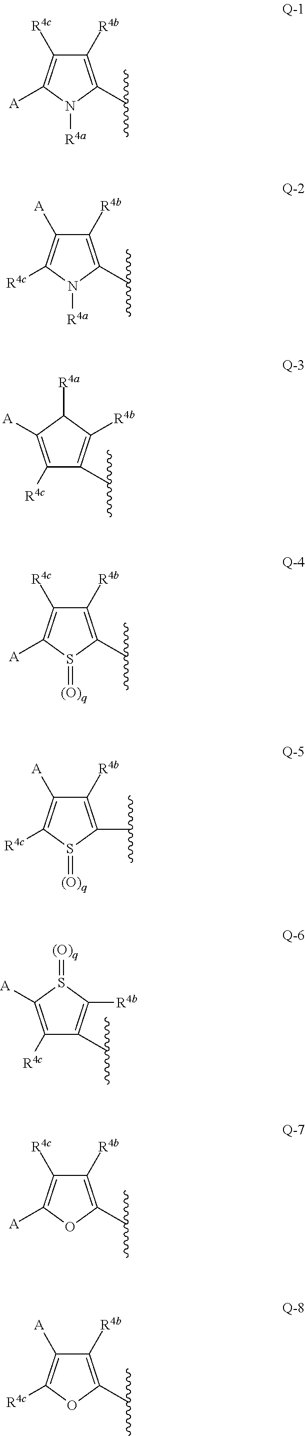

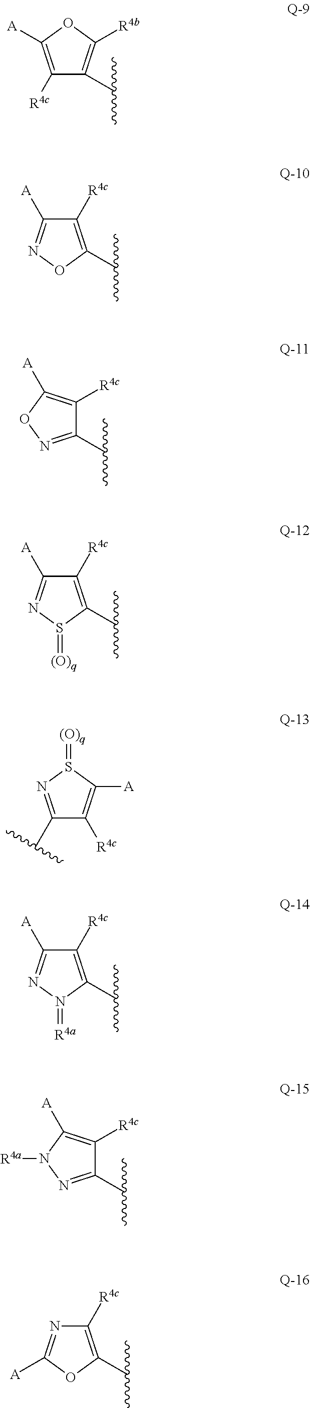

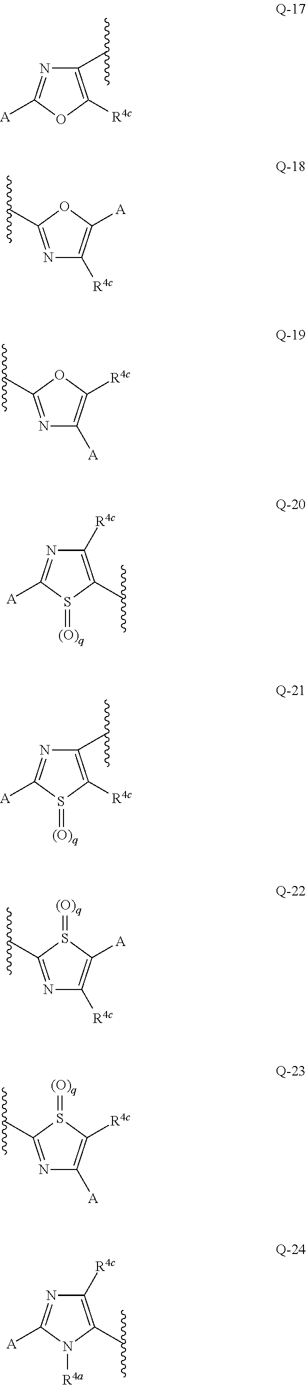

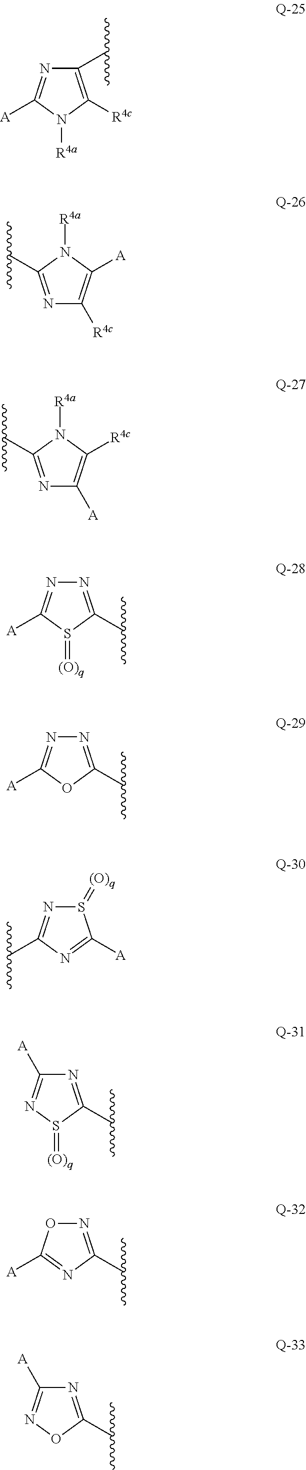

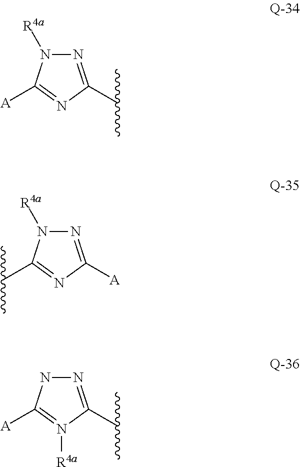

wherein
[symbol] denotes the bonding point to the remainder of the molecule,
A is as defined in claim 1;
R^{4a} is as defined in claim 1;
R^{4b} and R^{4c}, independently of each other, have one of the meanings given in claim 1 for R⁴ or are hydrogen; and
q is 0, 1 or 2.

In a preferred embodiment, R^{4b} and R^{4c}, independently of each other, are selected from hydrogen, halogen, cyano, C₁-C₄-haloalkyl, C₃-C₅-cycloalkyl, C₃-C₅-halocycloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₆-alkoxy-C₁-C₆-alkyl-, C₁-C₄-alkylthio and C₁-C₄-haloalkylthio. More preferably, R^{4b} and R^{4c}, independently of each other, are selected from hydrogen, halogen, C₁-C₄-alkyl, C₁-C₄-haloalkyl, C₁-C₄-alkoxy, C₁-C₄-haloalkoxy, C₁-C₄-alkylthio and C₁-C₄-haloalkylthio.

Even more preferably, R^{4b} is hydrogen and R^{4c} is selected from hydrogen, halogen, cyano, C₁-C₄-haloalkyl, C₃-C₅-cycloalkyl, C₃-C₅-halocycloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, more preferably from hydrogen, halogen, C₁-C₄-alkyl, C₁-C₄-haloalkyl, C₁-C₄-alkoxy, C₁-C₄-haloalkoxy, C₁-C₄-alkylthio and C₁-C₄-haloalkylthio, and in particular from C₁-C₄-alkyl. Specifically, R^{4b} is hydrogen and R^{4c} is hydrogen or, in particular, methyl.

Preferably, the moiety -Q-A is selected from Q-4 and Q-5. Preferably, q is 0; i.e. Q is preferably -thien-2-yl. Particularly preferred is group Q-4, wherein q is 0.

In groups Q-4 and Q-5, R^{4b} is preferably hydrogen and R^{4c} is preferably selected from the group consisting of hydrogen, halogen, C₁-C₆-alkyl, C₁-C₆-haloalkyl, C₁-C₆-alkoxy, C₁-C₆-haloalkoxy, C₁-C₆-alkylthio and C₁-C₆-haloalkylthio. Specifically, R^{4b} is hydrogen and R^{4c} is hydrogen or, preferably, methyl.

In one preferred embodiment, A is A¹ and A¹ is selected from —C(═NR⁶)R⁸ and —N(R⁵)R⁶ and is more preferably —C(═NR⁶)R⁸; wherein R⁵, R⁶ and R⁸ have one of the above general meanings, or, in particular, one of the below preferred meanings.

More preferably, A¹ is —C(═NR⁶)R⁸; wherein R⁵, R⁶ and R⁸ have one of the above general meanings, or, in particular, one of the below preferred meanings

R⁶ as a radical in the group —C(═NR⁶)R⁸ is preferably selected from hydrogen, cyano, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals each independently may be partially or fully halogenated and/or may be substituted with 1, 2, 3, 4, 5 or 6 substituents R⁸; —OR⁹ and —N(R^{10a})R^{10b}; wherein R⁸, R⁹, R^{10a} and R^{10b} have one of the above general meanings, or, in particular, one of the below preferred meanings.

More preferably, R⁶ in —C(═NR⁶)R⁸ is selected from hydrogen, C₁-C₁₀-alkyl, C₃-C₈-cycloalkyl, wherein the two last-mentioned aliphatic and cycloaliphatic radicals each independently may be partially or fully halogenated and/or may be substituted with 1, 2 or 3, preferably 1 or 2, in particular 1, substituents R⁸; OR⁹ and NR^{10a}R^{10b}; wherein R⁸, R⁹, R^{10a} and R^{10b} have one of the above general meanings, or, in particular, one of the below preferred meanings.

Even more preferably, R⁶ in —C(═NR⁶)R⁸ is selected from OR⁹ and NR^{10a}R^{10b}; wherein R⁸, R⁹, R^{10a} and R^{10b} have one of the above general meanings, or, in particular, one of the below preferred meanings. Specifically, R⁶ in —C(═NR⁶)R⁸ is NR^{10a}R^{10b}.

In OR⁹ as a preferred meaning of R⁶ in —C(═NR⁶)R⁸, R⁹ is preferably selected from C₁-C₆-alkyl, C₁-C₆-haloalkyl, C₃-C₈-cycloalkyl, C₃-C₈-halocycloalkyl, C₃-C₈-cycloalkyl-C₁-C₄-alkyl-, C₂-C₆-alkenyl, C₂-C₆-haloalkenyl, C₂-C₆-alkynyl and C₂-C₆-haloalkynyl, and more preferably from C₁-C₆-alkyl, C₁-C₆-haloalkyl, C₃-C₈-cycloalkyl, C₃-C₈-halocycloalkyl and C₃-C₈-cycloalkyl-C₁-C₄-alkyl-.

In NR^{10a}R^{10b} as a preferred meaning of R⁶ in —C(═NR⁶)R⁸, R^{10a} and R^{10b}, independently of each other, are preferably selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkylcarbonyl, C₁-C₆-haloalkylcarbonyl, —C(═O)N(R^{14a})R^{14b}, —C(═S)N(R^{14a})R^{14b}, phenyl which is optionally substituted with 1, 2, 3 or 4, substituents R¹⁶, and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂, as ring members, where the heterocyclic ring is optionally substituted with one or more substituents R¹⁶;

or R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂, as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl;
wherein R^{14a}, R^{14b} and R¹⁶ have one of the above general meanings, or, in particular, one of the below preferred meanings.

More preferably,
R^{10a} is selected from hydrogen, C₁-C₆-alkyl and C₁-C₆-haloalkyl; and
R^{10b} is selected from —C(═O)N(R^{14a})R^{14b}, —C(═S)N(R^{14a})R^{14b}, phenyl which is optionally substituted with 1, 2, 3 or 4, substituents R¹⁶, and a 5- or 6-membered heteroaromatic ring comprising 1, 2 or 3 heteroatoms selected from N, O and S, as ring members, where the heteroaromatic ring is optionally substituted with one or more substituents R¹⁶;
   wherein R^{14a}, R^{14b} and R¹⁶ have one of the above general meanings, or, in particular, one of the below preferred meanings.

Even more preferably,
R^{10a} is selected from hydrogen, C₁-C₆-alkyl and C₁-C₆-haloalkyl and preferably from hydrogen and C₁-C₆-alkyl; and
R^{10b} is selected from —C(═O)N(R^{14a})R^{14b} and —C(═S)N(R^{14a})R^{14b};
   wherein R^{14a} and R^{14b} have one of the above general meanings, or, in particular, one of the below preferred meanings.

In the above radicals R^{10a} and R^{10b},
R^{14a} is preferably selected from hydrogen, C₁-C₆-alkyl and C₁-C₆-haloalkyl; and
R^{14b} is preferably selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkyl-C₁-C₄-alkyl-, where the cycloalkyl moieties in the three last-mentioned radicals may carry a CN group; C₁-C₆-alkyl substituted with a CN group, phenyl which is optionally substituted with 1, 2, 3 or 4, substituents each independently selected from the group consisting of halogen, cyano, nitro, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51

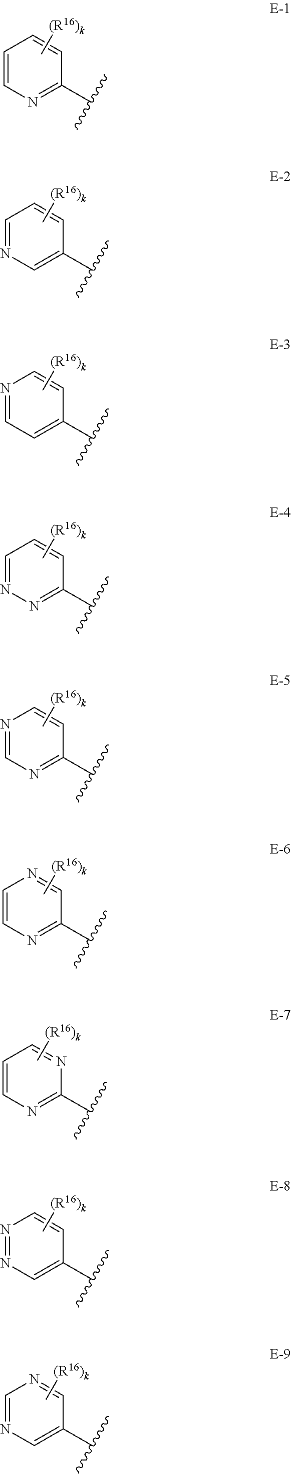

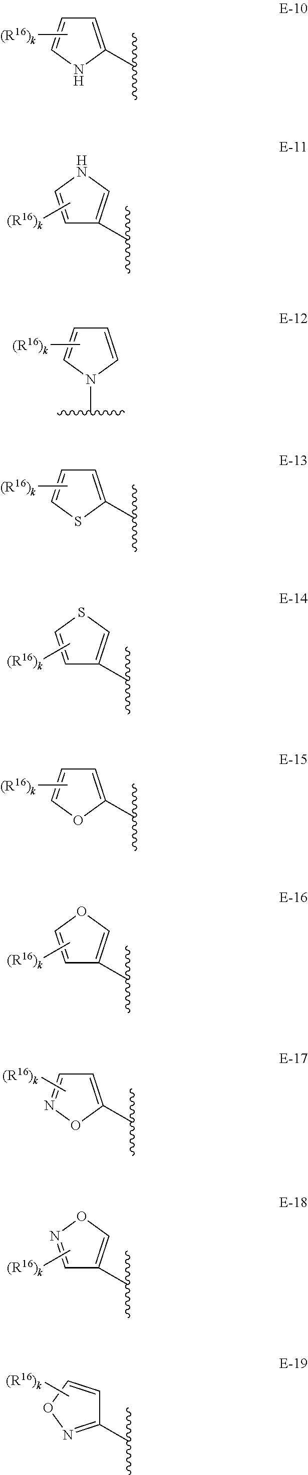

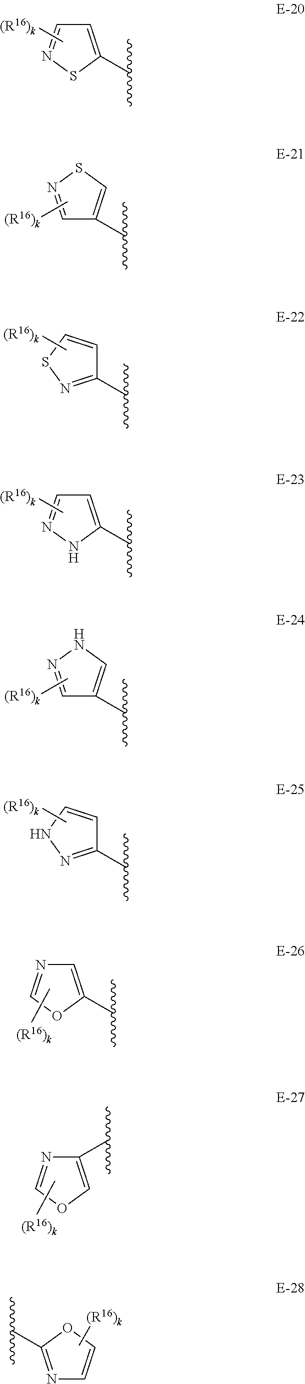

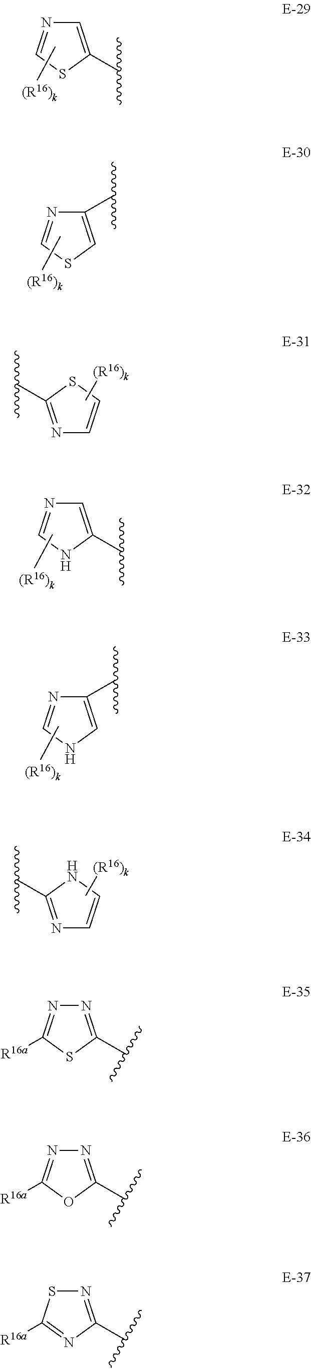

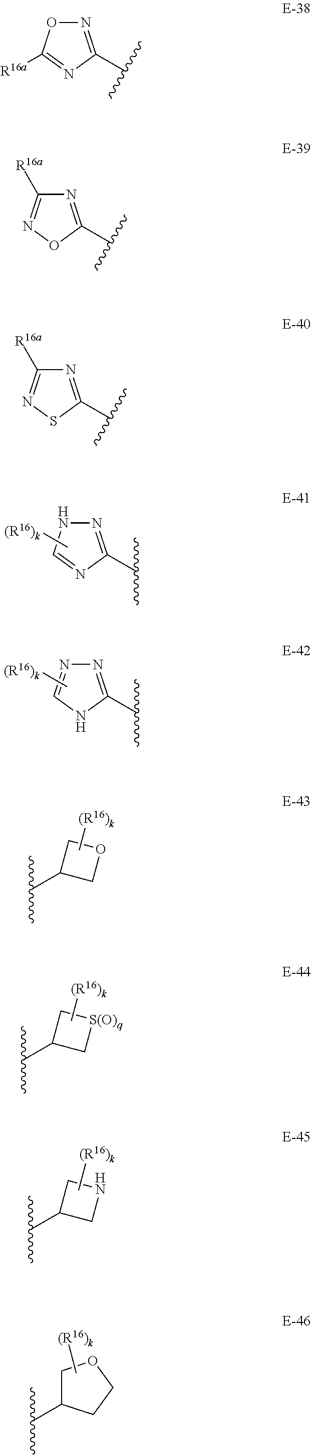

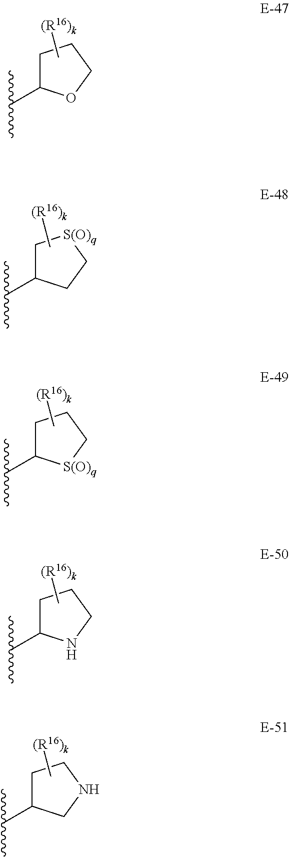

wherein
[symbol] denotes the bonding point to the remainder of the molecule,
k is 0, 1, 2 or 3, n is 0, 1 or 2,
q is 0, 1 or 2;
each R^{16a} is independently hydrogen or has one of the meanings given below for R¹⁶; and
each R¹⁶ is independently selected from the group consisting of halogen, cyano, nitro, C₁-C₄-alkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or two R¹⁶ present on the same carbon atom of a saturated ring may form together a group ═O or ═S.

More preferably, in the above radicals R^{10a} and R^{10b},
R^{14a} is selected from hydrogen and methyl; and
R^{14b} is selected from hydrogen, C₁-C₄-alkyl, C₁-C₄-haloalkyl, C₂-C₄-alkynyl, C₂-C₄-haloalkynyl, C₃-C₆-cycloalkyl, C₃-C₆-halocycloalkyl, C₃-C₆-cycloalkyl-methyl-, where the cycloalkyl moieties in the three last-mentioned radicals may carry a CN group; C₁-C₆-alkyl substituted with a CN group, and a 4-membered saturated heterocyclic ring comprising one heteroatom or heteroatom group selected from S, SO and SO₂ as ring member (ring E-44), where the heterocyclic ring is optionally substituted with one or more, preferably 1 or 2, in particular 1, substituents R¹⁶;
   wherein each R¹⁶ is independently selected from the group consisting of halogen, cyano, nitro, C₁-C₄-alkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or two R¹⁶ present on the same carbon atom may form together a group ═O or ═S.

Preferably, in the above radicals, each R¹⁶ is independently selected from halogen, CN, C₁-C₄-alkyl, G-C₄-haloalkyl, C₁-C₄-alkoxy and C₁-C₄-haloalkoxy.

Specifically, in the above radicals R^{10a} and R^{10b}, R^{14a} is selected from hydrogen and methyl and is specifically hydrogen, and R^{14b} is selected from hydrogen, C₁-C₆-alkyl and C₁-C₆-haloalkyl.

R⁸ as a radical in the group —C(═NR⁶)R⁸ is preferably selected from hydrogen, C₁-C₄-alkyl, C₁-C₄-haloalkyl and NR^{10a}R^{10b}, and more preferably from hydrogen and N(R^{10a})R^{10b}, and is specifically hydrogen.

In this case (i.e. in NR^{10a}R^{10b} as a meaning of R⁸), R^{10a} and R^{10b} are preferably selected, independently of each other, from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkylcarbonyl, C₁-C₆-haloalkylcarbonyl, —C(═O)N(R^{14a})R^{14b}, —C(═S)N(R^{14a})R^{14b}, phenyl which is optionally substituted with 1, 2, 3 or 4, substituents R¹⁶, and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂, as ring members, where the heterocyclic ring is optionally substituted with one or more substituents R¹⁶; or R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂, as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; wherein R^{14a}, R^{14b} and R¹⁶ have one of the above general meanings, or, in particular, one of the below preferred meanings.

More preferably, R^{10a} and R^{10b} are in this case selected, independently of each other, from hydrogen, C₁-C₄-alkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkylcarbonyl, C₁-C₄-haloalkylcarbonyl, C₁-C₄-alkylaminocarbonyl, C₁-C₄-haloalkylaminocarbonyl, C₃-C₆-cycloalkylaminocarbonyl and C₃-C₆-halocycloalkylaminocarbonyl, or, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, CN, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio.

Even more preferably, R^{10a} and R^{10b} are in this case selected, independently of each other, from hydrogen, C₁-C₄-alkyl, C₃-C₆-cycloalkyl, C₃-C₆-halocycloalkyl, C₁-C₄-alkylaminocarbonyl and C₁-C₄-haloalkylaminocarbonyl; and are specifically hydrogen or C₁-C₆-alkyl.

In an alternatively more preferred embodiment of the invention, A¹ is N(R⁵)R⁶, wherein
R⁵ is selected from hydrogen and C₁-C₆-alkyl; and
R⁶ is N(R^{10a})R^{10b}, wherein
   R^{10a} is selected from hydrogen and C₁-C₆-alkyl; and
   R^{10b} is —C(═O)R¹³, wherein
      R¹³ is selected from the group consisting of hydrogen, halogen, C₁-C₆-alkyl, C₃-C₈-cycloalkyl, C₂-C₆-alkenyl and C₂-C₆-alkynyl, wherein the four last-mentioned aliphatic or cycloaliphatic radicals may be unsubstituted, partially or fully halogenated and/or may carry 1 or 2 radicals selected from CN, C₃-C₄-cycloalkyl, C₁-C₄-alkoxy, C₁-C₄-haloalkoxy and oxo.

In an alternative embodiment of the invention, A is A².

In A², W is preferably O.

In A², Y is preferably N(R⁵)R⁶; wherein R⁵ and R⁶ have one of the above general meanings, or, in particular, one of the below preferred meanings.

More preferably, in A², W is O and Y is N(R⁵)R⁶; wherein R⁵ and R⁶ have one of the above general meanings, or, in particular, one of the below preferred meanings.

In N(R⁵)R⁶ as a radical Y,
R⁵ is preferably selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl and C₃-C₈-halocycloalkyl, where the aforementioned aliphatic and cycloaliphatic radicals may be substituted by 1, 2 or 3 radicals R⁸; and
R⁶ is preferably selected from hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted by one or more substituents R⁸,
   —OR⁹, —N(R^{10a})R^{10b}, —S(O)ₙR⁹, C(═O)N(R^{10a})N(R^{10a})R^{10b}, —C(═O)R⁸, —P(═O)(OR⁹)₂, —P(═S)(OR⁹)₂,
   phenyl which may be substituted with 1, 2, 3, 4, or 5 substituents R¹¹, and a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹;
   or
   R⁵ and R⁶, together with the nitrogen atom to which they are bound, form a 3-, 4-, 5-, 6-, 7- or 8-membered saturated, partially unsaturated or maximally unsaturated heteromonocyclic ring, where the ring may further contain 1, 2, 3 or 4 heteroatoms or heteroatom-containing groups selected from O, S, SO, SO₂, N, NH, C═O and C═S as ring members, or form a 7-, 8-, 9- or 10-membered saturated, partially unsaturated or maximally unsaturated heterobicyclic ring, where the ring may further contain 1, 2, 3 or 4 heteroatoms or heteroatom groups selected from N, O, S, NO, SO, SO₂, C═O and C═S, wherein the heteromonocyclic or heterobicyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
   or
   R⁵ and R⁶ together form a group ═C(R⁸)₂, ═S(O)ₘ(R⁹)₂, ═NR^{10a} or ═NOR⁹;
   wherein R⁸, R⁹, R^{10a}, R^{10b} and R¹¹ are have one of the above general meanings, or, in particular, one of the below preferred meanings.

More preferably, in N(R⁵)R⁶ as a radical Y,
R⁵ is selected from hydrogen, C₁-C₆-alkyl, C₂-C₃-alkynyl, —CH₂—CN and C₁-C₆-alkoxy-methyl-; and
R⁶ is selected from hydrogen, C₁-C₆-alkyl, C₁-C₆-haloalkyl, C₃-C₆-cycloalkyl, C₃-C₆-halocycloalkyl, where the four last-mentioned aliphatic and cycloaliphatic radicals may carry 1, 2 or 3 radicals R⁸; C₂-C₆-alkynyl, —C(═O)R⁸, —S(O)ₙR⁹, —P(═O)(OR⁹)₂, —P(═S)(OR⁹)₂, phenyl which may be substituted with 1, 2, 3, 4, or 5 substituents R¹¹, and a 3-, 4-, 5-, 6- or 7-membered saturated, partially unsaturated or maximally unsaturated heteromonocyclic ring containing 1, 2 or 3 heteroatoms or heteroatom groups independently selected from N, O, S, NO, SO and SO₂, as ring members, where the heteromonocyclic ring may be substituted with one or more substituents R¹¹;
   and especially from hydrogen, C₁-C₆-alkyl, C₁-C₆-haloalkyl, C₃-C₆-cycloalkyl, C₃-C₆-halocycloalkyl, where the four last-mentioned aliphatic and cycloaliphatic radicals may carry 1, 2 or 3 radicals R⁸; —C(═O)R⁸, —S(O)ₙR⁹, —P(═O)(OR⁹)₂, —P(═S)(OR⁹)₂, phenyl which may be substituted with 1, 2, 3, 4, or 5 substituents R¹¹, and a 3-, 4-, 5-, 6- or 7-membered saturated, partially unsaturated or maximally unsaturated heteromonocyclic ring containing 1, 2 or 3 heteroatoms or heteroatom groups independently selected from N, O, S, NO, SO and SO₂, as ring members, where the heteromonocyclic ring may be substituted with one or more substituents R¹¹;
   wherein R⁸ and R¹¹ have one of the above general meanings, or, in particular, one of the below preferred meanings and wherein each R⁹ is independently selected from C₁-C₆-alkyl, C₁-C₆-haloalkyl, C₃-C₆-cycloalkyl, phenyl and —N(R^{10a})R^{10b}, wherein R^{10a} and R^{10b}, independently of each other, are hydrogen or C₁-C₆-alkyl;
   or
   R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, or form a 7-, 8-, 9- or 10-membered saturated heterobicyclic ring, where the ring may further contain 1, 2, 3 or 4 heteroatoms or heteroatom groups selected from O, S, SO, SO₂, NH and C═O, wherein the heteromonocyclic or heterobicyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy and C₁-C₆-haloalkoxy;
   or
   R⁵ and R⁶ together form a group ═S(O)ₘ(R⁹)₂;
   wherein each R⁹ is independently selected from C₁-C₆-alkyl, C₁-C₆-haloalkyl, C₃-C₆-cycloalkyl, C₃-C₆-halocycloalkyl and C₃-C₆-cycloalkyl-C₁-C₄-alkyl.

Even more preferably, in N(R⁵)R⁶ as a radical Y,
R⁵ is selected from hydrogen, C₁-C₆-alkyl, C₂-C₃-alkynyl, CH₂—CN and C₁-C₆-alkoxy-methyl-; and
R⁶ is selected from C₁-C₆-alkyl, C₁-C₆-haloalkyl, C₁-C₄-alkyl which carries one radical R⁸, C₃-C₆-cycloalkyl which may be substituted by 1 or 2 substituents selected from F, CN, methyl and oxo,
   C₂-C₆-alkynyl, —C(═O)R⁸, phenyl which may be substituted with 1, 2, 3, 4, or 5 substituents R¹¹, and a 3-, 4-, 5- or 6-membered saturated, partially unsaturated or maximally unsaturated heteromonocyclic ring containing 1, 2 or 3 heteroatoms or heteroatom groups independently selected from N, O, S, NO, SO and SO₂, as ring members, where the heteromonocyclic ring may be substituted with one or more substituents R¹¹;
   and especially from C₁-C₆-alkyl, C₁-C₆-haloalkyl, C₁-C₄-alkyl which carries one radical R⁸, C₃-C₆-cycloalkyl which may be substituted by 1 or 2 substituents selected from F, CN, methyl and oxo,
   —C(═O)R⁸, phenyl which may be substituted with 1, 2, 3, 4, or 5 substituents R¹¹, and a 3-, 4-, 5- or 6-membered saturated, partially unsaturated or maximally unsaturated heteromonocyclic ring containing 1, 2 or 3 heteroatoms or heteroatom groups independently selected from N, O, S, NO, SO and SO₂, as ring members, where the heteromonocyclic ring may be substituted with one or more substituents R¹¹;
   wherein R⁸ and R¹¹ have one of the above general meanings, or, in particular, one of the below preferred meanings;
   or
   R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, or form a 7-, 8-, 9- or 10-membered saturated heterobicyclic ring, where the ring may further contain 1, 2, 3 or 4 heteroatoms or heteroatom groups selected from O, S, SO, SO₂, NH and C═O, wherein the heteromonocyclic or heterobicyclic ring may be substituted with 1, 2 or 3 substituents independently selected from the group consisting of halogen, cyano, C₁-C₆-alkyl, C₁-C₆-haloalkyl, C₁-C₆-alkoxy and C₁-C₆-haloalkoxy;
   or
   R⁵ and R⁶ together form a group ═S(O)ₘ(R⁹)₂;
   wherein each R⁹ is independently selected from C₁-C₆-alkyl, C₁-C₆-haloalkyl, C₃-C₆-cycloalkyl, C₃-C₆-halocycloalkyl and C₃-C₆-cycloalkyl-C₁-C₄-alkyl.

Preferably, the 3-, 4-, 5-, 6- or 7-membered saturated, partially unsaturated or maximally unsaturated heteromonocyclic ring R⁶ containing 1, 2 or 3 heteroatoms or heteroatom groups independently selected from N, O, S, NO, SO and SO₂, as ring members is selected from rings D-1 to D-173 listed below in context with A⁴, and more preferably from rings F-1 to F-51 (among which rings F-1, F-2, F-3, F-44, F-46 and F-47 are preferred) listed below. The ring may be substituted with one or more, preferably 1, 2 or 3, in particular 1, substituents R¹¹.

In N(R⁵)R⁶ as a radical Y,
R⁸ as a substituent on an aliphatic or cycloaliphatic group is preferably selected from cyano, C₃-C₈-cycloalkyl which may be substituted by 1 or 2 substituents selected from CN, methyl and oxo, C₃-C₈-halocycloalkyl, —OR⁹, —S(O)ₙR⁹, —N(R^{10a})R^{10b}, C(═O)R¹³, —C(═O)N(R^{10a})R^{10b}, —C(═O)OR⁹, phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶; and
R⁸ in the group —C(═O)R⁸ is preferably selected from hydrogen, C₁-C₆-alkyl, C₁-C₆-haloalkyl, —OR⁹ and —N(R^{10a})R^{10b};
   wherein R⁹, R^{10a}, R^{10b}, R¹³ and R¹⁶ have one of the above general meanings, or, in particular, one of the below preferred meanings.

More preferably, in N(R⁵)R⁶ as a radical Y,
R⁸ as a substituent on an aliphatic or cycloaliphatic group is selected from cyano, C₃-C₈-cycloalkyl which may be substituted by 1 or 2 substituents selected from CN; C₃-C₈-halocycloalkyl, C₁-C₄-alkylcarbonyl, C₁-C₄-haloalkylcarbonyl, phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶; and
R⁸ in the group —C(═O)R⁸ is preferably selected from hydrogen, C₁-C₆-alkyl, C₁-C₆-haloalkyl, —OR⁹ and —N(R^{10a})R^{10b};
   wherein R⁹, R^{10a}, R^{10b} and R¹⁶ have one of the above general meanings, or, in particular, one of the below preferred meanings.

In this case,
R^{10a} and R^{10b} are preferably selected, independently of each other, from hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkylcarbonyl, C₁-C₄-haloalkylcarbonyl, C₁-C₄-alkylaminocarbonyl, C₁-C₄-haloalkylaminocarbonyl, C₃-C₆-cycloalkylaminocarbonyl and C₃-C₆-halocycloalkylaminocarbonyl, or, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio.

More preferably, R^{10a} and R^{10b} are in this case selected, independently of each other, from hydrogen, C₁-C₄-alkyl, C₁-C₄-haloalkyl, C₃-C₆-cycloalkyl, C₃-C₆-halocycloalkyl, C₁-C₄-alkylaminocarbonyl and C₁-C₄-haloalkylaminocarbonyl. Specifically, they are selected, independently of each other, from hydrogen, C₁-C₄-alkyl, C₁-C₄-haloalkyl and C₃-C₆-cycloalkyl. Very specifically, one of R^{10a} and R^{10b} is hydrogen and the other is C₁-C₄-alkyl, C₁-C₄-haloalkyl or C₃-C₆-cycloalkyl.

In a particular embodiment, in N(R⁵)R⁶ as Y,
R⁵ is selected from hydrogen and C₁-C₄-alkyl;
R⁶ is selected from C₁-C₆-alkyl, C₁-C₆-haloalkyl, C₁-C₄-alkyl which carries one radical R⁸, C₃-C₆-cycloalkyl which may be substituted by 1 or 2 substituents selected from F, CN, methyl and oxo, and specifically by 1 CN substituent; C₂-C₆-alkynyl, phenyl which may be substituted with 1, 2, 3, 4, or 5 substituents R¹¹, and a heteromonocyclic ring selected from rings of formulae F-1 to F-51 (among which rings F-1, F-2, F-3, F-44, F-46 and F-47 are preferred)

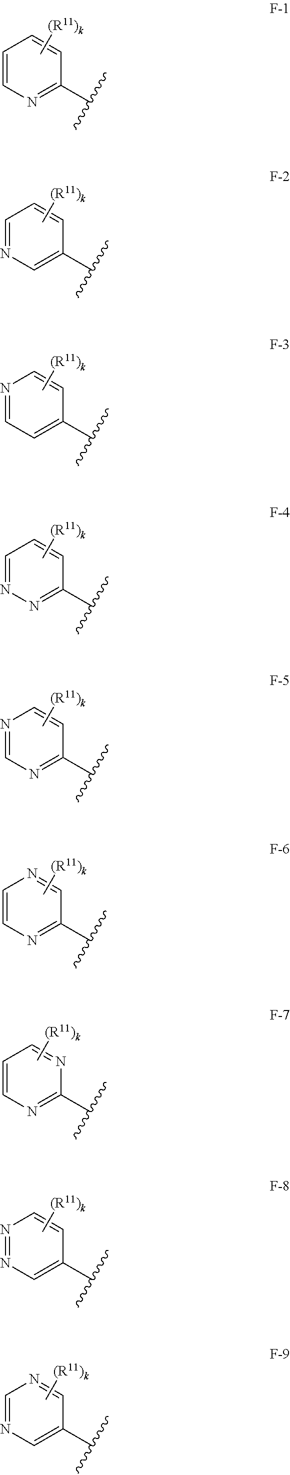

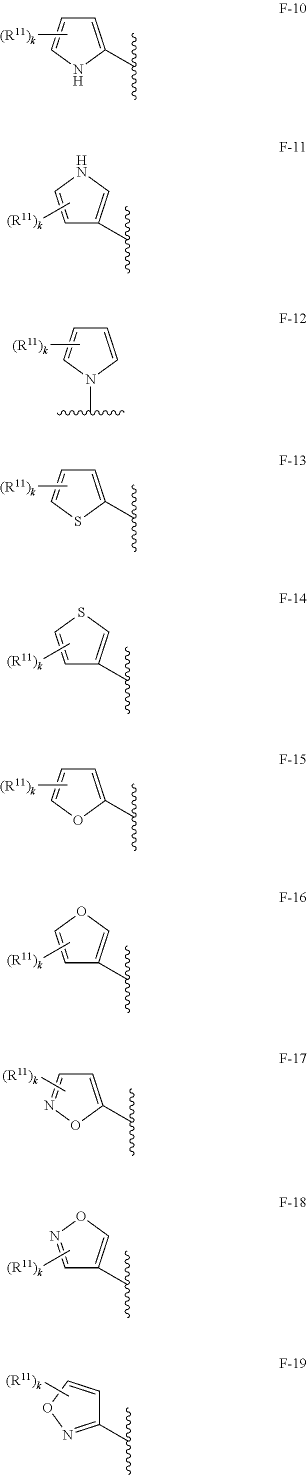

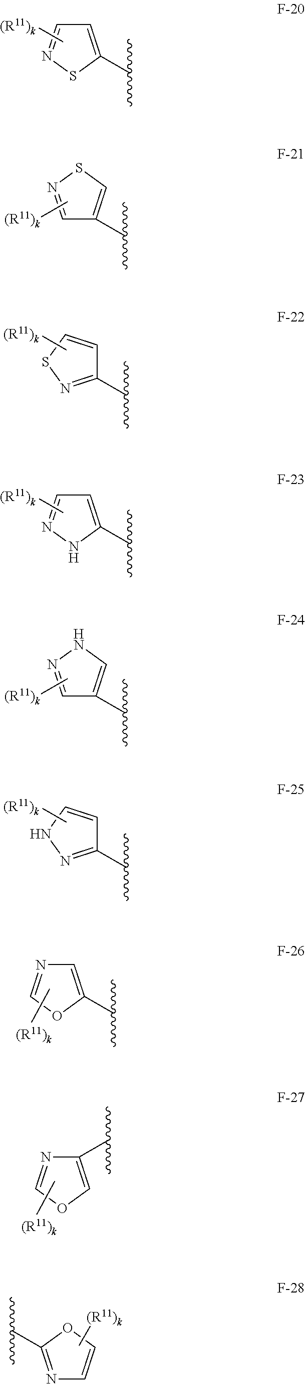

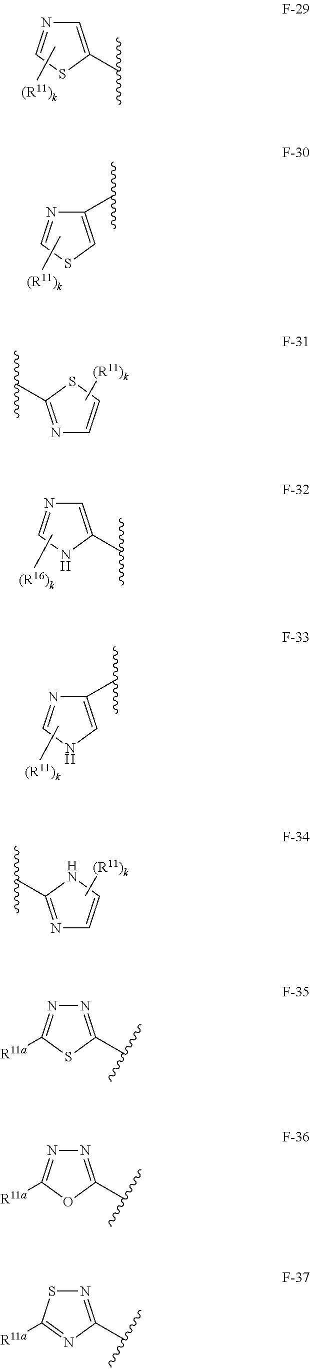

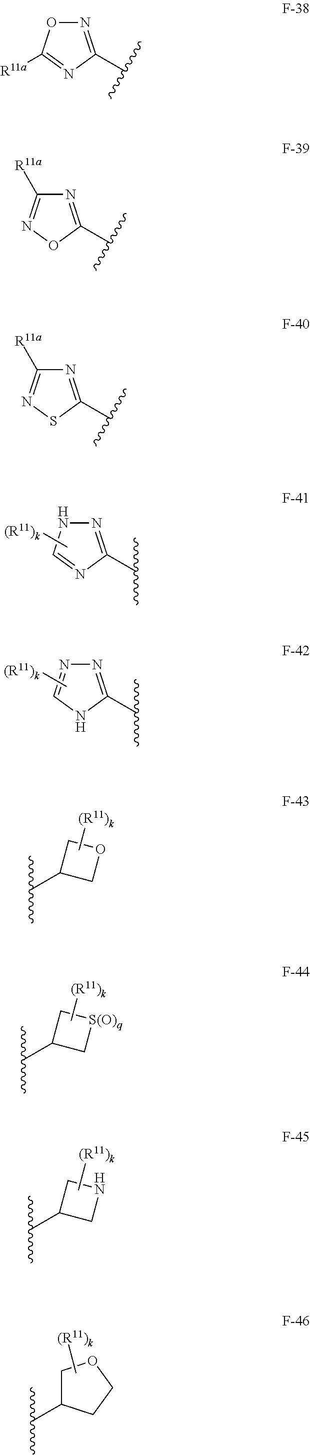

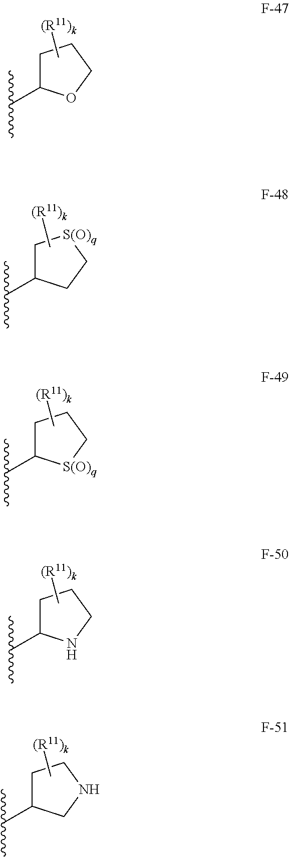

wherein
[symbol] denotes the bonding point to the remainder of the molecule,
k is 0, 1, 2 or 3,
q is 0, 1 or 2,
each R^{11a} is independently hydrogen or has one of the meanings given below for R¹¹; and
each R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or two R¹¹ present on the same carbon atom of a saturated heterocyclic ring may form together ═O or ═S;
R⁸ is selected from CN, C₃-C₈-cycloalkyl which may be substituted by 1 or 2 substituents selected from F, CN, methyl and oxo, and specifically by 1 CN substituent; C₃-C₈-halocycloalkyl, C₁-C₆-alkylthio, C₁-C₆-haloalkylthio, —C(═O)R¹³, —C(═O)N(R^{10a})R^{10b}, phenyl, optionally substituted with 1, 2, 3, 4 or 5 substituents R¹⁶, and a heterocyclic ring selected from rings of formulae E-1 to E-51 as defined above;
   wherein
   R^{10a} is selected from the group consisting of hydrogen, C₁-C₆-alkyl, C₂-C₃-alkynyl, —CH₂—CN and C₁-C₆-alkoxy-methyl;
   R^{10b} is selected from the group consisting of hydrogen, C₁-C₆-alkyl, C₁-C₆-haloalkyl, C₃-C₆-cycloalkyl, C₃-C₆-halocycloalkyl, phenyl which is optionally substituted with 1, 2, 3, 4 or 5 substituents selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 as defined in claim 10;
   R¹³ is selected from the group consisting of hydrogen, C₁-C₆-alkyl, C₁-C₆-haloalkyl, C₃-C₆-cycloalkyl and C₃-C₆-halocycloalkyl; and
   each R¹⁶ as a substituent on phenyl or heterocyclic rings of formulae E-1 to E-51 is independently selected from the group consisting of halogen, cyano, nitro, C₁-C₄-alkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
      two R¹⁶ present on the same carbon atom of a saturated heterocyclic ring may form together ═O or ═S;
   or
   R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, wherein the heterocyclic ring may be substituted with 1, 2 or 3 substituents independently selected from the group consisting of halogen, cyano, C₁-C₆-alkyl, C₁-C₆-haloalkyl, C₁-C₆-alkoxy and C₁-C₆-haloalkoxy;
   or
   R⁵ and R⁶ together form a group ═S(O)ₘ(R⁹)₂;
   wherein each R⁹ is independently selected from C₁-C₆-alkyl, C₁-C₆-haloalkyl, C₃-C₆-cycloalkyl, C₃-C₆-halocycloalkyl and C₃-C₆-cycloalkyl-C₁-C₄-alkyl and specifically from C₁-C₆-alkyl, and m is 0, 1 or 2 and specifically 0.

Especially,
R⁵ is hydrogen;
R⁶ is selected from C₁-C₆-alkyl, C₁-C₆-haloalkyl, C₁-C₄-alkyl which carries one radical R⁸, C₃-C₆-cycloalkyl,
   phenyl which may be substituted with 1, 2, 3, 4, or 5 substituents R¹¹, and a heteromonocyclic ring selected from rings of formulae F-1 to F-51 (among which rings F-1, F-2, F-3, F-44, F-46 and F-47 are preferred) as defined above;
   wherein
   [symbol] denotes the bonding point to the remainder of the molecule,
   k is 0, 1, 2 or 3,
   q is 0, 1 or 2,
   each R^{11a} is independently hydrogen or has one of the meanings given below for R¹¹; and
   each R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or two R¹¹ present on the same carbon atom of a saturated heterocyclic ring may form together ═O or ═S;
   R⁸ is selected from C₃-C₈-cycloalkyl, C₃-C₈-halocycloalkyl, C₁-C₆-haloalkylthio, —C(═O)R¹³, —C(═O)N(R^{10a})R^{10b}, phenyl, optionally substituted with 1, 2, 3, 4 or 5 substituents R¹⁶, and a heterocyclic ring selected from rings of formulae E-1 to E-51 as defined in claim 10; wherein
      R^{10a} is selected from the group consisting of hydrogen, C₁-C₆-alkyl, C₂-C₃-alkynyl, —CH₂—CN and C₁-C₆-alkoxy-methyl;
      R^{10b} is selected from the group consisting of hydrogen, C₁-C₆-alkyl, C₁-C₆-haloalkyl, C₃-C₆-cycloalkyl, C₃-C₆-halocycloalkyl, phenyl which is optionally substituted with 1, 2, 3, 4 or 5 substituents selected from the group consisting of halogen, cyano, nitro, C₁-C₄-alkyl, C₁-C₄-haloalkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 as defined in claim 10;
      R¹³ is selected from the group consisting of hydrogen, C₁-C₆-alkyl, C₁-C₆-haloalkyl, C₃-C₆-cycloalkyl and C₃-C₆-halocycloalkyl; and
      each R¹⁶ as a substituent on phenyl or heterocyclic rings of formulae E-1 to E-51 is independently selected from the group consisting of halogen, cyano, nitro, C₁-C₄-alkyl, C₁-C₄-haloalkoxy, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
         two R¹⁶ present on the same carbon atom of a saturated heterocyclic ring may form together ═O or ═S;
      or
   R⁵ and R⁶, together with the nitrogen atom to which they are bound, form a 3-, 4-, 5- or 6-membered saturated heterocyclic ring, where the ring may further contain 1 or 2 heteroatoms or heteroatom-containing groups selected from O, S, SO, SO₂, NH and C═O as ring members, wherein the heterocyclic ring may be substituted with 1, 2 or 3 substituents independently selected from the group consisting of halogen, cyano, C₁-C₆-alkyl, C₁-C₆-haloalkyl, C₁-C₆-alkoxy and C₁-C₆-haloalkoxy.

Specifically,
R⁵ is selected from hydrogen and C₁-C₄-alkyl;
R⁶ is selected from C₁-C₆-alkyl, C₁-C₆-haloalkyl, C₁-C₄-alkyl which carries one radical R⁸, C₃-C₆-cycloalkyl which may be substituted by 1 CN substituent, C₂-C₆-alkynyl, phenyl which may be substituted with 1, 2, 3, 4, or 5 substituents R¹¹, and a heteromonocyclic ring selected from rings of formulae F-1 to F-51 as defined above, and specifically from rings F-1, F-2, F-3, F-44, F-46 and F-47;
   wherein
   R⁸ is selected from CN, C₃-C₈-cycloalkyl which may be substituted by 1 CN substituent; C₃-C₈-halocycloalkyl, —C(═O)R¹³, and a heterocyclic ring selected from rings of formulae E-1 to E-51, preferably rings E-1 to E-42 as defined above;
      wherein
      R¹³ is selected from the group consisting of hydrogen, C₁-C₆-alkyl and C₁-C₆-haloalkyl; and
      each R¹⁶ as a substituent on phenyl or heterocyclic rings of formulae E-1 to E-51 is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
         two R¹⁶ present on the same carbon atom of a saturated heterocyclic ring may form together ═O or ═S;
or
R⁵ and R⁶, together with the nitrogen atom to which they are bound, form a 3-, 4-, 5- or 6-membered, specifically a 3- or 4-membered, saturated heteromonocyclic ring (i.e. R⁵ and R⁶ form together a group —(CH₂)₂—, —(CH₂)₃—, —(CH₂)₄— or —(CH₂)₅—, preferably a group —(CH₂)₂— or —(CH₂)₃—);
   or
   R⁵ and R⁶ together form a group ═S(O)ₘ(R⁹)₂;
   wherein each R⁹ is independently selected from C₁-C₆-alkyl and C₁-C₆-haloalkyl and specifically from C₁-C₆-alkyl, and m is 0.

In an alternative embodiment of the invention, A is A³.

Preferably, R^{7a} and R^{7b} in the group A³ are independently of each other selected from hydrogen, C₁-C₄-alkyl and C₁-C₄-haloalkyl, and more preferably one of R^{7a} and R^{7b} is hydrogen and the other is hydrogen or methyl. Specifically, both are hydrogen.

In the group A³,
R⁵ is preferably selected from hydrogen, C₁-C₆-alkyl, C₃-C₆-cycloalkyl, C₂-C₆-alkenyl, C₂-C₆-alkynyl, wherein the four last-mentioned aliphatic and cycloaliphatic radicals may be partially or fully halogenated and/or may be substituted with one or more substituents R⁸; and
R⁶ is preferably selected from hydrogen, C₁-C₆-alkyl, C₃-C₆-cycloalkyl, C₂-C₆-alkenyl, C₂-C₆-alkynyl, wherein the four last-mentioned aliphatic and cycloaliphatic radicals may be partially or fully halogenated and/or may be substituted by one or more substituents R⁸,
   —OR⁹, —N(R^{10a})R^{10b}, —S(O)ₙR⁹, —C(═O)N(R^{10a})N(R^{10a})R^{10b}, —C(═O)R⁸,
   and a 3-, 4-, 5-, 6- or 7-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹;
   or
   R⁵ and R⁶, together with the nitrogen atom to which they are bound, form a 3-, 4-, 5- or 6-membered saturated, partially unsaturated or maximally unsaturated heterocyclic ring, where the ring may further contain 1, 2, 3 or 4 heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
   or
   R⁵ and R⁶ together form a group ═C(R⁸)₂, ═S(O)ₘ(R⁹)₂, ═NR^{10a} or ═NOR⁹
   wherein R⁸, R⁹, R^{10a}, R^{10b} and R¹¹ have one of the above general meanings, or, in particular, one of the below preferred meanings.

More preferably, in the group A³,
R⁵ is selected from hydrogen, C₁-C₄-alkyl, C₂-C₃-alkynyl, —CH₂—CN and C₁-C₆-alkoxy-methyl- and preferably from hydrogen and C₁-C₄-alkyl; and
R⁶ is —C(═O)R⁸;
   wherein R⁸ has one of the above general meanings, or, in particular, one of the below preferred meanings.

R⁸ in —C(═O)R⁸ as a meaning of the radicals R⁵ and R⁶ of the group A³ is preferably selected from the group consisting of C₁-C₆-alkyl, C₁-C₆-haloalkyl, C₃-C₈-cycloalkyl, C₃-C₈-halocycloalkyl, where the aliphatic and cycloaliphatic moieties in the four last-mentioned radicals may be substituted by one or more radicals R¹³; —OR⁹, —S(O)ₙR⁹, —N(R^{10a})R^{10b}, phenyl, optionally substituted with 1, 2, 3, 4 or 5 substituents R¹⁶, and a 3-, 4-, 5- or 6-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,

wherein R⁹, R^{10a}, R^{10b}, R¹³ and R¹⁶ have one of the above general meanings, or, in particular, one of the below preferred meanings.

More preferably, R⁸ in —C(═O)R⁸ as a meaning of the radicals R⁵ and R⁶ of the group A³ is selected from the group consisting of C₁-C₆-alkyl, C₁-C₆-haloalkyl, substituted by one radical R¹³, C₃-C₈-cycloalkyl, C₃-C₈-halocycloalkyl, —N(R^{10a})R^{10b}, phenyl which is optionally substituted with 1, 2, 3, 4 or 5 substituents each independently selected from the group consisting of halogen, cyano, nitro, C₁-C₄-alkyl, C₁-C₄-haloalkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 as defined above.

R⁹ in —OR⁹ as a meaning of R⁸ in the group —C(═O)R⁸ as a meaning of the radicals R⁵ and R⁶ of the group A³ is preferably selected from C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl and C₃-C₈-cycloalkyl-C₁-C₄-alkyl-, and more preferably from C₁-C₆-alkyl, C₁-C₆-haloalkyl, C₃-C₈-cycloalkyl, C₃-C₈-halocycloalkyl and C₃-C₈-cycloalkyl-C₁-C₄-alkyl-.

R^{10a} and R^{10b} in —N(R^{10a})R^{10b} as a meaning of R⁸ in the group —C(═O)R⁸ as a meaning of the radicals R⁵ and R⁶ of the group A³ are, independently of each other, preferably selected from hydrogen, C₁-C₄-alkyl, C₁-C₄-haloalkyl, C₁-C₄-alkyl substituted by one radical R¹³, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkylcarbonyl, C₁-C₄-haloalkylcarbonyl, C₁-C₄-alkylaminocarbonyl, C₁-C₄-haloalkylaminocarbonyl, C₃-C₆-cycloalkylaminocarbonyl, C₃-C₆-halocycloalkylaminocarbonyl, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more, preferably 1, 2 or 3, in particular 1, substituents selected from halogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio; or, R^{10a} and R^{10b}, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio.

More preferably, R^{10a} and R^{10b} in R⁸ in the radicals R⁵ and R⁶ of the group A³ are, independently of each other, selected from hydrogen, C₁-C₄-alkyl, C₁-C₄-haloalkyl, alkyl substituted by one radical R¹³, C₃-C₆-cycloalkyl, C₃-C₆-halocycloalkyl, and a 3- or 4-membered saturated heterocyclic ring comprising 1 heteroatom or heteroatom group selected from N, O, S, NO, SO and SO₂, as ring member, where the heterocyclic ring is optionally substituted with one or more, preferably 1, 2 or 3, in particular 1, substituents selected from halogen, CN, C₁-C₄-haloalkyl, C₁-C₄-alkoxy and C₁-C₄-haloalkoxy; and are specifically, independently of each other, selected from hydrogen, C₁-C₄-alkyl and C₁-C₄-haloalkyl.

R¹³ in R⁸ in the radicals R⁵ and R⁶ of the group A³ is preferably selected from CN, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl and C₁-C₆-haloalkylsulfonyl.

R¹⁶ in R⁸ in the radicals R⁵ and R⁶ of the group A³ is preferably selected from halogen, C₁-C₄-alkyl, C₁-C₄-haloalkyl, C₁-C₄-alkoxy and C₁-C₄-haloalkoxy.

In particular, R⁸ in —C(═O)R⁸ as a meaning of the radicals R⁵ and R⁶ of the group A³ is selected from the group consisting of C₁-C₆-alkyl, C₁-C₆-haloalkyl, substituted by one radical R¹³, C₃-C₈-cycloalkyl, C₃-C₈-halocycloalkyl, —N(R^{10a})R^{10b}, phenyl which is optionally substituted with 1, 2, 3, 4 or 5 substituents each independently selected from the group consisting of halogen, cyano, nitro, C₁-C₄-alkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 as defined above;

wherein
R^{10a} and R^{10b}, independently of each other, are selected from hydrogen, C₁-C₄-alkyl, C₁-C₄-haloalkyl and C₃-C₆-cycloalkyl; and
R¹³ is selected from CN, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl and a heterocyclic ring selected from rings of formulae E-1 to E-51 as defined in claim 10; and
each R¹⁶ as a substituent on heterocyclic rings of formulae E-1 to E-51 is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
two R¹⁶ present on the same carbon atom of a saturated heterocyclic ring may form together ═O or ═S.

More particularly, R⁸ in —C(═O)R⁸ as a meaning of the radicals R⁵ and R⁶ of the group A³ is selected from the group consisting of C₁-C₆-alkyl, C₁-C₆-haloalkyl, substituted by one radical R¹³, C₃-C₈-cycloalkyl, C₃-C₈-halocycloalkyl and —N(R^{10a})R^{10b}; wherein
R^{10a} and R^{10b}, independently of each other, are selected from hydrogen and C₁-C₄-alkyl; and
R¹³ is selected from C₁-C₆-alkylthio, C₁-C₆-haloalkylthio, C₁-C₆-alkylsulfinyl, C₁-C₆-haloalkylsulfinyl, C₁-C₆-alkylsulfonyl and C₁-C₆-haloalkylsulfonyl.

In an alternative embodiment of the invention, A is A⁴.

A⁴ is preferably selected from a 3-, 4-, 5-, 6- or 7-membered saturated, partially unsaturated or maximally unsaturated heteromonocyclic ring containing 1, 2, 3 or 4 heteroatoms or heteroatom groups selected from N, O, S, NO, SO and SO₂, as ring members, where the heteromonocyclic ring is optionally substituted with one or more, preferably 1, 2 or 3, in particular 1, substituents R¹¹, where R¹¹ has one of the above general meanings, or, in particular, one of the below preferred meanings.

More preferably, A⁴ is selected from a 3-, 4-, 5-, 6- or 7-membered saturated heteromonocyclic ring containing 1 or 2 heteroatoms or heteroatom groups selected from N, O, S, NO, SO and SO₂, as ring members, a 5-, 6- or 7-membered partially unsaturated heteromonocyclic ring containing 1, 2 or 3 heteroatoms or heteroatom groups selected from N, O, S, NO, SO and SO₂, as ring members, and a 5- or 6-membered aromatic heteromonocyclic ring containing 1, 2, 3 or 4 heteroatoms selected from N, O and S as ring members, where the heteromonocyclic ring is optionally substituted with one or more, preferably 1, 2 or 3, in particular 1, substituents R¹¹, where R¹¹ has one of the above general meanings, or, in particular, one of the below preferred meanings.

A⁴ is even more preferably selected from rings of formulae D-1 to D-173

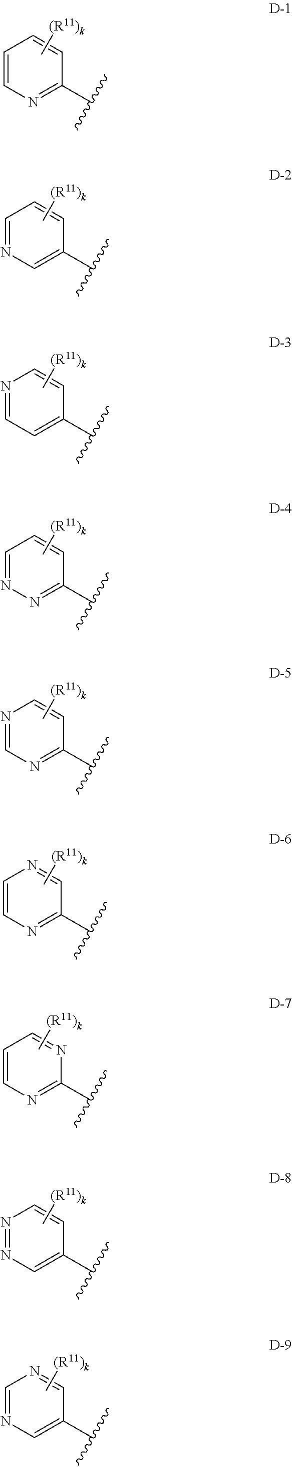

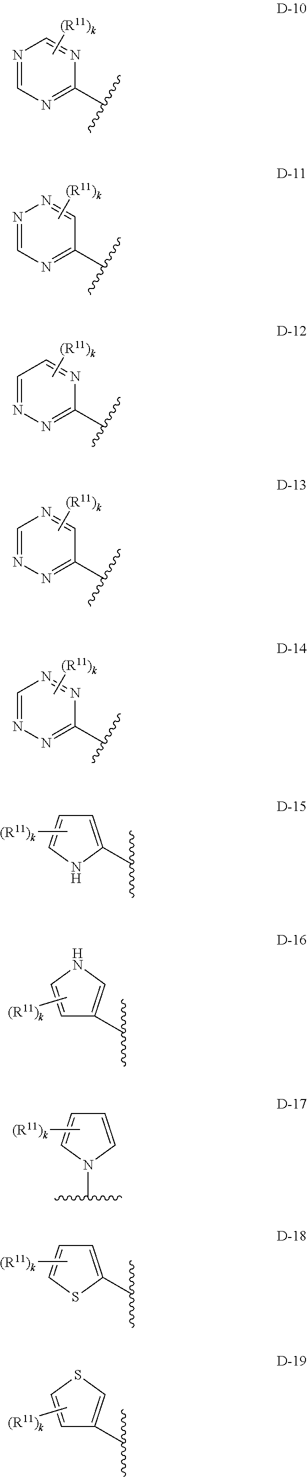

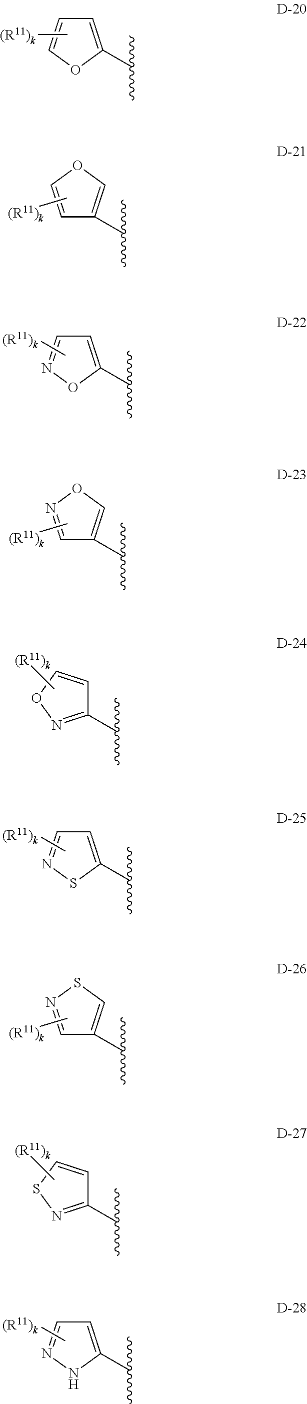

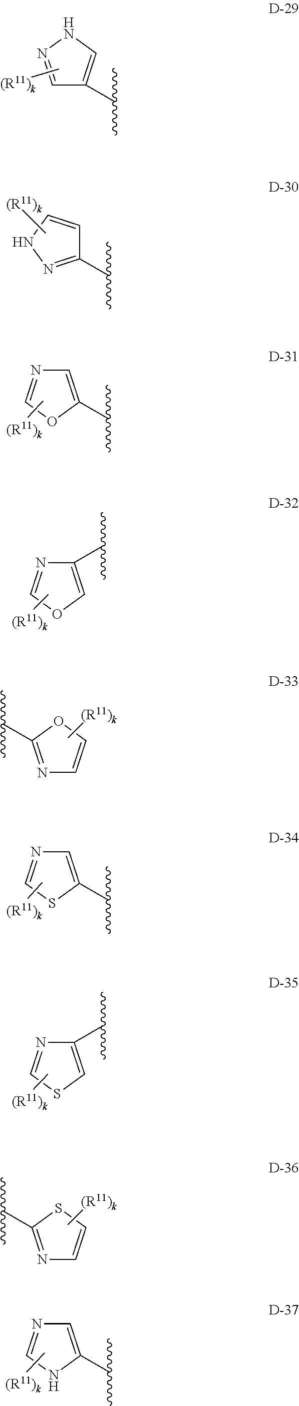

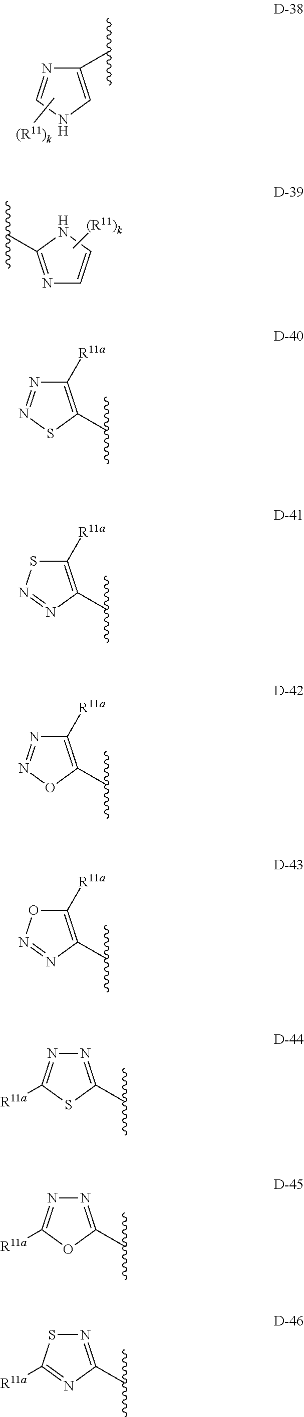

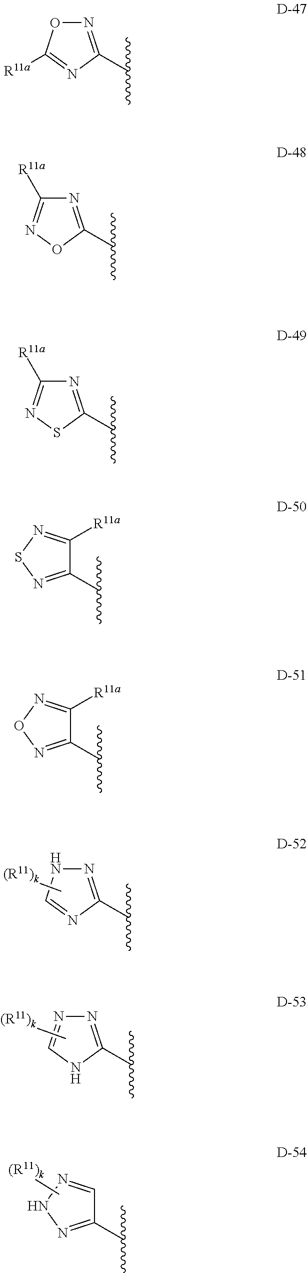

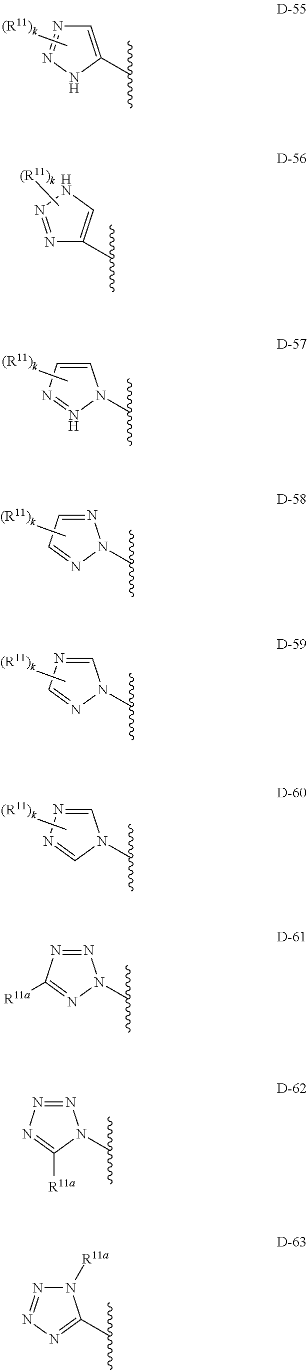

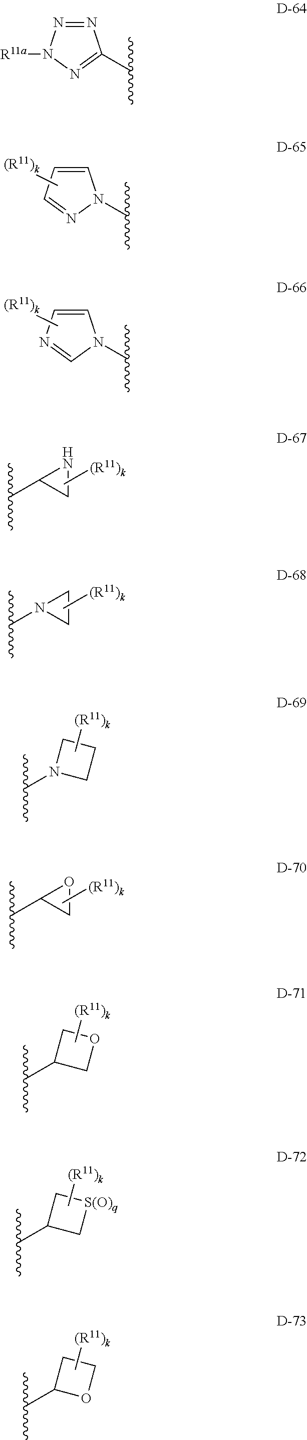

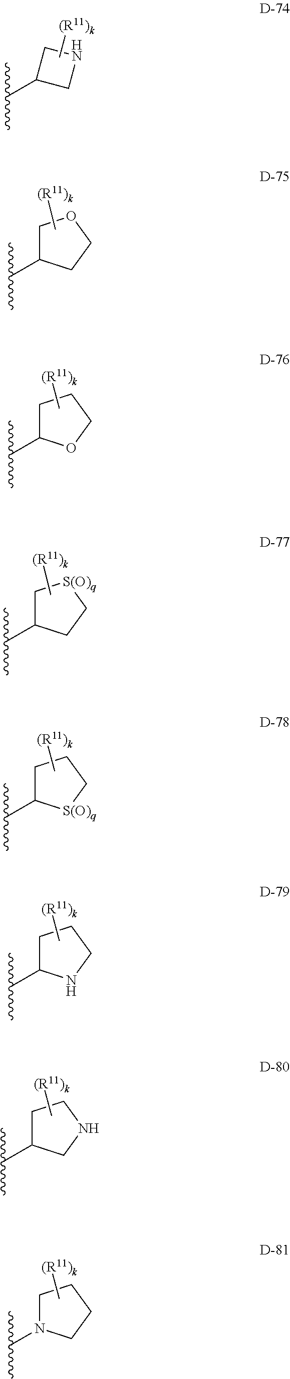

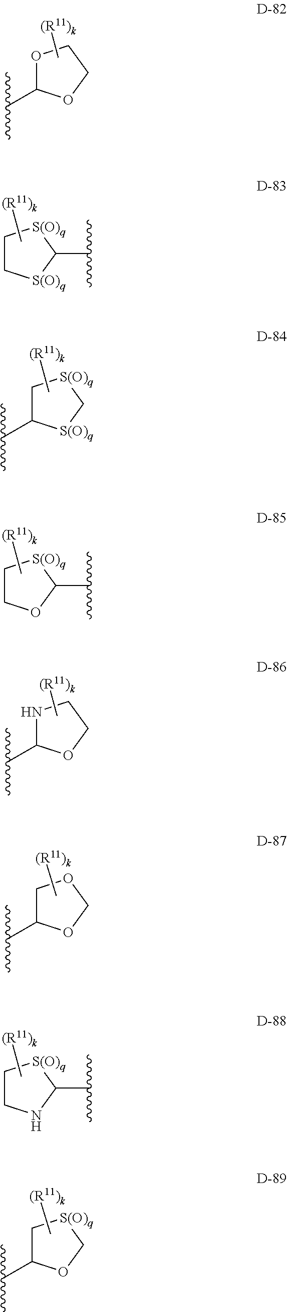

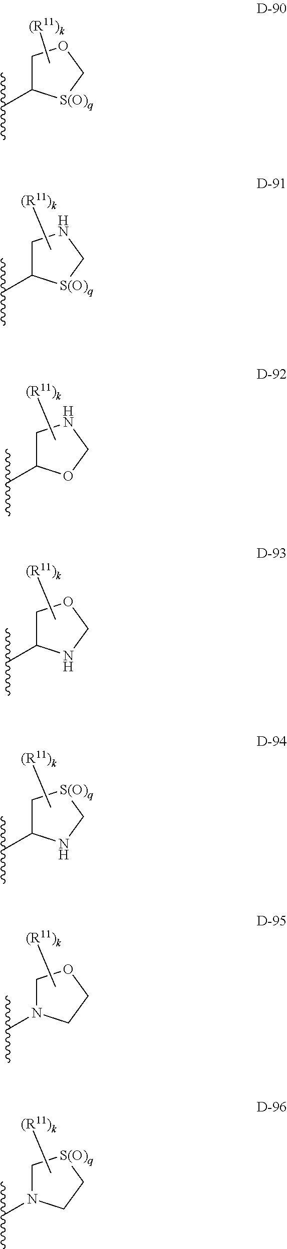

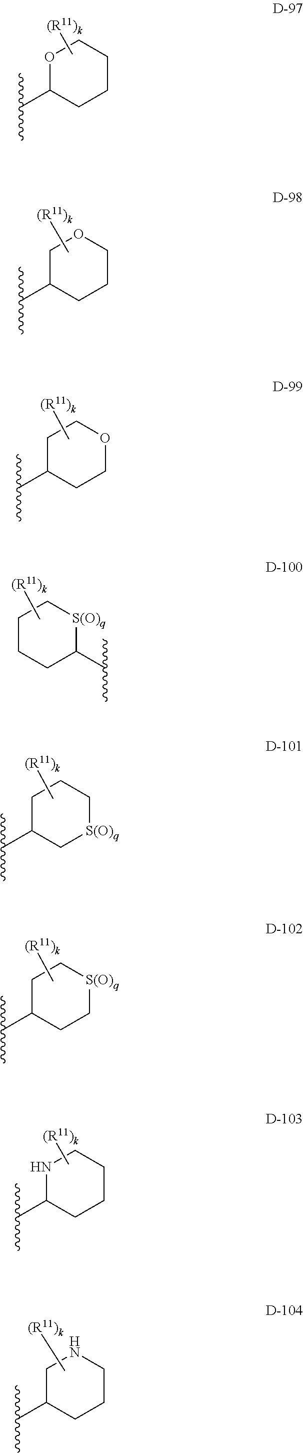

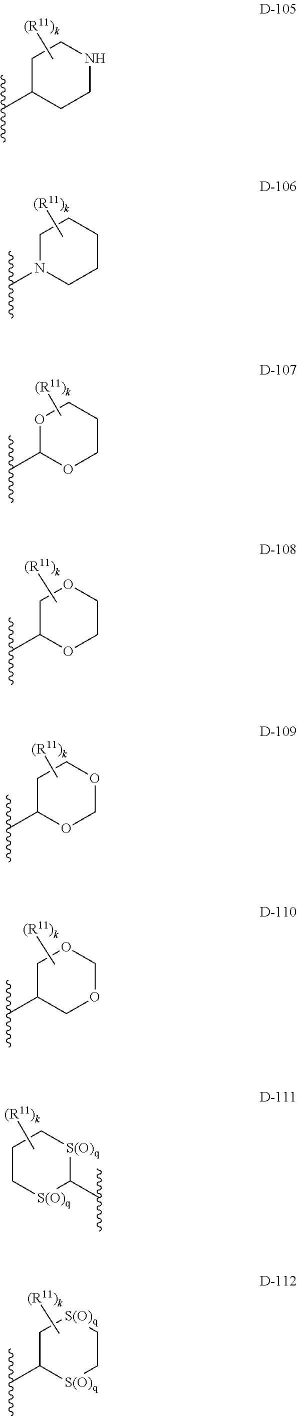

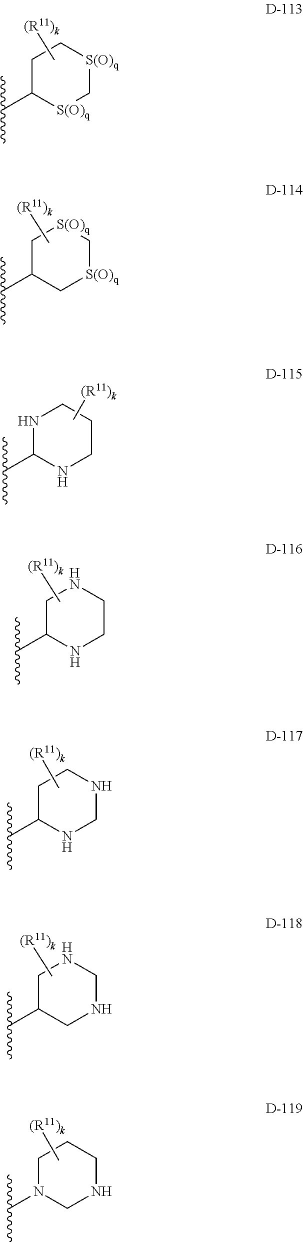

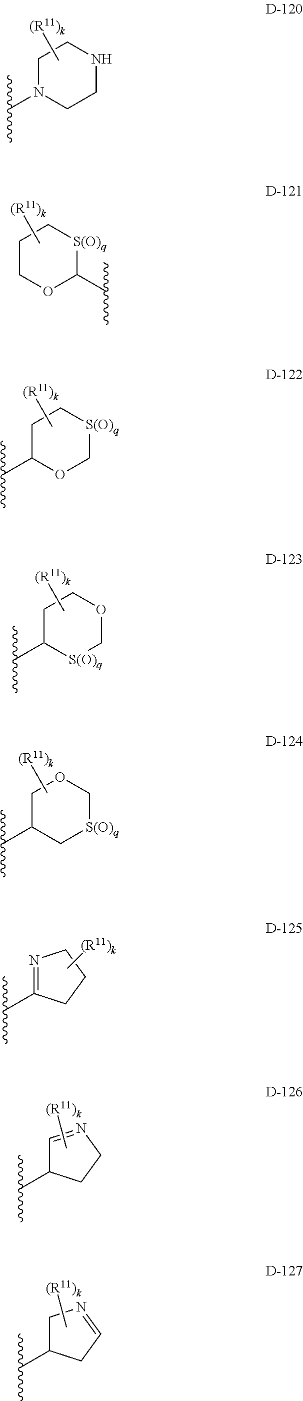

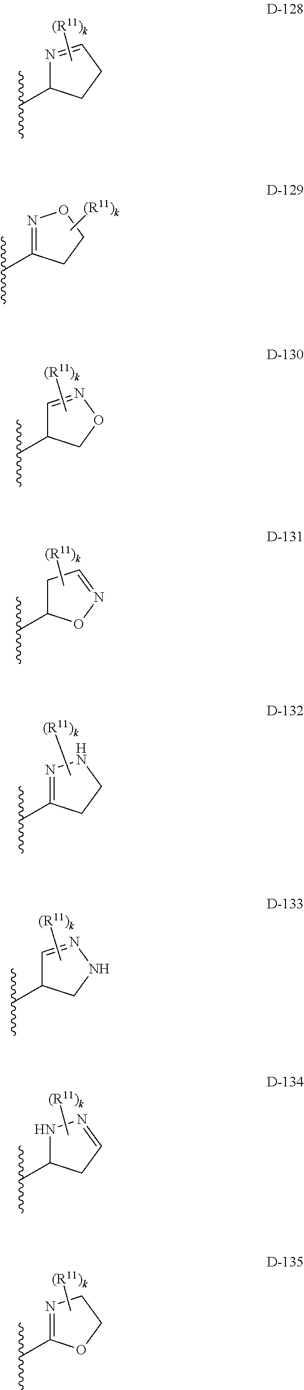

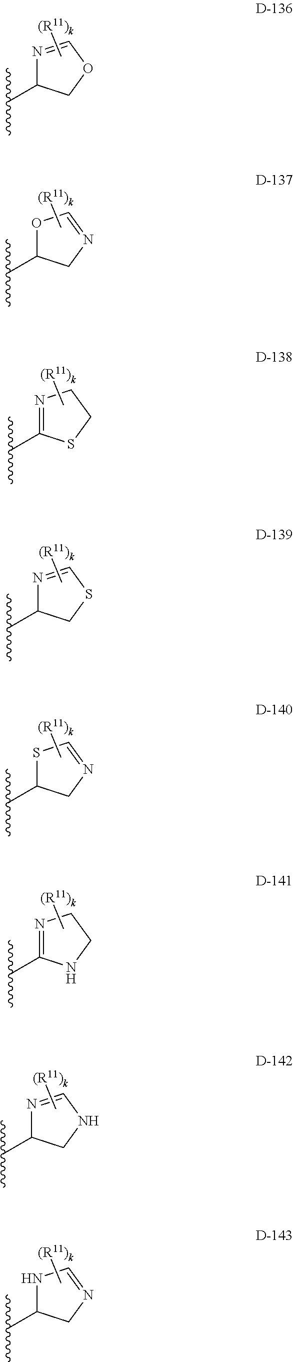

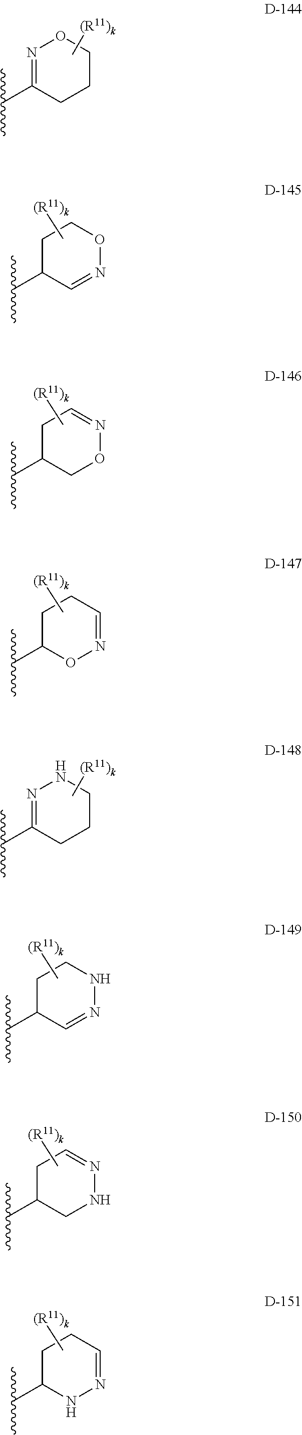

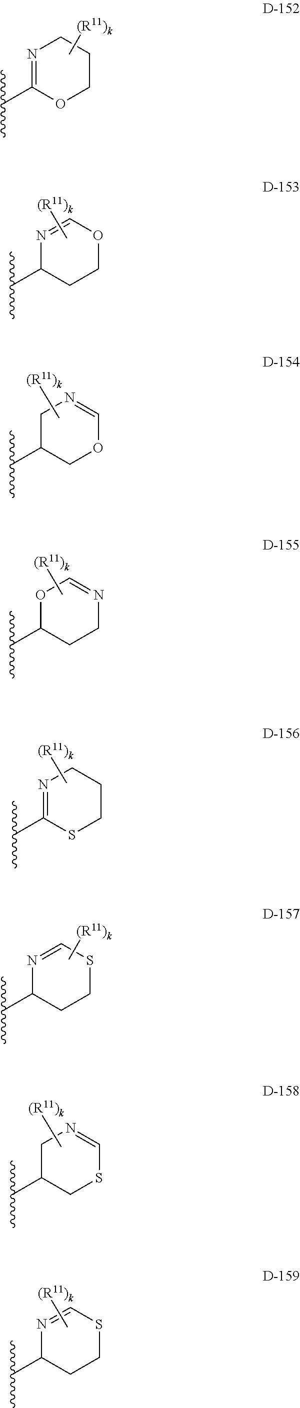

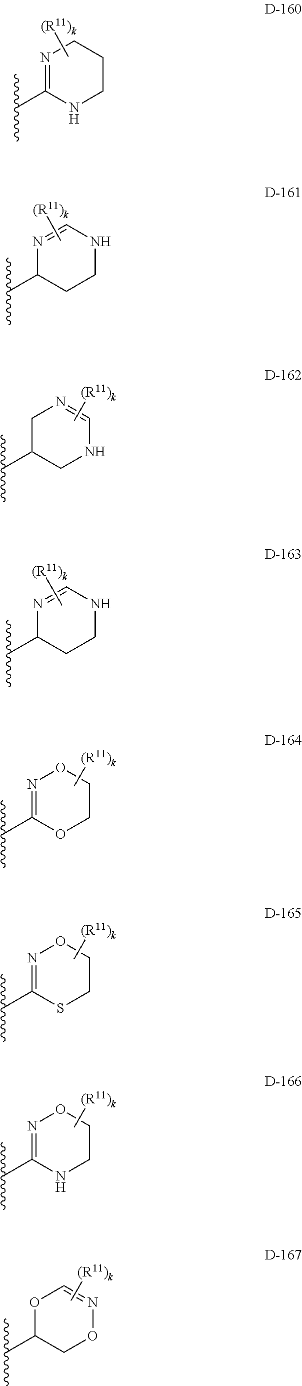

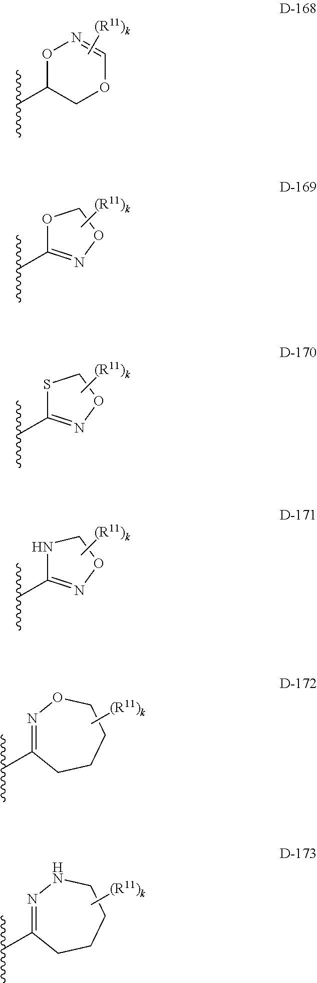

wherein
[symbol] denotes the bonding point to the remainder of the molecule,
k is 0, 1, 2 or 3;
q is 0, 1 or 2;
each R^{11a} is independently hydrogen or has one of the above general meanings, or, in particular, one of the below preferred meanings given for R¹¹; and
each R¹¹ has independently one of the above general meanings, or, in particular, one of the below preferred meanings;
and is preferably selected from D-59, D-65 and D-66 and is in particular D-59.

Preferably, in the above rings D-1 to D-173,
each R¹¹ is independently selected from the group consisting of halogen, cyano, nitro, C₁-C₄-haloalkyl, C₁-C₄-alkoxy, C₁-C₄-haloalkoxy, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
   two R¹¹ present on the same carbon atom of a saturated or partially unsaturated ring may form together ═O or ═S.

Among the radicals A¹, A², A³ and A⁴, preference is given to A².

In a preferred embodiment, B¹ and B⁵ are CH and B², B³ and B⁴ are CR², where R² has one of the above general meanings, or, in particular, one of the below preferred meanings.

More preferably, B¹ and B⁵ are CH, B² is CR², where R² is not hydrogen, and B³ and B⁴ are CR², where R² has one of the above general meanings, or, in particular, one of the below preferred meanings. More specifically, B² is CR², where R² is not hydrogen and is preferably selected from CF₃, F and Cl and is specifically CF₃, and B¹, B³, B⁴ and B⁵ are CH.

Preferably, R² is selected from hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last-mentioned aliphatic and cycloaliphatic radicals may be partially or fully halogenated and/or may be substituted by one or more radicals R⁸, —OR⁹, —S(O)ₙR⁹ and —N(R^{10a})R^{10b},

wherein R⁸, R⁹, R^{10a} and R^{10b} have one of the above general meanings, or, in particular, one of the below preferred meanings.

More preferably, R² is selected from hydrogen, halogen and C₁-C₂-haloalkyl, preferably from hydrogen, F, Cl, Br and CF₃, in particular from hydrogen, CF₃, F and Cl, and specifically from hydrogen and CF₃.

Specifically, B¹ and B⁵ are CH, B² is CR², where R² is selected from CF₃, F and Cl, and B³ and B⁴ are CR², where R² is selected from hydrogen, CF₃, F and Cl. More specifically, B² is CR², where R² is selected from CF₃, F and Cl and is specifically CF₃, and B¹, B³, B⁴ and B⁵ are CH.

Preferably, R¹ is selected from C₁-C₄-alkyl, C₁-C₄-haloalkoxy-C₁-C₄-alkyl, C₃-C₆-cycloalkyl C₃-C₆-halocycloalkyl or C₁-C₄-alkoxycarbonyl, more preferably, from C₁-C₄-alkyl, C₃-C₆-cycloalkyl, C₃-C₆-halocycloalkyl and C₁-C₄-alkoxycarbonyl, even more preferably from C₁-C₄-alkyl, C₁-C₄-haloalkyl and C₁-C₄-alkoxycarbonyl, and particularly preferably from C₁-C₄-haloalkyl and C₁-C₄-alkoxycarbonyl. In particular, R¹ is C₁-C₄-haloalkyl, specifically C₁-C₂-haloalkyl and more specifically halomethyl, in particular fluoromethyl, such as fluoromethyl, difluoromethyl and trifluoromethyl, and is very specifically trifluoromethyl.

Preferably, L¹ is selected from hydrogen and C₁-C₄-alkyl, and is more preferably hydrogen.

Preferably, E is selected from hydrogen, halogen and C₁-C₄-alkyl, and is more preferably hydrogen.

Preferably, X is selected from hydrogen, halogen and C₁-C₄-alkyl, and is more preferably hydrogen.

Preferably, R³ is selected from hydrogen, C₁-C₄-alkyl, C₂-C₃-alkynyl, —CH₂—CN and C₁-C₆-alkoxy-methyl-, more preferably from hydrogen and C₁-C₄-alkyl and is specifically hydrogen.

Z is preferably O.

If not specified otherwise above, R⁸, R⁹, R^{10a}, R^{10b}, R¹¹, R¹², R¹³, R¹⁴, R^{14a}, R^{14b}, R¹⁵ and R¹⁶ have following preferred meanings:

In case R⁸ is a substituent on an alkyl, alkenyl or alkynyl group, it is preferably selected from the group consisting of cyano, C₃-C₈-cycloalkyl, C₃-C₈-halocycloalkyl, —OR⁹, —SR⁹, —C(═O)N(R^{10a})R^{10b}, —C(═S)N(R^{10a})R^{10b}, —C(═O)OR⁹, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁶; where R⁹, R^{10a}, R^{10b} and R¹⁶ have one of the meanings given above or in particular one of the preferred meanings given below.

In case R⁸ is a substituent on an alkyl, alkenyl or alkynyl group, it is even more preferably selected from the group consisting of cyano, C₃-C₆-cycloalkyl, C₃-C₆-halocycloalkyl, C₁-C₄-alkoxy, C₁-C₄-haloalkoxy, C₁-C₄-haloalkylthio, —C(═O)N(R^{10a})R^{10b}, —C(═S)N(R^{10a})R^{10b}, —C(═O)OR⁹, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁶; where R⁹, R^{10a}, R^{10b} and R¹⁶ have one of the meanings given above or in particular one of the preferred meanings given below. In particular it is selected from the group consisting of cyano, C₃-C₆-cycloalkyl, C₃-C₆-halocycloalkyl, —C(═O)N(R^{10a})R^{10b}, —C(═S)N(R^{10a})R^{10b}, —C(═O)OR⁹, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁶; where R⁹, R^{10a}, R^{10b} and R¹⁶ have one of the meanings given above or in particular one of the preferred meanings given below.

In case R⁸ is a substituent on a cycloalkyl group, it is preferably selected from the group consisting of cyano, C₁-C₆-alkyl, C₁-C₆-haloalkyl, C₁-C₆-alkoxy-C₁-C₆-alkyl, —OR⁹, —OSO₂R⁹, —SR⁹, —N(R^{10a})R^{10b}, —C(═O)N(R^{10a})R^{10b}, —C(═S)N(R^{10a})R^{10b}, —C(═O)OR⁹, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁶; where R⁹, R^{10a}, R^{10b} and R¹⁶ have one of the meanings given above or in particular one of the preferred meanings given below.

In case R⁸ is a substituent on a cycloalkyl group, it is even more preferably selected from the group consisting of halogen, C₁-C₄-alkyl, C₁-C₃-haloalkyl, C₁-C₄-alkoxy and C₁-C₃-haloalkoxy. In particular, R⁸ as a substituent on a cycloalkyl group is selected from halogen, C₁-C₄-alkyl and C₁-C₃-haloalkyl.

In case of R⁸ in a group —C(═O)R⁸, ═C(R⁸)₂ or —C(═NR⁶)R⁸, R⁸ is preferably selected from the group consisting of hydrogen, C₁-C₆-alkyl, C₁-C₆-haloalkyl, C₁-C₆-alkoxy-C₁-C₆-alkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, —OR⁹, —SR⁹, —N(R^{10a})R^{10b}, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁶; where R⁹, R^{10a}, R^{10b} and R¹⁶ have one of the meanings given above or in particular one of the preferred meanings given below.

In case of R⁸ in a group —C(═O)R⁸, ═C(R⁸)₂ or —C(═NR⁶)R⁸, R⁸ is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, —N(R^{10a})R^{10b}, phenyl which may be substituted by 1, 2, 3, 4 or 5 radicals R¹⁶, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁶; where R^{10a}, R^{10b} and R¹⁶ have has one of the meanings given above or in particular one of the preferred meanings given below.

Preferably, each R⁹ is independently selected from the group consisting of hydrogen, C₁-C₆-alkyl, C₁-C₆-haloalkyl, C₃-C₈-cycloalkyl, C₃-C₈-halocycloalkyl, C₃-C₈-cycloalkyl-C₁-C₄-alkyl, phenyl which may be substituted by 1, 2, 3, 4 or 5 radicals R¹⁶; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁶, where R¹⁶ has one of the meanings given above or in particular one of the preferred meanings given below.

More preferably, each R⁹ is independently selected from the group consisting of hydrogen, C₁-C₆-alkyl, C₁-C₆-haloalkyl, phenyl which may be substituted by 1, 2, 3, 4 or 5 radicals R¹⁶; and a 5- or 6-membered heteroaromatic ring containing 1, 2 or 3 heteroatoms selected from N, O and S, as ring members, where the heteroaromatic ring may be substituted by one or more radicals R¹⁶; where R¹⁶ has one of the meanings given above or in particular one of the preferred meanings given below.

R^{10a} and R^{10b} are, independently of each other, preferably selected from hydrogen, C₁-C₄-alkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkylcarbonyl, C₁-C₄-haloalkylcarbonyl, C₁-C₄-alkylaminocarbonyl, C₁-C₄-haloalkylaminocarbonyl, C₃-C₆-cycloalkylaminocarbonyl, C₃-C₆-halocycloalkylaminocarbonyl, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more, preferably 1, 2 or 3, in particular 1, substituents selected from halogen, CN, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio; or, R^{10a} and R^{10b}, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, CN, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio.

More preferably, R^{10a} and R^{10b} are, independently of each other, selected from hydrogen, C₁-C₄-alkyl, C₁-C₄-haloalkyl, C₃-C₆-cycloalkyl, C₃-C₆-halocycloalkyl, and a 3- or 4-membered saturated heterocyclic ring comprising 1 heteroatom or heteroatom group selected from N, O, S, NO, SO and SO₂, as ring member, where the heterocyclic ring is optionally substituted with one or more, preferably 1, 2 or 3, in particular 1, substituents selected from halogen, CN, C₁-C₄-haloalkyl, C₁-C₄-alkoxy and C₁-C₄-haloalkoxy; and are specifically, independently of each other, selected from hydrogen, C₁-C₄-alkyl and C₁-C₄-haloalkyl.

Each R¹¹ and each R¹⁶ are independently of each occurrence and independently of each other preferably selected from halogen, CN, C₁-C₄-alkyl, C₁-C₄-alkoxy, C₁-C₄-haloalkoxy, C₁-C₄-alkylthio, C₁-C₄-haloalkylthio, C₁-C₄-haloalkylsulfinyl, C₁-C₄-alkylsulfonyl and C₁-C₄-haloalkylsulfonyl, and more preferably from halogen, CN, C₁-C₄-alkoxy and C₁-C₄-haloalkoxy.

Each R¹² is preferably selected from C₁-C₄-alkyl and is in particular methyl.

In case R¹³ is a substituent on an alkyl, alkenyl or alkynyl group, it is preferably selected from the group consisting of cyano, C₃-C₈-cycloalkyl, C₃-C₈-halocycloalkyl, —OH, —SH, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl and phenyl which may be substituted by 1, 2 or 3 radicals selected from halogen, C₁-C₄-alkyl, C₁-C₄-haloalkyl, C₁-C₄-alkoxy and C₁-C₄-haloalkoxy.

In case R¹³ is a substituent on a cycloalkyl group, it is preferably selected from the group consisting of cyano, C₁-C₆-alkyl, C₁-C₆-haloalkyl, C₃-C₈-cycloalkyl, C₃-C₈-halocycloalkyl, —OH, —SH, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl and phenyl which may be substituted by 1, 2 or 3 radicals selected from halogen, C₁-C₄-alkyl, C₁-C₄-alkoxy and C₁-C₄-haloalkoxy.

In case R¹³ is a substituent on a cycloalkyl group, it is even more preferably selected from the group consisting of halogen, C₁-C₄-alkyl, C₁-C₃-haloalkyl, C₁-C₄-alkoxy and C₁-C₃-haloalkoxy. In particular, R¹³ as a substituent on a cycloalkyl group is selected from halogen, C₁-C₄-alkyl and C₁-C₃-haloalkyl.

In case of R¹³ in a group —C(═O)R¹³, —C(═S)R¹³, ═C(R¹³)₂ or —C(═NR¹⁴)R¹³, R⁸ is preferably selected from the group consisting of hydrogen, C₁-C₆-alkyl, C₁-C₆-haloalkyl, C₃-C₈-cycloalkyl, C₃-C₈-halocycloalkyl, —OH, —SH, C₁-C₆-alkoxy, C₁-C₆-haloalkoxy and phenyl which may be substituted by 1, 2 or 3 radicals selected from halogen, C₁-C₄-alkyl, C₁-C₄-haloalkyl, C₁-C₄-alkoxy and C₁-C₄-haloalkoxy.

R¹⁴, R^{14a} and R^{14b} are, independently of each other, preferably selected from hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl and benzyl, where the phenyl ring in benzyl is optionally substituted 1, 2 or 3, in particular 1, substituents selected from halogen, C₁-C₄-alkyl, C₁-C₄-haloalkyl, C₁-C₄-alkoxy and C₁-C₄-haloalkoxy;

or, R^{14a} and R^{14b}, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, C₁-C₄-alkyl, C₁-C₄-haloalkyl, C₁-C₄-alkoxy and C₁-C₄-haloalkoxy.

More preferably, R¹⁴, R^{14a} and R^{14b} are, independently of each other, selected from hydrogen, C₁-C₄-alkyl, C₁-C₄-haloalkyl, C₃-C₆-cycloalkyl, C₃-C₆-halocycloalkyl and benzyl, where the phenyl ring in benzyl is optionally substituted 1, 2 or 3, in particular 1, substituents selected from halogen, C₁-C₄-alkyl, C₁-C₄-haloalkyl, C₁-C₄-alkoxy and C₁-C₄-haloalkoxy;

or, R^{14a} and R^{14b}, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, C₁-C₄-alkyl, C₁-C₄-haloalkyl, C₁-C₄-alkoxy and C₁-C₄-haloalkoxy.

Each R¹⁵ is preferably selected from hydrogen, C₁-C₆-alkyl, C₁-C₆-haloalkyl, phenyl, benzyl, pyridyl and phenoxy, wherein the four last-mentioned radicals may be unsubstituted and/or carry 1, 2 or 3 substituents selected from C₁-C₆-alkyl, C₁-C₆-haloalkyl, C₁-C₆-alkoxy and C₁-C₆-haloalkoxy.

Each R¹⁶ is preferably selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or

two R¹¹ present on the same carbon atom may form together a group ═O or ═S. More preferably, each R¹⁶ is independently selected from halogen, CN, C₁-C₄-alkyl C₁-C₄-haloalkyl, C₁-C₄-alkoxy and C₁-C₄-haloalkoxy.

In a particular embodiment, the invention relates to a compound I.1

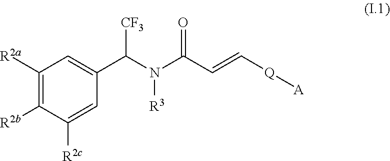

wherein R³, Q and A have one of the general, or in particular, one of the preferred meanings given above and R^{2a}, R^{2b} and R^{2c}, independently of each other, have one of the general, or in particular, one of the preferred meanings given above for R².

In particular, Q is thien-2-yl or thien-3-yl, preferably thien-2-yl.

The group Q-A is in particular a group Q-4 or Q-5, preferably Q-4.

In particular, R^{2b}, R^{2c} and R³ are hydrogen and R^{2a} is CF₃.

Examples of preferred compounds are compounds of the following formulae Ia.1 to Ia.44, where the variables have one of the general or preferred meanings given above. Examples of preferred compounds are the individual compounds compiled in the tables 1 to 2744 below, Moreover, the meanings mentioned below for the individual variables in the tables are per se, independently of the combination in which they are mentioned, a particularly preferred embodiment of the substituents in question.

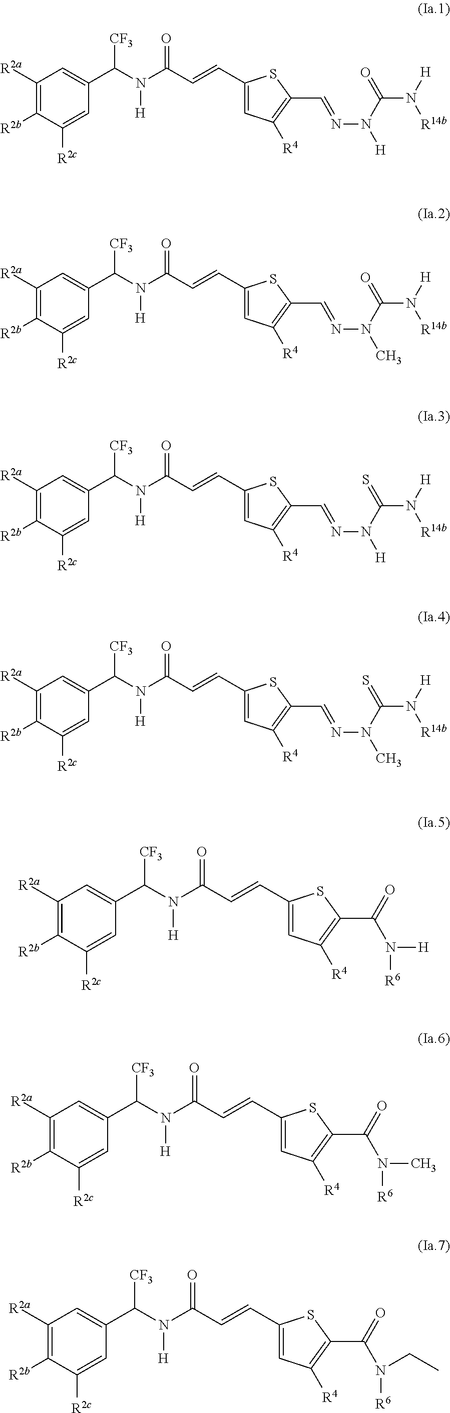

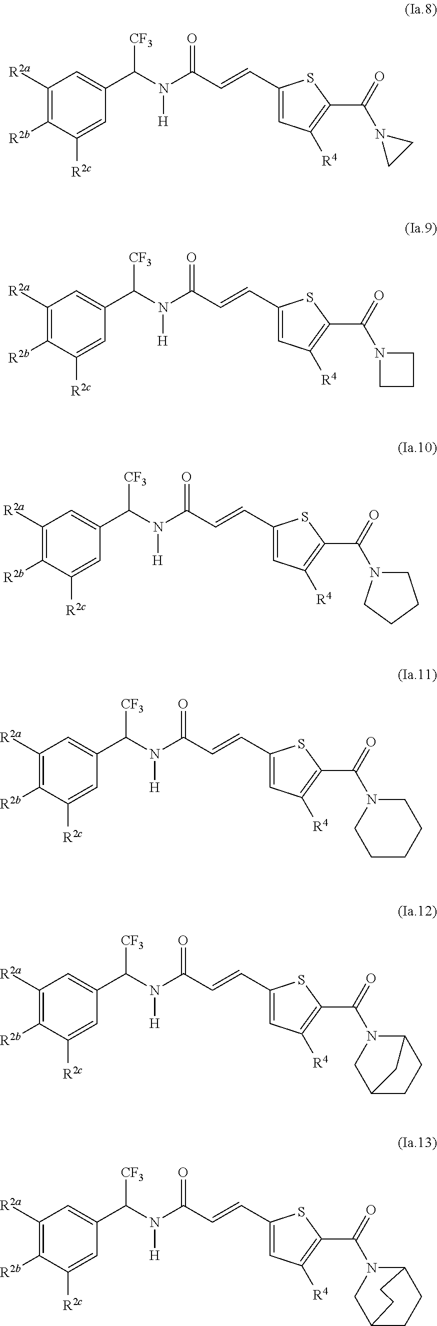

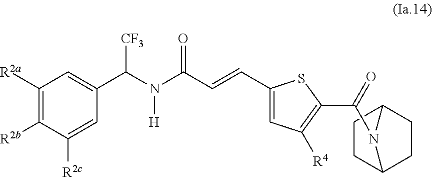

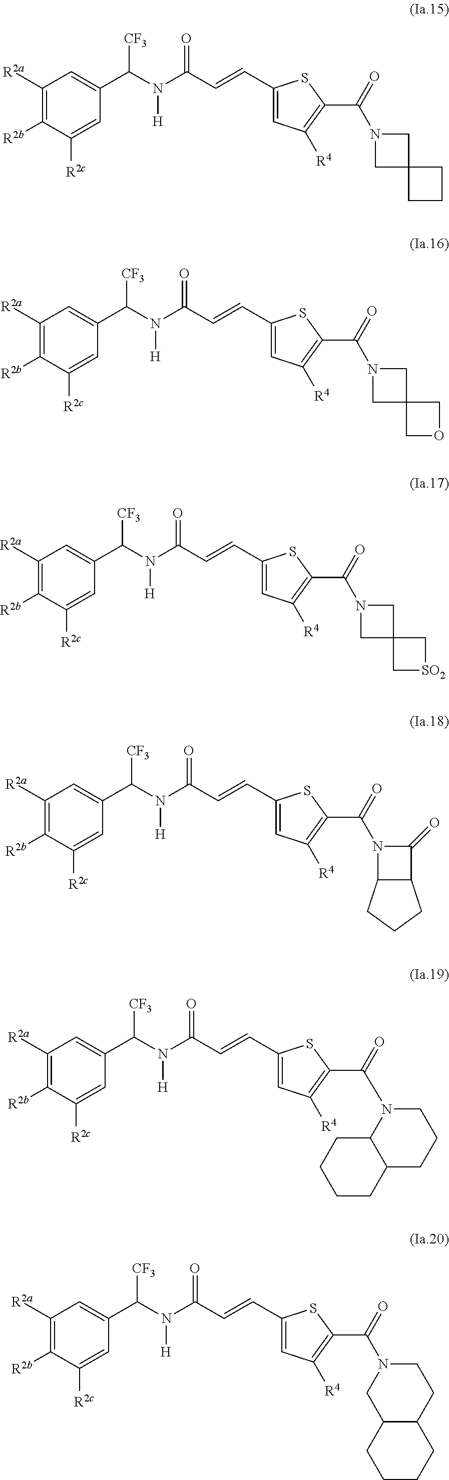

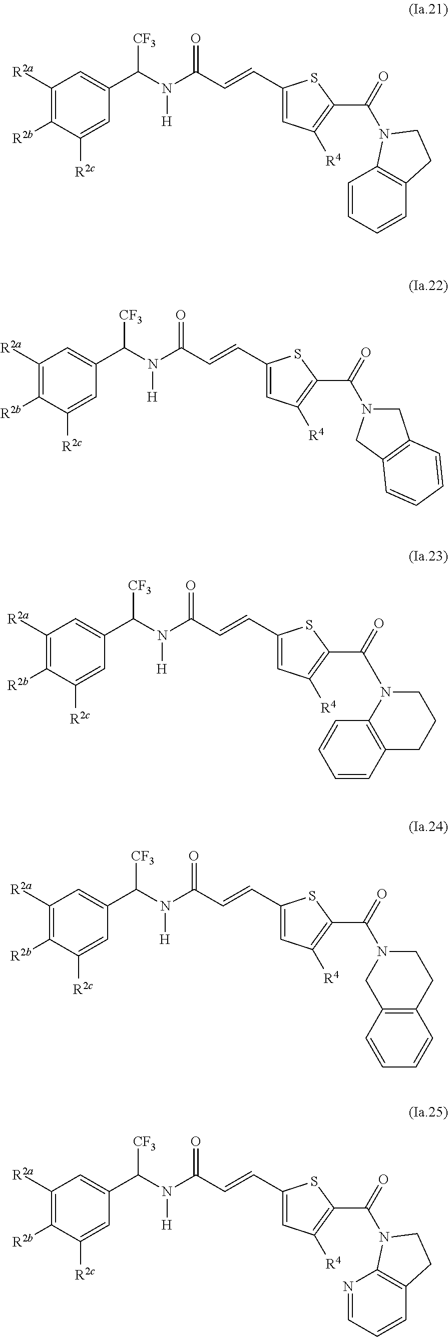

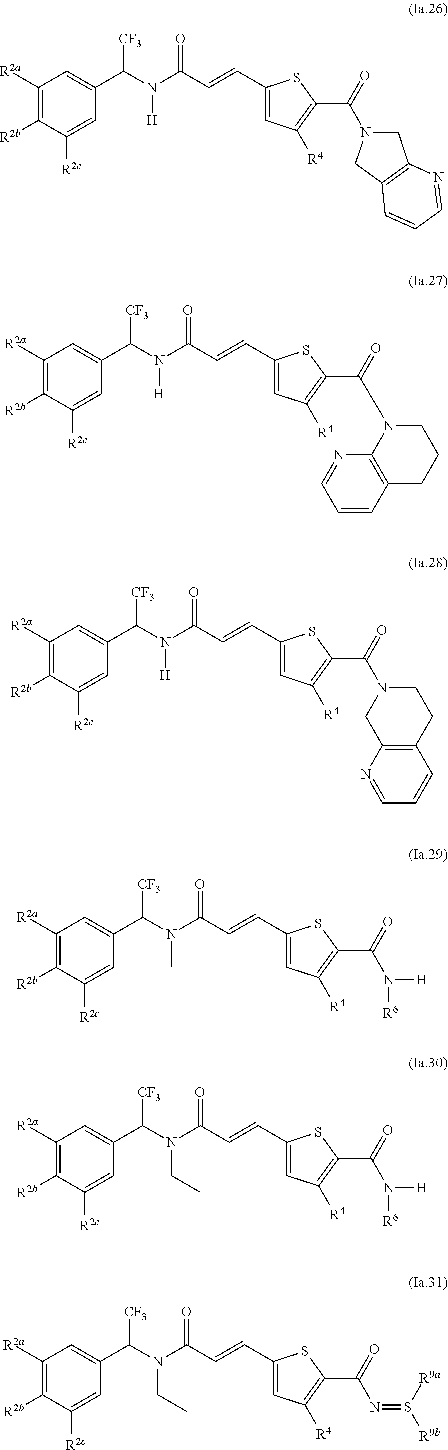

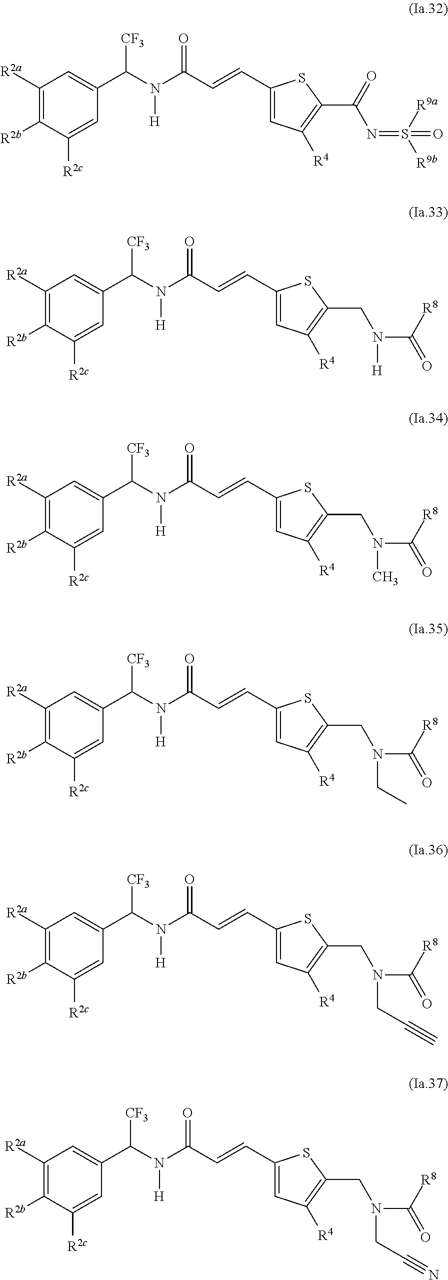

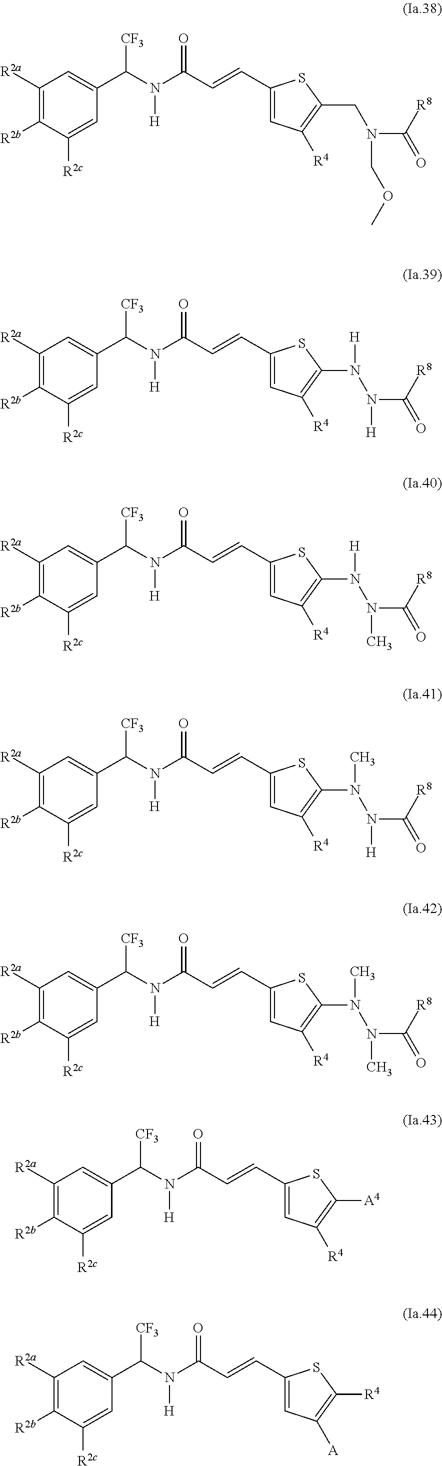

### Table 1

Compounds of the formula Ia.1 in which Rub is hydrogen, and the combination of R^{2a}, R^{2b}, R^{2c} and R⁴ for a compound corresponds in each case to one row of Table A

### Table 2

Compounds of the formula Ia.1 in which R^{14b} is methyl, and the combination of R^{2a}, R^{2b}, R^{2c} and R⁴ for a compound corresponds in each case to one row of Table A

### Table 3

Compounds of the formula Ia.1 in which R^{14b} is ethyl, and the combination of R^{2a}, R^{2b}, R^{2c} and R⁴ for a compound corresponds in each case to one row of Table A

### Table 4

Compounds of the formula Ia.1 in which R^{14b} is propyl, and the combination of R^{2a}, R^{2b}, R^{2b} and R⁴ for a compound corresponds in each case to one row of Table A

### Table 5

Compounds of the formula Ia.1 in which R^{14b} is isopropyl, and the combination of R^{2a}, R^{2b}, R^{2c} and R⁴ for a compound corresponds in each case to one row of Table A

### Table 6

Compounds of the formula Ia.1 in which R^{14b} is n-butyl, and the combination of R^{2a}, R^{2b}, R^{2c} and R⁴ for a compound corresponds in each case to one row of Table A

### Table 7

Compounds of the formula Ia.1 in which R^{14b} is sec-butyl, and the combination of R^{2a}, R^{2b}, R^{2c} and R⁴ for a compound corresponds in each case to one row of Table A

### Table 8

Compounds of the formula Ia.1 in which R^{14b} is isobutyl, and the combination of R^{2a}, R^{2b}, R^{2c} and R⁴ for a compound corresponds in each case to one row of Table A

### Table 9

Compounds of the formula Ia.1 in which R^{14b} is tert-butyl, and the combination of R^{2a}, R^{2b}, R^{2c} and R⁴ for a compound corresponds in each case to one row of Table A

### Table 10

Compounds of the formula Ia.1 in which R^{14b} is propargyl, and the combination of R^{2a}, R^{2b}, R^{2c} and R⁴ for a compound corresponds in each case to one row of Table A

### Table 11

Compounds of the formula Ia.1 in which R^{14b} is cyclopropyl, and the combination of R^{2a}, R^{2b}, R^{2c} and R⁴ for a compound corresponds in each case to one row of Table A

### Table 12

Compounds of the formula Ia.1 in which R^{14b} is 1-cyanocycloprop-1-yl, and the combination of R^{2a}, R^{2b}, R^{2c} and R⁴ for a compound corresponds in each case to one row of Table A

### Table 13

Compounds of the formula Ia.1 in which R^{14b} is 2,2-difluoroethyl, and the combination of R^{2a}, R^{2b}, R^{2c} and R⁴ for a compound corresponds in each case to one row of Table A

### Table 14

Compounds of the formula Ia.1 in which R^{14b} is 2,2,2-trifluoroethyl, and the combination of R^{2a}, R^{2b}, R^{2c} and R⁴ for a compound corresponds in each case to one row of Table A

### Table 15

Compounds of the formula Ia.1 in which R^{14b} is CH₂—CN, and the combination of R^{2a}, R^{2b}, R^{2c} and R⁴ for a compound corresponds in each case to one row of Table A

### Table 16

Compounds of the formula Ia.1 in which R^{14b} is CH₂-cyclopropyl, and the combination of R^{2a}, R^{2b}, R^{2c} and R⁴ for a compound corresponds in each case to one row of Table A

### Table 17

Compounds of the formula Ia.1 in which R^{14b} is CH₂-(1-cyanocycloprop-1-yl), and the combination of R^{2a}, R^{2b}, R^{2c} and R⁴ for a compound corresponds in each case to one row of Table A

### Table 18

Compounds of the formula Ia.1 in which R^{14b} is thietan-3-yl, and the combination of R^{2a}, R^{2b}, R^{2c} and R⁴ for a compound corresponds in each case to one row of Table A

### Table 19

Compounds of the formula Ia.1 in which R^{14b} is 1-oxo-thietan-3-yl, and the combination of R^{2a}, R^{2b}, R^{2c} and R⁴ for a compound corresponds in each case to one row of Table A

### Table 20

Compounds of the formula Ia.1 in which R^{14b} is 1,1-dioxo-thietan-3-yl, and the combination of R^{2a}, R^{2b}, R^{2c} and R⁴ for a compound corresponds in each case to one row of Table A

### Tables 21 to 40

Compounds of the formula Ia.2 in which R^{14b} is as defined in any of tables 1 to 20 and the combination of R^{2a}, R^{2b}, R^{2c} and R⁴ for a compound corresponds in each case to one row of Table A

### Tables 41 to 60

Compounds of the formula Ia.3 in which R^{14b} is as defined in any of tables 1 to 20 and the combination of R^{2a}, R^{2b}, R^{2c} and R⁴ for a compound corresponds in each case to one row of Table A

### Tables 61 to 80

Compounds of the formula Ia.4 in which R^{14b} is as defined in any of tables 1 to 20 and the combination of R^{2a}, R^{2b}, R^{2c} and R⁴ for a compound corresponds in each case to one row of Table A

### Table 81

Compounds of the formula Ia.5 in which R⁶ is methyl and the combination of R^{2a}, R^{2b}, R^{2c} and R⁴ for a compound corresponds in each case to one row of Table A

### Table 82

Compounds of the formula Ia.5 in which R⁶ is ethyl and the combination of R^{2a}, R^{2b}, R^{2c} and R⁴ for a compound corresponds in each case to one row of Table A

### Table 83

Compounds of the formula Ia.5 in which R⁶ is n-propyl and the combination of R^{2a}, R^{2b}, R^{2c} and R⁴ for a compound corresponds in each case to one row of Table A

### Table 84

Compounds of the formula Ia.5 in which R⁶ is isopropyl and the combination of R^{2a}, R^{2b}, R^{2c} and R⁴ for a compound corresponds in each case to one row of Table A

### Table 85

Compounds of the formula Ia.5 in which R⁶ is n-butyl and the combination of R^{2a}, R^{2b}, R^{2c} and R⁴ for a compound corresponds in each case to one row of Table A

### Table 86

Compounds of the formula Ia.5 in which R⁶ is sec-butyl and the combination of R^{2a}, R^{2b}, R^{2c} and R⁴ for a compound corresponds in each case to one row of Table A

### Table 87

Compounds of the formula Ia.5 in which R⁶ is isobutyl and the combination of R^{2a}, R^{2b}, R^{2c} and R⁴ for a compound corresponds in each case to one row of Table A

### Table 88

Compounds of the formula Ia.5 in which R⁶ is tert-butyl and the combination of R^{2a}, R^{2b}, R^{2c} and R⁴ for a compound corresponds in each case to one row of Table A

### Table 89

Compounds of the formula Ia.5 in which R⁶ is —CH₂CN and the combination of R^{2a}, R^{2b}, R^{2c} and R⁴ for a compound corresponds in each case to one row of Table A

### Table 90

Compounds of the formula Ia.5 in which R⁶ is —CH₂C≡CH and the combination of R^{2a}, R^{2b}, R^{2c} and R⁴ for a compound corresponds in each case to one row of Table A

### Table 91

Compounds of the formula Ia.5 in which R⁶ is —CH₂CH₂OCH₃ and the combination of R^{2a}, R^{2b}, R^{2c} and R⁴ for a compound corresponds in each case to one row of Table A

### Table 92

Compounds of the formula Ia.5 in which R⁶ is —CH₂CH₂OCH₂CH₃ and the combination of R^{2a}, R^{2b}, R^{2c} and R⁴ for a compound corresponds in each case to one row of Table A

### Table 93

Compounds of the formula Ia.5 in which R⁶ is —CH₂CH₂SCH₃ and the combination of R^{2a}, R^{2b}, R^{2c} and R⁴ for a compound corresponds in each case to one row of Table A

### Table 94

Compounds of the formula Ia.5 in which R⁶ is —CH₂CH₂S(O)CH₃ and the combination of R^{2a}, R^{2b}, R^{2c} and R⁴ for a compound corresponds in each case to one row of Table A

### Table 95

Compounds of the formula Ia.5 in which R⁶ is —CH₂CH₂S(O)₂CH₃ and the combination of R^{2a}, R^{2b}, R^{2c} and R⁴ for a compound corresponds in each case to one row of Table A

### Table 96

Compounds of the formula Ia.5 in which R⁶ is —CH₂CH₂SCH₂CH₃ and the combination of R^{2a}, R^{2b}, R^{2c} and R⁴ for a compound corresponds in each case to one row of Table A

### Table 97

Compounds of the formula Ia.5 in which R⁶ is —CH₂CH₂S(O)CH₂CH₃ and the combination of R^{2a}, R^{2b}, R^{2c} and R⁴ for a compound corresponds in each case to one row of Table A

### Table 98

Compounds of the formula Ia.5 in which R⁶ is —CH₂CH₂S(O)₂CH₂CH₃ and the combination of R^{2a}, R^{2b}, R^{2c} and R⁴ for a compound corresponds in each case to one row of Table A

### Table 99

Compounds of the formula Ia.5 in which R⁶ is —CH(CH₃)CH₂SCH₃ and the combination of R^{2a}, R^{2b}, R^{2c} and R⁴ for a compound corresponds in each case to one row of Table A

### Table 100

Compounds of the formula Ia.5 in which R⁶ is —CH(CH₃)CH₂S(O)CH₃ and the combination of R^{2a}, R^{2b}, R^{2c} and R⁴ for a compound corresponds in each case to one row of Table A

### Table 101

Compounds of the formula Ia.5 in which R⁶ is —CH(CH₃)CH₂S(O)₂CH₃ and the combination of R^{2a}, R^{2b}, R^{2c} and R⁴ for a compound corresponds in each case to one row of Table A

### Table 102

Compounds of the formula Ia.5 in which R⁶ is —C(CH₃)₂CH₂SCH₃ and the combination of R^{2a}, R^{2b}, R^{2c} and R⁴ for a compound corresponds in each case to one row of Table A

### Table 103

Compounds of the formula Ia.5 in which R⁶ is —C(CH₃)₂CH₂S(O)CH₃ and the combination of R^{2a}, R^{2b}, R^{2c} and R⁴ for a compound corresponds in each case to one row of Table A

### Table 104

Compounds of the formula Ia.5 in which R⁶ is —C(CH₃)₂CH₂S(O)₂CH₃ and the combination of R^{2a}, R^{2b}, R^{2c} and R⁴ for a compound corresponds in each case to one row of Table A

### Table 105

Compounds of the formula Ia.5 in which R⁶ is CF₃ and the combination of R^{2a}, R^{2b}, R^{2c} and R⁴ for a compound corresponds in each case to one row of Table A

### Table 106

Compounds of the formula Ia.5 in which R⁶ is CH₂CHF₂ and the combination of R^{2a}, R^{2b}, R^{2c} and R⁴ for a compound corresponds in each case to one row of Table A

### Table 107

Compounds of the formula Ia.5 in which R⁶ is CH₂CF₃ and the combination of R^{2a}, R^{2b}, R^{2c} and R⁴ for a compound corresponds in each case to one row of Table A

### Table 108

Compounds of the formula Ia.5 in which R⁶ is CH₂CH₂CF₃ and the combination of R^{2a}, R^{2b}, R^{2c} and R⁴ for a compound corresponds in each case to one row of Table A

### Table 109

Compounds of the formula Ia.5 in which R⁶ is CH(CH₃)CH₂F and the combination of R^{2a}, R^{2b}, R^{2b} and R⁴ for a compound corresponds in each case to one row of Table A

### Table 110

Compounds of the formula Ia.5 in which R⁶ is CH(CH₃)CHF₂ and the combination of R^{2a}, R^{2b}, R^{2c} and R⁴ for a compound corresponds in each case to one row of Table A

### Table 111

Compounds of the formula Ia.5 in which R⁶ is CH(CH₃)CF₃ and the combination of R^{2a}, R^{2b}, R^{2c} and R⁴ for a compound corresponds in each case to one row of Table A

### Table 112

Compounds of the formula Ia.5 in which R⁶ is CH(CF₃)₂ and the combination of R^{2a}, R^{2b}, R^{2c} and R⁴ for a compound corresponds in each case to one row of Table A

### Table 113

Compounds of the formula Ia.5 in which R⁶ is cyclopropyl and the combination of R^{2a}, R^{2b}, R^{2c} and R⁴ for a compound corresponds in each case to one row of Table A

### Table 114

Compounds of the formula Ia.5 in which R⁶ is 1-cyanocyclopropyl and the combination of R^{2a}, R^{2b}, R^{2c} and R⁴ for a compound corresponds in each case to one row of Table A

### Table 115

Compounds of the formula Ia.5 in which R⁶ is cyclobutyl and the combination of R^{2a}, R^{2b}, R^{2c} and R⁴ for a compound corresponds in each case to one row of Table A

### Table 116

Compounds of the formula Ia.5 in which R⁶ is cyclopentyl and the combination of R^{2a}, R^{2b}, R^{2c} and R⁴ for a compound corresponds in each case to one row of Table A

### Table 117

Compounds of the formula Ia.5 in which R⁶ is cyclohexyl and the combination of R^{2a}, R^{2b}, R^{2c} and R⁴ for a compound corresponds in each case to one row of Table A

### Table 118

Compounds of the formula Ia.5 in which R⁶ is —CH₂-cyclopropyl and the combination of R^{2a}, R^{2b}, R^{2c} and R⁴ for a compound corresponds in each case to one row of Table A

### Table 119

Compounds of the formula Ia.5 in which R⁶ is —CH₂-1-cyanocyclopropyl and the combination of R^{2a}, R^{2b}, R^{2c} and R⁴ for a compound corresponds in each case to one row of Table A

### Table 120

Compounds of the formula Ia.5 in which R⁶ is —CH₂-1-fluorocyclopropyl and the combination of R^{2a}, R^{2b}, R^{2c} and R⁴ for a compound corresponds in each case to one row of Table A

### Table 121

Compounds of the formula Ia.5 in which R⁶ is —CH₂-cyclobutyl and the combination of R^{2a}, R^{2b}, R^{2c} and R⁴ for a compound corresponds in each case to one row of Table A

### Table 122

Compounds of the formula Ia.5 in which R⁶ is —CH₂-cyclopentyl and the combination of R^{2a}, R^{2b}, R^{2b} and R⁴ for a compound corresponds in each case to one row of Table A

### Table 123

Compounds of the formula Ia.5 in which R⁶ is —CH₂-cyclohexyl and the combination of R^{2a}, R^{2b}, R^{2c} and R⁴ for a compound corresponds in each case to one row of Table A

### Table 124

Compounds of the formula Ia.5 in which R⁶ is 1,1-difluorocyclobut-3-yl and the combination of R^{2a}, R^{2b}, R^{2c} and R⁴ for a compound corresponds in each case to one row of Table A

### Table 125

Compounds of the formula Ia.5 in which R⁶ is —CH₂-1,1-difluorocyclobut-3-yl and the combination of R^{2a}, R^{2b}, R^{2c} and R⁴ for a compound corresponds in each case to one row of Table A

### Table 126

Compounds of the formula Ia.5 in which R⁶ is thietan-3-yl and the combination of R^{2a}, R^{2b}, R^{2c} and R⁴ for a compound corresponds in each case to one row of Table A

### Table 127

Compounds of the formula Ia.5 in which R⁶ is 1-oxo-thietan-3-yl and the combination of R^{2a}, R^{2b}, R^{2c} and R⁴ for a compound corresponds in each case to one row of Table A

### Table 128

Compounds of the formula Ia.5 in which R⁶ is 1,1-dioxo-thietan-3-yl and the combination of R^{2a}, R^{2b}, R^{2c} and R⁴ for a compound corresponds in each case to one row of Table A

### Table 129

Compounds of the formula Ia.5 in which R⁶ is 3-methyl-thietan-3-yl and the combination of R^{2a}, R^{2b}, R^{2c} and R⁴ for a compound corresponds in each case to one row of Table A

### Table 130

Compounds of the formula Ia.5 in which R⁶ is 3-methyl-1-oxo-thietan-3-yl and the combination of R^{2a}, R^{2b}, R^{2c} and R⁴ for a compound corresponds in each case to one row of Table A

### Table 131

Compounds of the formula Ia.5 in which R⁶ is 3-methyl-1,1-dioxo-thietan-3-yl and the combination of R^{2a}, R^{2b}, R^{2c} and R⁴ for a compound corresponds in each case to one row of Table A

### Table 132

Compounds of the formula Ia.5 in which R⁶ is —CH₂-thietan-3-yl and the combination of R^{2a}, R^{2b}, R^{2c} and R⁴ for a compound corresponds in each case to one row of Table A

### Table 133

Compounds of the formula Ia.5 in which R⁶ is —CH₂-(1-oxo-thietan-3-yl) and the combination of R^{2a}, R^{2b}, R^{2c} and R⁴ for a compound corresponds in each case to one row of Table A

### Table 134

Compounds of the formula Ia.5 in which R⁶ is —CH₂-(1,1-dioxo-thietan-3-yl) and the combination of R^{2a}, R^{2b}, R^{2c} and R⁴ for a compound corresponds in each case to one row of Table A

### Table 135

Compounds of the formula Ia.5 in which R⁶ is tetrahydrothiophen-3-yl and the combination of R^{2a}, R^{2b}, R^{2c} and R⁴ for a compound corresponds in each case to one row of Table A

### Table 136

Compounds of the formula Ia.5 in which R⁶ is 1-oxo-tetrahydrothiphen-3-yl and the combination of R^{2a}, R^{2b}, R^{2c} and R⁴ for a compound corresponds in each case to one row of Table A

### Table 137

Compounds of the formula Ia.5 in which R⁶ is 1,1-dioxo-tetrahydrothiophen-3-yl and the combination of R^{2a}, R^{2b}, R^{2c} and R⁴ for a compound corresponds in each case to one row of Table A

### Table 138

Compounds of the formula Ia.5 in which R⁶ is phenyl and the combination of R^{2a}, R^{2b}, R^{2c} and R⁴ for a compound corresponds in each case to one row of Table A

### Table 139

Compounds of the formula Ia.5 in which R⁶ is pyridin-2-yl and the combination of R^{2a}, R^{2b}, R^{2c} and R⁴ for a compound corresponds in each case to one row of Table A

### Table 140

Compounds of the formula Ia.5 in which R⁶ is pyridin-3-yl and the combination of R^{2a}, R^{2b}, R^{2c} and R⁴ for a compound corresponds in each case to one row of Table A

### Table 141

Compounds of the formula Ia.5 in which R⁶ is pyridin-4-yl and the combination of R^{2a}, R^{2b}, R^{2c} and R⁴ for a compound corresponds in each case to one row of Table A

### Table 142

Compounds of the formula Ia.5 in which R⁶ is —NH-phenyl and the combination of R^{2a}, R^{2b}, R^{2c} and R⁴ for a compound corresponds in each case to one row of Table A

### Table 143

Compounds of the formula Ia.5 in which R⁶ is —NH-pyridin-2-yl and the combination of R^{2a}, R^{2b}, R^{2c} and R⁴ for a compound corresponds in each case to one row of Table A

### Table 144

Compounds of the formula Ia.5 in which R⁶ is —NH-pyridin-3-yl and the combination of R^{2a}, R^{2b}, R^{2c} and R⁴ for a compound corresponds in each case to one row of Table A

### Table 145

Compounds of the formula Ia.5 in which R⁶ is —NH-pyridin-4-yl and the combination of R^{2a}, R^{2b}, R^{2c} and R⁴ for a compound corresponds in each case to one row of Table A

### Table 146

Compounds of the formula Ia.5 in which R⁶ is —CH₂—COOCH₃ and the combination of R^{2a}, R^{2b}, R^{2c} and R⁴ for a compound corresponds in each case to one row of Table A

### Table 147

Compounds of the formula Ia.5 in which R⁶ is —CH₂—COO—CH₂CH₃ and the combination of R^{2a}, R^{2b}, R^{2c} and R⁴ for a compound corresponds in each case to one row of Table A

### Table 148

Compounds of the formula Ia.5 in which R⁶ is —CH₂—CONH—CH₃ and the combination of R^{2a}, R^{2b}, R^{2c} and R⁴ for a compound corresponds in each case to one row of Table A

### Table 149

Compounds of the formula Ia.5 in which R⁶ is —CH₂—CONH—CH₂CH₃ and the combination of R^{2a}, R^{2b}, R^{2c} and R⁴ for a compound corresponds in each case to one row of Table A

### Table 150

Compounds of the formula Ia.5 in which R⁶ is —CH₂—CONH—CH₂CN and the combination of R^{2a}, R^{2b}, R^{2c} and R⁴ for a compound corresponds in each case to one row of Table A

### Table 151

Compounds of the formula Ia.5 in which R⁶ is —CH₂—CONH—CH₂CF₃ and the combination of R^{2a}, R^{2b}, R^{2c} and R⁴ for a compound corresponds in each case to one row of Table A

### Table 152

Compounds of the formula Ia.5 in which R⁶ is —CH₂—CONH-cyclopropyl and the combination of R^{2a}, R^{2b}, R^{2c} and R⁴ for a compound corresponds in each case to one row of Table A

### Table 153

Compounds of the formula Ia.5 in which R⁶ is —CH₂—CONH-isopropyl and the combination of R^{2a}, R^{2b}, R^{2c} and R⁴ for a compound corresponds in each case to one row of Table A

### Table 154

Compounds of the formula Ia.5 in which R⁶ is —CH₂—CONH—CH(CF₃)CH₃ and the combination of R^{2a}, R^{2b}, R^{2c} and R⁴ for a compound corresponds in each case to one row of Table A

### Table 155

Compounds of the formula Ia.5 in which R⁶ is —CH₂—CONH—CH(CF₃)₂ and the combination of R^{2a}, R^{2b}, R^{2c} and R⁴ for a compound corresponds in each case to one row of Table A

### Table 156

Compounds of the formula Ia.5 in which R⁶ is —CH₂—CONH-thietan-3-yl and the combination of R^{2a}, R^{2b}, R^{2c} and R⁴ for a compound corresponds in each case to one row of Table A

### Table 157

Compounds of the formula Ia.5 in which R⁶ is —CH₂—CONH-1-oxo-thietan-3-yl and the combination of R^{2a}, R^{2b}, R^{2c} and R⁴ for a compound corresponds in each case to one row of Table A

### Table 158

Compounds of the formula Ia.5 in which R⁶ is —CH₂—CONH-1,1-dioxo-thietan-3-yl and the combination of R^{2a}, R^{2b}, R^{2c} and R⁴ for a compound corresponds in each case to one row of Table A

### Table 159

Compounds of the formula Ia.5 in which R⁶ is benzyl and the combination of R^{2a}, R^{2b}, R^{2c} and R⁴ for a compound corresponds in each case to one row of Table A

### Table 160

Compounds of the formula Ia.5 in which R⁶ is 2-fluorobenzyl and the combination of R^{2a}, R^{2b}, R^{2c} and R⁴ for a compound corresponds in each case to one row of Table A

### Table 161

Compounds of the formula Ia.5 in which R⁶ is 3-fluorobenzyl and the combination of R^{2a}, R^{2b}, R^{2c} and R⁴ for a compound corresponds in each case to one row of Table A

### Table 162

Compounds of the formula Ia.5 in which R⁶ is 4-fluorobenzyl and the combination of R^{2a}, R^{2b}, R^{2c} and R⁴ for a compound corresponds in each case to one row of Table A

### Table 163

Compounds of the formula Ia.5 in which R⁶ is 2-chlorobenzyl and the combination of R^{2a}, R^{2b}, R^{2c} and R⁴ for a compound corresponds in each case to one row of Table A

### Table 164

Compounds of the formula Ia.5 in which R⁶ is 3-chlorobenzyl and the combination of R^{2a}, R^{2b}, R^{2c} and R⁴ for a compound corresponds in each case to one row of Table A

### Table 165

Compounds of the formula Ia.5 in which R⁶ is 4-chlorobenzyl and the combination of R^{2a}, R^{2b}, R^{2c} and R⁴ for a compound corresponds in each case to one row of Table A

### Table 166

Compounds of the formula Ia.5 in which R⁶ is 2-methoxybenzyl and the combination of R^{2a}, R^{2b}, R^{2c} and R⁴ for a compound corresponds in each case to one row of Table A

### Table 167

Compounds of the formula Ia.5 in which R⁶ is 3-methoxybenzyl and the combination of R^{2a}, R^{2b}, R^{2c} and R⁴ for a compound corresponds in each case to one row of Table A

### Table 168

Compounds of the formula Ia.5 in which R⁶ is 4-methoxybenzyl and the combination of R^{2a}, R^{2b}, R^{2c} and R⁴ for a compound corresponds in each case to one row of Table A

### Table 169

Compounds of the formula Ia.5 in which R⁶ is pyridin-2-yl-methyl and the combination of R^{2a}, R^{2b}, R^{2c} and R⁴ for a compound corresponds in each case to one row of Table A

### Table 170

Compounds of the formula Ia.5 in which R⁶ is pyridin-3-yl-methyl and the combination of R^{2a}, R^{2b}, R^{2c} and R⁴ for a compound corresponds in each case to one row of Table A

### Table 171

Compounds of the formula Ia.5 in which R⁶ is 6-chloro-pyridin-3-yl-methyl and the combination of R^{2a}, R^{2b}, R^{2c} and R⁴ for a compound corresponds in each case to one row of Table A

### Table 172

Compounds of the formula Ia.5 in which R⁶ is pyridin-4-yl-methyl and the combination of R^{2a}, R^{2b}, R^{2c} and R⁴ for a compound corresponds in each case to one row of Table A

### Table 173

Compounds of the formula Ia.5 in which R⁶ is thien-2-yl-methyl and the combination of R^{2a}, R^{2b}, R^{2c} and R⁴ for a compound corresponds in each case to one row of Table A

### Table 174

Compounds of the formula Ia.5 in which R⁶ is thien-3-yl-methyl and the combination of R^{2a}, R^{2b}, R^{2c} and R⁴ for a compound corresponds in each case to one row of Table A

### Table 175

Compounds of the formula Ia.5 in which R⁶ is thiazol-2-yl-methyl and the combination of R^{2a}, R^{2b}, R^{2c} and R⁴ for a compound corresponds in each case to one row of Table A

### Table 176

Compounds of the formula Ia.5 in which R⁶ is thiazol-4-yl-methyl and the combination of R^{2a}, R^{2b}, R^{2c} and R⁴ for a compound corresponds in each case to one row of Table A

### Table 177

Compounds of the formula Ia.5 in which R⁶ is thiazol-5-yl-methyl and the combination of R^{2a}, R^{2b}, R^{2c} and R⁴ for a compound corresponds in each case to one row of Table A

### Table 178

Compounds of the formula Ia.5 in which R⁶ is 2-chloro-thiazol-5-yl-methyl and the combination of R^{2a}, R^{2b}, R^{2c} and R⁴ for a compound corresponds in each case to one row of Table A

### Table 179

Compounds of the formula Ia.5 in which R⁶ is isothiazol-3-yl-methyl and the combination of R^{2a}, R^{2b}, R^{2c} and R⁴ for a compound corresponds in each case to one row of Table A

### Table 180

Compounds of the formula Ia.5 in which R⁶ is isothiazol-4-yl-methyl and the combination of R^{2a}, R^{2b}, R^{2c} and R⁴ for a compound corresponds in each case to one row of Table A

### Table 181

Compounds of the formula Ia.5 in which R⁶ is isothiazol-5-yl-methyl and the combination of R^{2a}, R^{2b}, R^{2c} and R⁴ for a compound corresponds in each case to one row of Table A

### Table 182

Compounds of the formula Ia.5 in which R⁶ is oxazol-2-yl-methyl and the combination of R^{2a}, R^{2b}, R^{2c} and R⁴ for a compound corresponds in each case to one row of Table A

### Table 183

Compounds of the formula Ia.5 in which R⁶ is oxazol-4-yl-methyl and the combination of R^{2a}, R^{2b}, R^{2c} and R⁴ for a compound corresponds in each case to one row of Table A

### Table 184

Compounds of the formula Ia.5 in which R⁶ is oxazol-5-yl-methyl and the combination of R^{2a}, R^{2b}, R^{2c} and R⁴ for a compound corresponds in each case to one row of Table A

### Table 185

Compounds of the formula Ia.5 in which R⁶ is isoxazol-3-yl-methyl and the combination of R^{2a}, R^{2b}, R^{2c} and R⁴ for a compound corresponds in each case to one row of Table A

### Table 186

Compounds of the formula Ia.5 in which R⁶ is isoxazol-4-yl-methyl and the combination of R^{2a}, R^{2b}, R^{2c} and R⁴ for a compound corresponds in each case to one row of Table A

### Table 187

Compounds of the formula Ia.5 in which R⁶ is isoxazol-5-yl-methyl and the combination of R^{2a}, R^{2b}, R^{2c} and R⁴ for a compound corresponds in each case to one row of Table A

### Table 188

Compounds of the formula Ia.5 in which R⁶ is —CONH₂ and the combination of R^{2a}, R^{2b}, R^{2c} and R⁴ for a compound corresponds in each case to one row of Table A

### Table 189

Compounds of the formula Ia.5 in which R⁶ is —CONH—CH₃ and the combination of R^{2a}, R^{2b}, R^{2c} and R⁴ for a compound corresponds in each case to one row of Table A

### Table 190

Compounds of the formula Ia.5 in which R⁶ is —CONH—CH₂CH₃ and the combination of R^{2a}, R^{2b}, R^{2c} and R⁴ for a compound corresponds in each case to one row of Table A

### Table 191

Compounds of the formula Ia.5 in which R⁶ is —CONH—CH₂CH₂CH₃ and the combination of R^{2a}, R^{2b}, R^{2c} and R⁴ for a compound corresponds in each case to one row of Table A

### Table 192

Compounds of the formula Ia.5 in which R⁶ is —CONH-cyclopropyl and the combination of R^{2a}, R^{2b}, R^{2c} and R⁴ for a compound corresponds in each case to one row of Table A

### Table 193

Compounds of the formula Ia.5 in which R⁶ is —CONH—CH₂-cyclopropyl and the combination of R^{2a}, R^{2b}, R^{2c} and R⁴ for a compound corresponds in each case to one row of Table A

### Table 194

Compounds of the formula Ia.5 in which R⁶ is —CONH-phenyl and the combination of R^{2a}, R^{2b}, R^{2c} and R⁴ for a compound corresponds in each case to one row of Table A

### Table 195

Compounds of the formula Ia.5 in which R⁶ is —CONH-benzyl and the combination of R^{2a}, R^{2b}, R^{2c} and R⁴ for a compound corresponds in each case to one row of Table A

### Table 196

Compounds of the formula Ia.5 in which R⁶ is tetrahydrofuran-2-on-3-yl and the combination of R^{2a}, R^{2b}, R^{2c} and R⁴ for a compound corresponds in each case to one row of Table A

### Table 197

Compounds of the formula Ia.5 in which R⁶ is —CH₂—CO—CH₃ and the combination of R^{2a}, R^{2b}, R^{2c} and R⁴ for a compound corresponds in each case to one row of Table A

### Table 198

Compounds of the formula Ia.5 in which R⁶ is —CH₂—CO—CH₂CH₃ and the combination of R^{2a}, R^{2b}, R^{2c} and R⁴ for a compound corresponds in each case to one row of Table A

### Table 199

Compounds of the formula Ia.5 in which R⁶ is —CH₂—CO—CH₂CH₂CH₃ and the combination of R^{2a}, R^{2b}, R^{2c} and R⁴ for a compound corresponds in each case to one row of Table A

### Table 200

Compounds of the formula Ia.5 in which R⁶ is —CH₂—CO—CH(CH₃)₂ and the combination of R^{2a}, R^{2b}, R^{2c} and R⁴ for a compound corresponds in each case to one row of Table A

### Table 201

Compounds of the formula Ia.5 in which R⁶ is —CH₂—CO—CF₃ and the combination of R^{2a}, R^{2b}, R^{2c} and R⁴ for a compound corresponds in each case to one row of Table A

### Table 202

Compounds of the formula Ia.5 in which R⁶ is —CH₂—CO—CH₂CF₃ and the combination of R^{2a}, R^{2b}, R^{2c} and R⁴ for a compound corresponds in each case to one row of Table A

### Table 203

Compounds of the formula Ia.5 in which R⁶ is —CH₂—CO—CH₂CHF₂ and the combination of R^{2a}, R^{2b}, R^{2c} and R⁴ for a compound corresponds in each case to one row of Table A

### Table 204

Compounds of the formula Ia.5 in which R⁶ is —S(═O)₂—CH₃ and the combination of R^{2a}, R^{2b}, R^{2c} and R⁴ for a compound corresponds in each case to one row of Table A

### Table 205

Compounds of the formula Ia.5 in which R⁶ is —S(═O)₂—CH₂CH₃ and the combination of R^{2a}, R^{2b}, R^{2c} and R⁴ for a compound corresponds in each case to one row of Table A

### Table 206

Compounds of the formula Ia.5 in which R⁶ is —S(═O)₂—CH₂CH₂CH₃ and the combination of R^{2a}, R^{2b}, R^{2c} and R⁴ for a compound corresponds in each case to one row of Table A

### Table 207

Compounds of the formula Ia.5 in which R⁶ is —S(═O)₂—CH(CH₃)₂ and the combination of R^{2a}, R^{2b}, R^{2c} and R⁴ for a compound corresponds in each case to one row of Table A

### Table 208

Compounds of the formula Ia.5 in which R⁶ is —S(═O)₂—N(CH₃)CH₃ and the combination of R^{2a}, R^{2b}, R^{2c} and R⁴ for a compound corresponds in each case to one row of Table A

### Table 209

Compounds of the formula Ia.5 in which R⁶ is —S(═O)₂—N(CH₃)CH₂CH₃ and the combination of R^{2a}, R^{2b}, R^{2c} and R⁴ for a compound corresponds in each case to one row of Table A

### Table 210

Compounds of the formula Ia.5 in which R⁶ is —S(═O)₂—N(CH₃)CH₂CH₂CH₃ and the combination of R^{2a}, R^{2b}, R^{2c} and R⁴ for a compound corresponds in each case to one row of Table A

### Table 211

Compounds of the formula Ia.5 in which R⁶ is —S(═O)₂—N(CH₃)CH(CH₃)₂ and the combination of R^{2a}, R^{2b}, R^{2c} and R⁴ for a compound corresponds in each case to one row of Table A

### Table 212

Compounds of the formula Ia.5 in which R⁶ is —S(═O)₂—N(CH₂CH₃)CH₂CH₃ and the combination of R^{2a}, R^{2b}, R^{2c} and R⁴ for a compound corresponds in each case to one row of Table A

### Table 213

Compounds of the formula Ia.5 in which R⁶ is —S(═O)₂—N(CH₂CH₃)CH₂CH₂CH₃ and the combination of R^{2a}, R^{2b}, R^{2c} and R⁴ for a compound corresponds in each case to one row of Table A

### Table 214

Compounds of the formula Ia.5 in which R⁶ is —S(═O)₂—N(CH₂CH₃)CH(CH₃)₂ and the combination of R^{2a}, R^{2b}, R^{2c} and R⁴ for a compound corresponds in each case to one row of Table A

### Table 215

Compounds of the formula Ia.5 in which R⁶ is —S(═O)₂—N(CH₂CH₂CH₃)CH₂CH₂CH₃ and the combination of R^{2a}, R^{2b}, R^{2c} and R⁴ for a compound corresponds in each case to one row of Table A

### Table 216

Compounds of the formula Ia.5 in which R⁶ is —S(═O)₂—N(CH₂CH₂CH₃)CH(CH₃)₂ and the combination of R^{2a}, R^{2b}, R^{2c} and R⁴ for a compound corresponds in each case to one row of Table A

### Table 217

Compounds of the formula Ia.5 in which R⁶ is —S(═O)₂—N(CH(CH₃)₂)CH(CH₃)₂ and the combination of R^{2a}, R^{2b}, R^{2c} and R⁴ for a compound corresponds in each case to one row of Table A

### Table 218

Compounds of the formula Ia.5 in which R⁶ is —P(═O)(OCH₃)₂ and the combination of R^{2a}, R^{2b}, R^{2c} and R⁴ for a compound corresponds in each case to one row of Table A

### Table 219

Compounds of the formula Ia.5 in which R⁶ is —P(═O)(OCH₂CH₃)₂ and the combination of R^{2a}, R^{2b}, R^{2c} and R⁴ for a compound corresponds in each case to one row of Table A

### Table 220

Compounds of the formula Ia.5 in which R⁶ is —P(═O)(OCH₂CH₂CH₃)₂ and the combination of R^{2a}, R^{2b}, R^{2c} and R⁴ for a compound corresponds in each case to one row of Table A

### Table 221

Compounds of the formula Ia.5 in which R⁶ is —P(═O)(O-phenyl)₂ and the combination of R^{2a}, R^{2b}, R^{2c} and R⁴ for a compound corresponds in each case to one row of Table A

### Table 222

Compounds of the formula Ia.5 in which R⁶ is —P(═S)(OCH₃)₂ and the combination of R^{2a}, R^{2b}, R^{2c} and R⁴ for a compound corresponds in each case to one row of Table A

### Table 223

Compounds of the formula Ia.5 in which R⁶ is —P(═S)(OCH₂CH₃)₂ and the combination of R^{2a}, R^{2b}, R^{2c} and R⁴ for a compound corresponds in each case to one row of Table A

### Table 224

Compounds of the formula Ia.5 in which R⁶ is —P(═S)(OCH₂CH₂CH₃)₂ and the combination of R^{2a}, R^{2b}, R^{2c} and R⁴ for a compound corresponds in each case to one row of Table A

### Table 225

Compounds of the formula Ia.5 in which R⁶ is —P(═S)(O-phenyl)₂ and the combination of R^{2a}, R^{2b}, R^{2c} and R⁴ for a compound corresponds in each case to one row of Table A

### Tables 226 to 370

Compounds of the formula Ia.6 in which R⁶ is as defined in any of tables 81 to 225 and the combination of R^{2a}, R^{2b}, R^{2c} and R⁴ for a compound corresponds in each case to one row of Table A

### Tables 371 to 515

Compounds of the formula Ia.7 in which R⁶ is as defined in any of tables 81 to 225 and the combination of R^{2a}, R^{2b}, R^{2c} and R⁴ for a compound corresponds in each case to one row of Table A

### Table 516

Compounds of the formula Ia.8 in which the combination of R^{2a}, R^{2b}, R^{2c} and R⁴ for a compound corresponds in each case to one row of Table A

### Table 517

Compounds of the formula Ia.9 in which the combination of R^{2a}, R^{2b}, R^{2c} and R⁴ for a compound corresponds in each case to one row of Table A

### Table 518

Compounds of the formula Ia.10 in which the combination of R^{2a}, R^{2b}, R^{2c} and R⁴ for a compound corresponds in each case to one row of Table A

### Table 519

Compounds of the formula Ia.11 in which the combination of R^{2a}, R^{2b}, R^{2c} and R⁴ for a compound corresponds in each case to one row of Table A

### Table 520

Compounds of the formula Ia.12 in which the combination of R^{2a}, R^{2b}, R^{2c} and R⁴ for a compound corresponds in each case to one row of Table A

### Table 521

Compounds of the formula Ia.13 in which the combination of R^{2a}, R^{2b}, R^{2c} and R⁴ for a compound corresponds in each case to one row of Table A

### Table 522

Compounds of the formula Ia.14 in which the combination of R^{2a}, R^{2b}, R^{2c} and R⁴ for a compound corresponds in each case to one row of Table A

### Table 523

Compounds of the formula Ia.15 in which the combination of R^{2a}, R^{2b}, R^{2c} and R⁴ for a compound corresponds in each case to one row of Table A

### Table 524

Compounds of the formula Ia.16 in which the combination of R^{2a}, R^{2b}, R^{2c} and R⁴ for a compound corresponds in each case to one row of Table A

### Table 525

Compounds of the formula Ia.17 in which the combination of R^{2a}, R^{2b}, R^{2c} and R⁴ for a compound corresponds in each case to one row of Table A

### Table 526

Compounds of the formula Ia.18 in which the combination of R^{2a}, R^{2b}, R^{2c} and R⁴ for a compound corresponds in each case to one row of Table A

### Table 527

Compounds of the formula Ia.19 in which the combination of R^{2a}, R^{2b}, R^{2c} and R⁴ for a compound corresponds in each case to one row of Table A

### Table 528

Compounds of the formula Ia.20 in which the combination of R^{2a}, R^{2b}, R^{2c} and R⁴ for a compound corresponds in each case to one row of Table A

### Table 529

Compounds of the formula Ia.21 in which the combination of R^{2a}, R^{2b}, R^{2c} and R⁴ for a compound corresponds in each case to one row of Table A

### Table 530

Compounds of the formula Ia.22 in which the combination of R^{2a}, R^{2b}, R^{2c} and R⁴ for a compound corresponds in each case to one row of Table A

### Table 531

Compounds of the formula Ia.23 in which the combination of R^{2a}, R^{2b}, R^{2c} and R⁴ for a compound corresponds in each case to one row of Table A

### Table 532

Compounds of the formula Ia.24 in which the combination of R^{2a}, R^{2b}, R^{2c} and R⁴ for a compound corresponds in each case to one row of Table A

### Table 533

Compounds of the formula Ia.25 in which the combination of R^{2a}, R^{2b}, R^{2c} and R⁴ for a compound corresponds in each case to one row of Table A

### Table 534

Compounds of the formula Ia.26 in which the combination of R^{2a}, R^{2b}, R^{2c} and R⁴ for a compound corresponds in each case to one row of Table A

### Table 535

Compounds of the formula Ia.27 in which the combination of R^{2a}, R^{2b}, R^{2c} and R⁴ for a compound corresponds in each case to one row of Table A

### Table 536

Compounds of the formula Ia.28 in which the combination of R^{2a}, R^{2b}, R^{2c} and R⁴ for a compound corresponds in each case to one row of Table A

### Tables 537 to 681

Compounds of the formula Ia.29 in which R⁶ is as defined in any of tables 81 to 225 and the combination of R^{2a}, R^{2b}, R^{2c} and R⁴ for a compound corresponds in each case to one row of Table A

### Tables 682 to 826

Compounds of the formula Ia.30 in which R⁶ is as defined in any of tables 81 to 225 and the combination of R^{2a}, R^{2b}, R^{2c} and R⁴ for a compound corresponds in each case to one row of Table A

### Table 827

Compounds of the formula Ia.31 in which R^{9a} and R^{9b} are methyl and the combination of R^{2a}, R^{2b}, R^{2c} and R⁴ for a compound corresponds in each case to one row of Table A

### Table 828

Compounds of the formula Ia.31 in which R^{9a} and R^{9b} are ethyl and the combination of R^{2a}, R^{2b}, R^{2c} and R⁴ for a compound corresponds in each case to one row of Table A

### Table 829

Compounds of the formula Ia.31 in which R^{9a} and R^{9b} are n-propyl and the combination of R^{2a}, R^{2b}, R^{2c} and R⁴ for a compound corresponds in each case to one row of Table A

### Table 830

Compounds of the formula Ia.31 in which R^{9a} and R^{9b} are isopropyl and the combination of R^{2a}, R^{2b}, R^{2c} and R⁴ for a compound corresponds in each case to one row of Table A

### Table 831

Compounds of the formula Ia.31 in which R^{9a} and R^{9b} are n-butyl and the combination of R^{2a}, R^{2b}, R^{2c} and R⁴ for a compound corresponds in each case to one row of Table A

### Table 832

Compounds of the formula Ia.31 in which R^{9a} and R^{9b} are sec-butyl and the combination of R^{2a}, R^{2b}, R^{2c} and R⁴ for a compound corresponds in each case to one row of Table A

### Table 833

Compounds of the formula Ia.31 in which R^{9a} and R^{9b} are isobutyl and the combination of R^{2a}, R^{2b}, R^{2c} and R⁴ for a compound corresponds in each case to one row of Table A

### Table 834

Compounds of the formula Ia.31 in which R^{9a} and R^{9b} are tert-butyl and the combination of R^{2a}, R^{2b}, R^{2c} and R⁴ for a compound corresponds in each case to one row of Table A

### Table 835

Compounds of the formula Ia.31 in which R^{9a} and R^{9b} are cyclopropyl and the combination of R^{2a}, R^{2b}, R^{2c} and R⁴ for a compound corresponds in each case to one row of Table A

### Table 836

Compounds of the formula Ia.31 in which R^{9a} and R^{9b} are —CH₂-cyclopropyl and the combination of R^{2a}, R^{2b}, R^{2c} and R⁴ for a compound corresponds in each case to one row of Table A

### Table 837

Compounds of the formula Ia.31 in which R^{9a} is methyl, R^{9b} is ethyl and the combination of R^{2a}, R^{2b}, R^{2c} and R⁴ for a compound corresponds in each case to one row of Table A

### Table 838

Compounds of the formula Ia.31 in which R^{9a} is methyl, R^{9b} is isopropyl and the combination of R^{2a}, R^{2b}, R^{2c} and R⁴ for a compound corresponds in each case to one row of Table A

### Table 839

Compounds of the formula Ia.31 in which R^{9a} is methyl, R^{9b} is isobutyl and the combination of R^{2a}, R^{2b}, R^{2c} and R⁴ for a compound corresponds in each case to one row of Table A

### Table 840

Compounds of the formula Ia.31 in which R^{9a} is methyl, R^{9b} is cyclopropyl and the combination of R^{2a}, R^{2b}, R^{2c} and R⁴ for a compound corresponds in each case to one row of Table A

### Table 841

Compounds of the formula Ia.31 in which R^{9a} is methyl, R^{9b} is —CH₂-cyclopropyl and the combination of R^{2a}, R^{2b}, R^{2c} and R⁴ for a compound corresponds in each case to one row of Table A

### Table 842

Compounds of the formula Ia.31 in which R^{9a} is ethyl, R^{9b} is ethyl and the combination of R^{2a}, R^{2b}, R^{2c} and R⁴ for a compound corresponds in each case to one row of Table A

### Table 843

Compounds of the formula Ia.31 in which R^{9a} is ethyl, R^{9b} is isopropyl and the combination of R^{2a}, R^{2b}, R^{2c} and R⁴ for a compound corresponds in each case to one row of Table A

### Table 844

Compounds of the formula Ia.31 in which R^{9a} is ethyl, R^{9b} is isobutyl and the combination of R^{2a}, R^{2b}, R^{2c} and R⁴ for a compound corresponds in each case to one row of Table A

### Table 845

Compounds of the formula Ia.31 in which R^{9a} is ethyl, R^{9b} is cyclopropyl and the combination of R^{2a}, R^{2b}, R^{2c} and R⁴ for a compound corresponds in each case to one row of Table A

### Table 846

Compounds of the formula Ia.31 in which R^{9a} is ethyl, R^{9b} is —CH₂-cyclopropyl and the combination of R^{2a}, R^{2b}, R^{2c} and R⁴ for a compound corresponds in each case to one row of Table A

### Table 847

Compounds of the formula Ia.31 in which R^{9a} and R^{9b} together form a bridging group —(CH₂)₄— and the combination of R^{2a}, R^{2b}, R^{2c} and R⁴ for a compound corresponds in each case to one row of Table A

### Tables 848 to 868

Compounds of the formula Ia.32 in which the combination of R^{9a} and R^{9b} is as defined in any of tables 827 to 847 and the combination of R^{2a}, R^{2b}, R^{2c} and R⁴ for a compound corresponds in each case to one row of Table A

### Table 869

Compounds of the formula Ia.33 in which R⁸ is methyl and the combination of R^{2a}, R^{2b}, R^{2c} and R⁴ for a compound corresponds in each case to one row of Table A

### Table 870

Compounds of the formula Ia.33 in which R⁸ is ethyl and the combination of R^{2a}, R^{2b}, R^{2c} and R⁴ for a compound corresponds in each case to one row of Table A

### Table 871

Compounds of the formula Ia.33 in which R⁸ is propyl and the combination of R^{2a}, R^{2b}, R^{2c} and R⁴ for a compound corresponds in each case to one row of Table A

### Table 872

Compounds of the formula Ia.33 in which R⁸ is isopropyl and the combination of R^{2a}, R^{2b}, R^{2c} and R⁴ for a compound corresponds in each case to one row of Table A

### Table 873

Compounds of the formula Ia.33 in which R⁸ is n-butyl and the combination of R^{2a}, R^{2b}, R^{2c} and R⁴ for a compound corresponds in each case to one row of Table A

### Table 874

Compounds of the formula Ia.33 in which R⁸ is sec-butyl and the combination of R^{2a}, R^{2b}, R^{2c} and R⁴ for a compound corresponds in each case to one row of Table A

### Table 875

Compounds of the formula Ia.33 in which R⁸ is isobutyl and the combination of R^{2a}, R^{2b}, R^{2c} and R⁴ for a compound corresponds in each case to one row of Table A

### Table 876

Compounds of the formula Ia.33 in which R⁸ is tert-butyl and the combination of R^{2a}, R^{2b}, R^{2c} and R⁴ for a compound corresponds in each case to one row of Table A

### Table 877

Compounds of the formula Ia.33 in which R⁸ is CF₃ and the combination of R^{2a}, R^{2b}, R^{2b} and R⁴ for a compound corresponds in each case to one row of Table A

### Table 878

Compounds of the formula Ia.33 in which R⁸ is CH₂CHF₂ and the combination of R^{2a}, R^{2b}, R^{2c} and R⁴ for a compound corresponds in each case to one row of Table A

### Table 879

Compounds of the formula Ia.33 in which R⁸ is CH₂CF₃ and the combination of R^{2a}, R^{2b}, R^{2c} and R⁴ for a compound corresponds in each case to one row of Table A

### Table 880

Compounds of the formula Ia.33 in which R⁸ is CH₂CH₂CF₃ and the combination of R^{2a}, R^{2b}, R^{2c} and R⁴ for a compound corresponds in each case to one row of Table A

### Table 881

Compounds of the formula Ia.33 in which R⁸ is cyclopropyl and the combination of R^{2a}, R^{2b}, R^{2c} and R⁴ for a compound corresponds in each case to one row of Table A

### Table 882

Compounds of the formula Ia.33 in which R⁸ is methylthiomethyl and the combination of R^{2a}, R^{2b}, R^{2c} and R⁴ for a compound corresponds in each case to one row of Table A

### Table 883

Compounds of the formula Ia.33 in which R⁸ is ethylthiomethyl and the combination of R^{2a}, R^{2b}, R^{2c} and R⁴ for a compound corresponds in each case to one row of Table A

### Table 884

Compounds of the formula Ia.33 in which R⁸ is methylsulfinylmethyl and the combination of R^{2a}, R^{2b}, R^{2c} and R⁴ for a compound corresponds in each case to one row of Table A

### Table 885

Compounds of the formula Ia.33 in which R⁸ is ethylsulfinylmethyl and the combination of R^{2a}, R^{2b}, R^{2c} and R⁴ for a compound corresponds in each case to one row of Table A

### Table 886

Compounds of the formula Ia.33 in which R⁸ is methylsulfonylmethyl and the combination of R^{2a}, R^{2b}, R^{2c} and R⁴ for a compound corresponds in each case to one row of Table A

### Table 887

Compounds of the formula Ia.33 in which R⁸ is ethylsulfonylmethyl and the combination of R^{2a}, R^{2b}, R^{2c} and R⁴ for a compound corresponds in each case to one row of Table A

### Table 888

Compounds of the formula Ia.33 in which R⁸ is phenyl and the combination of R^{2a}, R^{2b}, R^{2c} and R⁴ for a compound corresponds in each case to one row of Table A

### Table 889

Compounds of the formula Ia.33 in which R⁸ is 2-fluorophenyl and the combination of R^{2a}, R^{2b}, R^{2c} and R⁴ for a compound corresponds in each case to one row of Table A

### Table 890

Compounds of the formula Ia.33 in which R⁸ is 3-fluorophenyl and the combination of R^{2a}, R^{2b}, R^{2c} and R⁴ for a compound corresponds in each case to one row of Table A

### Table 891

Compounds of the formula Ia.33 in which R⁸ is 4-fluorophenyl and the combination of R^{2a}, R^{2b}, R^{2c} and R⁴ for a compound corresponds in each case to one row of Table A

### Table 892

Compounds of the formula Ia.33 in which R⁸ is 2,3-difluorophenyl and the combination of R^{2a}, R^{2b}, R^{2c} and R⁴ for a compound corresponds in each case to one row of Table A

### Table 893

Compounds of the formula Ia.33 in which R⁸ is 2,4-difluorophenyl and the combination of R^{2a}, R^{2b}, R^{2c} and R⁴ for a compound corresponds in each case to one row of Table A

### Table 894

Compounds of the formula Ia.33 in which R⁸ is 2,5-difluorophenyl and the combination of R^{2a}, R^{2b}, R^{2c} and R⁴ for a compound corresponds in each case to one row of Table A

### Table 895

Compounds of the formula Ia.33 in which R⁸ is 2,6-difluorophenyl and the combination of R^{2a}, R^{2b}, R^{2c} and R⁴ for a compound corresponds in each case to one row of Table A

### Table 896

Compounds of the formula Ia.33 in which R⁸ is 3,4-difluorophenyl and the combination of R^{2a}, R^{2b}, R^{2c} and R⁴ for a compound corresponds in each case to one row of Table A

### Table 897

Compounds of the formula Ia.33 in which R⁸ is 3,5-difluorophenyl and the combination of R^{2a}, R^{2b}, R^{2c} and R⁴ for a compound corresponds in each case to one row of Table A

### Table 898

Compounds of the formula Ia.33 in which R⁸ is 2-chlorophenyl and the combination of R^{2a}, R^{2b}, R^{2b} and R⁴ for a compound corresponds in each case to one row of Table A

### Table 899

Compounds of the formula Ia.33 in which R⁸ is 3-chlorophenyl and the combination of R^{2a}, R^{2b}, R^{2c} and R⁴ for a compound corresponds in each case to one row of Table A

### Table 900

Compounds of the formula Ia.33 in which R⁸ is 4-chlorophenyl and the combination of R^{2a}, R^{2b}, R^{2c} and R⁴ for a compound corresponds in each case to one row of Table A

### Table 901

Compounds of the formula Ia.33 in which R⁸ is 2-methoxyphenyl and the combination of R^{2a}, R^{2b}, R^{2c} and R⁴ for a compound corresponds in each case to one row of Table A

### Table 902

Compounds of the formula Ia.33 in which R⁸ is 3-methoxyphenyl and the combination of R^{2a}, R^{2b}, R^{2c} and R⁴ for a compound corresponds in each case to one row of Table A

### Table 903

Compounds of the formula Ia.33 in which R⁸ is 4-methoxyphenyl and the combination of R^{2a}, R^{2b}, R^{2c} and R⁴ for a compound corresponds in each case to one row of Table A

### Table 904

Compounds of the formula Ia.33 in which R⁸ is thietan-3-yl and the combination of R^{2a}, R^{2b}, R^{2c} and R⁴ for a compound corresponds in each case to one row of Table A

### Table 905

Compounds of the formula Ia.33 in which R⁸ is 1-oxo-thietan-3-yl and the combination of R^{2a}, R^{2b}, R^{2c} and R⁴ for a compound corresponds in each case to one row of Table A

### Table 906

Compounds of the formula Ia.33 in which R⁸ is 1,1-dioxo-thietan-3-yl and the combination of R^{2a}, R^{2b}, R^{2c} and R⁴ for a compound corresponds in each case to one row of Table A

### Table 907

Compounds of the formula Ia.33 in which R⁸ is pyridin-2-yl and the combination of R^{2a}, R^{2b}, R^{2c} and R⁴ for a compound corresponds in each case to one row of Table A

### Table 908

Compounds of the formula Ia.33 in which R⁸ is pyridin-3-yl and the combination of R^{2a}, R^{2b}, R^{2c} and R⁴ for a compound corresponds in each case to one row of Table A

### Table 909

Compounds of the formula Ia.33 in which R⁸ is pyridin-4-yl and the combination of R^{2a}, R^{2b}, R^{2c} and R⁴ for a compound corresponds in each case to one row of Table A

### Table 910

Compounds of the formula Ia.33 in which R⁸ is 4-chloropyridin-3-yl and the combination of R^{2a}, R^{2b}, R^{2c} and R⁴ for a compound corresponds in each case to one row of Table A

### Table 911

Compounds of the formula Ia.33 in which R⁸ is —NH—CH₃ and the combination of R^{2a}, R^{2b}, R^{2c} and R⁴ for a compound corresponds in each case to one row of Table A

### Table 912

Compounds of the formula Ia.33 in which R⁸ is —NH—CH₂CH₃ and the combination of R^{2a}, R^{2b}, R^{2c} and R⁴ for a compound corresponds in each case to one row of Table A

### Table 913

Compounds of the formula Ia.33 in which R⁸ is —NH—CH₂CH₂CH₃ and the combination of R^{2a}, R^{2b}, R^{2c} and R⁴ for a compound corresponds in each case to one row of Table A

### Table 914

Compounds of the formula Ia.33 in which R⁸ is —NH—CH₂CN and the combination of R^{2a}, R^{2b}, R^{2c} and R⁴ for a compound corresponds in each case to one row of Table A

### Table 915

Compounds of the formula Ia.33 in which R⁸ is —NH—CH₂CHF₂ and the combination of R^{2a}, R^{2b}, R^{2c} and R⁴ for a compound corresponds in each case to one row of Table A

### Table 916

Compounds of the formula Ia.33 in which R⁸ is —NH—CH₂CF₃ and the combination of R^{2a}, R^{2b}, R^{2c} and R⁴ for a compound corresponds in each case to one row of Table A

### Table 917

Compounds of the formula Ia.33 in which R⁸ is —NH-phenyl and the combination of R^{2a}, R^{2b}, R^{2c} and R⁴ for a compound corresponds in each case to one row of Table A

### Table 918

Compounds of the formula Ia.33 in which R⁸ is —NH-benzyl and the combination of R^{2a}, R^{2b}, R^{2c} and R⁴ for a compound corresponds in each case to one row of Table A

### Tables 919 to 968

Compounds of the formula Ia.34 in which R⁸ is as defined in any of tables 869 to 918 and the combination of R^{2a}, R^{2b}, R^{2c} and R⁴ for a compound corresponds in each case to one row of Table A

### Tables 969 to 1018

Compounds of the formula Ia.35 in which R⁸ is as defined in any of tables 869 to 918 and the combination of R^{2a}, R^{2b}, R^{2c} and R⁴ for a compound corresponds in each case to one row of Table A

### Tables 1019 to 1068

Compounds of the formula Ia.36 in which R⁸ is as defined in any of tables 869 to 918 and the combination of R^{2a}, R^{2b}, R^{2c} and R⁴ for a compound corresponds in each case to one row of Table A

### Tables 1069 to 1118

Compounds of the formula Ia.37 in which R⁸ is as defined in any of tables 869 to 918 and the combination of R^{2a}, R^{2b}, R^{2c} and R⁴ for a compound corresponds in each case to one row of Table A

### Tables 1119 to 1168

Compounds of the formula Ia.38 in which R⁸ is as defined in any of tables 869 to 918 and the combination of R^{2a}, R^{2b}, R^{2c} and R⁴ for a compound corresponds in each case to one row of Table A

### Tables 1169 to 1218

Compounds of the formula Ia.39 in which R⁸ is as defined in any of tables 869 to 918 and the combination of R^{2a}, R^{2b}, R^{2c} and R⁴ for a compound corresponds in each case to one row of Table A

### Tables 1219 to 1268

Compounds of the formula Ia.40 in which R⁸ is as defined in any of tables 869 to 918 and the combination of R^{2a}, R^{2b}, R^{2c} and R⁴ for a compound corresponds in each case to one row of Table A

### Tables 1269 to 1318

Compounds of the formula Ia.41 in which R⁸ is as defined in any of tables 869 to 918 and the combination of R^{2a}, R^{2b}, R^{2c} and R⁴ for a compound corresponds in each case to one row of Table A

### Tables 1319 to 1368

Compounds of the formula Ia.42 in which R⁸ is as defined in any of tables 869 to 918 and the combination of R^{2a}, R^{2b}, R^{2c} and R⁴ for a compound corresponds in each case to one row of Table A

### Table 1369

Compounds of the formula Ia.43 in which A⁴ is pyrazol-1-yl and the combination of R^{2a}, R^{2b}, R^{2c} and R⁴ for a compound corresponds in each case to one row of Table A

### Table 1370

Compounds of the formula Ia.43 in which A⁴ is 1H-[1,2,4]-triazol-1-yl and the combination of R^{2a}, R^{2b}, R^{2c} and R⁴ for a compound corresponds in each case to one row of Table A

### Table 1371

Compounds of the formula Ia.43 in which A⁴ is 3-chloro-1H-[1,2,4]-triazol-1-yl and the combination of R^{2a}, R^{2b}, R^{2c} and R⁴ for a compound corresponds in each case to one row of Table A

### Table 1372

Compounds of the formula Ia.43 in which A⁴ is 3-cyano-1H-[1,2,4]-triazol-1-yl and the combination of R^{2a}, R^{2b}, R^{2c} and R⁴ for a compound corresponds in each case to one row of Table A

### Tables 1373 to 2744

Compounds of the formula Ia.44 in which A is as defined in formulae Ia.1 to Ia.43 in combination with tables 1 to 1372 and the combination of R^{2a}, R^{2b}, R^{2c} and R⁴ for a compound corresponds in each case to one row of Table A.

TABLE A

| No. | R^{2a} | R^{2b} | R^{2c} | R⁴ |
|---|---|---|---|---|
| A-1 | F | H | F | H |
| A-2 | F | F | F | H |
| A-3 | F | Cl | F | H |
| A-4 | F | Br | F | H |
| A-5 | F | H | Cl | H |
| A-6 | F | H | Br | H |
| A-7 | Cl | H | Cl | H |
| A-8 | Cl | Cl | Cl | H |
| A-9 | Cl | F | Cl | H |
| A-10 | Cl | Br | Cl | H |
| A-11 | Cl | H | Br | H |
| A-12 | Br | H | Br | H |
| A-13 | Br | F | Br | H |
| A-14 | Br | Cl | Br | H |
| A-15 | CF₃ | H | F | H |
| A-16 | CF₃ | H | Cl | H |
| A-17 | CF₃ | H | Br | H |
| A-18 | CF₃ | H | CF₃ | H |
| A-19 | CF₃ | F | F | H |
| A-20 | CF₃ | Cl | Cl | H |
| A-21 | CF₃ | Br | Br | H |
| A-22 | H | H | F | H |
| A-23 | H | H | Cl | H |
| A-24 | H | H | Br | H |
| A-25 | H | H | CF₃ | H |
| A-26 | F | H | F | CH₃ |
| A-27 | F | F | F | CH₃ |
| A-28 | F | Cl | F | CH₃ |
| A-29 | F | Br | F | CH₃ |
| A-30 | F | H | Cl | CH₃ |
| A-31 | F | H | Br | CH₃ |
| A-32 | Cl | H | Cl | CH₃ |
| A-33 | Cl | Cl | Cl | CH₃ |
| A-34 | Cl | F | Cl | CH₃ |
| A-35 | Cl | Br | Cl | CH₃ |
| A-36 | Cl | H | Br | CH₃ |
| A-37 | Br | H | Br | CH₃ |
| A-38 | Br | F | Br | CH₃ |
| A-39 | Br | Cl | Br | CH₃ |
| A-40 | CF₃ | H | F | CH₃ |
| A-41 | CF₃ | H | Cl | CH₃ |
| A-42 | CF₃ | H | Br | CH₃ |
| A-43 | CF₃ | H | CF₃ | CH₃ |
| A-44 | CF₃ | F | F | CH₃ |
| A-45 | CF₃ | Cl | Cl | CH₃ |
| A-46 | CF₃ | Br | Br | CH₃ |
| A-47 | H | H | F | CH₃ |
| A-48 | H | H | Cl | CH₃ |
| A-49 | H | H | Br | CH₃ |
| A-50 | H | H | CF₃ | CH₃ |
| A-51 | F | H | F | CH₂CH₃ |
| A-52 | F | F | F | CH₂CH₃ |
| A-53 | F | Cl | F | CH₂CH₃ |
| A-54 | F | Br | F | CH₂CH₃ |
| A-55 | F | H | Cl | CH₂CH₃ |
| A-56 | F | H | Br | CH₂CH₃ |
| A-57 | Cl | H | Cl | CH₂CH₃ |
| A-58 | Cl | Cl | Cl | CH₂CH₃ |
| A-59 | Cl | F | Cl | CH₂CH₃ |
| A-60 | Cl | Br | Cl | CH₂CH₃ |
| A-61 | Cl | H | Br | CH₂CH₃ |
| A-62 | Br | H | Br | CH₂CH₃ |
| A-63 | Br | F | Br | CH₂CH₃ |
| A-64 | Br | Cl | Br | CH₂CH₃ |
| A-65 | CF₃ | H | F | CH₂CH₃ |
| A-66 | CF₃ | H | Cl | CH₂CH₃ |
| A-67 | CF₃ | H | Br | CH₂CH₃ |
| A-68 | CF₃ | H | CF₃ | CH₂CH₃ |
| A-69 | CF₃ | F | F | CH₂CH₃ |
| A-70 | CF₃ | Cl | Cl | CH₂CH₃ |
| A-71 | CF₃ | Br | Br | CH₂CH₃ |
| A-72 | H | H | F | CH₂CH₃ |
| A-73 | H | H | Cl | CH₂CH₃ |
| A-74 | H | H | Br | CH₂CH₃ |
| A-75 | H | H | CF₃ | CH₂CH₃ |
| A-76 | F | H | F | CH(CH₃)₂ |
| A-77 | F | F | F | CH(CH₃)₂ |
| A-78 | F | Cl | F | CH(CH₃)₂ |
| A-79 | F | Br | F | CH(CH₃)₂ |
| A-80 | F | H | Cl | CH(CH₃)₂ |
| A-81 | F | H | Br | CH(CH₃)₂ |
| A-82 | Cl | H | Cl | CH(CH₃)₂ |
| A-83 | Cl | Cl | Cl | CH(CH₃)₂ |
| A-84 | Cl | F | Cl | CH(CH₃)₂ |
| A-85 | Cl | Br | Cl | CH(CH₃)₂ |
| A-86 | Cl | H | Br | CH(CH₃)₂ |
| A-87 | Br | H | Br | CH(CH₃)₂ |
| A-88 | Br | F | Br | CH(CH₃)₂ |
| A-89 | Br | Cl | Br | CH(CH₃)₂ |
| A-90 | CF₃ | H | F | CH(CH₃)₂ |
| A-91 | CF₃ | H | Cl | CH(CH₃)₂ |
| A-92 | CF₃ | H | Br | CH(CH₃)₂ |
| A-93 | CF₃ | H | CF₃ | CH(CH₃)₂ |
| A-94 | CF₃ | F | F | CH(CH₃)₂ |
| A-95 | CF₃ | Cl | Cl | CH(CH₃)₂ |
| A-96 | CF₃ | Br | Br | CH(CH₃)₂ |
| A-97 | H | H | F | CH(CH₃)₂ |
| A-98 | H | H | Cl | CH(CH₃)₂ |
| A-99 | H | H | Br | CH(CH₃)₂ |
| A-100 | H | H | CF₃ | CH(CH₃)₂ |
| A-101 | F | H | F | CHF₂ |
| A-102 | F | F | F | CHF₂ |
| A-103 | F | Cl | F | CHF₂ |
| A-104 | F | Br | F | CHF₂ |
| A-105 | F | H | Cl | CHF₂ |
| A-106 | F | H | Br | CHF₂ |
| A-107 | Cl | H | Cl | CHF₂ |
| A-108 | Cl | Cl | Cl | CHF₂ |
| A-109 | Cl | F | Cl | CHF₂ |
| A-110 | Cl | Br | Cl | CHF₂ |
| A-111 | Cl | H | Br | CHF₂ |
| A-112 | Br | H | Br | CHF₂ |
| A-113 | Br | F | Br | CHF₂ |
| A-114 | Br | Cl | Br | CHF₂ |
| A-115 | CF₃ | H | F | CHF₂ |
| A-116 | CF₃ | H | Cl | CHF₂ |
| A-117 | CF₃ | H | Br | CHF₂ |
| A-118 | CF₃ | H | CF₃ | CHF₂ |
| A-119 | CF₃ | F | F | CHF₂ |
| A-120 | CF₃ | Cl | Cl | CHF₂ |
| A-121 | CF₃ | Br | Br | CHF₂ |
| A-122 | H | H | F | CHF₂ |
| A-123 | H | H | Cl | CHF₂ |
| A-124 | H | H | Br | CHF₂ |
| A-125 | H | H | CF₃ | CHF₂ |
| A-126 | F | H | F | CF₃ |
| A-127 | F | F | F | CF₃ |
| A-128 | F | Cl | F | CF₃ |
| A-129 | F | Br | F | CF₃ |
| A-130 | F | H | Cl | CF₃ |
| A-131 | F | H | Br | CF₃ |
| A-132 | Cl | H | Cl | CF₃ |
| A-133 | Cl | Cl | Cl | CF₃ |
| A-134 | Cl | F | Cl | CF₃ |
| A-135 | Cl | Br | Cl | CF₃ |
| A-136 | Cl | H | Br | CF₃ |
| A-137 | Br | H | Br | CF₃ |
| A-138 | Br | F | Br | CF₃ |
| A-139 | Br | Cl | Br | CF₃ |
| A-140 | CF₃ | H | F | CF₃ |
| A-141 | CF₃ | H | Cl | CF₃ |
| A-142 | CF₃ | H | Br | CF₃ |
| A-143 | CF₃ | H | CF₃ | CF₃ |
| A-144 | CF₃ | F | F | CF₃ |
| A-145 | CF₃ | Cl | Cl | CF₃ |
| A-146 | CF₃ | Br | Br | CF₃ |
| A-147 | H | H | F | CF₃ |
| A-148 | H | H | Cl | CF₃ |
| A-149 | H | H | Br | CF₃ |
| A-150 | H | H | CF₃ | CF₃ |
| A-151 | F | H | F | CH₂—CH═CH₂ |
| A-152 | F | F | F | CH₂—CH═CH₂ |
| A-153 | F | Cl | F | CH₂—CH═CH₂ |
| A-154 | F | Br | F | CH₂—CH═CH₂ |
| A-155 | F | H | Cl | CH₂—CH═CH₂ |
| A-156 | F | H | Br | CH₂—CH═CH₂ |
| A-157 | Cl | H | Cl | CH₂—CH═CH₂ |
| A-158 | Cl | Cl | Cl | CH₂—CH═CH₂ |
| A-159 | Cl | F | Cl | CH₂—CH═CH₂ |
| A-160 | Cl | Br | Cl | CH₂—CH═CH₂ |
| A-161 | Cl | H | Br | CH₂—CH═CH₂ |
| A-162 | Br | H | Br | CH₂—CH═CH₂ |
| A-163 | Br | F | Br | CH₂—CH═CH₂ |
| A-164 | Br | Cl | Br | CH₂—CH═CH₂ |
| A-165 | CF₃ | H | F | CH₂—CH═CH₂ |
| A-166 | CF₃ | H | Cl | CH₂—CH═CH₂ |
| A-167 | CF₃ | H | Br | CH₂—CH═CH₂ |
| A-168 | CF₃ | H | CF₃ | CH₂—CH═CH₂ |
| A-169 | CF₃ | F | F | CH₂—CH═CH₂ |
| A-170 | CF₃ | Cl | Cl | CH₂—CH═CH₂ |
| A-171 | CF₃ | Br | Br | CH₂—CH═CH₂ |
| A-172 | H | H | F | CH₂—CH═CH₂ |
| A-173 | H | H | Cl | CH₂—CH═CH₂ |
| A-174 | H | H | Br | CH₂—CH═CH₂ |
| A-175 | H | H | CF₃ | CH₂—CH═CH₂ |
| A-176 | F | H | F | CH═CH₂ |
| A-177 | F | F | F | CH═CH₂ |
| A-178 | F | Cl | F | CH═CH₂ |
| A-179 | F | Br | F | CH═CH₂ |
| A-180 | F | H | Cl | CH═CH₂ |
| A-181 | F | H | Br | CH═CH₂ |
| A-182 | Cl | H | Cl | CH═CH₂ |
| A-183 | Cl | Cl | Cl | CH═CH₂ |
| A-184 | Cl | F | Cl | CH═CH₂ |
| A-185 | Cl | Br | Cl | CH═CH₂ |
| A-186 | Cl | H | Br | CH═CH₂ |
| A-187 | Br | H | Br | CH═CH₂ |
| A-188 | Br | F | Br | CH═CH₂ |
| A-189 | Br | Cl | Br | CH═CH₂ |
| A-190 | CF₃ | H | F | CH═CH₂ |
| A-191 | CF₃ | H | Cl | CH═CH₂ |
| A-192 | CF₃ | H | Br | CH═CH₂ |
| A-193 | CF₃ | H | CF₃ | CH═CH₂ |
| A-194 | CF₃ | F | F | CH═CH₂ |
| A-195 | CF₃ | Cl | Cl | CH═CH₂ |
| A-196 | CF₃ | Br | Br | CH═CH₂ |
| A-197 | H | H | F | CH═CH₂ |
| A-198 | H | H | Cl | CH═CH₂ |
| A-199 | H | H | Br | CH═CH₂ |
| A-200 | H | H | CF₃ | CH═CH₂ |
| A-201 | F | H | F | C≡CH |
| A-202 | F | F | F | C≡CH |
| A-203 | F | Cl | F | C≡CH |
| A-204 | F | Br | F | C≡CH |
| A-205 | F | H | Cl | C≡CH |
| A-206 | F | H | Br | C≡CH |
| A-207 | Cl | H | Cl | C≡CH |
| A-208 | Cl | Cl | Cl | C≡CH |
| A-209 | Cl | F | Cl | C≡CH |
| A-210 | Cl | Br | Cl | C≡CH |
| A-211 | Cl | H | Br | C≡CH |
| A-212 | Br | H | Br | C≡CH |
| A-213 | Br | F | Br | C≡CH |
| A-214 | Br | Cl | Br | C≡CH |
| A-215 | CF₃ | H | F | C≡CH |
| A-216 | CF₃ | H | Cl | C≡CH |
| A-217 | CF₃ | H | Br | C≡CH |
| A-218 | CF₃ | H | CF₃ | C≡CH |
| A-219 | CF₃ | F | F | C≡CH |
| A-220 | CF₃ | Cl | Cl | C≡CH |
| A-221 | CF₃ | Br | Br | C≡CH |
| A-222 | H | H | F | C≡CH |
| A-223 | H | H | Cl | C≡CH |
| A-224 | H | H | Br | C≡CH |
| A-225 | H | H | CF₃ | C≡CH |
| A-226 | F | H | F | ^{c}C₃H₅* |
| A-227 | F | F | F | ^{c}C₃H₅* |
| A-228 | F | Cl | F | ^{c}C₃H₅* |
| A-229 | F | Br | F | ^{c}C₃H₅* |
| A-230 | F | H | Cl | ^{c}C₃H₅* |
| A-231 | F | H | Br | ^{c}C₃H₅* |
| A-232 | Cl | H | Cl | ^{c}C₃H₅* |
| A-233 | Cl | Cl | Cl | ^{c}C₃H₅* |
| A-234 | Cl | F | Cl | ^{c}C₃H₅* |
| A-235 | Cl | Br | Cl | ^{c}C₃H₅* |
| A-236 | Cl | H | Br | ^{c}C₃H₅* |
| A-237 | Br | H | Br | ^{c}C₃H₅* |
| A-238 | Br | F | Br | ^{c}C₃H₅* |
| A-239 | Br | Cl | Br | ^{c}C₃H₅* |
| A-240 | CF₃ | H | F | ^{c}C₃H₅* |
| A-241 | CF₃ | H | Cl | ^{c}C₃H₅* |
| A-242 | CF₃ | H | Br | ^{c}C₃H₅* |
| A-243 | CF₃ | H | CF₃ | ^{c}C₃H₅* |
| A-244 | CF₃ | F | F | ^{c}C₃H₅* |
| A-245 | CF₃ | Cl | Cl | ^{c}C₃H₅* |
| A-246 | CF₃ | Br | Br | ^{c}C₃H₅* |
| A-247 | H | H | F | ^{c}C₃H₅* |
| A-248 | H | H | Cl | ^{c}C₃H₅* |
| A-249 | H | H | Br | ^{c}C₃H₅* |
| A-250 | H | H | CF₃ | ^{c}C₃H₅* |
| A-251 | F | H | F | F |
| A-252 | F | F | F | F |
| A-253 | F | Cl | F | F |
| A-254 | F | Br | F | F |
| A-255 | F | H | Cl | F |
| A-256 | F | H | Br | F |
| A-257 | Cl | H | Cl | F |
| A-258 | Cl | Cl | Cl | F |
| A-259 | Cl | F | Cl | F |
| A-260 | Cl | Br | Cl | F |
| A-261 | Cl | H | Br | F |
| A-262 | Br | H | Br | F |
| A-263 | Br | F | Br | F |
| A-264 | Br | Cl | Br | F |
| A-265 | CF₃ | H | F | F |
| A-266 | CF₃ | H | Cl | F |
| A-267 | CF₃ | H | Br | F |
| A-268 | CF₃ | H | CF₃ | F |
| A-269 | CF₃ | F | F | F |
| A-270 | CF₃ | Cl | Cl | F |
| A-271 | CF₃ | Br | Br | F |
| A-272 | H | H | F | F |
| A-273 | H | H | Cl | F |
| A-274 | H | H | Br | F |
| A-275 | H | H | CF₃ | F |
| A-276 | F | H | F | Cl |
| A-277 | F | F | F | Cl |
| A-278 | F | Cl | F | Cl |
| A-279 | F | Br | F | Cl |
| A-280 | F | H | Cl | Cl |
| A-281 | F | H | Br | Cl |
| A-282 | Cl | H | Cl | Cl |
| A-283 | Cl | Cl | Cl | Cl |
| A-284 | Cl | F | Cl | Cl |
| A-285 | Cl | Br | Cl | Cl |
| A-286 | Cl | H | Br | Cl |
| A-287 | Br | H | Br | Cl |
| A-288 | Br | F | Br | Cl |
| A-289 | Br | Cl | Br | Cl |
| A-290 | CF₃ | H | F | Cl |
| A-291 | CF₃ | H | Cl | Cl |
| A-292 | CF₃ | H | Br | Cl |
| A-293 | CF₃ | H | CF₃ | Cl |
| A-294 | CF₃ | F | F | Cl |
| A-295 | CF₃ | Cl | Cl | Cl |
| A-296 | CF₃ | Br | Br | Cl |
| A-297 | H | H | F | Cl |
| A-298 | H | H | Cl | Cl |
| A-299 | H | H | Br | Cl |
| A-300 | H | H | CF₃ | Cl |
| A-301 | F | H | F | Br |
| A-302 | F | F | F | Br |
| A-303 | F | Cl | F | Br |
| A-304 | F | Br | F | Br |
| A-305 | F | H | Cl | Br |
| A-306 | F | H | Br | Br |
| A-307 | Cl | H | Cl | Br |
| A-308 | Cl | Cl | Cl | Br |
| A-309 | Cl | F | Cl | Br |
| A-310 | Cl | Br | Cl | Br |
| A-311 | Cl | H | Br | Br |
| A-312 | Br | H | Br | Br |
| A-313 | Br | F | Br | Br |
| A-314 | Br | Cl | Br | Br |
| A-315 | CF₃ | H | F | Br |
| A-316 | CF₃ | H | Cl | Br |
| A-317 | CF₃ | H | Br | Br |
| A-318 | CF₃ | H | CF₃ | Br |
| A-319 | CF₃ | F | F | Br |
| A-320 | CF₃ | Cl | Cl | Br |
| A-321 | CF₃ | Br | Br | Br |
| A-322 | H | H | F | Br |
| A-323 | H | H | Cl | Br |
| A-324 | H | H | Br | Br |
| A-325 | H | H | CF₃ | Br |
| A-326 | F | H | F | CN |
| A-327 | F | F | F | CN |
| A-328 | F | Cl | F | CN |
| A-329 | F | Br | F | CN |
| A-330 | F | H | Cl | CN |
| A-331 | F | H | Br | CN |
| A-332 | Cl | H | Cl | CN |
| A-333 | Cl | Cl | Cl | CN |
| A-334 | Cl | F | Cl | CN |
| A-335 | Cl | Br | Cl | CN |
| A-336 | Cl | H | Br | CN |
| A-337 | Br | H | Br | CN |
| A-338 | Br | F | Br | CN |
| A-339 | Br | Cl | Br | CN |
| A-340 | CF₃ | H | F | CN |
| A-341 | CF₃ | H | Cl | CN |
| A-342 | CF₃ | H | Br | CN |
| A-343 | CF₃ | H | CF₃ | CN |
| A-344 | CF₃ | F | F | CN |
| A-345 | CF₃ | Cl | Cl | CN |
| A-346 | CF₃ | Br | Br | CN |
| A-347 | H | H | F | CN |
| A-348 | H | H | Cl | CN |
| A-349 | H | H | Br | CN |
| A-350 | H | H | CF₃ | CN |
| A-351 | F | H | F | OCH₃ |
| A-352 | F | F | F | OCH₃ |
| A-353 | F | Cl | F | OCH₃ |
| A-354 | F | Br | F | OCH₃ |
| A-355 | F | H | Cl | OCH₃ |
| A-356 | F | H | Br | OCH₃ |
| A-357 | Cl | H | Cl | OCH₃ |
| A-358 | Cl | Cl | Cl | OCH₃ |
| A-359 | Cl | F | Cl | OCH₃ |
| A-360 | Cl | Br | Cl | OCH₃ |
| A-361 | Cl | H | Br | OCH₃ |
| A-362 | Br | H | Br | OCH₃ |
| A-363 | Br | F | Br | OCH₃ |
| A-364 | Br | Cl | Br | OCH₃ |
| A-365 | CF₃ | H | F | OCH₃ |
| A-366 | CF₃ | H | Cl | OCH₃ |
| A-367 | CF₃ | H | Br | OCH₃ |
| A-368 | CF₃ | H | CF₃ | OCH₃ |
| A-369 | CF₃ | F | F | OCH₃ |
| A-370 | CF₃ | Cl | Cl | OCH₃ |
| A-371 | CF₃ | Br | Br | OCH₃ |
| A-372 | H | H | F | OCH₃ |
| A-373 | H | H | Cl | OCH₃ |
| A-374 | H | H | Br | OCH₃ |
| A-375 | H | H | CF₃ | OCH₃ |
| A-376 | F | H | F | OCH₂CH₃ |
| A-377 | F | F | F | OCH₂CH₃ |
| A-378 | F | Cl | F | OCH₂CH₃ |
| A-379 | F | Br | F | OCH₂CH₃ |
| A-380 | F | H | Cl | OCH₂CH₃ |
| A-381 | F | H | Br | OCH₂CH₃ |
| A-382 | Cl | H | Cl | OCH₂CH₃ |
| A-383 | Cl | Cl | Cl | OCH₂CH₃ |
| A-384 | Cl | F | Cl | OCH₂CH₃ |
| A-385 | Cl | Br | Cl | OCH₂CH₃ |
| A-386 | Cl | H | Br | OCH₂CH₃ |
| A-387 | Br | H | Br | OCH₂CH₃ |
| A-388 | Br | F | Br | OCH₂CH₃ |
| A-389 | Br | Cl | Br | OCH₂CH₃ |
| A-390 | CF₃ | H | F | OCH₂CH₃ |
| A-391 | CF₃ | H | Cl | OCH₂CH₃ |
| A-392 | CF₃ | H | Br | OCH₂CH₃ |
| A-393 | CF₃ | H | CF₃ | OCH₂CH₃ |
| A-394 | CF₃ | F | F | OCH₂CH₃ |
| A-395 | CF₃ | Cl | Cl | OCH₂CH₃ |
| A-396 | CF₃ | Br | Br | OCH₂CH₃ |
| A-397 | H | H | F | OCH₂CH₃ |
| A-398 | H | H | Cl | OCH₂CH₃ |
| A-399 | H | H | Br | OCH₂CH₃ |
| A-400 | H | H | CF₃ | OCH₂CH₃ |
| A-401 | F | H | F | OCH(CH₃)₂ |
| A-402 | F | F | F | OCH(CH₃)₂ |
| A-403 | F | Cl | F | OCH(CH₃)₂ |
| A-404 | F | Br | F | OCH(CH₃)₂ |
| A-405 | F | H | Cl | OCH(CH₃)₂ |
| A-406 | F | H | Br | OCH(CH₃)₂ |
| A-407 | Cl | H | Cl | OCH(CH₃)₂ |
| A-408 | Cl | Cl | Cl | OCH(CH₃)₂ |
| A-409 | Cl | F | Cl | OCH(CH₃)₂ |
| A-410 | Cl | Br | Cl | OCH(CH₃)₂ |
| A-411 | Cl | H | Br | OCH(CH₃)₂ |
| A-412 | Br | H | Br | OCH(CH₃)₂ |
| A-413 | Br | F | Br | OCH(CH₃)₂ |
| A-414 | Br | Cl | Br | OCH(CH₃)₂ |
| A-415 | CF₃ | H | F | OCH(CH₃)₂ |
| A-416 | CF₃ | H | Cl | OCH(CH₃)₂ |
| A-417 | CF₃ | H | Br | OCH(CH₃)₂ |
| A-418 | CF₃ | H | CF₃ | OCH(CH₃)₂ |
| A-419 | CF₃ | F | F | OCH(CH₃)₂ |
| A-420 | CF₃ | Cl | Cl | OCH(CH₃)₂ |
| A-421 | CF₃ | Br | Br | OCH(CH₃)₂ |
| A-422 | H | H | F | OCH(CH₃)₂ |
| A-423 | H | H | Cl | OCH(CH₃)₂ |
| A-424 | H | H | Br | OCH(CH₃)₂ |
| A-425 | H | H | CF₃ | OCH(CH₃)₂ |
| A-426 | F | H | F | OCH₂CH═CH₂ |
| A-427 | F | F | F | OCH₂CH═CH₂ |
| A-428 | F | Cl | F | OCH₂CH═CH₂ |
| A-429 | F | Br | F | OCH₂CH═CH₂ |
| A-430 | F | H | Cl | OCH₂CH═CH₂ |
| A-431 | F | H | Br | OCH₂CH═CH₂ |
| A-432 | Cl | H | Cl | OCH₂CH═CH₂ |
| A-433 | Cl | Cl | Cl | OCH₂CH═CH₂ |
| A-434 | Cl | F | Cl | OCH₂CH═CH₂ |
| A-435 | Cl | Br | Cl | OCH₂CH═CH₂ |
| A-436 | Cl | H | Br | OCH₂CH═CH₂ |
| A-437 | Br | H | Br | OCH₂CH═CH₂ |
| A-438 | Br | F | Br | OCH₂CH═CH₂ |
| A-439 | Br | Cl | Br | OCH₂CH═CH₂ |
| A-440 | CF₃ | H | F | OCH₂CH═CH₂ |
| A-441 | CF₃ | H | Cl | OCH₂CH═CH₂ |
| A-442 | CF₃ | H | Br | OCH₂CH═CH₂ |
| A-443 | CF₃ | H | CF₃ | OCH₂CH═CH₂ |
| A-444 | CF₃ | F | F | OCH₂CH═CH₂ |
| A-445 | CF₃ | Cl | Cl | OCH₂CH═CH₂ |
| A-446 | CF₃ | Br | Br | OCH₂CH═CH₂ |
| A-447 | H | H | F | OCH₂CH═CH₂ |
| A-448 | H | H | Cl | OCH₂CH═CH₂ |
| A-449 | H | H | Br | OCH₂CH═CH₂ |
| A-450 | H | H | CF₃ | OCH₂CH═CH₂ |
| A-451 | F | H | F | O—^{c}C₃H₅* |
| A-452 | F | F | F | O—^{c}C₃H₅* |
| A-453 | F | Cl | F | O—^{c}C₃H₅* |
| A-454 | F | Br | F | O—^{c}C₃H₅* |
| A-455 | F | H | Cl | O—^{c}C₃H₅* |
| A-456 | F | H | Br | O—^{c}C₃H₅* |
| A-457 | Cl | H | Cl | O—^{c}C₃H₅* |
| A-458 | Cl | Cl | Cl | O—^{c}C₃H₅* |
| A-459 | Cl | F | Cl | O—^{c}C₃H₅* |
| A-460 | Cl | Br | Cl | O—^{c}C₃H₅* |
| A-461 | Cl | H | Br | O—^{c}C₃H₅* |
| A-462 | Br | H | Br | O—^{c}C₃H₅* |
| A-463 | Br | F | Br | O—^{c}C₃H₅* |
| A-464 | Br | Cl | Br | O—^{c}C₃H₅* |
| A-465 | CF₃ | H | F | O—^{c}C₃H₅* |
| A-466 | CF₃ | H | Cl | O—^{c}C₃H₅* |
| A-467 | CF₃ | H | Br | O—^{c}C₃H₅* |
| A-468 | CF₃ | H | CF₃ | O—^{c}C₃H₅* |
| A-469 | CF₃ | F | F | O—^{c}C₃H₅* |
| A-470 | CF₃ | Cl | Cl | O—^{c}C₃H₅* |
| A-471 | CF₃ | Br | Br | O—^{c}C₃H₅* |
| A-472 | H | H | F | O—^{c}C₃H₅* |
| A-473 | H | H | Cl | O—^{c}C₃H₅* |
| A-474 | H | H | Br | O—^{c}C₃H₅* |
| A-475 | H | H | CF₃ | O—^{c}C₃H₅* |
| A-476 | F | H | F | OCHF₂ |
| A-477 | F | F | F | OCHF₂ |
| A-478 | F | Cl | F | OCHF₂ |
| A-479 | F | Br | F | OCHF₂ |
| A-480 | F | H | Cl | OCHF₂ |
| A-481 | F | H | Br | OCHF₂ |
| A-482 | Cl | H | Cl | OCHF₂ |
| A-483 | Cl | Cl | Cl | OCHF₂ |
| A-484 | Cl | F | Cl | OCHF₂ |
| A-485 | Cl | Br | Cl | OCHF₂ |
| A-486 | Cl | H | Br | OCHF₂ |
| A-487 | Br | H | Br | OCHF₂ |
| A-488 | Br | F | Br | OCHF₂ |
| A-489 | Br | Cl | Br | OCHF₂ |
| A-490 | CF₃ | H | F | OCHF₂ |
| A-491 | CF₃ | H | Cl | OCHF₂ |
| A-492 | CF₃ | H | Br | OCHF₂ |
| A-493 | CF₃ | H | CF₃ | OCHF₂ |
| A-494 | CF₃ | F | F | OCHF₂ |
| A-495 | CF₃ | Cl | Cl | OCHF₂ |
| A-496 | CF₃ | Br | Br | OCHF₂ |
| A-497 | H | H | F | OCHF₂ |
| A-498 | H | H | Cl | OCHF₂ |
| A-499 | H | H | Br | OCHF₂ |
| A-500 | H | H | CF₃ | OCHF₂ |
| A-501 | F | H | F | OCF₃ |
| A-502 | F | F | F | OCF₃ |
| A-503 | F | Cl | F | OCF₃ |
| A-504 | F | Br | F | OCF₃ |
| A-505 | F | H | Cl | OCF₃ |
| A-506 | F | H | Br | OCF₃ |
| A-507 | Cl | H | Cl | OCF₃ |
| A-508 | Cl | Cl | Cl | OCF₃ |
| A-509 | Cl | F | Cl | OCF₃ |
| A-510 | Cl | Br | Cl | OCF₃ |
| A-511 | Cl | H | Br | OCF₃ |
| A-512 | Br | H | Br | OCF₃ |
| A-513 | Br | F | Br | OCF₃ |
| A-514 | Br | Cl | Br | OCF₃ |
| A-515 | CF₃ | H | F | OCF₃ |
| A-516 | CF₃ | H | Cl | OCF₃ |
| A-517 | CF₃ | H | Br | OCF₃ |
| A-518 | CF₃ | H | CF₃ | OCF₃ |
| A-519 | CF₃ | F | F | OCF₃ |
| A-520 | CF₃ | Cl | Cl | OCF₃ |
| A-521 | CF₃ | Br | Br | OCF₃ |
| A-522 | H | H | F | OCF₃ |
| A-523 | H | H | Cl | OCF₃ |
| A-524 | H | H | Br | OCF₃ |
| A-525 | H | H | CF₃ | OCF₃ |
| A-526 | F | H | F | OCH₂CF₃ |
| A-527 | F | F | F | OCH₂CF₃ |
| A-528 | F | Cl | F | OCH₂CF₃ |
| A-529 | F | Br | F | OCH₂CF₃ |
| A-530 | F | H | Cl | OCH₂CF₃ |
| A-531 | F | H | Br | OCH₂CF₃ |
| A-532 | Cl | H | Cl | OCH₂CF₃ |
| A-533 | Cl | Cl | Cl | OCH₂CF₃ |
| A-534 | Cl | F | Cl | OCH₂CF₃ |
| A-535 | Cl | Br | Cl | OCH₂CF₃ |
| A-536 | Cl | H | Br | OCH₂CF₃ |
| A-537 | Br | H | Br | OCH₂CF₃ |
| A-538 | Br | F | Br | OCH₂CF₃ |
| A-539 | Br | Cl | Br | OCH₂CF₃ |
| A-540 | CF₃ | H | F | OCH₂CF₃ |
| A-541 | CF₃ | H | Cl | OCH₂CF₃ |
| A-542 | CF₃ | H | Br | OCH₂CF₃ |
| A-543 | CF₃ | H | CF₃ | OCH₂CF₃ |
| A-544 | CF₃ | F | F | OCH₂CF₃ |
| A-545 | CF₃ | Cl | Cl | OCH₂CF₃ |
| A-546 | CF₃ | Br | Br | OCH₂CF₃ |
| A-547 | H | H | F | OCH₂CF₃ |
| A-548 | H | H | Cl | OCH₂CF₃ |
| A-549 | H | H | Br | OCH₂CF₃ |
| A-550 | H | H | CF₃ | OCH₂CF₃ |
| A-551 | F | H | F | SCH₃ |
| A-552 | F | F | F | SCH₃ |
| A-553 | F | Cl | F | SCH₃ |
| A-554 | F | Br | F | SCH₃ |
| A-555 | F | H | Cl | SCH₃ |
| A-556 | F | H | Br | SCH₃ |
| A-557 | Cl | H | Cl | SCH₃ |
| A-558 | Cl | Cl | Cl | SCH₃ |
| A-559 | Cl | F | Cl | SCH₃ |
| A-560 | Cl | Br | Cl | SCH₃ |
| A-561 | Cl | H | Br | SCH₃ |
| A-562 | Br | H | Br | SCH₃ |
| A-563 | Br | F | Br | SCH₃ |
| A-564 | Br | Cl | Br | SCH₃ |
| A-565 | CF₃ | H | F | SCH₃ |
| A-566 | CF₃ | H | Cl | SCH₃ |
| A-567 | CF₃ | H | Br | SCH₃ |
| A-568 | CF₃ | H | CF₃ | SCH₃ |
| A-569 | CF₃ | F | F | SCH₃ |
| A-570 | CF₃ | Cl | Cl | SCH₃ |
| A-571 | CF₃ | Br | Br | SCH₃ |
| A-572 | H | H | F | SCH₃ |
| A-573 | H | H | Cl | SCH₃ |
| A-574 | H | H | Br | SCH₃ |
| A-575 | H | H | CF₃ | SCH₃ |
| A-576 | F | H | F | SCH₂CH₃ |
| A-577 | F | F | F | SCH₂CH₃ |
| A-578 | F | Cl | F | SCH₂CH₃ |
| A-579 | F | Br | F | SCH₂CH₃ |
| A-580 | F | H | Cl | SCH₂CH₃ |
| A-581 | F | H | Br | SCH₂CH₃ |
| A-582 | Cl | H | Cl | SCH₂CH₃ |
| A-583 | Cl | Cl | Cl | SCH₂CH₃ |
| A-584 | Cl | F | Cl | SCH₂CH₃ |
| A-585 | Cl | Br | Cl | SCH₂CH₃ |
| A-586 | Cl | H | Br | SCH₂CH₃ |
| A-587 | Br | H | Br | SCH₂CH₃ |
| A-588 | Br | F | Br | SCH₂CH₃ |
| A-589 | Br | Cl | Br | SCH₂CH₃ |
| A-590 | CF₃ | H | F | SCH₂CH₃ |
| A-591 | CF₃ | H | Cl | SCH₂CH₃ |
| A-592 | CF₃ | H | Br | SCH₂CH₃ |
| A-593 | CF₃ | H | CF₃ | SCH₂CH₃ |
| A-594 | CF₃ | F | F | SCH₂CH₃ |
| A-595 | CF₃ | Cl | Cl | SCH₂CH₃ |
| A-596 | CF₃ | Br | Br | SCH₂CH₃ |
| A-597 | H | H | F | SCH₂CH₃ |
| A-598 | H | H | Cl | SCH₂CH₃ |
| A-599 | H | H | Br | SCH₂CH₃ |
| A-600 | H | H | CF₃ | SCH₂CH₃ |
| A-601 | F | H | F | SCH(CH₃)₂ |
| A-602 | F | F | F | SCH(CH₃)₂ |
| A-603 | F | Cl | F | SCH(CH₃)₂ |
| A-604 | F | Br | F | SCH(CH₃)₂ |
| A-605 | F | H | Cl | SCH(CH₃)₂ |
| A-606 | F | H | Br | SCH(CH₃)₂ |
| A-607 | Cl | H | Cl | SCH(CH₃)₂ |
| A-608 | Cl | Cl | Cl | SCH(CH₃)₂ |
| A-609 | Cl | F | Cl | SCH(CH₃)₂ |
| A-610 | Cl | Br | Cl | SCH(CH₃)₂ |
| A-611 | Cl | H | Br | SCH(CH₃)₂ |
| A-612 | Br | H | Br | SCH(CH₃)₂ |
| A-613 | Br | F | Br | SCH(CH₃)₂ |
| A-614 | Br | Cl | Br | SCH(CH₃)₂ |
| A-615 | CF₃ | H | F | SCH(CH₃)₂ |
| A-616 | CF₃ | H | Cl | SCH(CH₃)₂ |
| A-617 | CF₃ | H | Br | SCH(CH₃)₂ |
| A-618 | CF₃ | H | CF₃ | SCH(CH₃)₂ |
| A-619 | CF₃ | F | F | SCH(CH₃)₂ |
| A-620 | CF₃ | Cl | Cl | SCH(CH₃)₂ |
| A-621 | CF₃ | Br | Br | SCH(CH₃)₂ |
| A-622 | H | H | F | SCH(CH₃)₂ |
| A-623 | H | H | Cl | SCH(CH₃)₂ |
| A-624 | H | H | Br | SCH(CH₃)₂ |
| A-625 | H | H | CF₃ | SCH(CH₃)₂ |
| A-626 | F | H | F | SCH₂CH═CH₂ |
| A-627 | F | F | F | SCH₂CH═CH₂ |
| A-628 | F | Cl | F | SCH₂CH═CH₂ |
| A-629 | F | Br | F | SCH₂CH═CH₂ |
| A-630 | F | H | Cl | SCH₂CH═CH₂ |
| A-631 | F | H | Br | SCH₂CH═CH₂ |
| A-632 | Cl | H | Cl | SCH₂CH═CH₂ |
| A-633 | Cl | Cl | Cl | SCH₂CH═CH₂ |
| A-634 | Cl | F | Cl | SCH₂CH═CH₂ |
| A-635 | Cl | Br | Cl | SCH₂CH═CH₂ |
| A-636 | Cl | H | Br | SCH₂CH═CH₂ |
| A-637 | Br | H | Br | SCH₂CH═CH₂ |
| A-638 | Br | F | Br | SCH₂CH═CH₂ |
| A-639 | Br | Cl | Br | SCH₂CH═CH₂ |
| A-640 | CF₃ | H | F | SCH₂CH═CH₂ |
| A-641 | CF₃ | H | Cl | SCH₂CH═CH₂ |
| A-642 | CF₃ | H | Br | SCH₂CH═CH₂ |
| A-643 | CF₃ | H | CF₃ | SCH₂CH═CH₂ |
| A-644 | CF₃ | F | F | SCH₂CH═CH₂ |
| A-645 | CF₃ | Cl | Cl | SCH₂CH═CH₂ |
| A-646 | CF₃ | Br | Br | SCH₂CH═CH₂ |
| A-647 | H | H | F | SCH₂CH═CH₂ |
| A-648 | H | H | Cl | SCH₂CH═CH₂ |
| A-649 | H | H | Br | SCH₂CH═CH₂ |
| A-650 | H | H | CF₃ | SCH₂CH═CH₂ |
| A-651 | F | H | F | S—^{c}C₃H₅* |
| A-652 | F | F | F | S—^{c}C₃H₅* |
| A-653 | F | Cl | F | S—^{c}C₃H₅* |
| A-654 | F | Br | F | S—^{c}C₃H₅* |
| A-655 | F | H | Cl | S—^{c}C₃H₅* |
| A-656 | F | H | Br | S—^{c}C₃H₅* |
| A-657 | Cl | H | Cl | S—^{c}C₃H₅* |
| A-658 | Cl | Cl | Cl | S—^{c}C₃H₅* |
| A-659 | Cl | F | Cl | S—^{c}C₃H₅* |
| A-660 | Cl | Br | Cl | S—^{c}C₃H₅* |
| A-661 | Cl | H | Br | S—^{c}C₃H₅* |
| A-662 | Br | H | Br | S—^{c}C₃H₅* |
| A-663 | Br | F | Br | S—^{c}C₃H₅* |
| A-664 | Br | Cl | Br | S—^{c}C₃H₅* |
| A-665 | CF₃ | H | F | S—^{c}C₃H₅* |
| A-666 | CF₃ | H | Cl | S—^{c}C₃H₅* |
| A-667 | CF₃ | H | Br | S—^{c}C₃H₅* |
| A-668 | CF₃ | H | CF₃ | S—^{c}C₃H₅* |
| A-669 | CF₃ | F | F | S—^{c}C₃H₅* |
| A-670 | CF₃ | Cl | Cl | S—^{c}C₃H₅* |
| A-671 | CF₃ | Br | Br | S—^{c}C₃H₅* |
| A-672 | H | H | F | S—^{c}C₃H₅* |
| A-673 | H | H | Cl | S—^{c}C₃H₅* |
| A-674 | H | H | Br | S—^{c}C₃H₅* |
| A-675 | H | H | CF₃ | S—^{c}C₃H₅* |
| A-676 | F | H | F | SCF₃ |
| A-677 | F | F | F | SCF₃ |
| A-678 | F | Cl | F | SCF₃ |
| A-679 | F | Br | F | SCF₃ |
| A-680 | F | H | Cl | SCF₃ |
| A-681 | F | H | Br | SCF₃ |
| A-682 | Cl | H | Cl | SCF₃ |
| A-683 | Cl | Cl | Cl | SCF₃ |
| A-684 | Cl | F | Cl | SCF₃ |
| A-685 | Cl | Br | Cl | SCF₃ |
| A-686 | Cl | H | Br | SCF₃ |
| A-687 | Br | H | Br | SCF₃ |
| A-688 | Br | F | Br | SCF₃ |
| A-689 | Br | Cl | Br | SCF₃ |
| A-690 | CF₃ | H | F | SCF₃ |
| A-691 | CF₃ | H | Cl | SCF₃ |
| A-692 | CF₃ | H | Br | SCF₃ |
| A-693 | CF₃ | H | CF₃ | SCF₃ |
| A-694 | CF₃ | F | F | SCF₃ |
| A-695 | CF₃ | Cl | Cl | SCF₃ |
| A-696 | CF₃ | Br | Br | SCF₃ |
| A-697 | H | H | F | SCF₃ |
| A-698 | H | H | Cl | SCF₃ |
| A-699 | H | H | Br | SCF₃ |
| A-700 | H | H | CF₃ | SCF₃ |
| A-701 | F | H | F | SCH₂CF₃ |
| A-702 | F | F | F | SCH₂CF₃ |
| A-703 | F | Cl | F | SCH₂CF₃ |
| A-704 | F | Br | F | SCH₂CF₃ |
| A-705 | F | H | Cl | SCH₂CF₃ |
| A-706 | F | H | Br | SCH₂CF₃ |
| A-707 | Cl | H | Cl | SCH₂CF₃ |
| A-708 | Cl | Cl | Cl | SCH₂CF₃ |
| A-709 | Cl | F | Cl | SCH₂CF₃ |
| A-710 | Cl | Br | Cl | SCH₂CF₃ |
| A-711 | Cl | H | Br | SCH₂CF₃ |
| A-712 | Br | H | Br | SCH₂CF₃ |
| A-713 | Br | F | Br | SCH₂CF₃ |
| A-714 | Br | Cl | Br | SCH₂CF₃ |
| A-715 | CF₃ | H | F | SCH₂CF₃ |
| A-716 | CF₃ | H | Cl | SCH₂CF₃ |
| A-717 | CF₃ | H | Br | SCH₂CF₃ |
| A-718 | CF₃ | H | CF₃ | SCH₂CF₃ |
| A-719 | CF₃ | F | F | SCH₂CF₃ |
| A-720 | CF₃ | Cl | Cl | SCH₂CF₃ |
| A-721 | CF₃ | Br | Br | SCH₂CF₃ |
| A-722 | H | H | F | SCH₂CF₃ |
| A-723 | H | H | Cl | SCH₂CF₃ |
| A-724 | H | H | Br | SCH₂CF₃ |
| A-725 | H | H | CF₃ | SCH₂CF₃ |

*^{c}C₃H₅ ═ cyclopropyl

Among the above compounds, preference is given to compounds Ia.1 and Ia.5 to Ia.33. More preference is given to compounds Ia.1 and Ia.5 and especially to Ia.5.

The compounds of the formula (I) can be prepared by novel methods as described below or and in the synthesis descriptions of the working examples, or by standard methods of organic chemistry, e.g. by the methods described hereinafter or in the synthesis descriptions of the working examples. The substituents, variables and indices are as defined above for formula (I), if not otherwise specified.

The compounds (I) can be prepared by coupling a compound 2 with a heterocyclic compound 3, wherein A′ is A or a precursor of A and LG is a C-bound halide ion, preferably a chloride, bromide or iodide ion, for instance under the conditions of a Heck reaction via Pd-catalyzed cross coupling, generally in the presence of a base, as shown in scheme 1. A typical catalyst is tetrakis(triphenylphosphine)palladium(0). Solvents such as tetrahydrofuran, acetonitrile, diethyl ether and dioxane are suitable. Other methods for introduction of the heteroaromatic group Q-A are the Suzuki, Stille and Kumada coupling procedures; see for example Tetrahedron, 2004, 60, 8991-9016. If A′ is a precursor of A, this can be then converted into the final group A.

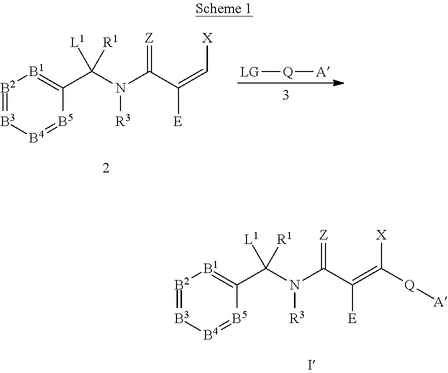

The compound 2 wherein Z is O can for example be prepared by an amidation reaction of the acrylic acid (derivative) 5 with the benzylic amine 4 as shown in scheme 2. LG is OH, Cl, Br, an anhydride residue (—O—C(O)—R) or an active ester residue (—OR′). —O—C(O)—R is a group which can be displaced easily by the amino group of 4. Suitable acid derivatives with which the carboxylic acid 5 (LG=OH) can form suitable mixed anhydrides are, for example, the esters of chloroformic acid, for example isopropyl chloroformate and isobutyl chloroformate, or of chloroacetic acid. If 5 with LG=OR′ forms a so-called active ester, this is obtained in a formal sense by the reaction of the acid 5 (LG=OH) with an active ester-forming alcohol, such as p-nitrophenol, N-hydroxybenzotriazole (HOBt), N-hydroxysuccinimide or OPfp (pentafluorophenol). In case that LG is OH, amidation is preferably carried out by first activating the carboxylic acid with oxalylchloride [(COCl)₂] or thionylchloride (SOCl₂) to the respective acid chlorides, followed by reaction with amine 4. Alternatively, amidation is carried out in the presence of a coupling reagent. Suitable coupling reagents (activators) are well known and are for instance selected from carbodiimides, such as DCC (dicyclohexylcarbodiimide) and DIPC (diisopropylcarbodiimide), benzotriazol derivatives, such as HATU (O-(7-azabenzotriazol-1-yl)-N,N,N′,N′-tetramethyluronium hexafluorophosphate), HBTU ((O-benzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate). Generally, the activator is used in excess. The benzotriazol and phosphonium coupling reagents are generally used in a basic medium.

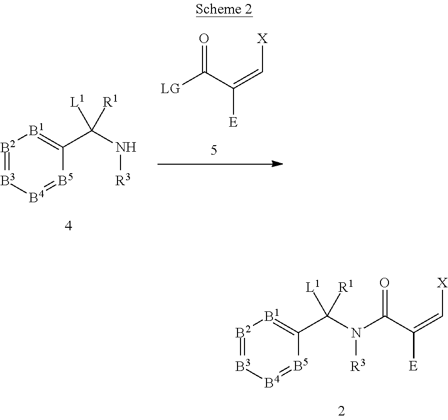

The benzylic amine 4^{a}, wherein L¹ is H, can be prepared by reaction the aldehyde or ketone 7 with hydroxylamine to the oxime 6, as shown in scheme 3. Subsequent reduction and, in case that R³ is not hydrogen, reaction with a group X—R³, wherein X is a leaving group, such as a halogen atom, and R³ is not H, yields compound 4^{a}.

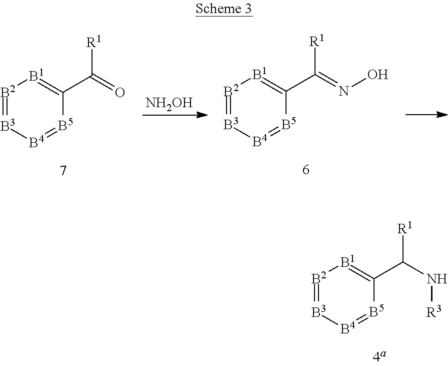

Alternatively, the benzylic amine 4 can be prepared by reaction the aldehyde or ketone 7 with an amine NH₂R³ to the imine 8, as shown in scheme 4. Standard reduction (hydrogenation) conditions give a compound 4^{a}, wherein L¹ is H. For obtaining compounds 4 wherein L¹ is a C-bound radical, such as alkyl, cycloalkyl, alkenyl, alkynyl, phenyl or a C-bound heterocyclyl, the imine 8 is reacted with a Grignard reagent L¹-MgX, wherein X is CI or Br, or with a lithium organic compound L¹-Li.

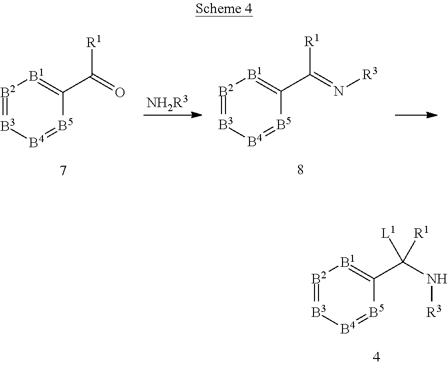

The benzylic amine 4^{b}, wherein L¹ is OH, can be prepared as shown in scheme 5 by reacting the imine 8 with a carbonic dichloride COCl₂ in the presence of a base, such as Hünig's base (N,N-diisopropylethylamine) and subsequent quenching with an aqueous solution, as described in WO 2004/013110. Reaction of 4^{b} with SOCl₂ in the presence of a base, such as triethylamine, yields an amine 4^{c} wherein L¹ is Cl.

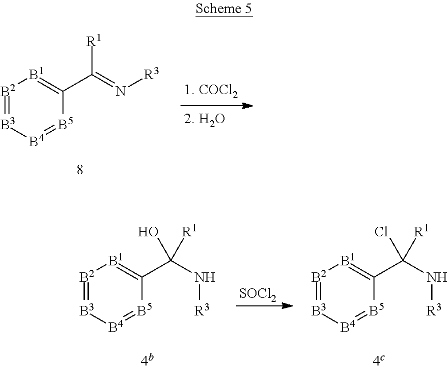

The benzylic amine 4^{d}, wherein L¹ is alkoxy, can be prepared as shown in scheme 6 in analogy to the reactions described in J. Org. Chem 1988, 53(4), 3358-3361, Zhurnal Organicheskoi Khimii 1981, 17(7), 1420-1429, 1263-1271, J. Org. Chem. USSR (English Translation) 1992, 28(19.1), 1635-1643 or Zhurnal Organicheskoi Khimii 1992, 28(10), 2042-2053 by reacting the acyl chloride 9 (R=alkyl) with sodium azide to 10. Curtius rearrangement yields the isocyanate 11. This is reacted with an alcohol R′OH (R′═C₁-C₄-alkyl or benzyl) to the carbamate 12, which is the hydrolysed to 4^{d}. Alternatively, isocyanate 11 can be reacted with water and thereby directly converted into the amine 4^{d}.

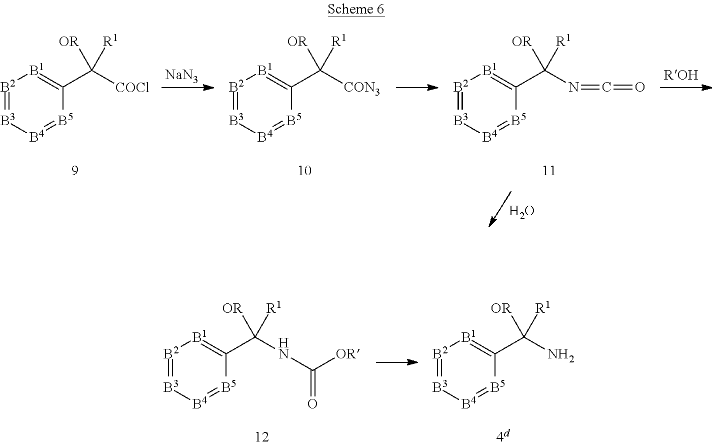

Amines 4^{e}, wherein L¹ is CN, can be prepared in analogy to the reaction described in Tetrahedron 1993, 49(8), 1541-1546 by reacting the amide 13 with phosphorus pentachloride to the nitrile 14. Grignard reaction with 15 yields the imine 16, which is then reacted with HCN to 4^{e}, as shown in scheme 7.

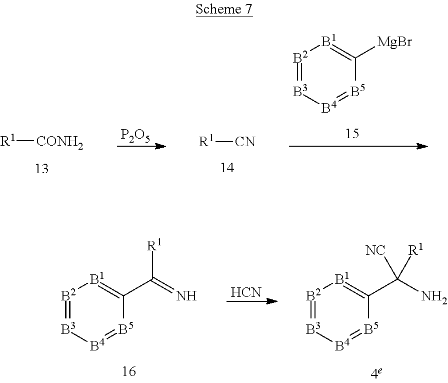

Amines 4^{f}, wherein L¹ is NH₂, can be prepared in analogy to the reaction described in Chemistry of heterocyclic compounds 2004, 40(3), 370-376 by reacting the imine 17 with and amine NH₂R³ to 18, as shown in scheme 8. Hydrolysis of the carbamate group yields 4^{f}.

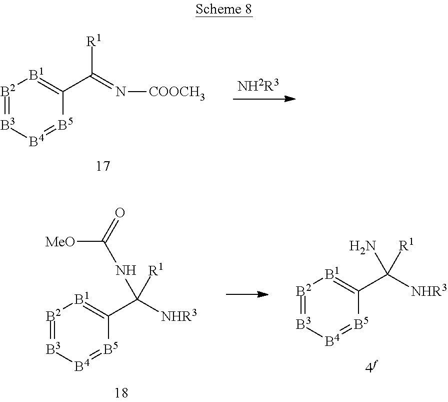

Compounds 4^{g}, wherein L¹ and R² form a ring, can be prepared in analogy to the synthesis described in EP-A-2042480 by reacting 19 with a Grignard or lithium organyl M-R¹, wherein M is Li or MgCl, to 20. Reaction with sodium azide in the presence of an acid, such as trifluoroacetic acid, yields 21, which is then reduced to 4^{g}, as shown in scheme 9.

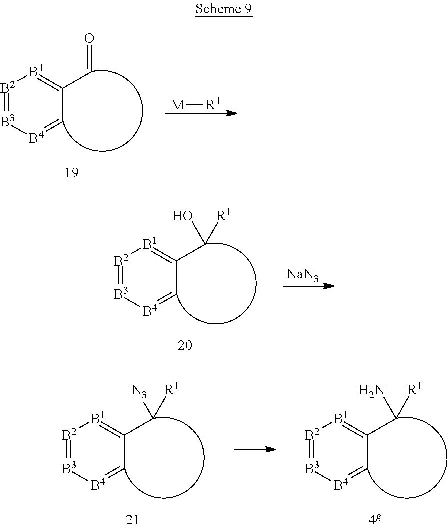

Compounds of formula I wherein Z is O can alternatively be prepared in an amidation reaction of the acrylic acid (derivative) 22 with the benzylic amine 4, as shown in scheme 10. LG is like above OH, Cl, Br, an anhydride residue (—O—C(O)—R) or an active ester residue (—OR′). Suitable amidation conditions correspond to those described above for scheme 2.

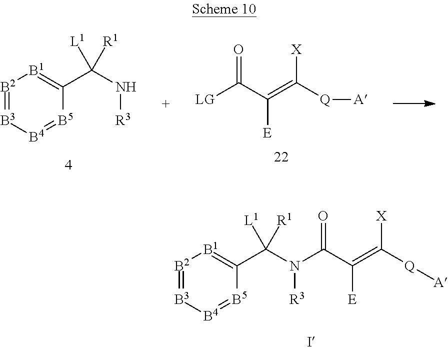

The acrylic acid (derivative) 22 can be prepared by reacting the acrylic acid (derivative) 5 with a heterocyclic compound 3, as shown in scheme 11, under the conditions of a Heck coupling reaction, as described above for scheme 1.

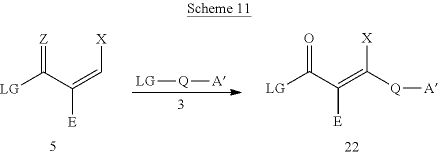

Compounds wherein Z is S can be prepared by reacting a compound I, I′ or 2 with Lawesson's reagent or P₂S₅.

Compounds wherein E and R⁴ or E and R^{4a} or two radicals R⁴ or R⁴ and R^{4a} or R⁴ and R⁵ or R^{4a} and R⁵, together with the atoms to which they are bound, form a ring, can be prepared by using respective starting material. This is either commercially available or can be prepared in analogy to known methods, such as described, for example, in WO 02/36583, WO 02/59099, WO 2005/034880, WO 2007/146758 or WO 2011/012538.

A′ as a precursor of A is typically a halogen atom, CN, carboxy, tert-butoxycarbonyl, an acetale group, a protected aldehyde group or —OSO₂—R^{z1}, where R^{z1} is C₁-C₄-alkyl, C₁-C₄-haloalkyl or phenyl which may be substituted by 1, 2 or 3 radicals selected from C₁-C₄-alkyl, C₁-C₄-haloalkyl C₁-C₄-alkoxy or C₁-C₄-haloalkoxy. A′ as a precursor of A is preferably a halogen atom or —OSO₂—R^{z1}, where R^{z1} is as defined above, and is more preferably a halogen atom or Otriflate.

Compounds I′, in which A′ is a precursor of A, can be converted as shown below into the different groups A¹ to A⁴.

Compounds I′, in which A′ is Cl, Br, I or —OSO₂—R^{z1}, where R^{z1} is as defined above, can be converted to compounds I wherein A is a group A¹, wherein A¹ is an imino group —C(═NR⁶)R⁸, by reaction with carbon monoxide and a hydride source, such as triethylsilane, in the presence of a transition metal complex catalyst, preferably a palladium catalyst, to a carbonyl compound II. This reaction converts the starting group A′ into a carbonyl group —C(═O)H.

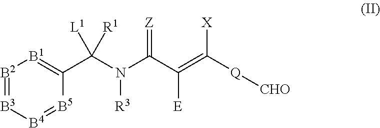

The aldehyde II can also be obtained by reducing the methyl ester V (see below; R=methyl) with diisobutylaluminium hydride (DIBAL-H) either directly to the aldehyde II or via the corresponding alcohol, which is then oxidized to the aldehyde.

For obtaining compounds in which R⁸ in the imino group is H, such carbonyl compounds II are then reacted with an amine (derivative) NH₂R⁶. Alternatively, the compound I′, in which A′ is Cl, Br, I or —OSO₂—R^{z1}, where R^{z1} is as defined above, can be reacted in a one pot reaction with carbon monoxide and hydrogen in the presence of a transition metal complex catalyst and the amine NH₂R⁶.

For obtaining compounds in which R⁸ in the imino group is not H, the carbonyl compounds are reacted with a Grignard reagent R⁸—MgHal, where Hal is Cl, Br or I, or an organolithium compound R⁸—Li to obtain an alcohol of formula III

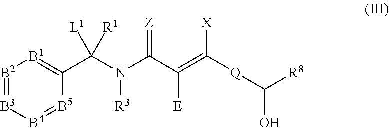

which is then oxidized to a carbonyl compound of the formula IV

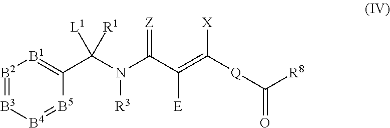

This is then reacted with an amine NH₂R⁶ to the respective imine compound.

These reactions can be carried out in analogy to the methods described in PCT/EP 2011/060388 or in WO 2010/072781 and the references cited therein, especially WO 2006135763, Fattorusso et al, J. Med. Chem. 2008, 51, 1333-1343 and WO 2008/122375.

Compounds I wherein A is a group A¹, wherein A¹ is —S(O)ₙR⁹ or —N(R⁵)R⁶, can for example be prepared by reacting a compound I′ wherein A′ is Cl, Br or I in a Ullmann-type reaction with an amine NHR⁵R⁶ or a thiol SHR⁹ in the presence of a Cu(I) catalyst. To obtain a compound wherein A¹ is —S(O)ₙR⁹ wherein n is not 0 the thiol can then be oxidized, e.g. with hydrogen peroxide. Amine groups can further be introduced in a Buchwald-Hartwig reaction by reacting a compound I′ wherein a′ is Cl, Br or I with an amine NHR⁵R⁶ in the presence of a palladium catalyst, such as PdCl₂(dppf) in the presence of a base, such as cesium carbonate.

Thioethers (A¹=—SR⁹) can further be introduced by reacting a compound I′ wherein A′ is F in an S_{N}Ar reaction (nucleophilic aromatic substitution reaction) with a thiol HSR⁹ in the presence of a base, such as potassium carbonate (K₂CO₃), or with a thiolate (e.g. NaSR⁹).

Compounds I wherein A is a group A², wherein W is O and Y is OR⁹ can be prepared by reacting a compound I′ wherein A′ is Cl, Br, I or Otriflate with carbon monoxide in the presence of a palladium catalyst and an alcohol R⁹OH. Compounds I wherein A is a group A², wherein W is O and Y is NR⁵R⁶ can be prepared by reacting a compound I′ wherein A′ is Cl, Br, I or Otriflate with carbon monoxide in the presence of a palladium catalyst and an alcohol ROH, wherein R is C₁-C₄-alkyl, to a compound of formula V. Suitable palladium catalysts are for example those described in PCT/EP 2011/060388.

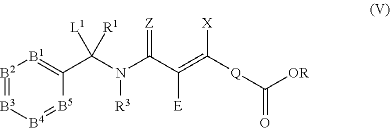

This ester is then hydrolyzed to the respective carboxylic acid, which is the reacted under standard amidation conditions with an amine NHR⁵R⁶. Hydrolyzation can be carried out under standard conditions, e.g. under acidic conditions using for example hydrochloric acid, sulfuric acid or trifluoroacetic acid, or under basic conditions using for example an alkali metal hydroxide, such as LiOH, NaOH or KOH. Amidation is preferably carried out by activation of the carboxylic acids with oxalylchloride [(COCl)₂] or thionylchloride (SOCl₂) to the respective acid chlorides, followed by reaction with an amine NHR⁵R⁶. Alternatively, amidation is carried out in the presence of a coupling reagent. Suitable coupling reagent (activators) are well known and are for instance selected from carbodiimides, such as DCC (dicyclohexylcarbodiimide) and DIPC (diisopropylcarbodiimide), benzotriazol derivatives, such as HATU (O-(7-azabenzotriazol-1-yl)N,N,N′,N′-tetramethyluronium hexafluorophosphate), HBTU ((O-benzotriazol-1-yl)N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate). Generally, the activator is used in excess. The benzotriazol and phosphonium coupling reagents are generally used in a basic medium.

Compounds I wherein A is a group A², wherein W is S and Y is NR⁵R⁶ or OR⁹, can be prepared by reacting the corresponding oxo-compound (W is O) with Lawesson's reagent (CAS 19172-47-5), see for example Jesberger et al., Synthesis, 2003, 1929-1958 and references therein. For compounds wherein Y is NR⁵R⁶, solvents such as HMPA or THF at an elevated temperature such as 60° C. to 100° C. can be used. Preferred reaction conditions are THF at 65° C. For compounds wherein Y is OR⁹, solvent free conditions or solvents such as toluene at temperatures such as 100° C. to 200° C., preferably 140° C., are suitable reaction conditions.

Compounds I wherein A is a group A³, wherein R^{7a} and R^{7b} are hydrogen, can be prepared by reducing a compound V or II for example with LAH (lithium aluminium hydride) or DIBAL-H (diisobutyl aluminium hydride) to a compound VI.

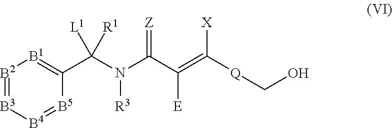

This is then reacted in an SN reaction with an amine NHR⁵R⁶. For this purpose, the OH group can first be converted into a better leaving group, e.g. into a sulfonate (for example mesylate, tosylate or a triflate group). If R⁶ is a group —C(O)R⁸, it is alternatively possible to react compound VI with an amine NH₂R⁵ and react then the resulting benzylic amine with an acid R⁸—COOH or a derivative thereof, such as its acid chloride R⁸—COCl, in an amidation reaction.

Compounds I wherein A is a group A³, wherein R^{7a} is optionally substituted alkyl or optionally substituted cycloalkyl and R^{7b} is hydrogen, can be prepared by subjecting a ketone IV, in which R⁸ corresponds to R^{7a} which is optionally substituted C₁-C₆-alkyl or optionally substituted C₃-C₈-cycloalkyl, to a reductive amination to furnish compounds VII. Typical conditions for the reductive amination are: Reacting ketone IV with an amine H₂NR⁵ to yield the corresponding imine which is reduced to amine VII with a reducing agent reagent such as Na(CN)BH₃. The reaction from ketone IV to amine VII may also be carried out as a one pot procedure.

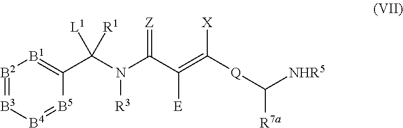

For obtaining compounds in which R^{7a} and R^{7b} are optionally substituted alkyl, optionally substituted cycloalkyl, optionally substituted alkenyl or optionally substituted alkynyl, carbonyl compounds such as IV, in which R⁸ corresponds to R^{7a} which is optionally substituted C₁-C₆-alkyl, optionally substituted C₃-C₈-cycloalkyl, optionally substituted C₂-C₆-alkenyl or optionally substituted C₂-C₆-alkynyl, is reacted with a Grignard reagent R^{7b}—MgHal, where Hal is Cl, Br or I, or an organolithium compound R^{7b}-Li, where R^{7b} is optionally substituted C₁-C₆-alkyl, optionally substituted C₃-C₈-cycloalkyl, optionally substituted C₂-C₆-alkenyl or optionally substituted C₂-C₆-alkynyl, to obtain an alcohol of formula VIII.

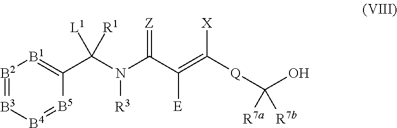

Alcohol VIII can then be converted into amine IX via the corresponding azide, as described, for example, in Organic Letters, 2001, 3(20), 3145-3148.

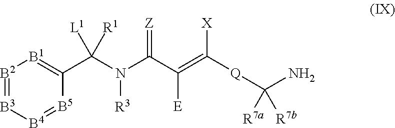

If desired, this can be converted into compounds I wherein R⁵ and R⁶ are different from hydrogen, for example by standard alkylation reactions.

Compounds I wherein A is a group A³, wherein R^{7a} is optionally substituted alkyl, alkenyl, alkynyl, cycloalkyl or CN and R^{7b} is hydrogen, can be prepared by converting an aldehyde II into an imine X by reaction with an amine derivative NH₂R⁶, wherein R⁶ is tert-butyl sulfinyl, or, for preparing a compound with R^{7a}=CN, tosylate.

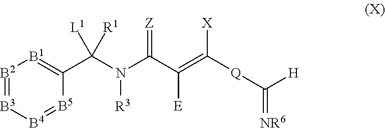

This imine is then reacted with a compound H—R^{7a} in an addition reaction under conditions as described for example in J. Am. Chem. Soc. 2009, 3850-3851 and the references cited therein, or, for introducing CN as a group R^{7a}, Chemistry—A European Journal 2009, 15, 11642-11659.

Compounds I wherein A is a group A³, wherein both R^{7a} and R^{7b} are optionally substituted alkyl, alkenyl, alkynyl, cycloalkyl or CN, can be prepared analogously by converting a ketone IV, wherein R⁸ is has the meaning desired for R^{7b} and is optionally substituted alkyl, alkenyl, alkynyl, cycloalkyl or CN, into an imine by reaction with an amine derivative NH₂R⁶, wherein R⁶ is tert-butyl sulfinyl, or, for preparing a compound with XI R^{7a}=CN, tosylate.

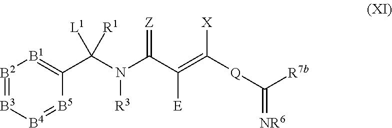

This imine is then reacted with a compound H—R^{7a} in an addition reaction under conditions as described for example in J. Org. Chem 2002, 67, 7819-7832 and the references cited therein, or, for introducing CN as a group R^{7a}, Chemistry—A European Journal 2009, 15, 11642-11659.

If desired, R⁶ can then be removed to yield an amino group NH₂.

Compounds I wherein A is A⁴ can be prepared by standard ring coupling reactions. For example, compounds, wherein A⁴ is an N-bound heterocyclic ring can be prepared by reacting a compound I′ wherein A is Cl, Br or I with the respective ring A⁴-H (H being on the nitrogen ring atom to be coupled) under Ullmann coupling conditions, such as described, for example, in WO 2007/075459. Typically, copper(I) iodide or copper(I) oxide and a ligand such as 1,2-cyclohexyldiamine is used, see for example Kanemasa et al., European Journal of Organic Chemistry, 2004, 695-709. If A′ is F, the reaction is typically run in a polar aprotic solvent such as N,N-dimethylformamide, N,N-dimethylacetamide or N-methylpyrrolidone, and in the presence of an inorganic base such as sodium, potassium or cesium carbonate.

Compounds, wherein A⁴ is a C-bound heterocyclic ring can be prepared by reacting a compound I′ wherein A′ is Br or I with the boronic acid of the respective ring A⁴-B(OH)₂ or the boronate ester of the respective ring A⁴-B(OR²) under Suzuki reaction conditions via Pd-catalyzed cross coupling, such as described, for example, in WO 2007/075459. A typical catalyst is tetrakis(triphenylphosphine)palladium(0). Solvents such as tetrahydrofuran, acetonitrile, diethyl ether and dioxane are suitable. The boronic acids A⁴-B(OH)₂ are either commercially available or can be prepared by known methods. Other methods for introduction of the heterocyclic groups A⁴ are the Heck, Stille, Kumada and Buchwald-Hartwig coupling procedures; see for example Tetrahedron, 2004, 60, 8991-9016.

As a rule, the compounds of formula (I) including their stereoisomers, salts, and N-oxides, and their precursors in the synthesis process, can be prepared by the methods described above. If individual compounds can not be prepared via the above-described routes, they can be prepared by derivatization of other compounds (I) or the respective precursor or by customary modifications of the synthesis routes described. For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

Due to their excellent activity, the compounds of the present invention may be used for controlling invertebrate pests.

Accordingly, the present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of the present invention or a composition as defined above.

Preferably, the method of the invention serves for protecting plant propagation material (such as seed) and the plant which grows therefrom from invertebrate pest attack or infestation and comprises treating the plant propagation material (such as seed) with a pesticidally effective amount of a compound of the present invention as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

In the sense of the present invention, “invertebrate pests” are preferably selected from arthropods and nematodes, more preferably from harmful insects, arachnids and nematodes, and even more preferably from insects, acarids and nematodes. In the sense of the present invention, “invertebrate pests” are most preferably insects.

The invention further provides an agricultural composition for combating invertebrate pests, which comprises such an amount of at least one compound according to the invention and at least one inert liquid and/or solid agronomically acceptable carrier that has a pesticidal action and, if desired, at least one surfactant.

Such a composition may comprise a single active compound of the present invention or a mixture of several active compounds of the present invention. The composition according to the present invention may comprise an individual isomer or mixtures of isomers or a salt as well as individual tautomers or mixtures of tautomers.

The compounds of the present invention, including their salts, stereoisomers and tautomers, are in particular suitable for efficiently controlling arthropodal pests such as arachnids, myriapedes and insects as well as nematodes. They are especially suitable for efficiently combating or controlling the following pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Pieris rapae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus cornmunis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria;

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;

bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus;

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;

arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

Collembola (springtails), e.g. Onychiurus ssp.

The compounds of the present invention, including their salts, stereoisomers and tautomers, are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the present invention, including their salts, stereoisomers and tautomers, are particularly useful for controlling insects, preferably sucking or piercing and chewing and biting insects such as insects from the genera Lepidoptera, Coleoptera and Hemiptera, in particular Lepidoptera, Coleoptera and true bugs.

The compounds of the present invention, including their salts, stereoisomers and tautomers, are moreover useful for controlling insects of the orders Thysanoptera, Diptera (especially flies, mosquitos), Hymenoptera (especially ants) and Isoptera (especially termites.

The compounds of the present invention, including their salts, stereoisomers and tautomers, are particularly useful for controlling insects of the orders Lepidoptera and Coleoptera.

The invention also relates to agrochemical compositions comprising an auxiliary and at least one compound I according to the invention.

An agrochemical composition comprises a pesticidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinyl-alcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

### ii) Dispersible Concentrates (DC)

5-25 wt % of a compound I according to the invention and 1-10 wt % dispersant (e. g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

### iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

### vi) Water-Dispersible Granules and Water-Soluble Granules (WG, SG)

50-80 wt % of a compound I according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

### iv) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water ad 100%. This mixture is stirred for 1 h to produce spontaneously a thermodynamically stable microemulsion.

### iv) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsules. The monomers amount to 1-10 wt %. The wt % relate to the total CS composition.

### ix) Dustable Powders (DP, DS)

1-10 wt % of a compound I according to the invention are ground finely and mixed intimately with solid carrier (e.g. finely divided kaolin) ad 100 wt %.

### x) Granules (GR, FG)

0.5-30 wt % of a compound I according to the invention is ground finely and associated with solid carrier (e.g. silicate) ad 100 wt %. Granulation is achieved by extrusion, spray-drying or the fluidized bed.

### xi) Ultra-Low Volume Liquids (UL)

1-50 wt % of a compound I according to the invention are dissolved in organic solvent (e.g. aromatic hydrocarbon) ad 100 wt %.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required. When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate. In a further embodiment, either individual components of the composition according to the invention or partially premixed components, e. g. components comprising compounds I and/or active substances from the groups M) or F) (see below), may be mixed by the user in a spray tank and further auxiliaries and additives may be added, if appropriate.

In a further embodiment, either individual components of the composition according to the invention or partially premixed components, e. g. components comprising compounds I and/or active substances from the groups M) or F) (see below), can be applied jointly (e.g. after tank mix) or consecutively.

The following categorized list M of pesticides represents insecticidal mixture partners, which are, whenever possible, classified according to the Insecticide Resistance Action Committee (IRAC), and together with which the compounds according to the present invention may be used. The combined use of the compounds of the present invention with the following pesticides may result in potential synergistic effects. The following examples of insecticidal mixing partners are provided with the intention to illustrate the possible combinations, but not to impose any limitation to the obtainable mixtures:

M.1 Acetylcholine esterase (AChE) inhibitors from the class of
M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
M.2. GABA-gated chloride channel antagonists such as:
M.2A cyclodiene organochlorine compounds, as for example endosulfan or chlordane; or
M.2B fiproles (phenylpyrazoles), as for example ethiprole, fipronil, flufiprole, pyrafluprole and pyriprole;
M.3 Sodium channel modulators from the class of
M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zetacypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
M.3B sodium channel modulators such as DDT or methoxychlor;
M.4 Nicotinic acetylcholine receptor agonists (nAChR) from the class of
M.4A neonicotinoids, for example acteamiprid, chlothianidin, dinotefuran, imidacloprid, nitenpyram, thiacloprid and thiamethoxam; or
M.4B nicotine.
M.5 Nicotinic acetylcholine receptor allosteric activators from the class of spinosyns, for example spinosad or spinetoram;
M.6 Chloride channel activators from the class of avermectins and milbemycins, for example abamectin, emamectin benzoate, ivermectin, lepimectin or milbemectin;
M.7 Juvenile hormone mimics, such as
M.7A juvenile hormone analogues as hydroprene, kinoprene and methoprene; or others as
M.7B fenoxycarb, or
M.7C pyriproxyfen;
M.8 miscellaneous non-specific (multi-site) inhibitors, for example
M.8A alkyl halides as methyl bromide and other alkyl halides, or
M.8B chloropicrin, or
M.8C sulfuryl fluoride, or
M.8D borax, or
M.8E tartar emetic;
M.9 Selective homopteran feeding blockers, for example
M.9B pymetrozine, or
M.9C flonicamid;
M.10 Mite growth inhibitors, for example
M.10A clofentezine, hexythiazox and diflovidazin, or
M.10B etoxazole;
M.11 Microbial disruptors of insect midgut membranes, for example bacillus thuringiensis or bacillus sphaericus and the insecticidal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebriosis, or the Bt crop proteins: Cry1Ab, Cry1Ac, Cry1Fa, Cry2Ab, mCry3A, Cry3Ab, Cry3Bb and Cry34/35Ab1;
M.12 Inhibitors of mitochondrial ATP synthase, for example
M.12A diafenthiuron, or
M.12B organotin miticides such as azocyclotin, cyhexatin or fenbutatin oxide, or M.12C propargite, or
M.12D tetradifon;
M.13 Uncouplers of oxidative phosphorylation via disruption of the proton gradient, for example chlorfenapyr, DNOC or sulfluramid;
M.14 Nicotinic acetylcholine receptor (nAChR) channel blockers, for example nereistoxin analogues as bensultap, cartap hydrochloride, thiocyclam or thiosultap sodium;
M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;
M.16 Inhibitors of the chitin biosynthesis type 1, as for example buprofezin;
M.17 Moulting disruptors, Dipteran, as for example cyromazine;
M.18 Ecdyson receptor agonists such as diacylhydrazines, for example methoxyfenozide, tebufenozide, halofenozide, fufenozide or chromafenozide;
M.19 Octopamin receptor agonists, as for example amitraz;
M.20 Mitochondrial complex III electron transport inhibitors, for example
M.20A hydramethylnon, or
M.20B acequinocyl, or
M.20C fluacrypyrim;
M.21 Mitochondrial complex I electron transport inhibitors, for example
M.21A METI acaricides and insecticides such as fenazaquin, fenpyroximate, pyrimidifen, pyridaben, tebufenpyrad or tolfenpyrad, or
M.21B rotenone;
M.22 Voltage-dependent sodium channel blockers, for example
M.22A indoxacarb, or
M.22B metaflumizone;
M.23 Inhibitors of the of acetyl CoA carboxylase, such as Tetronic and Tetramic acid derivatives, for example spirodiclofen, spiromesifen or spirotetramat;
M.24 Mitochondrial complex IV electron transport inhibitors, for example
M.24A phosphine such as aluminium phosphide, calcium phosphide, phosphine or zinc phosphide, or
M.24B cyanide.
M.25 Mitochondrial complex II electron transport inhibitors, such as beta-ketonitrile derivatives, for example cyenopyrafen or cyflumetofen;
M.26 Ryanodine receptor-modulators from the class of diamides, as for example flubendiamide, chlorantraniliprole (Rynaxypyr®), cyantraniliprole (Cyazypyr®), or
the phthalamide compounds
M.26.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
M.26.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
M.26.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound
M.26.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.26.5a) to M.26.5h):
M.26.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide;
M.26.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide;
M.26.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide;
M.26.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide;
M.26.5e) N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide;
M.26.5f) N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide;
M.26.5g) N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide;
M.26.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide;
M.X insecticidal active compounds of unknown or uncertain mode of action, as for example azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, or the compound
M.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound
M.X.3: 11-(4-chloro-2,6-dimethylphenyl)-12-hydroxy-1,4-dioxa-9-azadispiro[4.2.4.2]-tetradec-11-en-10-one, or the compound
M.X.4: 3-(4′-fluoro-2,4-dimethylbiphenyl-3-yl)-4-hydroxy-8-oxa-1-azaspiro[4.5]dec-3-en-2-one, or the compound
M.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, I-1582).

The commercially available compounds of the group M listed above may be found in The Pesticide Manual, 15th Edition, C. D. S. Tomlin, British Crop Protection Council (2011) among other publications.

The quinoline derivative flometoquin is shown in WO2006/013896. The aminofuranone compounds flupyradifurone is known from WO 2007/115644. The sulfoximine compound sulfoxaflor is known from WO2007/149134. The isoxazoline compound M.X.1 has been described in WO2005/085216. The pyripyropene derivative M.X.2 has been described in WO 2006/129714. The spiroketal-substituted cyclic ketoenol derivative M.X.3 is known from WO2006/089633 and the biphenyl-substituted spirocyclic ketoenol derivative M.X.4 from WO2008/067911. Triazoylphenylsulfide like M.X.5 have been described in WO2006/043635 and biological control agents on basis of bacillus firmus in WO2009/124707.

The phthalamides M.26.1 and M.26.2 are both known from WO 2007/101540. The anthranilamide M.26.3 has been described in WO2005/077934. The hydrazide compound M.26.4 has been described in WO 2007/043677. The anthranilamides M.26.5a) to M.26.5h) can be prepared as described in WO 2007/006670, PCT/EP2012/065650 and PCT/EP2012/065651.

The combined use of the compounds of formula (I) with other pesticides may result in potential synergistic effects. The examples of insecticidal mixing partners are provided with the intention to illustrate the possible combinations, but not to impose any limitation to the obtainable mixtures.

In another embodiment of the invention, the compounds of formula (I), or their stereoisomers, salts, tautomers and N-oxides, may also be applied with fungicides.

The following list F of active substances, in conjunction with which the compounds according to the invention can be used, is intended to illustrate the possible combinations but does not limit them:

### F.I) Respiration Inhibitors

F.I-1) Inhibitors of complex III at Qo site
strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)phenyl)-2-methoxyimino-N methyl-acetamide;
oxazolidinediones and imidazolinones: famoxadone, fenamidone;
F.I-2) Inhibitors of complex II (e.g. carboxamides):
carboxanilides: benodanil, benzovindiflupyr, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide (fluxapyroxad), N-(4′-trifluoromethylthiobiphenyl-2-yl)-3 difluoromethyl-1-methyl-1H pyrazole-4-carboxamide and N-(2-(1,3,3-trimethylbutyl)-phenyl)-1,3-dimethyl-5 fluoro-1H-pyrazole-4 carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide;
F.I-3) Inhibitors of complex III at Qi site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate, 3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl2-methylpropanoate;
F.I-4) Other respiration inhibitors (complex I, uncouplers)
diflumetorim; (5,8-difluoroquinazolin-4-yl)-{2-[2-fluoro-4-(4-trifluoromethylpyridin-2-yloxy)-phenyl]-ethyl}-amine; tecnazen; ametoctradin; silthiofam;
nitrophenyl derivates: binapacryl, dinobuton, dinocap, fluazinam, ferimzone, nitrthalisopropyl,
and including organometal compounds: fentin salts, such as fentin-acetate, fentin chloride or fentin hydroxide;
F.II) Sterol biosynthesis inhibitors (SBI fungicides)
F.II-1) C14 demethylase inhibitors (DMI fungicides, e.g. triazoles, imidazoles) triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2S,3R)-3-(2-chlorophenyl)-2-(2,4-difluorophenyl)-oxiranylmethyl]-5-thiocyanato-1H[1,2,4]triazole, 2-[rel-(2S,3R)-3-(2-chlorophenyl)-2-(2,4-difluorophenyl)-oxiranylmethyl]-2H-[1,2,4]triazole-3-thiol;
imidazoles: imazalil, pefurazoate, oxpoconazole, prochloraz, triflumizole;
pyrimidines, pyridines and piperazines: fenarimol, nuarimol, pyrifenox, triforine, 1-[rel-(2S,3R)-3-(2-chlorophenyl)-2-(2,4-difluorophenyl)-oxiranylmethyl]-5-thiocyanato-1H[1,2,4]triazole, 2-[rel-(2S;3R)-3-(2-chlorophenyl)-2-(2,4-difluorophenyl)-oxiranylmethyl]-2H-[1,2,4]triazole-3-thiol;
F.II-2) Delta14-reductase inhibitors (Amines, e.g. morpholines, piperidines)
morpholines: aldimorph, dodemorph, dodemorph-acetate, fenpropimorph, tridemorph;
piperidines: fenpropidin, piperalin;
spiroketalamines: spiroxamine;
F.II-3) Inhibitors of 3-keto reductase: hydroxyanilides: fenhexamid;
F.III) Nucleic acid synthesis inhibitors
F.III-1) RNA, DNA synthesis
phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;
F.III-2) DNA topisomerase inhibitors: oxolinic acid;
F.III-3) Nucleotide metabolism (e.g. adenosin-deaminase)
hydroxy (2-amino)-pyrimidines: bupirimate;
F.IV) Inhibitors of cell division and or cytoskeleton
F.IV-1) Tubulin inhibitors: benzimidazoles and thiophanates: benomyl, carbendazim, fuberidazole, thiabendazole, thiophanate-methyl;
triazolopyrimidines: 5-chloro-7 (4-methylpiperidin-1-yl)-6-(2,4,6-trifluorophenyl)[1,2,4]triazolo[1,5a]pyrimidine
F.IV-2) Other cell division inhibitors
benzamides and phenyl acetamides: diethofencarb, ethaboxam, pencycuron, fluopicolide, zoxamide;
F.IV-3) Actin inhibitors: benzophenones: metrafenone, pyriofenone;
F.V) Inhibitors of amino acid and protein synthesis
F.V-1) Methionine synthesis inhibitors (anilino-pyrimidines)
anilino-pyrimidines: cyprodinil, mepanipyrim, nitrapyrin, pyrimethanil;
F.V-2) Protein synthesis inhibitors (anilino-pyrimidines)
antibiotics: blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors (e.g. anilino-pyrimidines)
dicarboximides: fluoroimid, iprodione, procymidone, vinclozolin;
phenylpyrroles: fenpiclonil, fludioxonil;
F.VI-2) G protein inhibitors: quinolines: quinoxyfen;
F.VII) Lipid and membrane synthesis inhibitors
F.VII-1) Phospholipid biosynthesis inhibitors
organophosphorus compounds: edifenphos, iprobenfos, pyrazophos;
dithiolanes: isoprothiolane;
F.VII-2) Lipid peroxidation:
aromatic hydrocarbons: dicloran, quintozene, tecnazene, tolclofos-methyl, biphenyl, chloroneb, etridiazole;
F.VII-3) Carboxyl acid amides (CAA fungicides)
cinnamic or mandelic acid amides: dimethomorph, flumorph, mandiproamid, pyrimorph;
valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acids:
1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone,
carbamates: propamocarb, propamocarb-hydrochlorid,
F.VII-5) fatty acid amide hydrolase inhibitors: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone;
F.VIII) Inhibitors with Multi Site Action
F.VIII-1) Inorganic active substances: Bordeaux mixture, copper acetate, copper hydroxide, copper oxychloride, basic copper sulfate, sulfur;
F.VIII-2) Thio- and dithiocarbamates: ferbam, mancozeb, maneb, metam, methasulphocarb, metiram, propineb, thiram, zineb, ziram;
F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;
F.VIII-4) Guanidines and other: guanidine, dodine, dodine free base, guazatine, guazatine-acetate, iminoctadine, iminoctadine-triacetate, iminoctadine-tris(albesilate)), 2,6-dimethyl-1H,5H-[1,4]dithiino[2,3-c:5,6-c′]dipyrrole-1,3,5,7(2H,6H)-tetraone;
F.VIII-5) Ahtraquinones: dithianon;
F.IX) Cell wall synthesis inhibitors
F.IX-1) Inhibitors of glucan synthesis: validamycin, polyoxin B;
F.IX-2) Melanin synthesis inhibitors: pyroquilon, tricyclazole, carpropamide, dicyclomet, fenoxanil;
F.X) Plant defence inducers
F.X-1) Salicylic acid pathway: acibenzolar-S-methyl;
F.X-2) Others: probenazole, isotianil, tiadinil, prohexadione-calcium;
phosphonates: fosetyl, fosetyl-aluminum, phosphorous acid and its salts;
F.XI) Unknown mode of action:
bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxathiapiprolin, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluorophenyl)-methyl)-2-phenyl acetamide, N′-(4-(4-chloro-3-trifluoromethyl-phenoxy)-2,5-dimethyl-phenyl)-N-ethyl-N methyl formamidine, N′(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;
F.XI) Growth regulators:
abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;
F.XII) Biological control agents
Ampelomyces quisqualis (e.g. AQ 10® from Intrachem Bio GmbH & Co. KG, Germany), Aspergillus flavus (e.g. AFLAGUARD® from Syngenta, CH), Aureobasidium pullulans (e.g. BOTECTOR® from bio-ferm GmbH, Germany), Bacillus pumillus (e.g. NRRL Accession No. B-30087 in SONATA® and BALLAD® Plus from AgraQuest Inc., USA), Bacillus subtilis (e.g. isolate NRRL-Nr. B-21661 in RHAPSODY®, SERENADE® MAX and SERENADE® ASO from AgraQuest Inc., USA), Bacillus subtilis var. amyloliquefaciens FZB24 (e.g. TAEGRO® from Novozyme Biologicals, Inc., USA), Candida oleophila I-82 (e.g. ASPIRE® from Ecogen Inc., USA), Candida saitoana (e.g. BIOCURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans (e.g. CONTANS® from Prophyta, Germany), Cryphonectria parasitica (e.g. Endothia parasitica from CNICM, France), Cryptococcus albidus (e.g. YIELD PLUS® from Anchor Bio-Technologies, South Africa), Fusarium oxysporum (e.g. BIOFOX® from S.I.A.P.A., Italy, FUSACLEAN® from Natural Plant Protection, France), Metschnikowia fructicola (e.g. SHEMER® from Agrogreen, Israel), Microdochium dimerum (e.g. ANTIBOT® from Agrauxine, France), Phlebiopsis gigantea (e.g. ROTSOP® from Verdera, Finland), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis (e.g. REGALIA® from Marrone Biolnnovations, USA), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atroviride LC₅₂ (e.g. SENTINEL® from Agrimm Technologies Ltd, NZ), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-Innovation AB, Sweden), T. stromaticum (e.g. TRICOVAB® from C.E.P.L.A.C., Brazil), T. virens GL-21 (e.g. SOILGARD® from Certis LLC, USA), T. viride (e.g. TRIECO® from Ecosense Labs. (India) Pvt. Ltd., Indien, BIOCURE® F from T. Stanes & Co. Ltd., Indien), T. vinde TV1 (e.g. T. viride TV1 from Agribiotec srl, Italy), Ulocladium oudemansii HRU3 (e.g. BOTRY-ZEN® from Botry-Zen Ltd, NZ).

The commercially available compounds of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EPA 141 317; EP-A 152 031; EP-A 226 917; EPA 243 970; EPA 256 503; EP-A 428 941; EP-A 532 022; EP-A 1 028 125; EP-A 1 035 122; EPA 1 201 648; EP A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657).

The invertebrate pest (also referred to as “animal pest”), i.e. the insects, arachnids and nematodes, the plant, soil or water in which the plant is growing or may grow can be contacted with the compounds of the present invention or composition(s) comprising them by any application method known in the art. As such, “contacting” includes both direct contact (applying the compounds/compositions directly on the invertebrate pest or plant—typically to the foliage, stem or roots of the plant) and indirect contact (applying the compounds/compositions to the locus of the invertebrate pest or plant). The compounds of the present invention or the pesticidal compositions comprising them may be used to protect growing plants and crops from attack or infestation by animal pests, especially insects, acaridae or arachnids by contacting the plant/crop with a pesticidally effective amount of compounds of the present invention. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

Moreover, invertebrate pests may be controlled by contacting the target pest, its food supply, habitat, breeding ground or its locus with a pesticidally effective amount of compounds of the present invention. As such, the application may be carried out before or after the infection of the locus, growing crops, or harvested crops by the pest.

The compounds of the present invention can also be applied preventively to places at which occurrence of the pests is expected.

The compounds of the present invention may be also used to protect growing plants from attack or infestation by pests by contacting the plant with a pesticidally effective amount of compounds of the present invention. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 5 g to 500 g per hectare, more desirably from 5 g to 200 g per hectare.

The compounds of the present invention are effective through both contact (via soil, glass, wall, bed net, carpet, plant parts or animal parts), and ingestion (bait, or plant part).

The compounds of the present invention may also be applied against non-crop insect pests, such as ants, termites, wasps, flies, mosquitoes, crickets, or cockroaches. For use against said non-crop pests, compounds of the present invention are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics. The bait employed in the composition is a product, which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active ingredient.

Formulations of compounds of the present invention as aerosols (e.g in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets. The compounds of the present invention and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of the present invention are also suitable for the treatment of plant propagation material, especially seeds, in order to protect them from insect pest, in particular from soil-living insect pests and the resulting plant's roots and shoots against soil pests and foliar insects.

The compounds of the present invention are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the present invention, including a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A 242 236, EP-A 242 246) (WO 92/00377) (EP-A 257 993, U.S. Pat. No. 5,013,659) or in transgenic crop plants, for example cotton, with the capability of producing Bacillus thuringiensis toxins (Bt toxins) which make the plants resistant to certain pests (EP-A 142 924, EP-A 193 259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typically, a FS formulation may comprise 1-800 g/l of active ingredient, 1-200 g/I Surfactant, 0 to 200 g/I antifreezing agent, 0 to 400 g/l of binder, 0 to 200 g/l of a pigment and up to 1 liter of a solvent, preferably water.

Especially preferred FS formulations of compounds of the present invention for seed treatment usually comprise from 0.1 to 80% by weight (1 to 800 g/I) of the active ingredient, from 0.1 to 20% by weight (1 to 200 g/I) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight. Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds of the present invention are generally from 0.01 g to 10 kg per 100 kg of seed, preferably from 0.05 g to 5 kg per 100 kg of seed, more preferably from 0.1 g to 1000 g per 100 kg of seed and in particular from 0.1 g to 200 g per 100 kg of seed.

The invention therefore also relates to seed comprising a compound of the present invention, including an agriculturally useful salt of it, as defined herein. The amount of the compound of the present invention, including an agriculturally useful salt thereof will in general vary from 0.01 g to 10 kg per 100 kg of seed, preferably from 0.05 g to 5 kg per 100 kg of seed, in particular from 0.1 g to 1000 g per 100 kg of seed. For specific crops such as lettuce the rate can be higher.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of the present invention into contact with each other, and “seed dressing” to methods of seed treatment which provide the seeds with an amount of the compounds of the present invention, i.e. which generate a seed comprising a compound of the present invention. In principle, the treatment can be applied to the seed at any time from the harvest of the seed to the sowing of the seed. The seed can be treated immediately before, or during, the planting of the seed, for example using the “planters box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of the present invention, including their stereoisomers, veterinarily acceptable salts or N-oxides, are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions comprising a parasiticidally effective amount of compounds of the present invention, including their stereoisomers, veterinarily acceptable salts or N-oxides, and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of the present invention, including its stereoisomers, veterinarily acceptable salts or N-oxides, or a composition comprising it.

The invention also provides a process for the preparation of a composition for treating, controlling, preventing or protecting animals against infestation or infection by parasites which comprises a parasiticidally effective amount of a compound of the present invention, including its stereoisomers, veterinarily acceptable salts or N-oxides, or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula (I) and their stereoisomers, veterinarily acceptable salts, tautomers and N-oxides, are suitable for combating endo- and ectoparasites in and on animals.

The compounds of the present invention, especially compounds of formula (I) and their stereoisomers, veterinarily acceptable salts, tautomers and N-oxides, and compositions comprising them are preferably used for controlling and preventing infestations of and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels. Compounds of the present invention, including their stereoisomers, veterinarily acceptable salts or N-oxides, and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention, including their stereoisomers, veterinarily acceptable salts or N-oxides, and compositions comprising them are suitable for systemic and/or non-systemic control of ecto- and/or endoparasites. They are active against all or some stages of development.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
Bugs (Heteropterida): Cimex lectularius, Cimex hemipterus, Reduvius senilis, Triatoma spp., Rhodnius ssp., Panstrongylus ssp. and Arilus critatus, Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp,
Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The present invention relates to the therapeutic and the non-therapeutic use of compounds of the present invention and compositions comprising them for controlling and/or combating parasites in and/or on animals. The compounds of the present invention and compositions comprising them may be used to protect the animals from attack or infestation by parasites by contacting them with a parasiticidally effective amount of compounds of the present invention and compositions containing them.

The compounds of the present invention and compositions comprising them can be effective through both contact (via soil, glass, wall, bed net, carpet, blankets or animal parts) and ingestion (e.g. baits). As such, “contacting” includes both direct contact (applying the pesticidal mixtures/compositions containing the compounds of the present invention directly on the parasite, which may include an indirect contact at its locus-P, and optionally also administrating the pesticidal mixtures/composition directly on the animal to be protected) and indirect contact (applying the compounds/compositions to the locus of the parasite). The contact of the parasite through application to its locus is an example of a non-therapeutic use of compounds of the present invention. “Locus-P” as used above means the habitat, food supply, breeding ground, area, material or environment in which a parasite is growing or may grow outside of the animal.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions of the present invention. A parasiticidally effective amount of the compositions will also vary according to the prevailing conditions such as desired parasiticidal effect and duration, target species, mode of application, and the like.

The compounds of the present invention can also be applied preventively to places at which occurrence of the pests or parasites are expected.

Administration can be carried out both prophylactically and therapeutically.

Administration of the active compounds is carried out directly or in the form of suitable preparations, orally, topically/dermally or parenterally.

### EXAMPLES

The present invention is now illustrated in further details by the following examples, without imposing any limitation thereto.

### A. Preparation Examples

Compounds can be characterized e.g. by coupled High Performance Liquid Chromatography/mass spectrometry (HPLC/MS), by ¹H-NMR and/or by their melting points.

Analytical HPLC methods:
Method A: RP-18 column Chromolith Speed ROD, 50×4.6 mm, from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C. Flow: 1.8 mL/min.
Method B: Kinetex XB C18 1.7μ 50×2.1 mm from Phenomenex, Germany. Elution: acetonitrile+0.1% trifluoroacetic acid (TFA)/water+0.1% trifluoroacetic acid (TFA) in a ratio from 5:95 to 100:0 in 1.5 min at 60° C. Flow: 0.8 mL/min to 1 mL/min in 1.5 min. ¹H-NMR, respectively ¹³C-NMR: The signals are characterized by chemical shift (ppm, δ [delta]) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplet, q=quartet, t=triplet, d=doublet and s=singlet.

Abbreviations used are: h for hour(s), min for minute(s), r.t./room temperature for 20-25° C., THF for tetrahydrofuran, OAc for acetate, Et₃N for triethylamine, CH₃CN for acetonitrile, EtOAc for ethylacetate, HATU for O-(7-azabenzotriazol-1-yl)-N,N,N′,N′-tetramethyluronium hexafluorophosphate, EtOH for ethanol, MTBE for methyl tert-butyl ether.

### C.1 Compound Examples 1

Compound examples 1-1 to 1-5 correspond to compounds of formula C.1:

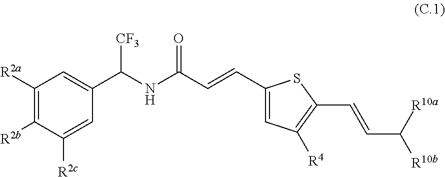

wherein R^{2a}, R^{2b}, R^{2c}, R⁴, R^{10a} and R^{10b} of each synthesized compound is defined in one row of table C.1 below.

The compounds were synthesized in analogy to Synthesis Example S.1.

TABLE C.1

| | | | | | HPLC-MS: | | |
|---|---|---|---|---|---|---|---|
| | R^{2a}, | | | | Method, | | |
| | R^{2b}, | | | | Rₜ (min) and | | |
| Ex. | R^{2c} | R⁴ | R^{10b} | R^{10a} | [M + H]⁺ | | |
| 1-1 | CF₃, | CH₃ | —C(═O)NHCH₃ | H | A | 3.519 | 493 |
| | H, H | | | | | | |
| 1-2 | CF₃, | CH₃ | —C(═O)NHCH₂CH₃ | H | A | 3.651 | 507 |
| | H, H | | | | | | |
| 1-3 | CF₃, | CH₃ | —C(═O)NHCH₂CF₃ | H | A | 3.814 | 561 |
| | H, H | | | | | | |
| 1-4 | CF₃, | CH₃ | —C(═O)NHCH₂CHF₂ | H | A | 3.707 | 543 |
| | H, H | | | | | | |
| 1-5 | CF₃, | CH₃ | —C(═S)NHCH₃ | H | A | 3.771 | 509 |
| | H, H | | | | | | |

### C.2 Compound Examples 2

Compound examples 2-1 to 2-29 correspond to compounds of formula C.2:

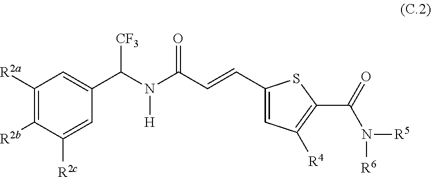

wherein R^{2a}, R^{2b}, R^{2c}, R⁴, R⁵ and R⁶ of each synthesized compound is defined in one row of table C.2 below.

The compounds were synthesized in analogy to Synthesis Example S.2.

TABLE C.2

| | R^{2a}, R^{2b}, | | | | HPLC-MS: Method, Rₜ (min) and [M + H]⁺ | | |
|---|---|---|---|---|---|---|---|
| Ex. | R^{2c} | R⁴ | R⁶ | R⁵ | or ¹H-NMR | | |
| 2-1 | CF₃, H, H | CH₃ | ^{c}C₃H₅* | H | HPLC-MS: A/3.271/477.1 | | |
| | | | | | ¹H NMR (400 MHz, CDCl₃): δ | | |
| | | | | | 7.72-7.55 (m, 5H), 7.01 (s, 1H), 6.66 (d, 1H), | | |
| | | | | | 6.36 (d, 1H), 5.98-5.92 (m, 2H), | | |
| | | | | | 2.89-2.88 (m, 1H), 2.49 (s, 3H), 0.90-0.88 (m, | | |
| | | | | | 2H), 0.64 (m, 2H). | | |
| 2-2 | CF₃, H, H | CH₃ | ^{c}C₅H₉*** | H | HPLC-MS: A/3.557/505.1 | | |
| | | | | | ¹H NMR (400 MHz, CDCl₃): δ | | |
| | | | | | 7.73-7.66 (m, 4H), 7.59-7.56 (m, 1H), 7.01 (s, 1H), | | |
| | | | | | 6.58-6.55 (d, 1H), 6.37-6.33 (d, 1H), | | |
| | | | | | 5.96-5.92 (m, 2H), 5.78-5.76 (m, 1H), | | |
| | | | | | 4.40-4.32 (m, 1H), 2.12-2.06 (m, 2H), | | |
| | | | | | 1.75-1.66 (m, 4H), 1.53-1.48 (m, 2H) | | |
| 2-3 | CF₃, H, H | CH₃ | CH₃ | H | HPLC-MS: A/3.555/451.1 | | |
| | | | | | ¹H NMR (400 MHz, CDCl₃): δ | | |
| | | | | | 7.74-7.66 (m, 4H), 7.59-7.55 (m, 1H), 7.02 (s, | | |
| | | | | | 1H), 6.65 (d, 1H), 6.39 (d, 1H), | | |
| | | | | | 5.99-5.92 (m, 1H), 5.86-5.84 (m, 1H), | | |
| | | | | | 3.01 (d, 3H), 2.47 (s, 3H) | | |
| 2-4 | CF₃, H, H | CH₃ | CH₂CH₃ | H | HPLC-MS: A/3.361/465.1 | | |
| | | | | | ¹H NMR (400 MHz, CDCl₃): δ | | |
| | | | | | 7.75-7.68 (m, 4H), 7.58-7.54 (m, 1H), | | |
| | | | | | 6.99-6.97 (m, 1H), 6.39 (d, 1H), 6.38-6.34 (m, | | |
| | | | | | 1H), 5.97-5.93 (m, 1H), 5.87-5.84 (m, | | |
| | | | | | 1H), 3.52-3.45 (m, 2H), 2.46 (s, 3H), | | |
| | | | | | 1.30-1.23 (m, 3H) | | |
| 2-5 | CF₃, H, H | CH₃ | CH₂-pyrid-2-yl | H | B | 1,013 | 528.3 |
| 2-6 | CF₃, H, H | CH₃ | CH₂CF₃ | H | B | 1.258 | 519.3 |
| 2-7 | CF₃, H, H | CH₃ | ═S(CH₂CH₃)₂ | | B | 1.187 | 525.3 |
| 2-8 | CF₃, H, H | CH₃ | —CH₂CH₂— | | B | 1.277 | 463.2 |
| 2-9 | CF₃, H, H | CH₃ | ^{c}C₄H₇** | H | B | 1.281 | 491.2 |
| 2-10 | CF₃, H, H | CH₃ | ═S(CH(CH₃)₂)₂ | | B | 1.266 | 553.3 |
| 2-11 | CF₃, H, H | CH₃ | —CH₂CH₂CH₂— | | HPLC-MS: A/3.307/477.1 | | |
| | | | | | ¹H NMR (400 MHz, CDCl₃): δ | | |
| | | | | | 7.82-7.77 (m, 2H), 7.66-7.61 (m, 4H), | | |
| | | | | | 7.51-7.47 (m, 1H), 6.95-6.92 (m, 1H), | | |
| | | | | | 6.44-6.37 (m, 1H), 4.21 (m, 4H), 2.36-2.28 (m, | | |
| | | | | | 3H) | | |
| 2-12 | CF₃, H, H | CH₃ | CH₃ | CH₃ | HPLC-MS: A/3.222/465.1 | | |
| | | | | | ¹H NMR (400 MHz, CD₃OD): δ | | |
| | | | | | 7.85-7.62 (m, 5H), 7.12 (s, 1H), 6.52 (d, 1H), | | |
| | | | | | 5.94-5.92 (m, 1H), 3.29/3.05 (s/s, 3H), | | |
| | | | | | 2.17/2.01 (s/s, 3H) | | |
| 2-13 | CF₃, H, H | CH₃ | CH(CH₃)₂ | H | HPLC-MS: A/3.386/479.1 | | |
| | | | | | ¹H NMR (400 MHz, CD₃OD): δ | | |
| | | | | | 7.85-7.60 (m, 5H), 7.11 (s, 1H), 6.55 (d, 1H), | | |
| | | | | | 5.94-5.92 (m, 1H), 4.14-4.10 (m, 1H), | | |
| | | | | | 2.37 (s, 3H), 1.21 (s, 6H) | | |
| 2-14 | CF₃, H, H | CH₃ | 3-tetrahydro- | H | HPLC-MS: A/3.148/452.1 | | |
| | | | furan-2-one | | ¹H NMR (400 MHz, CDCl₃): δ | | |
| | | | | | 7.78-7.49 (m, 6H), 5.93-5.92 (m, 1H), | | |
| | | | | | 5.83-5.82 (m, 1H), 6.32 (d, 1H), 5.91-5.86 (m, | | |
| | | | | | 1H), 4.74-4.70 (m, 1H), 4.55-4.51 (m, | | |
| | | | | | 1H), 4.37-4.31 (m, 1H), 2.82-2.75 (m, | | |
| | | | | | 1H), 2.41-2.35 (m, 4H) | | |
| 2-15 | CF₃, H, H | CH₃ | phenyl | H | HPLC-MS: A/3.619/513.1 | | |
| | | | | | ¹H NMR (400 MHz, CDCl₃): δ | | |
| | | | | | 7.73-7.55 (m, 8H), 7.41-7.35 (m, 2H), | | |
| | | | | | 7.21-7.17 (m, 1H), 7.04 (s, 1H), 6.80-6.78 (m, | | |
| | | | | | 1H), 6.38 (d, 1H), 5.98-5.91 (m, 1H), | | |
| | | | | | 2.54 (s, 3H) | | |
| 2-16 | CF₃, H, H | CH₃ | thietan-3-yl | H | HPLC-MS: A/3.408/509.1 | | |
| | | | | | ¹H NMR (400 MHz, CDCl₃): δ | | |
| | | | | | 7.74-7.65 (m, 4H), 7.60-7.56 (m, 1H), 7.04 (s, | | |
| | | | | | 1H), 6.44-6.42 (m, 1H), 6.35 (d, 1H), | | |
| | | | | | 6.24-6.22 (m, 1H), 5.96-5.90 (m, 1H), | | |
| | | | | | 5.43-5.36 (m, 1H), 3.51-3.40 (m, 4H), | | |
| | | | | | 2.50 (s, 3H) | | |
| 2-17 | CF₃, H, H | CH₃ | 1,1-dioxo- | H | HPLC-MS: A/3.146/541.1 | | |
| | | | thietan-3-yl | | ¹H NMR (400 MHz, d₆-DMSO): δ | | |
| | | | | | 9.51-9.49 (m, 1H), 8.79-8.78 (m, 1H), | | |
| | | | | | 8.04 (m, 1H), 7.95-7.93 (m, 1H), | | |
| | | | | | 7.83-7.81 (m, 1H), 7.74-7.72 (m, 1H), | | |
| | | | | | 7.70-7.67 (m, 1H), 7.31 (s, 1H), 6.64 (d, 1H), | | |
| | | | | | 6.18-6.13 (m, 1H), 4.58 (m, 3H), | | |
| | | | | | 4.30-4.27 (m, 2H), 2.51 (s, 3H) | | |
| 2-18 | CF₃, H, H | CH₃ | 1-oxothietan-3- | H | HPLC-MS: A/3.015/525.1 | | |
| | | | yl | | ¹H NMR (400 MHz, CD₃OD): δ | | |
| | | | | | 7.89-7.51 (m, 5H), 7.20 (s, 1H), | | |
| | | | | | 6.65-6.61 (m, 1H), 6.00-5.96 (m, 1H), | | |
| | | | | | 5.36-5.32 (m, 0.5H), 4.56-4.54 (m, 0.5H), | | |
| | | | | | 4.22-4.20 (m, 1H), 3.82-3.79 (m, 1H), | | |
| | | | | | 3.56-3.53 (m, 1H), 3.51-3.40 (m, 1H), | | |
| | | | | | 2.45 (s, 3H) | | |
| 2-19 | CF₃, H, H | CH₃ | 2-pyridyl | H | HPLC-MS: A/3.107/514.1 | | |
| | | | | | ¹H NMR (400 MHz, CDCl₃): δ 8.51 (s, | | |
| | | | | | 1H), 8.27-8.20 (m, 3H), 7.79-7.41 (m, | | |
| | | | | | 6H), 6.97 (s, 1H), 6.58-6.54 (m, 1H), | | |
| | | | | | 5.94-5.88 (m, 1H), 2.46 (s, 3H) | | |
| 2-20 | CF₃, H, H | CH₃ | CH(CH₃)CF₃ | H | HPLC-MS: A/3.556/533.1 | | |
| | | | | | ¹H NMR (400 MHz, CDCl₃): δ | | |
| | | | | | 7.82-7.65 (m, 4H), 7.02 (s, 1H), 6.74 (m, 1H), | | |
| | | | | | 6.38 (d, 1H), 5.96-5.88 (m, 1H), | | |
| | | | | | 4.93-4.84 (m, 1H), 2.47-2.33 (m, 3H), | | |
| | | | | | 1.45 (d, 3H) | | |
| 2-21 | CF₃, H, H | CH₃ | CH₂C(═O)CH₂CF₃ | H | HPLC-MS: A/3.249/576.1 | | |
| | | | | | ¹H NMR (400 MHz, CD₃OD): δ | | |
| | | | | | 7.86-7.64 (m, 5H), 7.03 (s, 1H), 6.63 (d, 1H), | | |
| | | | | | 5.96-5.90 (m, 1H), 4.07 (s, 2H), | | |
| | | | | | 3.98-3.91 (m, 2H), 2.46 (s, 3H) | | |
| 2-22 | CF₃, H, H | CH₃ | 3-pyridyl | H | HPLC-MS: A/2.901/514.1 | | |
| | | | | | ¹H NMR (400 MHz, CDCl₃): δ | | |
| | | | | | 8.68-8.67 (m, 1H), 8.41-8.40 (m, 1H), | | |
| | | | | | 8.23-8.21 (m, 1H), 7.97 (s, 1H), 7.77-7.35 (m, 7H), | | |
| | | | | | 7.02 (s, 1H), 6.37 (d, 1H), 5.97-5.91 (m, | | |
| | | | | | 1H), 2.52 (s, 3H) | | |
| 2-23 | CF₃, H, H | CH₃ | 4-pyridyl | H | HPLC-MS: A/2.934/514.1 | | |
| | | | | | ¹H NMR (400 MHz, CD₃OD): δ 8.45 (d, | | |
| | | | | | 1H), 7.90 (m, 1H), 7.81-7.65 (m, 6H), | | |
| | | | | | 7.26 (s, 1H), 6.68 (d, 1H), 6.03-5.97 (m, | | |
| | | | | | 1H), 2.51 (s, 3H) | | |
| 2-24 | CF₃, H, H | CH₃ | CH₂-thiazol-4- | H | HPLC-MS: A/3.220/534.1 | | |
| | | | yl | | ¹H NMR (400 MHz, CDCl₃): δ 8.80 (s, | | |
| | | | | | 1H), 8.05-8.03 (m, 1H), 7.81-7.51 (m, | | |
| | | | | | 5H), 7.28-6.88 (m, 2H), 6.39-6.35 (m, | | |
| | | | | | 1H), 5.97-5.93 (m, 1H), 4.74-4.73 (m, | | |
| | | | | | 2H), 2.41 (s, 3H) | | |
| 2-25 | CF₃, H, H | CH₃ | CH(CH₃)CH₂F | H | B | 1.221 | 497.4 |
| 2-26 | CF₃, H, H | CH₃ | 1- | H | B | 1.204 | 502.0 |
| | | | cyanocyclopropyl | | | | |
| 2-27 | CF₃, H, H | CH₃ | CH₂-(1- | H | B | 1.228 | 516.2 |
| | | | cyanocyclopropyl) | | | | |
| 2-28 | CF₃, H, H | CH₃ | CH₂CN | H | B | 1.278 | 476.1 |
| 2-29 | CF₃, H, H | CH₃ | CH₂—C≡CH | H | B | 1.213 | 475.1 |

*^{c}C₃H₅ = cyclopropyl
**^{c}C₄H₇ = cyclobutyl
***^{c}C₅H₉ = cyclopently

### C.3 Compound Examples 3

Compound examples 3-1 to 3-8 correspond to compounds of formula C.3:

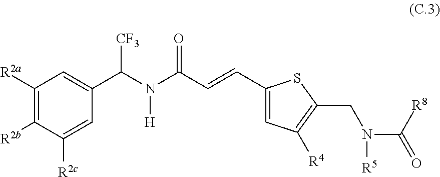

wherein R^{2a}, R^{2b}, R^{2c}, R⁴, R⁵ and R⁸ of each synthesized compound is defined in one row of table C.3 below.

The compounds were synthesized in analogy to Synthesis Examples S.3a (for R⁵=CH₂CH₃) or S.3b (for R⁵=H).

TABLE C.3

| | | | | | HPLC-MS: Method, Rₜ (min) | | |
|---|---|---|---|---|---|---|---|
| | R^{2a}, R^{2b}, | | | | and [M + H]⁺ | | |
| Ex. | R^{2c} | R⁴ | R⁸ | R⁵ | or ¹H-NMR | | |
| 3-1 | CF₃, H, H | CH₃ | CH₂SO₂CH₃ | CH₂CH₃ | HPLC-MS: A/3.297/571.1 | | |
| | | | | | ¹H NMR (400 MHz, CD₃CN): δ | | |
| | | | | | 7.86 (m, 1H), 7.81-7.77 (m, | | |
| | | | | | 3H), 7.69-7.62 (m, 2H), | | |
| | | | | | 7.06 (s, 1H), 6.40 (d, 1H), | | |
| | | | | | 6.01-5.96 (m, 1H), 4.72-4.67 (m, 2H), | | |
| | | | | | 4.21-4.19 (m, 2H), | | |
| | | | | | 3.47-3.42 (m, 2H), 3.13 (s, 3H), 2.21 (s, | | |
| | | | | | 3H), 1.21-1.11 (m, 3H) | | |
| 3-2 | CF₃, H, H | CH₃ | ^{c}C₃H₅* | CH₂CH₃ | HPLC-MS: A/3.573/519.1 | | |
| | | | | | ¹H NMR (400 MHz, CD₃OD): δ | | |
| | | | | | 7.89 (m, 1H), 7.81-7.74 (m, | | |
| | | | | | 2H), 7.68-7.63 (m, 2H), | | |
| | | | | | 7.06 (s, 1H), 6.45 (d, 1H), | | |
| | | | | | 5.97-5.95 (m, 1H), 4.90/4.70 (s/s, 2H), | | |
| | | | | | 3.63-3.44 (m, 2H), 2.29 (s, | | |
| | | | | | 3H), 1.97-1.95 (m, 1H), | | |
| | | | | | 1.28-1.12 (m, 3H), 0.98-0.96 (m, | | |
| | | | | | 2H), 0.95-0.89 (m, 2H) | | |
| 3-3 | CF₃, H, H | CH₃ | CH₃ | H | HPLC-MS: A/3.129/465.1 | | |
| | | | | | ¹H NMR (400 MHz, CD₃OD): δ | | |
| | | | | | 7.89 (m, 1H), 7.81-7.79 (m, | | |
| | | | | | 1H), 7.74-7.72 (m, 1H), | | |
| | | | | | 7.65-7.62 (m, 2H), 7.02 (s, 1H), | | |
| | | | | | 6.45 (d, 1H), 6.00-5.94 (m, | | |
| | | | | | 1H), 4.43 (s, 2H), 2.25 (s, 3H), | | |
| | | | | | 2.00 (s, 3H) | | |
| 3-4 | CF₃, H, H | CH₃ | CH₂CH₃ | H | HPLC-MS: A/3.236/479.1 | | |
| | | | | | ¹H NMR (400 MHz, CDCl₃): δ | | |
| | | | | | 7.76-7.66 (m, 4H), | | |
| | | | | | 7.58-7.54 (m, 1H), 7.08 (m, 1H), 6.91 (s, | | |
| | | | | | 1H), 6.24 (d, 1H), | | |
| | | | | | 5.96-5.92 (m, 1H), 4.53-4.52 (m, 2H), | | |
| | | | | | 2.34-2.27 (m, 2H), 2.17 (s, | | |
| | | | | | 3H), 1.23-1.19 (m, 3H) | | |
| 3-5 | CF₃, H, H | CH₃ | CH₂CF₃ | H | HPLC-MS: A/3.392/533.1 | | |
| | | | | | ¹H NMR (400 MHz, CDCl₃): δ | | |
| | | | | | 7.73-7.55 (m, 5H), 6.97 (s, | | |
| | | | | | 1H), 6.39-6.37 (m, 1H), | | |
| | | | | | 6.20 (d, 1H), 6.07-5.92 (m, 2H), | | |
| | | | | | 4.58-4.57 (m, 2H), 2.20 (s, 3H) | | |
| 3-6 | CF₃, H, H | CH₃ | CH₂SCH₃ | H | HPLC-MS: A/3.295/511.1 | | |
| | | | | | ¹H NMR (400 MHz, CDCl₃): δ | | |
| | | | | | 7.73-7.65 (m, 4H), | | |
| | | | | | 7.58-7.54 (m, 1H), 7.28-7.25 (m, 1H), | | |
| | | | | | 6.97 (s, 1H), 6.69-6.67 (m, | | |
| | | | | | 1H), 6.22 (d, 1H), | | |
| | | | | | 5.98-5.90 (m, 1H), 4.59-4.58 (m, 2H), | | |
| | | | | | 3.28 (s, 2H), 2.21 (s, 3H), | | |
| | | | | | 2.15 (s, 3H) | | |
| 3-7 | CF₃, H, H | CH₃ | NHCH₃ | H | HPLC-MS: A/3.295/480.1 | | |
| | | | | | ¹H NMR (400 MHz, CDCl₃): δ | | |
| | | | | | 8.47 (m, 1H), 7.82 (m, 1H), | | |
| | | | | | 7.70-7.64 (m, 2H), | | |
| | | | | | 7.51-7.51 (m, 1H), 7.43-7.39 (m, 1H), | | |
| | | | | | 6.64 (s, 1H), 6.26 (d, 1H), | | |
| | | | | | 5.91-5.87 (m, 1H), 4.29 (s, | | |
| | | | | | 2H), 2.78 (s, 3H), 2.00 (s, 3H) | | |
| 3-8 | CF₃, H, H | CH₃ | ^{c}C₃H₅ | H | A | 3.260 | 491.1 |

*^{c}C₃H₅ = cyclopropyl

### C.4 Compound Examples 4

Compound examples 4-1 to 4-8 correspond to compounds of formula C.4:

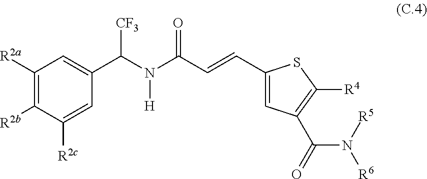

wherein R^{2a}, R^{2b}, R^{2c}, R⁴, R⁵ and R⁶ of each synthesized compound is defined in one row of table C.4 below.

The compounds were synthesized in analogy to Synthesis Example S.4.

TABLE C.4

| | | | | | HPLC-MS: Method, Rₜ (min) and |
|---|---|---|---|---|---|
| | R^{2a}, R^{2b,} | | | | [M + H]⁺ |
| Ex. | R^{2c} | R⁴ | R⁶ | R⁵ | or ¹H-NMR |
| 4-1 | CF₃, H, H | CH₃ | ^{c}C₃H₅* | H | HPLC-MS: A/3.255/477.1 |
| | | | | | ¹H NMR (400 MHz, CDCl₃): δ |
| | | | | | 7.70-7.53 (m, 4H), 7.15 (s, 1H), |
| | | | | | 6.24 (d, 1H), 6.16 (d, 1H), |
| | | | | | 5.93-5.87 (m, 2H), 2.84-2.83 (m, 1H), |
| | | | | | 2.71 (s, 3H), 0.87-0.84 (m, 2H), |
| | | | | | 0.61-0.58 (m, 2H). |
| 4-2 | CF₃, H, H | CH₃ | ^{c}C₄H₇** | H | HPLC-MS: A/3.421/491.1 |
| | | | | | ¹H NMR (400 MHz, CDCl₃): δ |
| | | | | | 7.71-7.54 (m, 4H), 7.20 (s, 1H), |
| | | | | | 6.28 (d, 1H), 6.17 (d, 1H), |
| | | | | | 5.93-5.87 (m, 2H), 4.55-4.49 (m, 1H), |
| | | | | | 2.69 (s, 3H), 2.43-2.42 (m, 2H), |
| | | | | | 1.96-1.91 (m, 2H), 1.81-1.76 (m, |
| | | | | | 2H) |
| 4-3 | CF₃, H, H | CH₃ | CH₂CF₃ | H | HPLC-MS: A/3.441/519.1 |
| | | | | | ¹H NMR (400 MHz, CDCl₃): δ |
| | | | | | 7.70-7.53 (m, 4H), 7.22 (s, 1H), |
| | | | | | 6.35-6.33 (m, 1H), 6.19 (d, 1H), |
| | | | | | 6.07-6.04 (m, 1H), 5.93-5.89 (m, |
| | | | | | 1H), 4.11-4.03 (m, 1H), 2.71 (s, |
| | | | | | 3H) |
| 4-4 | CF₃, H, H | CH₃ | CH(CH₃)CF₃ | H | HPLC-MS: A/3.593/533.1 |
| | | | | | ¹H NMR (400 MHz, CDCl₃): δ |
| | | | | | 7.70-7.55 (m, 5H), 7.21 (s, 1H), |
| | | | | | 6.36 (d, 1H), 6.19 (d, 1H), |
| | | | | | 5.95-5.91 (m, 1H), 5.89-5.79 (m, 1H), |
| | | | | | 4.92-4.82 (m, 1H), 2.69 (s, 3H), |
| | | | | | 1.41 (d, 3H) |
| 4-5 | CF₃, H, H | CH₃ | CH₂-pyrid-2-yl | H | HPLC-MS: A/2.885/528.1 |
| | | | | | ¹H NMR (400 MHz, CDCl₃): δ |
| | | | | | 8.73 (m, 1H), 8.32-8.31 (m, 1H), |
| | | | | | 7.79 (m, 1H), 7.66-7.49 (m, 5H), |
| | | | | | 7.34-7.23 (m, 2H), 7.19-7.07 (m, |
| | | | | | 1H), 5.85-5.77 (m, 1H), 5.70 (d, |
| | | | | | 1H), 4.67-4.66 (m, 2H), 2.55 (s, |
| | | | | | 3H) |
| 4-6 | CF₃, H, H | CH₃ | thietan-3-yl | H | HPLC-MS: A/3.383/509.1 |
| | | | | | ¹H NMR (400 MHz, CDCl₃): δ |
| | | | | | 7.69-7.53 (m, 5H), 7.18 (s, |
| | | | | | 1H), 6.54 (d, 1H), 6.31 (d, 1H), |
| | | | | | 6.19 (d, 1H), 5.93-5.89 (m, 1H), |
| | | | | | 5.40-5.33 (m, 1H), 3.47-3.36 (m, |
| | | | | | 4H), 2.67 (s, 3H) |
| 4-7 | CF₃, H, H | CH₃ | 1,1-dioxothietan-3- | H | HPLC-MS: A/3.147/541.1 |
| | | | yl | | ¹H NMR (400 MHz, CDCl₃): δ |
| | | | | | 7.71-7.55 (m, 5H), 7.32 (s, 1H), |
| | | | | | 6.81 (d, 1H), 6.61 (d, 1H), 6.23 (d, |
| | | | | | 1H), 5.93-5.87 (m, 1H), |
| | | | | | 4.88-4.86 (m, 1H), 4.65-4.59 (m, 2H), |
| | | | | | 4.12-4.08 (m, 2H), 2.71 (s, 3H) |
| 4-8 | CF₃, H, H | CH₃ | 2-pyridyl | H | HPLC-MS: A/3.037/514.1 |
| | | | | | ¹H NMR (400 MHz, CDCl₃): δ |
| | | | | | 8.45 (m, 1H), 8.30-8.28 (m, 2H), |
| | | | | | 7.74-7.67 (m, 5H), 7.64-7.56 (m, |
| | | | | | 1H), 7.41 (s, 1H), 7.09-7.06 (m, |
| | | | | | 1H), 6.42 (d, 1H), 6.20 (d, 1H), |
| | | | | | 5.95-5.91 (m, 1H), 2.78 (s, 3H) |

*^{c}C₃H₅ = cyclopropyl
**^{c}C₄H₇ = cyclobutyl

### Synthesis Examples

### Synthesis Example S1

### (E)-3-[4-methyl-5-[(E)-(methylcarbamoylhydrazono)methyl]-2-thienyl]-N-[2,2,2-trifluoro-1-[3-(trifluoromethyl)phenyl]ethyl]prop-2-enamide

(Compound example 1-1; compound of formula C.1, wherein R^{2a} is CF₃; R^{2b}, R^{2c} and R^{10a} are H; R⁴ is CH₃; —R^{10b} is —C(═O)NHCH₃)

### Step 1: tert-Butyl(E)-3-(5-formyl-4-methyl-2-thienyl)prop-2-enoate

A mixture of 5-bromo-3-methyl-thiophene-2-carbaldehyde (31 g, 0.151 mol, CAS 189331-47-3), tert-butyl prop-2-enoate (29 g, 0.227 mol), tris-ortho-tolylphosphine (POT, 9.2 g, 30.2 mmol), Pd(OAc)₂ (1.69 g, 7.56 mmol), Et₃N (30.5 g, 0.30 mol) in anhydrous CH₃CN (400 mL) under N₂ was heated to reflux overnight. Then the reaction was cooled to room temperature, filtered and the filtrate was concentrated and purified by column chromatography on silica gel (Petrol ether:EtOAc=250:1) to afford the product (15.1 g, 40%).

¹H NMR (400 MHz, CDCI₃): δ 10.0 (s, 1H), 7.57 (d, 1H), 7.05 (s, 1H), 6.32 (d, 1H), 2.54 (s, 3H), 1.53 (s, 9H).

### Step 2: (E)-3-(5-Formyl-4-methyl-2-thienyl)prop-2-enoic acid

The product of step 1 (15.1 g, 60 mmol) was stirred in a solution of HCl in EtOAc (4M, 200 mL) overnight at room temperature. Then the solution was concentrated to give the product (9.4 g, 80%).

¹H NMR (400 MHz, CD₃OD): δ 10.0 (s, 1H), 7.68 (d, 1H), 7.27 (s, 1H), 6.38 (d, 1H), 2.53 (s, 3H).

### Step 3: (E)-3-(5-Formyl-4-methyl-2-thienyl)-N-[2,2,2-trifluoro-1-[3-(trifluoromethyl)phenyl]ethyl]prop-2-enamide

A mixture of the product of step 2 (9.3 g, 47.4 mmol), 2,2,2-trifluoro-1-[3-(trifluoromethyl)phenyl]ethanamine (11.5 g, 47.4 mmol, CAS 65686-68-2) and HATU (21.6 g, 56.9 mmol) in THF (400 mL) was stirred overnight at room temperature. Then water was added, and the mixture extracted with EtOAc (3×200 mL). The combined organic layers were dried over Na₂SO₄ and concentrated. The residue was purified by column chromatography on silica gel (Petrol ether:EtOAc=20:1) to afford the product (7.7 g, 52%).

¹H NMR (400 MHz, CD₃OD): δ 9.99 (s, 1H), 7.96-7.72 (m, 6H), 7.05 (s, 1H), 6.79 (d, 1H), 5.93-5.87 (m, 1H), 2.54 (s, 3H).

### Step 4: (E)-3-[4-methyl-5-[(E)-(methylcarbamoylhydrazono)methyl]-2-thienyl]-N-[2,2,2-trifluoro-1-[3-(trifluoromethyl)phenyl]ethyl]prop-2-enamide

To a mixture of the product of step 3 (0.15 g, 0.32 mmol) and 1-amino-3-methyl-urea hydrochloride (0.05 g, 0.38 mmol) in EtOH (15 mL) was added 1 drop of glacial acid. The reaction was refluxed for 3 h, stirred overnight at room temperature and concentrated. The residue was purified by column chromatography on silica gel (Petrol ether/EtOAc) to afford the product (0.15 g, 87%). For analytical data of the product, see table C.1.

### Synthesis Example S2

### N-cyclopropyl-3-methyl-5-[(E)-3-oxo-3-[[2,2,2-trifluoro-1-[3-(trifluoromethyl)phenyl]-ethyl]amino]prop-1-enyl]thiophene-2-carboxamide

(Compound example 2-1; compound of formula C.2, wherein R^{2a} is CF₃; R^{2b}, R^{2c} and R⁵ are H; R⁴ is CH₃; R⁶ is cycopropyl)

For the preparation of ethyl 5-bromo-3-methyl-thiophene-2-carboxylate (starting material of step 1), see: patent application US 2004/0266796, p. 10, compound 7a.

### Step 1: Ethyl 5-[(E)-3-tert-butoxy-3-oxo-prop-1-enyl]-3-methyl-thiophene-2-carboxylate

A mixture of ethyl 5-bromo-3-methyl-thiophene-2-carboxylate (17 g, 68.3 mmol), tert-butyl prop-2-enoate (13.1 g, 0.102 mol), tris-ortho-tolylphosphine (POT, 4.6 g, 15.1 mmol), Pd(OAc)₂ (0.85 g, 3.8 mmol) and Et₃N (20.7 g, 0.205 mol) in anhydrous CH₃CN (400 mL) under N₂ was heated to reflux overnight. Then the reaction was cooled to room temperature, filtered and the filtrate was concentrated and purified by column chromatography on silica gel (Petrol ether: EtOAc=5:1) to afford the product (9.5 g, 47%).

¹H NMR (400 MHz, CDCl₃): δ 7.58-7.54 (d, 1H), 7.06 (s, 1H), 6.29-6.25 (d, 1H), 4.34 (q, 2H), 2.52 (s, 3H), 1.53 (s, 9H), 1.39 (t, 3H).

### Step 2: (E)-3-(5-Ethoxycarbonyl-4-methyl-2-thienyl)prop-2-enoic acid

The product of step 1 (9.5 g, 32.1 mmol) was stirred in a solution of HCl in EtOAc (4M, 200 mL) overnight at room temperature. Then the solution was concentrated to give the product (7.8 g, 99%).

¹H NMR (400 MHz, CD₃OD): δ 7.71-7.67 (d, 1H), 7.24 (s, 1H), 6.34 (d, 1H), 4.33 (q, 2H), 2.51 (s, 3H), 1.36 (t, 3H).

### Step 3: Ethyl 3-methyl-5-[(E)-3-oxo-3-[[2,2,2-trifluoro-1-[3-(trifluoromethyl)phenyl]ethyl]amino]prop-1-enyl]thiophene-2-carboxylate

A mixture of the product of step 2 (7.6 g, 31.6 mmol), 2,2,2-trifluoro-1-[3-(trifluoromethyl)phenyl]ethanamine (7.7 g, 31.6 mmol, CAS 65686-68-2) and HATU (9.1 g, 38 mmol) in THF (250 mL) was stirred overnight at room temperature. Then water was added, and the mixture extracted with EtOAc (3×200 mL). The combined organic layers were dried over Na₂SO₄ and concentrated. The residue was purified by column chromatography on silica gel (petrol ether: EtOAc=20:1 to 5:1) to afford the product (7.7 g, 52%).

¹H NMR (400 MHz, CD₃OD): δ 7.69-7.60 (m, 3H), 7.51-7.47 (m, 1H), 7.04 (d, 1H), 6.39 (d, 1H), 5.92-5.87 (m, 1H), 4.30 (q, 2H), 2.32 (s, 3H), 1.33 (t, 3H).

### Step 4: 3-Methyl-5-[(E)-3-oxo-3-[[2,2,2-trifluoro-1-[3-(trifluoromethyl)phenyl]-ethyl]amino]prop-1-enyl]thiophene-2-carboxylic acid

The product of step 3 (2.7 g, 5.8 mmol) and NaOH (0.46 g, 11.6 mmol) in a mixture of THF/water (1:1, 160 mL) were stirred overnight at 45° C. Then the pH was adjusted to 4-5 with aqueous HCl solution and the mixture extracted with EtOAc (3×200 mL). The combined organic layers were dried (Na₂SO₄), and concentrated. The residue was purified by preparative HPLC to give the product (2.2 g, 87%).

¹H NMR (400 MHz, CD₃OD): δ 7.95-7.60 (m, 5H), 7.20 (s, 1H), 6.66 (d, 1H), 6.01-5.95 (m, 1H), 2.50 (s, 3H).

### Step 5: N-Cyclopropyl-3-methyl-5-[(E)-3-oxo-3-[[2,2,2-trifluoro-1-[3-(trifluoromethyl)phenyl]ethyl]amino]prop-1-enyl]thiophene-2-carboxamide

To a solution of the product of step 4 (0.4 g, 0.92 mmol) in THF (50 mL) was added HATU (0.42 g, 1.1 mmol), cyclopropylamine (0.17 g, 1.84 mmol) and triethylamine (0.27 g, 2.7 mmol). The mixture was stirred at room temperature overnight. Then water was added and the mixture extracted with MTBE. The organic layer was separated, washed with brine and concentrated. The residue was purified by preparative HPLC to give the product (0.209 g, 48%).

¹H NMR (400 MHz, CDCl₃): δ 7.72-7.55 (m, 5H), 7.01 (s, 1H), 6.66 (d, 1H), 6.36 (d, 1H), 5.98-5.92 (m, 2H), 2.89-2.88 (m, 1H), 2.49 (s, 3H), 0.90-0.88 (m, 2H), 0.64 (m, 2H).

### Synthesis Example S3a

### (E)-3-[5-[[Ethyl-(2-methylsulfonylacetyl)amino]methyl]-4-methyl-2-thienyl]-N-[2,2,2-trifluoro-1-[3-(trifluoromethyl)phenyl]ethyl]prop-2-enamide

(Compound example 3-1; compound of formula C.3, wherein R^{2a} is CF₃; R^{2b} and R^{2c} are H; R⁴ is CH₃; R⁵ is —CH₂CH₃; R⁸ is CH₂SO₂CH₃)

### Step 1: (E)-3-[5-(hydroxymethyl)-4-methyl-2-thienyl]-N-[2,2,2-trifluoro-1-[3-(trifluoromethyl)phenyl]ethyl]prop-2-enamide

To (E)-3-(5-formyl-4-methyl-2-thienyl)-N-[2,2,2-trifluoro-1-[3-(trifluoromethyl)phenyl]-ethyl]prop-2-enamide (i.e. the product of Synthesis Example S1, step 3; 4.0 g, 9.5 mmol) in MeOH (100 mL) at 0° C. was added NaBH₄ (360 mg, 9.5 mmol) and the mixture was stirred at 0° C. for 3 h. Then, the reaction was concentrated and added to a saturated aqueous NH₄Cl solution. The aqueous layer was extracted twice with EtOAc. The combined organic layers were dried (Na₂SO₄), concentrated and purified by preparative HPLC to afford the product (2.2 g, 55%).

¹H NMR (400 MHz, CD₃OD): δ 7.85-7.59 (m, 5H), 7.03 (s, 1H), 6.43 (d, 1H), 5.93-5.91 (m, 1H), 4.65 (s, 2H), 2.15 (s, 3H).

### Step 2: (E)-3-[5-(Ethylaminomethyl)-4-methyl-2-thienyl]-N-[2,2,2-trifluoro-1-[3-(trifluoromethyl)phenyl]ethyl]prop-2-enamide

The product of step 1 (1 g, 2.3 mmol) in DMF at 0° C. was treated with N,N-diisopropylethylamine (DIEA, 1 mL) and methanesulfonyl chloride (MsCl, 1 mL). The reaction was stirred for 30 min. Then ethylamine (5 mL) was added and the mixture stirred at room temperature overnight. Brine was added and the aqueous layer extracted with EtOAc. The organic layer was dried (Na₂SO₄), concentrated and purified by column chromatography on silica to give the product (1.3 g, crude), which was used in the next step without further purification.

### Step 3: (E)-3-[5-[[Ethyl-(2-methylsulfonylacetyl)amino]methyl]-4-methyl-2-thienyl]-N-[2,2,2-trifluoro-1-[3-(trifluoromethyl)phenyl]ethyl]prop-2-enamide

To a solution of the product of step 2 (600 mg, crude) in THF (50 mL) was added HATU (1.2 g, 3 mmol), 2-methylsulfonylacetic acid (0.4 g, 2.5 mmol) and Et₃N (0.3 g, 3 mmol). The reaction was stirred overnight at room temperature. Then brine was added and the mixture extracted with EtOAc (2×). The combined organic layers were dried (Na₂SO₄) and concentrated. The residue was purified by preparative HPLC to afford the product (220 mg, 34% over steps 2 & 3).

¹H NMR (400 MHz, CD₃CN): δ 7.86 (m, 1H), 7.81-7.77 (m, 3H), 7.69-7.62 (m, 2H), 7.06 (s, 1H), 6.40 (d, 1H), 6.01-5.96 (m, 1H), 4.72-4.67 (m, 2H), 4.21-4.19 (m, 2H), 3.47-3.42 (m, 2H), 3.13 (s, 3H), 2.21 (s, 3H), 1.21-1.11 (m, 3H).

### Synthesis Example S3b

### (E)-3-[4-Methyl-5-[[(2-methylsulfanylacetyl)amino]methyl]-2-thienyl]-N-[2,2,2-trifluoro-1-[3-(trifluoromethyl)phenyl]ethyl]prop-2-enamide

(Compound example 3-6; compound of formula C.3, wherein R^{2a} is CF₃; R^{2b}, R^{2c} and R⁵ are H; R⁴ is CH₃; —R⁸ is —CH₂SCH₃)

### Step 1: (E)-3-[5-(Aminomethyl)-4-methyl-2-thienyl]-N-[2,2,2-trifluoro-1-[3-(trifluoromethyl)phenyl]ethyl]prop-2-enamide

Under N₂, (E)-3-[5-(hydroxymethyl)-4-methyl-2-thienyl]-N-[2,2,2-trifluoro-1-[3-(trifluoromethyl)phenyl]ethyl]prop-2-enamide (i.e. the product from Synthesis Example S3, step 1) (8 g, 19 mmol) was dissolved in THF (100 mL). At 0° C., 1,8-diazabicyclo[5.4.0]-undec-7-ene (DBU, 5.7 g, 38 mmol) and diphenylphosphoryl azide (DPPA, 9.3 g, 38 mmol) were added and the mixture was stirred at room temperature for 2 h. Then water (30 mL) and Ph₃P (15 g, 60 mmol) were added into the solution at room temperature, and the reaction was heated at reflux overnight. The mixture was then partitioned between EtOAc and water. The organic layer was dried (Na₂SO₄) and concentrated. The residue was purified by column chromatography on silica (petrol ether:EtOAc=1:2) to afford the product (3 g, 39%).

¹H NMR (400 MHz, CD₃OD): δ 7.87-7.54 (m, 5H), 7.03 (s, 1H), 6.43 (d, 1H), 3.89 (s, 2H), 2.21 (s, 3H).

### Step 2: (E)-3-[4-Methyl-5-[[(2-methylsulfanylacetyl)amino]methyl]-2-thienyl]-N-[2,2,2-trifluoro-1-[3-(trifluoromethyl)phenyl]ethyl]prop-2-enamide

To a solution of the product of step 1 (0.6 g, 1.4 mmol) in THF (50 mL) was added HATU (1 g, 2.8 mmol), 2-methylsulfanylacetic acid (250 mg, 2.8 mmol) and triethylamine (0.5 g, 5 mmol). The mixture was stirred at room temperature overnight. Then water was added and the mixture extracted with EtOAc. The organic layer was separated, dried (Na₂SO₄) and concentrated. The residue was purified by preparative HPLC to give the product (0.26 g, 36%) as a solid.

¹H NMR (400 MHz, CDCl₃): δ 7.73-7.65 (m, 4H), 7.58-7.54 (m, 1H), 7.28-7.25 (m, 1H), 6.97 (s, 1H), 6.68 (d, 1H), 6.22 (d, 1H), 5.98-5.90 (m, 1H), 4.59-4.58 (m, 2H), 3.28 (s, 2H), 2.21 (s, 3H), 2.15 (s, 3H).

### Synthesis Example S4

### N-Cyclopropyl-2-methyl-5-[(E)-3-oxo-3-[[2,2,2-trifluoro-1-[3-(trifluoromethyl)phenyl]-ethyl]amino]prop-1-enyl]thiophene-3-carboxamide

(Compound example 4-1; compound of formula C.4, wherein R^{2a} is CF₃; R^{2b}, R^{2c} and R⁵ are H; R⁴ is CH₃; R⁶ is cyclopropyl)

For the preparation of methyl 5-bromo-2-methyl-thiophene-3-carboxylate (starting material of step 1), see S. H. Lee et al., Bioorg. Med. Chem. 2011, Vol. 19, p. 5812-5832, compound 8).

### Step 1 & 2: (E)-3-(4-methoxycarbonyl-5-methyl-2-thienyl)prop-2-enoic acid

A mixture of methyl 5-bromo-2-methyl-thiophene-3-carboxylate (71 g, 0.302 mol), tert-butyl prop-2-enoate (47.9 g, 0.373 mol), tris-ortho-tolylphosphine (POT, 9 g, 29.6 mmol), Pd(OAc)₂ (3 g, 13.4 mmol) and Et₃N (89 mL, 0.64 mol) in anhydrous DMF (1 L) under N₂ was heated to reflux overnight. Then the reaction was cooled to room temperature, filtered, and the filtrate was concentrated and purified by column chromatography on silica gel (Petrol ether:EtOAc=5:1) to afford methyl 5-[(E)-3-tert-butoxy-3-oxo-prop-1-enyl]-2-methyl-thiophene-3-carboxylate (60 g, product of step 1).

The product of step 1 (61 g, 21 mmol) was stirred in a solution of HCl in EtOAc (4M, 600 mL) overnight at room temperature. Then, the solution was concentrated to give the product of step 2 (47.5 g, 46% over steps 1 & 2).

¹H NMR (400 MHz, d₆-DMSO): δ 12.44 (s, 1H), 7.71-7.67 (m, 2H), 6.11 (d, 1H), 3.79 (s, 3H), 2.70 (s, 3H).

### Step 3 & 4: 2-Methyl-5-[(E)-3-oxo-3-[[2,2,2-trifluoro-1-[3-(trifluoromethyl)phenyl]ethyl]-amino]prop-1-enyl]thiophene-3-carboxylic acid

A mixture of the product of step 2 (20 g, 70 mmol), 2,2,2-trifluoro-1-[3-(trifluoromethyl)phenyl]ethanamine (16.2 g, 70 mmol, CAS 65686-68-2) and HATU (40 g, 107 mmol) in THF (500 mL) was stirred overnight at room temperature. Then water was added, and the mixture extracted with EtOAc (3×200 mL). The combined organic layers were dried over Na₂SO₄ and concentrated. The residue was purified by column chromatography on silica gel (Petrol ether:EtOAc=20:1) to afford the methyl 2-methyl-5-[(E)-3-oxo-3-[[2,2,2-trifluoro-1-[3-(trifluoromethyl)phenyl]ethyl]amino]prop-1-enyl]thiophene-3-carboxylate (20 g, product of step 3).

The product of step 3 (20 g, 44 mmol) and LiOH (1.6 g, 66 mmol) in a mixture of THF/water (2:5, 700 mL) were stirred overnight at room temperature. Then the pH was adjusted to 4-5 with aqueous HCl solution and the mixture extracted with EtOAc (3×200 mL). The combined organic layers were dried (Na₂SO₄), and concentrated. The residue was purified by trituration with CH₂Cl₂ to yield the product of step 4 (11 g, 30% over steps 3 & 4).

¹H NMR (400 MHz, CD₃OD): δ 7.87-7.62 (m, 5H), 7.55 (s, 1H), 6.45 (d, 1H), 5.98-5.92 (m, 1H), 2.72 (s, 3H).

### Step 5: N-Cyclopropyl-2-methyl-5-[(E)-3-oxo-3-[[2,2,2-trifluoro-1-[3-(trifluoromethyl)phenyl]ethyl]amino]prop-1-enyl]thiophene-3-carboxamide

To a solution of the product of step 4 (0.4 g, 0.92 mmol) in THF (50 mL) were added HATU (0.42 g, 1.1 mmol), cyclopropylamine (0.17 g, 1.84 mmol) and Et₃N (0.27 g, 2.7 mmol). The mixture was stirred at room temperature overnight. Then water was added and the mixture extracted with MTBE (3×30 mL). The combined organic layers were dried (Na₂SO₄) and concentrated. The residue was purified by preparative HPLC to give the product (0.156 g, 38%).

¹H NMR (400 MHz, CDCl₃): δ 7.70-7.53 (m, 4H), 7.15 (s, 1H), 6.24 (d, 1H), 6.16 (d, 1H), 5.93-5.87 (m, 2H), 2.84-2.83 (m, 1H), 2.71 (s, 3H), 0.87-0.84 (m, 2H), 0.61-0.58 (m, 2H).

### B. Evaluation of Pesticidal Activity:

The activity of the compounds of formula I of the present invention can be demonstrated and evaluated by the following biological test.

B.1 Cowpea aphid (Aphis craccivora)

Potted cowpea plants colonized with approximately 100-150 aphids of various stages were sprayed after the pest population had been recorded. Population reduction was assessed after 24, 72, and 120 hours.

In this test, the compounds 1-2, 1-4, 1-5, 2-3, 2-27 and 4-8 at 500 ppm, respectively, showed a mortality of at least 75% in comparison with untreated controls.

B.2 Diamond back moth (Plutella xylostella)

Leaves of Chinese cabbage were dipped in test solution and air-dried. Treated leaves were placed in petri dished lined with moist filter paper. Mortality was recorded 24, 72, and 120 hours after treatment.

In this test, the compounds 1-1, 2-1, 2-3, 2-4, 2-6, 2-7, 2-12, 2-15, 2-19, 2-20, 2-21, 2-22, 2-23, 2-25, 2-26, 2-27, 2-28, 2-29, 3-4 and 3-8 at 500 ppm, respectively, showed a mortality of at least 75% in comparison with untreated controls.

B.3 Green peach aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23+1° C. and about 50+5% relative humidity for 3 days. Aphid mortality and fecundity was then visually assessed.

In this test, the compounds 1-1, 1-4, 2-1, 2-3, 2-4, 2-6, 2-11, 2-14, 2-15, 2-16, 2-18, 2-22, 2-24, 2-25, 2-26, 2-27, 2-28, 2-29, 3-1, 3-2, 3-3, 3-4, 3-5, 3-6, 3-7, 3-8, 4-1, 4-2 and 4-5 at 2500 ppm, respectively, showed a mortality of at least 75% in comparison with untreated controls.

B.4 Mediterranean fruitfly (Ceratitis capitata)

For evaluating control of Mediterranean fruitfly (Ceratitis capitata) the test unit consisted of microtiter plates containing an insect diet and 50-80 C. capitate eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 28±1° C. and about 80±5% relative humidity for 5 days. Egg and larval mortality was then visually assessed.

In this test, the compounds 1-1, 2-1, 2-3, 2-4, 2-17, 2-19, 2-20, 2-21, 2-23, 2-25, 2-29, 3-2, 3-4, 3-5, 4-1, 4-2, and 4-3 at 2500 ppm, respectively, showed a mortality of at least 75% in comparison with untreated controls.

B.5 Orchid thrips (dichromothrips corbetti)

Dichromothaps corbetti adults used for bioassay were obtained from a colony maintained continuously under laboratory conditions. For testing purposes, the test compound was diluted to a concentration of 500 ppm (wt compound: vol diluent) in a 1:1 mixture of acetone:water (vol:vol), plus 0.01% vol/vol Kinetic® surfactant.

Thrips potency of each compound was evaluated by using a floral-immersion technique. Plastic petri dishes were used as test arenas. All petals of individual, intact orchid flowers were dipped into treatment solution and allowed to dry. Treated flowers were placed into individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.

In this test, the compounds 1-1, 2-1, 2-4, 2-5, 2-6, 2-12, 2-14, 2-16, 2-17, 2-18, 2-19, 2-21, 2-22, 2-23, 2-26, 2-28, 2-29, 3-1, 3-3, 3-4, 3-6, 3-7 and 3-8 at 500 ppm, respectively, showed a mortality of at least 75% in comparison with untreated controls.

B.6 Silverleaf whitefly (bernisia argentifolii)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compounds 1-1, 2-2, 2-9, 3-5, 4-1, and 4-4 at 300 ppm, respectively, showed a mortality of at least 75% in comparison with untreated controls.

B.7 Southern armyworm (Spodoptera eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds 1-1, 1-2, 1-3, 2-1, 2-2, 2-3, 2-4, 2-5, 2-6, 2-7, 2-8, 2-9, 2-10, 2-11, 2-12, 2-13, 2-16, 2-19, 2-20, 2-21, 2-22, 2-23, 2-24, 2-25, 3-1, 3-2, 3-4, 3-5, 4-1, 4-2, 4-3, 4-4, 4-7 and 4-8 at 300 ppm, respectively, showed a mortality of at least 75% in comparison with untreated controls.

B.8 Vetch aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed. In this test, the compounds 1-1, 2-1, 2-6, 2-19, 2-26, 3-2 and 3-8 at 2500 ppm, respectively, showed a mortality of at least 75% in comparison with untreated controls.

B.9 Tobacco budworm (Heliothis virescens) HELVI

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed. In this test, the compounds 1-1, 1-5, 2-1, 2-2, 2-3, 2-4, 2-6, 2-19, 2-20, 2-26, 3-2, and 3-5 at 2500 ppm, respectively, showed a mortality of at least 75% in comparison with untreated controls.

B.10 Boll weevil (Anthonomus grandis)

For evaluating control of boll weevil (Anthonomus grandis) the test unit consisted of 24-well-microtiter plates containing an insect diet and 20-30 A. grandis eggs.

The compounds were formulated using a solution containing 75% v/v water and 25% v/v DMSO. Different concentrations of formulated compounds were sprayed onto the insect diet at 20 μl, using a custom built micro atomizer, at two replications.

After application, microtiter plates were incubated at about 23±1° C. and about 50±5% relative humidity for 5 days. Egg and larval mortality was then visually assessed.

In this test, the compounds 1-1, 1-3, 1-4, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 3-1, 3-2, 3-3, 3-4, 3-5, 3-6, 3-7, 3-8, 4-1, 4-2, 4-3, 4-5, 4-6, and 4-8 at 2500 ppm, respectively, showed a mortality of at least 75% in comparison with untreated controls.

B.11 Red spider mite (Tetranychus kanzawai)

The active compound was dissolved at the desired concentration in a mixture of 1:1 (v/v) distilled water: acetone. A surfactant (Alkamuls® EL 620) was added at the rate of 0.1% (v/v).

Potted cowpea beans of 7-10 days of age were cleaned with tap water and sprayed with 5 ml of the test solution using air driven hand atomizer. The treated plants were allowed to air dry and afterwards inculated with 20 or more mites by clipping a cassava leaf section with known mite population. Treated plants were placed inside a holding room at about 25-27° C. and about 50-60% relative humidity.

Mortality was determined by counting the live mites 72 HAT. Percent mortality was assessed after 72 h.

In this test, the compounds 1-2 and 4-3 at 500 ppm, respectively, showed a mortality of at least 75% in comparison with untreated controls.

1-53. (canceled)

54. A compound of formula (I) 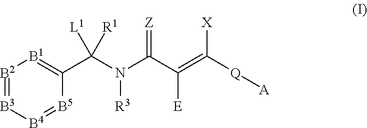
wherein
B¹, B², B³, B⁴ and B⁵ are each independently selected from the group consisting of N and CR², with the proviso that at most two of B¹, B², B³, B⁴ and B⁵ are N;
Z is selected from O and S;
Q is a 5-membered saturated, partially unsaturated or aromatic heteromonocyclic ring containing 1, 2, or 3 heteroatoms or heteroatom groups selected from N, NR^{4a}, O, S, NO, SO and SO₂ as ring members and optionally containing also 1 or 2 groups selected from C═O and C═S as ring members, where the heteromonocyclic ring is optionally substituted with 1, 2 or 3 substituents R⁴;
A is a group A¹, A², A³ or A⁴,
wherein
A¹ is selected from the group consisting of —C(═NR⁶)R⁸, —S(O)ₙR⁹ and —N(R⁵)R⁶;
A² is a group of following formula: 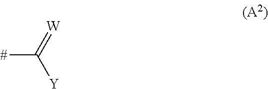
wherein
# denotes the bond to Q;
W is selected from O and S;
Y is selected from hydrogen, —N(R⁵)R⁶ and —OR⁹;
A³ is a group of following formula: 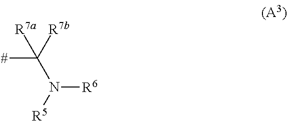
wherein
# denotes the bond to Q;
A⁴ is a 3-, 4-, 5-, 6- or 7-membered saturated, partially unsaturated or maximally unsaturated heteromonocyclic ring containing 1, 2, 3 or 4 heteroatoms or heteroatom groups selected from N, O, S, NO, SO and SO₂ as ring members, or is a 8-, 9- or 10-membered saturated, partially unsaturated or maximally unsaturated heterobicyclic ring containing 1, 2, 3 or 4 heteroatoms or heteroatom groups selected from N, O, S, NO, SO and SO₂ as ring members, where the heteromonocyclic or heterobicyclic ring is optionally substituted with one or more substituents R¹¹;
L¹ is selected from the group consisting of hydrogen, halogen, cyano, hydroxy, amino, C₁-C₆-alkyl, C₃-C₈-cycloalkyl, C₂-C₆-alkenyl, C₂-C₆-alkynyl, C₁-C₆-alkoxy, wherein the aliphatic and cycloaliphatic moieties in the five last-mentioned radicals may be partially or fully halogenated and/or may be substituted by one or more radicals R⁸,
phenyl which may be substituted by 1, 2, 3, 4 or 5 radicals R¹¹, and a 3-, 4-, 5-, 6- 7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂ as ring members, where the heteromono- or heterobicyclic ring may be substituted by one or more radicals R¹¹;
or
L¹ and a radical R² bound in the position of B¹ or B⁵, together with the carbon atoms they are bound to, form a 5-, 6-, or 7-membered saturated, partially unsaturated or maximally unsaturated ring, where the ring may contain 1 or 2 heteroatoms or heteroatom-containing groups selected from O, S, N, SO, SO₂, C═O and C═S as ring members, wherein the ring may be substituted with 1, 2, 3, 4 or 5 substituents independently selected from the group consisting of halogen, C₁-C₆-alkyl and C₁-C₆-haloalkyl;
E is selected from the group consisting of hydrogen, halogen, C₁-C₆-alkyl which may be partially or fully halogenated and/or may be substituted by one or more radicals selected from C₁-C₆-alkoxy, and C₃-C₆-cycloalkyl which may be partially or fully halogenated and/or may be substituted by one or more radicals selected from the group consisting of C₁-C₆-alkyl and C₁-C₆-alkoxy;
or
E and a radical R⁴ bound on the heteromonocyclic ring Q, together with the atoms to which they are bound, form a 5-, 6-, or 7-membered saturated, partially unsaturated or maximally unsaturated ring, where the ring may contain 1 or 2 heteroatoms or heteroatom-containing groups selected from O, S, N, SO, SO₂, C═O and C═S as ring members, wherein the ring may be substituted with 1, 2, 3, 4 or 5 substituents independently selected from the group consisting of halogen, C₁-C₆-alkyl, C₁-C₆-haloalkyl and C₁-C₆-alkoxy; or
or E and a radical R^{4a} bound on the heteromonocyclic ring Q, together with the atoms to which they are bound, form a 5-, 6-, or 7-membered saturated, partially unsaturated or maximally unsaturated heterocyclic ring, where the heterocyclic ring may contain 1 or 2 further heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, C₁-C₆-alkyl, C₁-C₆-haloalkyl and C₁-C₆-alkoxy;
X is selected from the group consisting of hydrogen, halogen, C₁-C₆-alkyl, C₃-C₈-cycloalkyl, C₁-C₆-alkoxy, C₃-C₈-cycloalkyl-C₁-C₆-alkyl- and C₁-C₆-alkoxy-C₁-C₆-alkyl-, wherein the aliphatic and cycloaliphatic moieties in the five last-mentioned radicals may be partially or fully halogenated;
R¹ is selected from the group consisting of C₁-C₄-alkyl, C₂-C₄-alkenyl, C₂-C₄-alkynyl, C₃-C₆-cycloalkyl, C₁-C₄-alkylsulfonyl and C₁-C₄-alkoxycarbonyl, wherein the aliphatic and cycloaliphatic moieties in the six last-mentioned radicals may be partially or fully halogenated and/or may be substituted by one or more radicals selected from the group consisting of hydroxy, cyano, C₁-C₄-alkoxy, C₁-C₄-haloalkoxy, C₁-C₄-alkoxycarbonyl, C₁-C₄-alkylaminocarbonyl and C₁-C₄-dialkylaminocarbonyl;
each R² is independently selected from the group consisting of hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last-mentioned aliphatic and cycloaliphatic radicals may be partially or fully halogenated and/or may be substituted by one or more radicals R⁸,
—Si(R¹²)₃, —OR⁹, —S(O)ₙR⁹, —N(R^{10a})R^{10b},
phenyl which may be substituted by 1, 2, 3, 4 or 5 radicals R¹¹, and a 3-, 4-, 5-, 6- 7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂ as ring members, where the heteromono- or heterobicyclic ring may be substituted by one or more radicals R¹¹;
R³ is selected from the group consisting of hydrogen, C₁-C₆-alkyl, C₃-C₆-cycloalkyl, C₂-C₆-alkenyl, C₂-C₆-alkynyl and C₁-C₆-alkylsulfonyl, wherein the aliphatic and cycloaliphatic moieties in the five last-mentioned radicals may be partially or fully halogenated and/or may be substituted with one or more substituents R⁸;
each R⁴ is independently selected from the group consisting of halogen, cyano, azido, nitro, —SCN, —SF₅, C₁-C₆-alkyl, C₃-C₈-cycloalkyl, C₂-C₆-alkenyl, C₂-C₆-alkynyl, wherein the four last-mentioned aliphatic and cycloaliphatic radicals may be partially or fully halogenated and/or may be substituted by one or more radicals R⁸,
—Si(R¹²)₃, —OR⁹, —S(O)ₙR⁹, —N(R^{10a})R^{10b},
phenyl which may be substituted by 1, 2, 3, 4 or 5 radicals R¹¹, and a 3-, 4-, 5-, 6- 7-, 8-, 9- or 10-membered saturated, partially unsaturated or maximally unsaturated heteromonocyclic or heterobicyclic ring containing 1, 2, 3 or 4 heteroatoms or heteroatom groups selected from N, O, S, NO, SO and SO₂ as ring members, where the heteromonocyclic or heterobicyclic ring may be substituted by one or more radicals R¹¹;
or
two radicals R⁴ bound on neighboring carbon atoms form together with the atoms to which they are bound a 5-, 6-, or 7-membered saturated, partially unsaturated or maximally unsaturated ring, where the ring may contain 1 or 2 heteroatoms or heteroatom-containing groups selected from O, S, N, SO, SO₂, C═O and C═S as ring members, wherein the ring may be substituted with 1, 2, 3, 4 or 5 substituents independently selected from the group consisting of halogen, C₁-C₆-alkyl, C₁-C₆-haloalkyl and C₁-C₆-alkoxy;
R^{4a} is selected from the group consisting of hydrogen, C₁-C₆-alkyl, C₃-C₆-cycloalkyl, C₂-C₆-alkenyl and C₂-C₆-alkynyl, wherein the four last-mentioned aliphatic and cycloaliphatic radicals may be partially or fully halogenated and/or may be substituted with one or more substituents R⁸,
and —S(O)ₙR⁹;
or R⁴ and R^{4a}, together with the atoms to which they are bound, form a 5-, 6-, or 7-membered saturated, partially unsaturated or maximally unsaturated heterocyclic ring, where the heterocyclic ring may contain 1 or 2 further heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, C₁-C₆-alkyl, C₁-C₆-haloalkyl and C₁-C₆-alkoxy;
each R⁵ is independently selected from the group consisting of hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more substituents R⁸, and —S(O)ₙR⁹,
or
R⁵ and a radical R⁴ or R⁵ and a radical R^{4a} bound on the heteromonocyclic ring Q, together with the atoms to which they are bound, form a 5-, 6-, or 7-membered saturated, partially unsaturated or maximally unsaturated heterocyclic ring, where the ring may contain 1 or 2 further heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, C₁-C₆-alkyl, C₁-C₆-haloalkyl and C₁-C₆-alkoxy;
each R⁶ is independently selected from the group consisting of hydrogen, cyano, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted by one or more substituents R⁸, —OR⁹, —N(R^{10a})R^{10b}, —S(O)ₙR⁹, —C(═O)N(R^{10a})N(R^{10a})R^{10b}), —Si(R¹²)₃, —C(═O)R⁸, —P(═O)(OR⁹)₂, —P(═S)(OR⁹)₂,
phenyl which may be substituted with 1, 2, 3, 4, or 5 substituents R¹¹, and
a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂ as ring members, where the heteromonocyclic or heterobicyclic ring may be substituted with one or more substituents R¹¹;
or R⁵ and R⁶, together with the nitrogen atom to which they are bound, form a 3-, 4-, 5-, 6-, 7- or 8-membered saturated, partially unsaturated or maximally unsaturated heteromonocyclic ring, where the ring may further contain 1, 2, 3 or 4 heteroatoms or heteroatom-containing groups selected from O, S, N, SO, SO₂, C═O and C═S as ring members, or form a 7-, 8-, 9- or 10-membered saturated, partially unsaturated or maximally unsaturated heterobicyclic ring, where the ring may further contain 1, 2, 3 or 4 heteroatoms or heteroatom groups selected from N, O, S, NO, SO, SO₂, C═O and C═S, wherein the heteromonocyclic or heterobicyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
or R⁵ and R⁶ together form a group ═C(R⁸)₂, ═S(O)ₘ(R⁹)₂, ═NR^{10a} or ═NOR⁹;
R^{7a}, R^{7b} are each independently selected from the group consisting of hydrogen, halogen, cyano, C₁-C₆-alkyl, C₃-C₈-cycloalkyl, C₂-C₆-alkenyl and C₂-C₆-alkynyl, wherein the four last-mentioned aliphatic and cycloaliphatic radicals may be partially or fully halogenated and/or may be substituted by one or more radicals R⁸;
each R⁸ is independently selected from the group consisting of cyano, azido, nitro, —SCN, —SF₅, C₃-C₈-cycloalkyl, C₃-C₈-halocycloalkyl, where the cycloaliphatic moieties in the two last-mentioned radicals may be substituted by one or more radicals R¹³;
—Si(R¹²)₃, —OR⁹, —OSO₂R⁹, —S(O)ₙR⁹, —N(R^{10a})R^{10b}, —C(═O)R¹³, —C(═O)N(R^{10a})R^{10b}, —C(═S)N(R^{10a})R^{10b}, —C(═O)OR⁹,
phenyl, optionally substituted with 1, 2, 3, 4 or 5 substituents R¹⁶, and
a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁶,
or
two R⁸ present on the same carbon atom of an alkyl, alkenyl, alkynyl or cycloalkyl group together form a group ═O, ═C(R¹³)₂; ═S; ═S(O)ₘ(R¹⁵)₂, ═S(O)ₘR¹⁵N(R^{14a})R^{14b}, ═NR^{10a}, ═NOR⁹; or ═NN(R^{10a})R^{10b};
or
two radicals R⁸, together with the carbon atoms of an alkyl, alkenyl, alkynyl or cycloalkyl group which they are bonded to, form a 3-, 4-, 5-, 6-, 7- or 8-membered saturated or partially unsaturated carbocyclic or heterocyclic ring, where the heterocyclic ring comprises 1, 2, 3 or 4 heteroatoms or heteroatom groups independently selected from N, O, S, NO, SO and SO₂ as ring members, and where the carbocyclic or heterocyclic ring is optionally substituted with one or more substituents R¹⁶; and
R⁸ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹³; and
R⁸ in the groups —C(═NR⁶)R⁸, —C(═O)R⁸ and ═C(R⁸)₂ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in the six last-mentioned radicals may be substituted by one or more radicals R¹³;
each R⁹ is independently selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the nine last-mentioned radicals may be substituted by one or more radicals R¹³, —C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —Si(R¹²)₃, —S(O)ₙR¹⁵, —S(O)ₙN(R^{14a})R^{14b}, —N(R^{10a})R^{10b}, —N═C(R¹³)₂, —C(═O)R¹³, —C(═O)N(R^{14a})R^{14b}, —C(═S)N(R^{14a})R^{14b}, —C(═O)OR¹⁵,
phenyl, optionally substituted with one or more substituents R¹⁶; and
a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁶; and R⁹ in the groups —S(O)ₙR⁹ and —OSO₂R⁹ is additionally selected from the group consisting of C₁-C₆-alkoxy and C₁-C₆-haloalkoxy;
R^{10a}, R^{10b} are selected independently from one another from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, where the aliphatic and cycloaliphatic moieties in the eight last-mentioned radicals may be substituted by one or more radicals R¹³;
—C₁-C₆-alkyl-C(═O)OR¹⁵, —C₁-C₆-alkyl-C(═O)N(R^{14a})R^{14b}, —C₁-C₆-alkyl-C(═S)N(R^{14a})R^{14b}, —C₁-C₆-alkyl-C(═NR¹⁴)N(R^{14a})R^{14b}, —C₁-C₆-alkoxy, C₁-C₆-haloalkoxy, C₁-C₆-alkylthio, C₁-C₆-haloalkylthio,
—S(O)ₙR¹⁵, —S(O)ₙN(R^{14a})R^{14b}, —C(═O)R¹³, —C(═O)OR¹⁵, —C(═O)N(R^{14a})R^{14b}, —C(═S)R¹³, —C(═S)SR¹⁵, —C(═S)N(R^{14a})R^{14b}, —C(═NR¹⁴)R¹³;
phenyl, optionally substituted with 1, 2, 3 or 4, substituents R¹⁶; and
a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R¹⁶;
or
R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂ as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl, optionally substituted with 1, 2, 3, 4 or 5 substituents R¹⁶, and a 3-, 4-, 5-, 6,- or 7-membered saturated, partially unsaturated or maximally unsaturated heterocyclic ring comprising 1, 2 or 3 heteroatoms or heteroatom groups selected from N, O, S, NO, SO and SO₂ as ring members, where the heterocyclic ring optionally carries one or more substituents R¹⁶;
or R^{10a} and R^{10b} together form a group ═C(R¹³)₂, ═S(O)ₘ(R¹⁵)₂, ═S(O)ₘR¹⁵N(R^{14a})R^{14b}, ═NR¹⁴ or ═NOR¹⁵;
each R¹¹ is independently selected from the group consisting of halogen, cyano, azido, nitro, —SCN, —SF₅, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁸,
—OR⁹, —N(R^{10a})R^{10b}, —S(O)ₙR⁹, —Si(R¹²)_{3;}
phenyl, optionally substituted with 1, 2, 3, 4, or 5 substituents selected independently from R¹⁶; and
a 3-, 4-, 5-, 6- or 7-membered saturated, partially unsaturated or maximally unsaturated aromatic heterocyclic ring comprising 1, 2, 3 or 4 heteroatoms or heteroatom groups selected from N, O, S, NO, SO and SO₂ as ring members, where the heterocyclic ring is optionally substituted with one or more substituents selected independently from R¹⁶;
or two R¹¹ present on the same ring carbon atom of an unsaturated or partially unsaturated heterocyclic ring may together form a group ═O, ═C(R¹³)₂; ═S; ═S(O)ₘ(R¹⁵)₂; ═S(O)ₘR¹⁵N(R^{14a})R^{14b}, ═NR¹⁴, ═NOR¹⁵, or ═NN(R^{14a})R^{14b};
or two R¹¹ bound on adjacent ring atoms form together with the ring atoms to which they are bound a saturated 3-, 4-, 5-, 6-, 7-, 8- or 9-membered ring, wherein the ring may contain 1 or 2 heteroatoms or heteroatom groups selected from O, S, N, NR¹⁴, NO, SO and SO₂ and/or 1 or 2 groups selected from C═O, C═S and C═NR¹⁴ as ring members, and wherein the ring may be substituted by one or more radicals selected from the group consisting of halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁶, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂ as ring members, where the heterocyclic ring may be substituted by one or more radicals R¹⁶;
each R¹² is independently selected from the group consisting of hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl-, C₁-C₆-haloalkoxy-C₁-C₆-alkyl-, and phenyl, optionally substituted with 1, 2, 3, 4, or 5 substituents R¹⁶;
each R¹³ is independently selected from the group consisting of cyano, nitro, —OH, —SH, —SCN, —SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy and oxo; phenyl, benzyl, phenoxy, where the phenyl moiety in the last three radicals may be unsubstituted or carry 1, 2, 3, 4 or 5 substituents R¹⁶; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂ as ring members, where the heterocyclic ring may be substituted by 1, 2 or 3 substituents R¹⁶;
or
two R¹³ present on the same carbon atom of an alkyl, alkenyl, alkynyl or cycloalkyl group may together be ═O, ═CH(C₁-C₄-alkyl), ═C(C₁-C₄-alkyl)C₁-C₄-alkyl, ═N(C₁-C₆-alkyl) or ═NO(C₁-C₆-alkyl);
and
R¹³ as a substituent on a cycloalkyl ring is additionally selected from the group consisting of C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 substituents selected from CN, C₃-C₄-cycloalkyl, C₁-C₄-alkoxy, C₁-C₄-haloalkoxy and oxo;
and
R¹³ in the groups ═C(R¹³)₂, —N═C(R¹³)₂, —C(═O)R¹³, —C(═S)R¹³ and —C(═NR¹⁴)R¹³ is additionally selected from the group consisting of hydrogen, halogen, C₁-C₆-alkyl, C₂-C₆-alkenyl and C₂-C₆-alkynyl, wherein the three last-mentioned aliphatic radicals may be unsubstituted, partially or fully halogenated and/or may carry 1 or 2 radicals selected from CN, C₃-C₄-cycloalkyl, C₁-C₄-alkoxy, C₁-C₄-haloalkoxy and oxo;
each R¹⁴ is independently selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from CN, C₁-C₄-alkoxy, C₁-C₄-haloalkoxy, C₁-C₄-alkylsulfinyl, C₁-C₄-alkylsulfonyl, C₃-C₄-cycloalkyl which may be substituted by 1 or 2 substituents selected from halogen and cyano; and oxo; C₃-C₈-cycloalkyl which may be unsubstituted, partially or fully halogenated and/or may carry 1 or 2 radicals selected from C₁-C₄-alkyl, C₁-C₄-alkoxy, C₁-C₄-haloalkoxy, C₁-C₄-alkylthio, C₁-C₄-alkylsulfinyl, C₁-C₄-alkylsulfonyl, C₃-C₄-cycloalkyl, C₃-C₄-cycloalkyl-C₁-C₄-alkyl-, where the cycloalkyl moiety in the two last-mentioned radicals may be substituted by 1 or 2 substituents selected from halogen and cyano; and oxo;
phenyl, benzyl, pyridyl, phenoxy, wherein the cyclic moieties in the four last-mentioned radicals may be unsubstituted and/or carry 1, 2 or 3 substituents selected from halogen, C₁-C₆-alkyl, C₁-C₆-haloalkyl, C₁-C₆-alkoxy, C₁-C₆-haloalkoxy and (C₁-C₆-alkoxy)carbonyl; and a 3-, 4-, 5- or 6-membered saturated, partially unsaturated or maximally unsaturated heterocyclic ring comprising 1 or 2 heteroatoms or heteroatom groups selected from N, O, S, NO, SO and SO₂ as ring members, where the heterocyclic ring is optionally substituted with one or more substituents R¹⁶;
R^{14a} and R^{14b}, independently of each other, have one of the meanings given for R¹⁴;
or
R^{14a} and R^{14b}, together with the nitrogen atom to which they are bound, form a 3-, 4-, 5-, 6- or 7-membered saturated, partially unsaturated or maximally unsaturated heterocyclic ring, wherein the heterocyclic ring may additionally contain 1 or 2 heteroatoms or heteroatom groups selected from N, O, S, NO, SO and SO₂ as ring members, where the heterocyclic ring optionally carries one or more substituents selected from halogen, C₁-C₄-alkyl, C₁-C₄-haloalkyl, C₁-C₄-alkoxy and C₁-C₄-haloalkoxy;
or
R^{14a} and R¹⁴ or R^{14b} and R¹⁴, together with the nitrogen atoms to which they are bound in the group —C(═NR¹⁴)N(R^{14a})R^{14b}, form a 3-, 4-, 5-, 6- or 7-membered partially unsaturated or maximally unsaturated heterocyclic ring, wherein the heterocyclic ring may additionally contain 1 or 2 heteroatoms or heteroatom groups selected from N, O, S, NO, SO and SO₂ as ring members, where the heterocyclic ring optionally carries one or more substituents selected from halogen, C₁-C₄-haloalkyl, C₁-C₄-alkoxy and C₁-C₄-haloalkoxy;
each R¹⁵ is independently selected from the group consisting of hydrogen, cyano, trimethylsilyl, triethylsilyl, tert-butyldimethylsilyl, C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy, C₁-C₄-alkylthio, C₁-C₄-alkylsulfinyl, C₁-C₄-alkylsulfonyl and oxo; C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy, C₁-C₄-alkylthio, C₁-C₄-alkylsulfinyl, alkylsulfonyl and oxo;
phenyl, benzyl, pyridyl and phenoxy, wherein the four last-mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
each R¹⁶ is independently selected from the group consisting of halogen, nitro, cyano, —OH, —SH, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl;
C₁-C₆-alkyl, C₂-C₆-alkenyl, C₂-C₆-alkynyl, wherein the three last-mentioned aliphatic radicals may be unsubstituted, partially or fully halogenated and/or may carry 1 or 2 radicals selected from C₃-C₄-cycloalkyl, C₁-C₄-alkoxy, C₁-C₄-haloalkoxy and oxo;
C₃-C₈-cycloalkyl which may be unsubstituted, partially or fully halogenated and/or may carry 1 or 2 radicals selected from C₁-C₄-alkyl, C₃-C₄-cycloalkyl, C₁-C₄-alkoxy, C₁-C₄-haloalkoxy and oxo;
phenyl, benzyl, pyridyl and phenoxy, wherein the four last-mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
or
two R¹⁶ present together on the same atom of an unsaturated or partially unsaturated ring may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or
two R¹⁶ on two adjacent carbon atoms form together with the carbon atoms they are bonded to a 4-, 5-, 6-, 7- or 8-membered saturated, partially unsaturated or maximally unsaturated ring, wherein the ring may contain 1 or 2 heteroatoms or heteroatom groups selected from N, O, S, NO, SO and SO₂ as ring members, and wherein the ring optionally carries one or more substituents selected from halogen, C₁-C₄-haloalkyl, C₁-C₄-alkoxy and C₁-C₄-haloalkoxy;
each n is independently 0, 1 or 2; and
each m is independently 0 or 1;
an N-oxide, stereoisomer, or an agriculturally or veterinarily acceptable salt thereof.

55. The compound as claimed in claim 54, where the moiety -Q-A is selected from the moieties Q-1 to Q-36 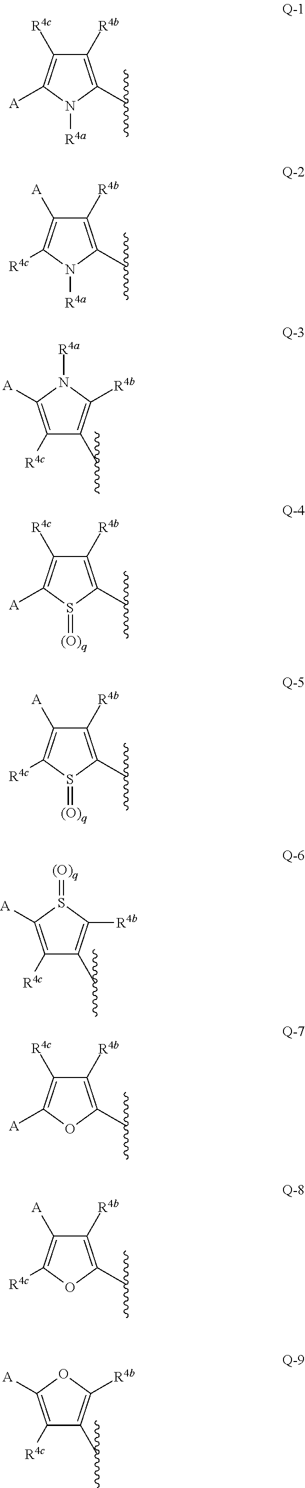 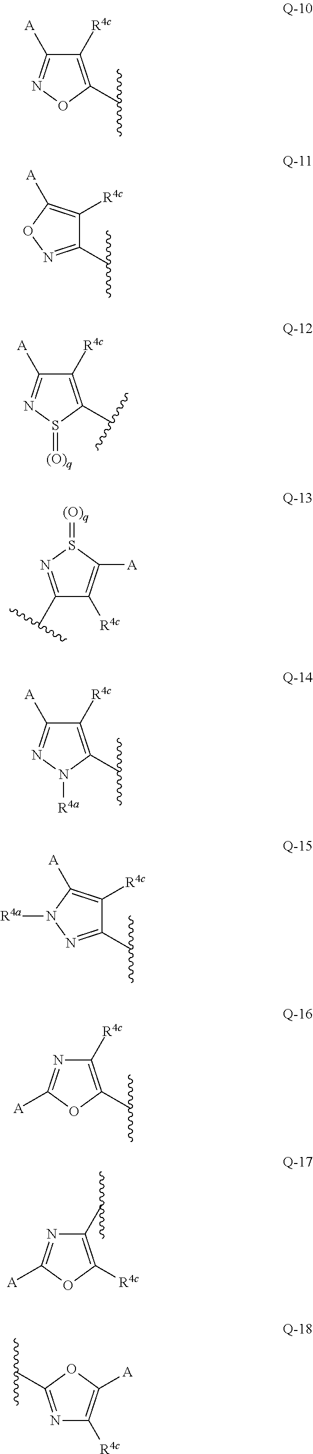 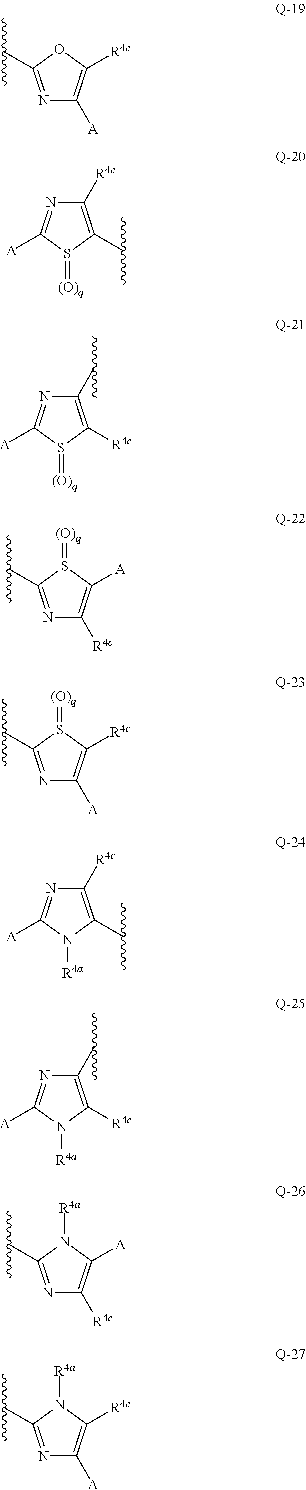 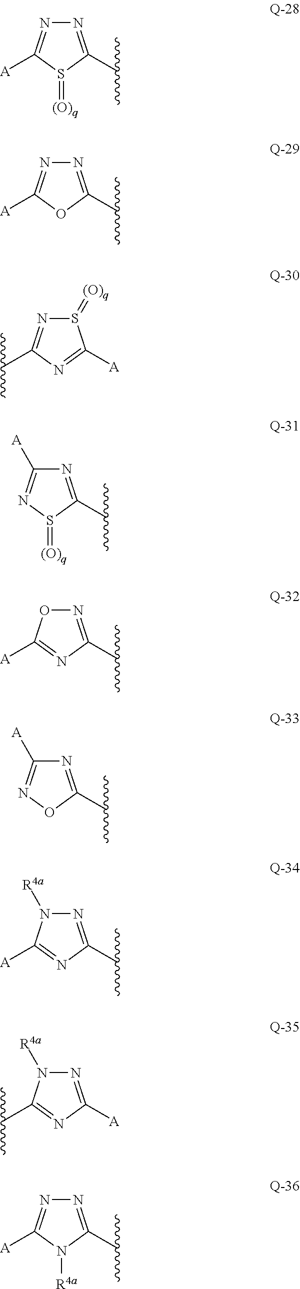
wherein [symbol]
denotes the bonding point to the remainder of the molecule,
A is as defined in claim 54;
R^{4a} is as defined in claim 54;
R^{4b} and R^{4c}, independently of each other, have one of the meanings given in claim 54 for R⁴ or are hydrogen; and
q is 0, 1 or 2.

56. The compound as claimed in claim 55, where the moiety -Q-A is selected from the group consisting of Q-4 and Q-5, wherein q is 0.

57. The compound as claimed in claim 54, where A is A¹ and A¹ is selected from —C(═NR⁶)R⁸ and —N(R⁵)R⁶; wherein R⁵, R⁶ and R⁸ are as defined in claim 54.

58. The compound as claimed in claim 57, where R⁶ in —C(═NR⁶)R⁸ is selected from hydrogen, cyano, C₃-C₈-cycloalkyl, C₂-C₁₀-alkenyl, C₂-C₁₀-alkynyl, wherein the four last-mentioned aliphatic and cycloaliphatic radicals each independently may be partially or fully halogenated and/or may be substituted with 1, 2, 3, 4, 5 or 6 substituents R⁸; —OR⁹ and —N(R^{10a})R^{10b}; wherein R⁸, R⁹, R^{10a} and R^{10b} are as defined in claim 54.

59. The compound as claimed in claim 58, where R⁶ in —C(═NR⁶)R⁸ is selected from —OR⁹ and —N(R^{10a})R^{10b} and specifically from —N(R^{10a})R^{10b}; wherein R⁹, R^{10a} and R^{10b} are as defined in claim 54.

60. The compound as claimed in claim 57, where R⁶ in —C(═NR⁶)R⁸ is —OR⁹ and R⁹ is selected from C₁-C₆-alkyl, C₁-C₆-haloalkyl, C₃-C₈-cycloalkyl, C₃-C₈-halocycloalkyl, C₃-C₈-cycloalkyl-C₁-C₄-alkyl-, C₂-C₆-alkenyl, C₂-C₆-haloalkenyl, C₂-C₆-alkynyl and C₂-C₆-haloalkynyl.

61. The compound as claimed in claim 57, where R⁶ in —C(═NR⁶)R⁸ is —N(R^{10a})R^{10b}, where
R^{10a} and R^{10b}, independently of each other, are selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkylcarbonyl, C₁-C₆-haloalkylcarbonyl,
—C(═O)N(R^{14a})R^{14b}, —C(═S)N(R^{14a})R^{14b}, phenyl which is optionally substituted with 1, 2, 3 or 4, substituents R¹⁶, and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂, as ring members, where the heterocyclic ring is optionally substituted with one or more substituents R¹⁶;
or R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂, as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl;
wherein R^{14a}, R^{14b} and R¹⁶ are as defined in claim 54.

62. The compound as claimed in claim 61, where
R^{10a} is selected from hydrogen, C₁-C₆-alkyl and C₁-C₆-haloalkyl; and
R^{10b} is selected from —C(═O)N(R^{14a})R^{14b}, —C(═S)N(R^{14a})R^{14b}, phenyl which is optionally substituted with 1, 2, 3 or 4, substituents R¹⁶, and a 5- or 6-membered heteroaromatic ring comprising 1, 2 or 3 heteroatoms selected from N, O and S, as ring members, where the heteroaromatic ring is optionally substituted with one or more substituents R¹⁶;
wherein R^{14a}, R^{14b} and R¹⁶ are as defined in claim 54.

63. The compound as claimed in claim 62, where
R^{10a} is selected from hydrogen and C₁-C₆-alkyl; and
R^{10b} is selected from —C(═O)N(R^{14a})R^{14b} and —C(═S)N(R^{14a})R^{14b};
wherein R^{14a} and R^{14b} are as defined in claim 54.

64. The compound as claimed in claim 61, where
R^{14a} is selected from hydrogen, C₁-C₆-alkyl and C₁-C₆-haloalkyl; and
R^{14b} is selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkyl-C₁-C₄-alkyl-, where the cycloalkyl moieties in the three last-mentioned radicals may carry a CN group; C₁-C₆-alkyl substituted with a CN group, phenyl which is optionally substituted with 1, 2, 3 or 4, substituents each independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 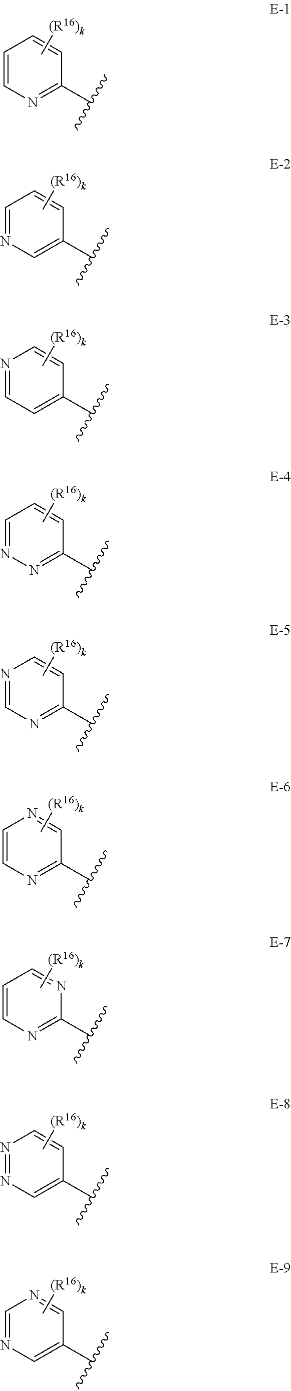 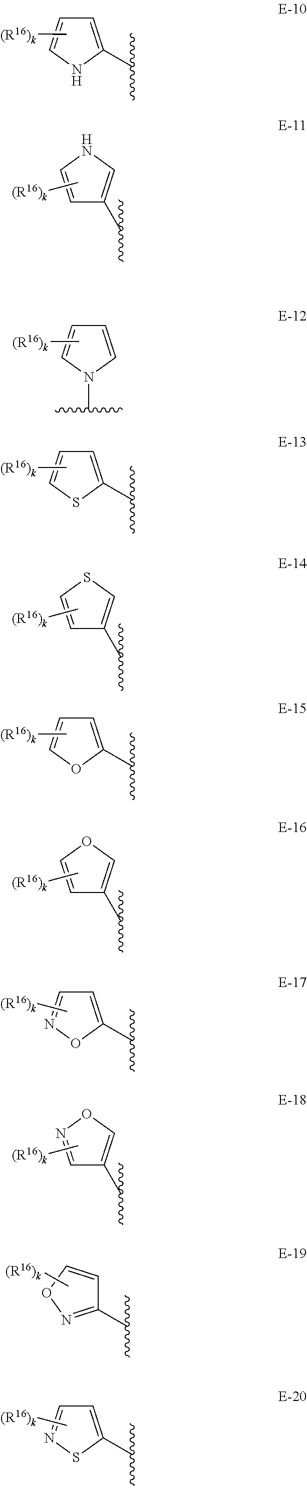 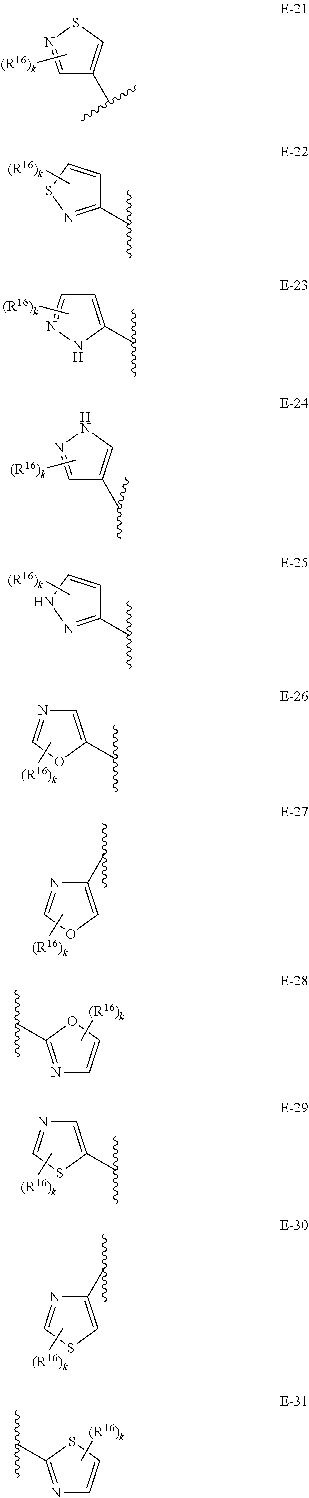 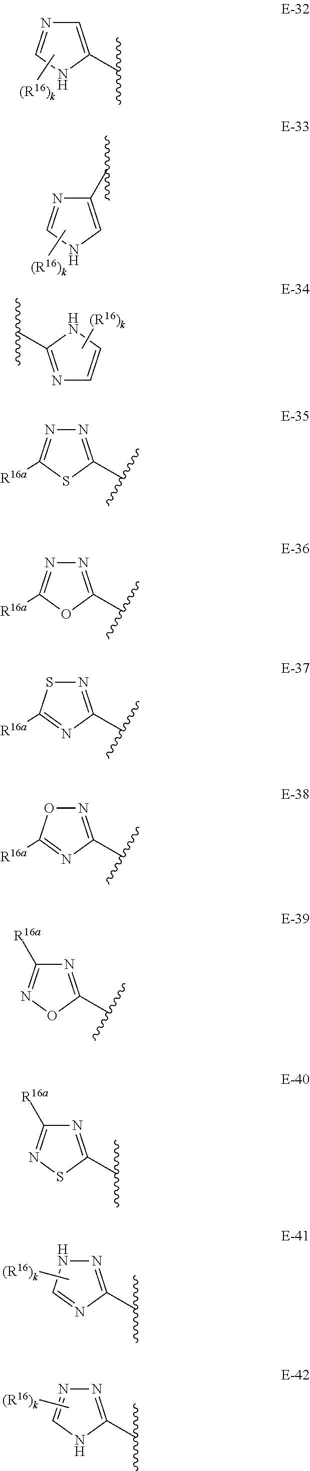 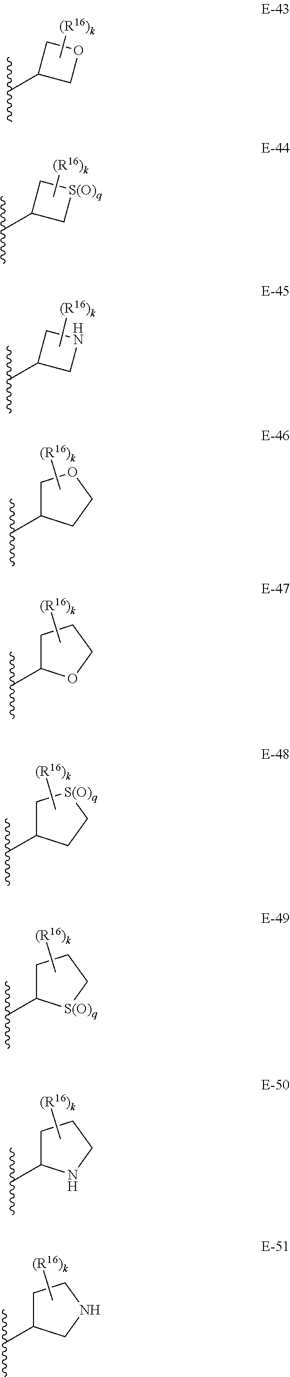
wherein [symbol]
denotes the bonding point to the remainder of the molecule;
k is 0, 1, 2 or 3,
q is 0, 1 or 2;
each R^{16a} is independently hydrogen or is R¹⁶; and
each R¹⁶ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or two R¹⁶ present on the same carbon atom of a saturated ring may form together ═O or ═S.

65. The compound as claimed in claim 64, where R^{14a} is selected from hydrogen and methyl and R^{14b} is selected from hydrogen, C₁-C₆-alkyl and C₁-C₆-haloalkyl.

66. The compound as claimed in claim 57, where R⁸ in —C(═NR⁶)R⁸ as a meaning for A¹ is selected from hydrogen and N(R^{10a})R^{10b}.

67. The compound as claimed in claim 66, where R^{10a} and R^{10b} in N(R^{10a})R^{10b} as a meaning for R⁸ are selected, independently of each other, from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkylcarbonyl, C₁-C₆-haloalkylcarbonyl, —C(═O)N(R^{14a})R^{14b}, —C(═S)N(R^{14a})R^{14b}, phenyl which is optionally substituted with 1, 2, 3 or 4, substituents R¹⁶, and a 3-, 4-, 5-, 6- or 7-membered saturated, partially unsaturated or maximally unsaturated heterocyclic ring comprising 1, 2, 3 or 4 heteroatoms or heteroatom groups selected from N, O, S, NO, SO and SO₂, as ring members, where the heterocyclic ring is optionally substituted with one or more substituents R¹⁶;
or R^{10a} and R^{10b} form together with the nitrogen atom they are bonded to a 3-, 4-, 5-, 6-, 7- or 8-membered saturated, partially unsaturated or maximally unsaturated heterocyclic ring, wherein the heterocyclic ring may additionally contain one or two heteroatoms or heteroatom groups selected from N, O, S, NO, SO and SO₂, as ring members, where the heterocyclic ring optionally carries one or more substituents selected from halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl;
wherein R^{14a}, R^{14b} and R¹⁶ are as defined in claim 54.

68. The compound as claimed in claim 57, where A¹ is N(R⁵)R⁶, wherein
R⁵ is selected from hydrogen and C₁-C₆-alkyl; and
R⁶ is N(R^{10a})R^{10b}, wherein
R^{10a} is selected from hydrogen and C₁-C₆-alkyl; and
R^{10b} is —C(═O)R¹³, wherein
R¹³ is selected from the group consisting of hydrogen, halogen, C₁-C₆-alkyl, C₃-C₈-cycloalkyl, C₂-C₆-alkenyl and C₂-C₆-alkynyl, wherein the four last-mentioned aliphatic or cycloaliphatic radicals may be unsubstituted, partially or fully halogenated and/or may carry 1 or 2 radicals selected from CN, C₃-C₄-cycloalkyl, C₁-C₄-alkoxy, C₁-C₄-haloalkoxy and oxo.

69. The compound as claimed in claim 53, where A is A² and in A² W is O.

70. The compound as claimed in claim 54, where in A² Y is —N(R⁵)R⁶; wherein R⁵ and R⁶ are as defined in claim 54.

71. The compound as claimed in claim 70, where
R⁵ is selected from hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl and C₃-C₈-halocycloalkyl, where the aforementioned aliphatic and cycloaliphatic radicals may be substituted by 1, 2 or 3 radicals R⁸; and
R⁶ is selected from hydrogen, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted by one or more substituents R⁸,
—OR⁹, —N(R^{10a})R^{10b}, —S(O)ₙR⁹, —C(═O)N(R^{10a})N(R^{10a})R^{10b}, —C(═O)R⁸, —P(═O)(OR⁹)₂, —P(═S)(OR⁹)₂,
phenyl which may be substituted with 1, 2, 3, 4, or 5 substituents R¹¹, and
a 3-, 4-, 5-, 6-, 7-, 8-, 9- or 10-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹;
or
R⁵ and R⁶, together with the nitrogen atom to which they are bound, form a 3-, 4-, 5-, 6-, 7- or 8-membered saturated, partially unsaturated or maximally unsaturated heteromonocyclic ring, where the ring may further contain 1, 2, 3 or 4 heteroatoms or heteroatom-containing groups selected from O, S, SO, SO₂, N, NH, C═O and C═S as ring members, or form a 7-, 8-, 9- or 10-membered saturated, partially unsaturated or maximally unsaturated heterobicyclic ring, where the ring may further contain 1, 2, 3 or 4 heteroatoms or heteroatom groups selected from N, O, S, NO, SO, SO₂, C═O and C═S, wherein the heteromonocyclic or heterobicyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
or
R⁵ and R⁶ together form a group ═C(R⁸)₂, ═S(O)ₘ(R⁹)₂, ═NR^{10a} or ═NOR⁹;
wherein R⁸, R⁹, R^{10a}, R^{10b} and R¹¹ are as defined in claim 54.

72. The compound as claimed in claim 71, where
R⁵ is selected from hydrogen, C₁-C₆-alkyl, C₂-C₃-alkynyl, —CH₂—CN and C₁-C₆-alkoxy-methyl-; and
R⁶ is selected from hydrogen, C₁-C₆-alkyl, C₁-C₆-haloalkyl, C₃-C₆-cycloalkyl, C₃-C₆-halocycloalkyl, where the four last-mentioned aliphatic and cycloaliphatic radicals may carry 1, 2 or 3 radicals R⁸; C₂-C₆-alkynyl, —C(═O)R⁸, —S(O)ₙR⁹, —P(═O)(OR⁹)₂, —P(═S)(OR⁹)₂, phenyl which may be substituted with 1, 2, 3, 4, or 5 substituents R¹¹, and a 3-, 4-, 5-, 6- or 7-membered saturated, partially unsaturated or maximally unsaturated heteromonocyclic ring containing 1, 2 or 3 heteroatoms or heteroatom groups independently selected from N, O, S, NO, SO and SO₂, as ring members, where the heteromonocyclic ring may be substituted with one or more substituents R¹¹;
wherein R⁸ and R¹¹ are as defined in claim 54 and wherein each R⁹ is independently selected from C₁-C₆-alkyl, C₁-C₆-haloalkyl, C₃-C₆-cycloalkyl, phenyl and —N(R^{10a})R^{10b}, wherein R^{10a} and R^{10b}, independently of each other, are hydrogen or C₁-C₆-alkyl;
or
R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, or form a 7-, 8-, 9- or 10-membered saturated heterobicyclic ring, where the ring may further contain 1, 2, 3 or 4 heteroatoms or heteroatom groups selected from O, S, SO, SO₂, NH and C═O, wherein the heteromonocyclic or heterobicyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy and C₁-C₆-haloalkoxy;
or
R⁵ and R⁶ together form a group ═S(O)ₘ(R⁹)_{2;}
wherein each R⁹ is independently selected from C₁-C₆-alkyl, C₁-C₆-haloalkyl, C₃-C₆-cycloalkyl, C₃-C₆-halocycloalkyl and C₃-C₆-cycloalkyl-C₁-C₄-alkyl.

73. The compound as claimed in claim 72, where
R⁵ is selected from hydrogen, C₁-C₆-alkyl, C₂-C₃-alkynyl, CH₂—CN and C₁-C₆-alkoxy-methyl-; and
R⁶ is selected from C₁-C₆-alkyl, C₁-C₆-haloalkyl, C₁-C₄-alkyl which carries one radical R⁸, C₃-C₆-cycloalkyl which may be substituted by 1 or 2 substituents selected from F, CN, methyl and oxo,
C₂-C₆-alkynyl, —C(═O)R⁸, phenyl which may be substituted with 1, 2, 3, 4, or 5 substituents R¹¹, and a 3-, 4-, 5- or 6-membered saturated, partially unsaturated or maximally unsaturated heteromonocyclic ring containing 1, 2 or 3 heteroatoms or heteroatom groups independently selected from N, O, S, NO, SO and SO₂, as ring members, where the heteromonocyclic ring may be substituted with one or more substituents R¹¹;
wherein R⁸ and R¹¹ are as defined in claim 54;
or
R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, or form a 7-, 8-, 9- or 10-membered saturated heterobicyclic ring, where the ring may further contain 1, 2, 3 or 4 heteroatoms or heteroatom groups selected from O, S, SO, SO₂, NH and C═O, wherein the heteromonocyclic or heterobicyclic ring may be substituted with 1, 2 or 3 substituents independently selected from the group consisting of halogen, cyano, C₁-C₆-alkyl, C₁-C₆-haloalkyl, C₁-C₆-alkoxy and C₁-C₆-haloalkoxy;
or
R⁵ and R⁶ together form a group ═S(O)ₘ(R⁹)_{2;}
wherein each R⁹ is independently selected from C₁-C₆-alkyl, C₁-C₆-haloalkyl, C₃-C₆-cycloalkyl, C₃-C₆-halocycloalkyl and C₃-C₆-cycloalkyl-C₁-C₄-alkyl.

74. The compound as claimed in claim 71, where
R⁸ as a substituent on an aliphatic or cycloaliphatic group is selected from cyano, C₃-C₈-cycloalkyl which may be substituted by 1 or 2 substituents selected from CN, methyl and oxo, C₃-C₈-halocycloalkyl, —OR⁹, —S(O)ₙR⁹, —N(R^{10a})R^{10b}, —C(═O)R¹³, —C(═O)N(R^{10a})R^{10b}, —C(═O)OR⁹, phenyl, optionally substituted with 1, 2, 3, 4 or 5 substituents R¹⁶, and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶; and
R⁸ in the group —C(═O)R⁸ is selected from hydrogen, C₁-C₆-alkyl, C₁-C₆-haloalkyl, —OR⁹ and —N(R^{10a})R^{10b};
wherein R⁹, R^{10a}, R^{10b}, R¹³ and R¹⁶ are as defined in claim 54.

75. The compound as claimed in claim 74, where
R⁵ is selected from hydrogen and C₁-C₄-alkyl;
R⁶ is selected from C₁-C₆-alkyl, C₁-C₆-haloalkyl, C₁-C₄-alkyl which carries one radical R⁸, C₃-C₆-cycloalkyl which may be substituted by 1 or 2 substituents selected from F, CN, methyl and oxo; C₂-C₆-alkynyl, phenyl which may be substituted with 1, 2, 3, 4, or 5 substituents R¹¹, and a heteromonocyclic ring selected from rings of formulae F-1 to F-51 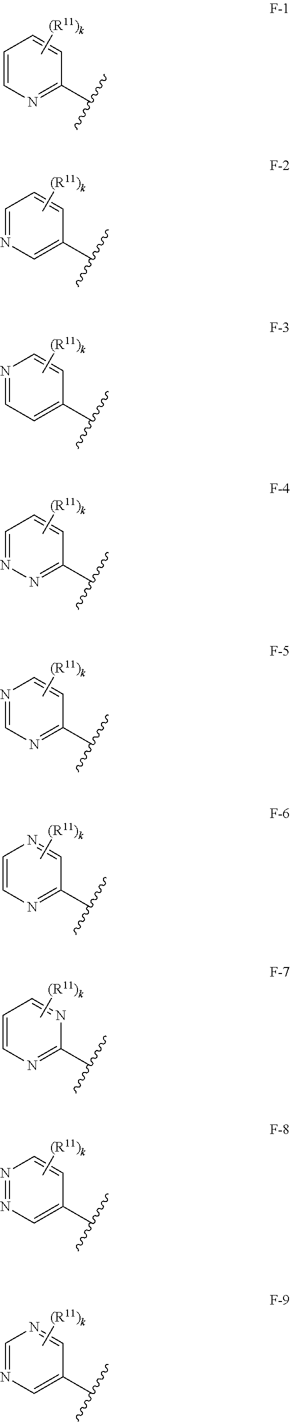 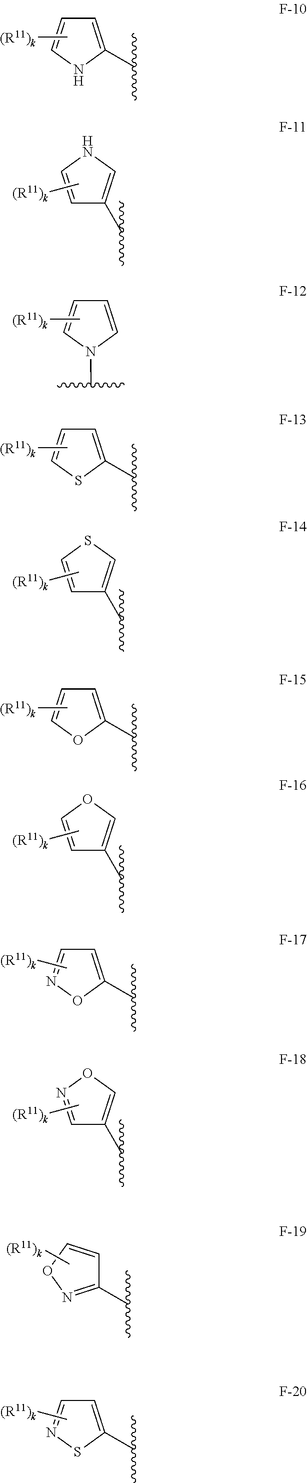 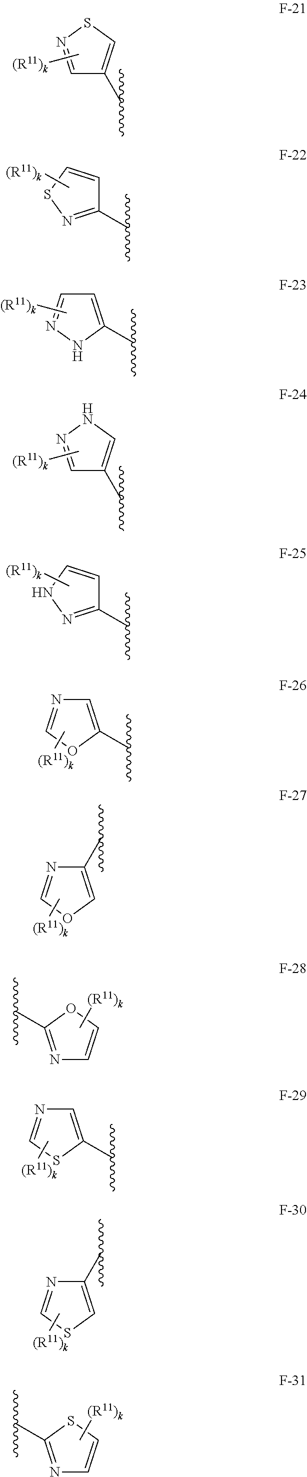 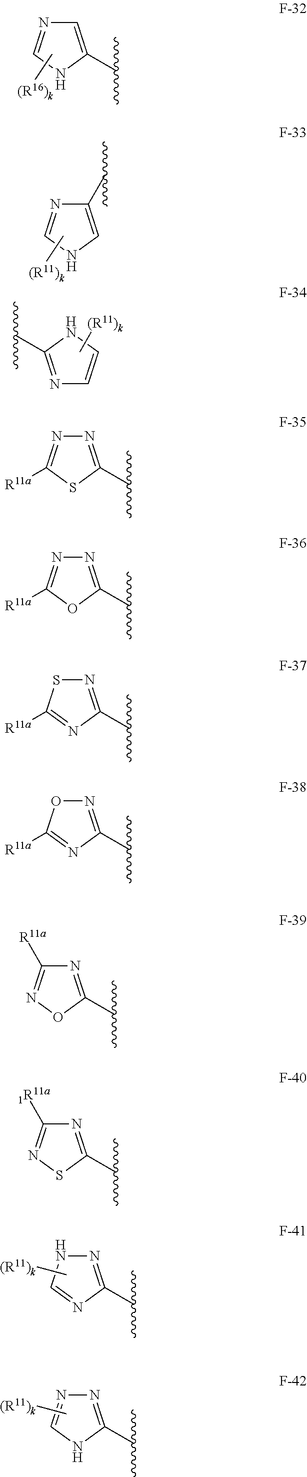 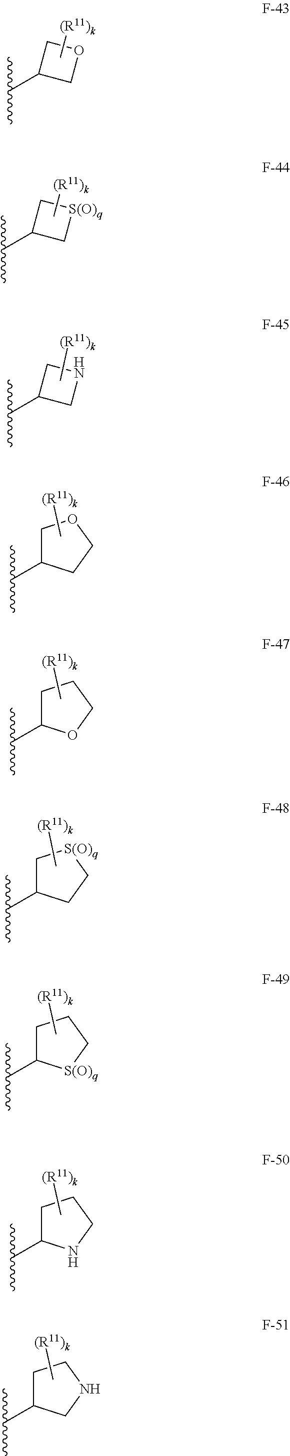
wherein [symbol]
denotes the bonding point to the remainder of the molecule,
k is 0, 1, 2 or 3,
q is 0, 1 or 2,
each R^{11a} is independently hydrogen or is R¹¹; and
each R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or two R¹¹ present on the same carbon atom of a saturated heterocyclic ring may form together ═O or ═S;
R⁸ is selected from CN, C₃-C₈-cycloalkyl which may be substituted by 1 or 2 substituents selected from F, CN, methyl and oxo; C₃-C₈-halocycloalkyl, C₁-C₆-alkylthio, C₁-C₆-haloalkylthio, —C(═O)R¹³, —C(═O)N(R^{10a})R^{10b}, phenyl, optionally substituted with 1, 2, 3, 4 or 5 substituents R¹⁶, and a heterocyclic ring selected from rings of formulae E-1 to E-51 as defined in claim 65;
wherein
R^{10a} is selected from the group consisting of hydrogen, C₁-C₆-alkyl, C₂-C₃-alkynyl, —CH₂—CN and C₁-C₆-alkoxy-methyl;
R^{10b} is selected from the group consisting of hydrogen, C₁-C₆-alkyl, C₁-C₆-haloalkyl, C₃-C₆-cycloalkyl, C₃-C₆-halocycloalkyl, phenyl which is optionally substituted with 1, 2, 3, 4 or 5 substituents selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 as defined in claim 65;
R¹³ is selected from the group consisting of hydrogen, C₁-C₆-alkyl, C₁-C₆-haloalkyl, C₃-C₆-cycloalkyl and C₃-C₆-halocycloalkyl; and
each R¹⁶ as a substituent on phenyl or heterocyclic rings of formulae E-1 to E-51 is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
two R¹⁶ present on the same carbon atom of a saturated heterocyclic ring may form together ═O or ═S;
or
R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, wherein the heterocyclic ring may be substituted with 1, 2 or 3 substituents independently selected from the group consisting of halogen, cyano, C₁-C₆-alkyl, C₁-C₆-haloalkyl, C₁-C₆-alkoxy and C₁-C₆-haloalkoxy;
or
R⁵ and R⁶ together form a group ═S(O)ₘ(R⁹)_{2;}
wherein each R⁹ is independently selected from C₁-C₆-alkyl, C₁-C₆-haloalkyl, C₃-C₆-cycloalkyl, C₃-C₆-halocycloalkyl and C₃-C₆-cycloalkyl-C₁-C₄-alkyl, and m is 0, 1 or 2.

76. The compound as claimed in claim 65, where
R⁵ is selected from hydrogen and C₁-C₄-alkyl;
R⁶ is selected from C₁-C₆-alkyl, C₁-C₆-haloalkyl, C₁-C₄-alkyl which carries one radical R⁸, C₃-C₆-cycloalkyl which may be substituted by 1 CN substituent, C₂-C₆-alkynyl,
phenyl which may be substituted with 1, 2, 3, 4, or 5 substituents R¹¹, and a heteromonocyclic ring selected from rings of formulae F-1 to F-51 as defined in claim 75;
wherein
R⁸ is selected from CN, C₃-C₈-cycloalkyl which may be substituted by 1 CN substituent; C₃-C₈-halocycloalkyl, —C(═O)R¹³, and a heterocyclic ring selected from rings of formulae E-1 to E-51;
wherein
R¹³ is selected from the group consisting of hydrogen, C₁-C₆-alkyl and C₁-C₆-haloalkyl; and
each R¹⁶ as a substituent on phenyl or heterocyclic rings of formulae E-1 to E-51 is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
two R¹⁶ present on the same carbon atom of a saturated heterocyclic ring may form together ═O or ═S;
or
R⁵ and R⁶, together with the nitrogen atom to which they are bound, form a 3-, 4-, 5- or 6-membered saturated heteromonocyclic ring, where the ring may further contain 1 or 2 heteroatoms or heteroatom-containing groups selected from O, S, SO, SO₂, NH and C═O as ring members, wherein the heterocyclic ring may be substituted with 1, 2 or 3 substituents independently selected from the group consisting of halogen, cyano, C₁-C₆-alkyl, C₁-C₆-haloalkyl, C₁-C₆-alkoxy and C₁-C₆-haloalkoxy;
or
R⁵ and R⁶ together form a group ═S(O)ₘ(R⁹)_{2;}
wherein each R⁹ is independently selected from C₁-C₆-alkyl and C₁-C₆-haloalkyl, and m is 0.

77. The compound as claimed in claim 54, where A is A³ and in A³ R^{7a} and R^{7b} are independently of each other selected from hydrogen, C₁-C₄-alkyl and C₁-C₄-haloalkyl.

78. The compound as claimed in claim 54, where in A³
R⁵ is selected from hydrogen, C₁-C₆-alkyl, C₃-C₆-cycloalkyl, C₂-C₆-alkenyl, C₂-C₆-alkynyl, wherein the four last-mentioned aliphatic and cycloaliphatic radicals may be partially or fully halogenated and/or may be substituted with one or more substituents R⁸; and
R⁶ is selected from hydrogen, C₁-C₆-alkyl, C₃-C₆-cycloalkyl, C₂-C₆-alkenyl, C₂-C₆-alkynyl, wherein the four last-mentioned aliphatic and cycloaliphatic radicals may be partially or fully halogenated and/or may be substituted by one or more substituents R⁸,
—OR⁹, —N(R^{10a})R^{10b}, —S(O)ₙR⁹, —C(═O)N(R^{10a})N(R^{10a})R^{10b}, —C(═O)R⁸,
and a 3-, 4-, 5-, 6- or 7-membered saturated, partially unsaturated or maximally unsaturated heteromonocyclic or heterobicyclic ring containing 1, 2, 3 or 4 heteroatoms or heteroatom groups independently selected from N, O, S, NO, SO and SO₂, as ring members, where the heteromonocyclic or heterobicyclic ring may be substituted with one or more substituents R¹¹;
or
R⁵ and R⁶, together with the nitrogen atom to which they are bound, form a 3-, 4-, 5- or 6-membered saturated, partially unsaturated or maximally unsaturated heterocyclic ring, where the ring may further contain 1, 2, 3 or 4 heteroatoms or heteroatom-containing groups selected from O, S, N, SO, SO₂, C═O and C═S as ring members, wherein the heterocyclic ring may be substituted with 1, 2, 3, 4 or 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, wherein the aliphatic or cycloaliphatic moieties in the twelve last-mentioned radicals may be substituted by one or more radicals R⁸, and phenyl which may be substituted with 1, 2, 3, 4 or 5 substituents R¹¹;
or
R⁵ and R⁶ together form a group ═C(R⁸)₂, ═S(O)ₘ(R⁹)₂, ═NR^{10a} or ═NOR⁹
wherein R⁸, R⁹, R^{10a}, R^{10b} and R¹¹ are as defined in claim 54.

79. The compound as claimed in claim 78, where
R⁵ is selected from hydrogen, C₁-C₄-alkyl, C₂-C₃-alkynyl, —CH₂—CN and C₁-C₆-alkoxy-methyl; and
R⁶ is —C(═O)R⁸;
wherein R⁸ is as defined in claim 54.

80. The compound as claimed in claim 79, where R⁸ is selected from the group consisting of C₁-C₆-alkyl, C₁-C₆-haloalkyl, C₃-C₈-cycloalkyl, C₃-C₈-halocycloalkyl, where the aliphatic and cycloaliphatic moieties in the four last-mentioned radicals may be substituted by one or more radicals R¹³;
—OR⁹, —S(O)ₙR⁹, —N(R^{10a})R^{10b},
phenyl, optionally substituted with 1, 2, 3, 4 or 5 substituents R¹⁶, and a 3-, 4-, 5- or 6-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more substituents R¹⁶,
wherein R⁹, R^{10a}, R^{10b}, R¹³ and R¹⁶ are as defined in claim 54.

81. The compound as claimed in claim 80, where R⁸ is selected from the group consisting of C₁-C₆-alkyl, C₁-C₆-haloalkyl, C₁-C₄-alkyl substituted by one radical R¹³, C₃-C₈-cycloalkyl, C₃-C₈-halocycloalkyl, —N(R^{10a})R^{10b}, phenyl which is optionally substituted with 1, 2, 3, 4 or 5 substituents each independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; and a heterocyclic ring selected from rings of formulae E-1 to E-51 as defined in claim 63,
wherein
R^{10a} and R^{10b}, independently of each other, are selected from hydrogen, C₁-C₄-alkyl, C₁-C₄-haloalkyl and C₃-C₆-cycloalkyl; and
R¹³ is selected from CN, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl and a heterocyclic ring selected from rings of formulae E-1 to E-51 as defined in claim 63; and
each R¹⁶ as a substituent on heterocyclic rings of formulae E-1 to E-51 is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
two R¹⁶ present on the same carbon atom of a saturated heterocyclic ring may form together ═O or ═S.

82. The compound as claimed in claim 81, where R⁸ is selected from the group consisting of C₁-C₆-alkyl, C₁-C₆-haloalkyl, C₁-C₄-alkyl substituted by one radical R¹³, C₃-C₈-cycloalkyl, C₃-C₈-halocycloalkyl and —N(R^{10a})R^{10b};
wherein
R^{10a} and R^{10b}, independently of each other, are selected from hydrogen and C₁-C₄-alkyl; and
R¹³ is selected from C₁-C₆-alkylthio, C₁-C₆-haloalkylthio, C₁-C₆-alkylsulfinyl, C₁-C₆-alkylsulfonyl and C₁-C₆-haloalkylsulfonyl.

83. The compound as claimed in claim 54, where A is A⁴ and A⁴ is selected from rings of formulae D-1 to D-173 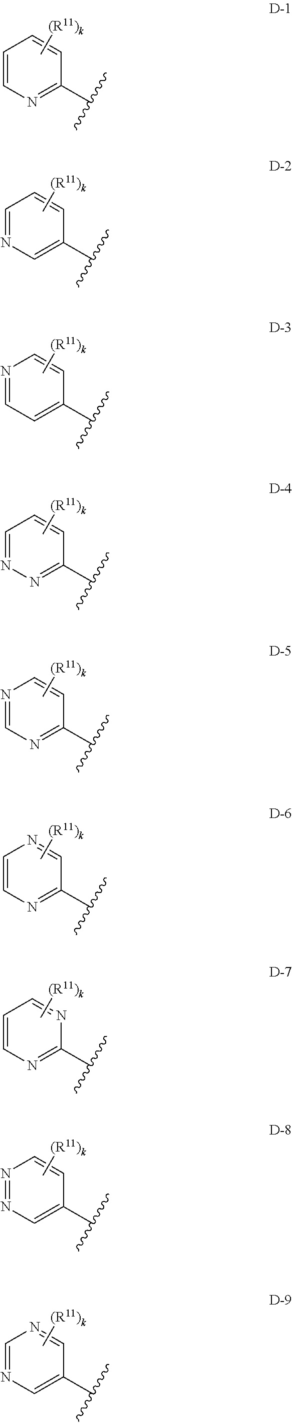 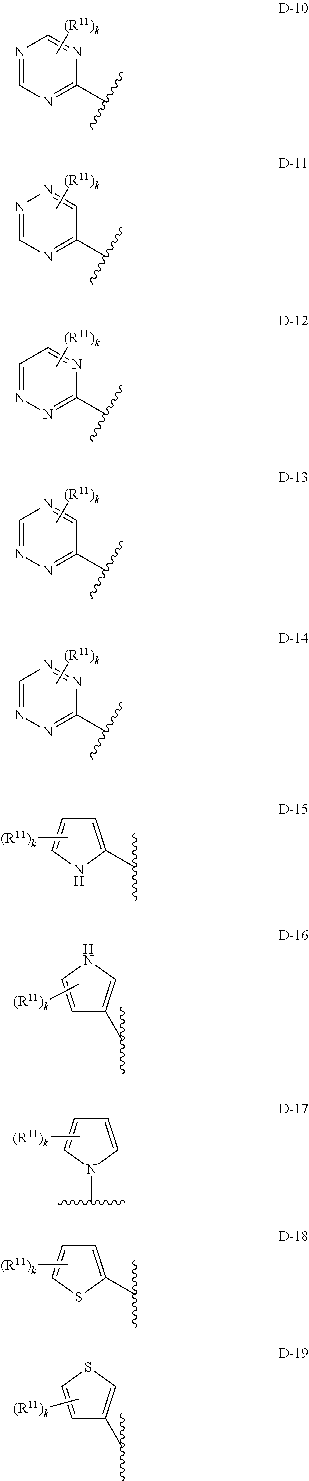 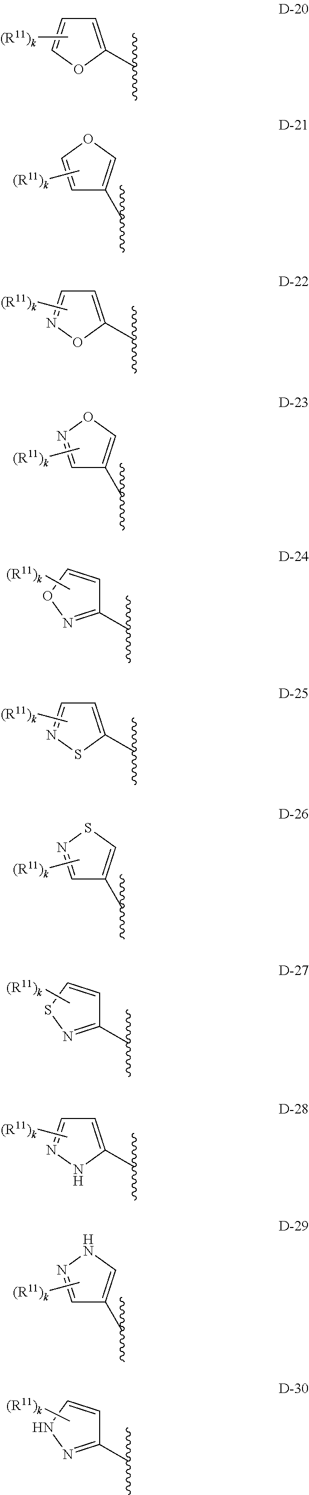 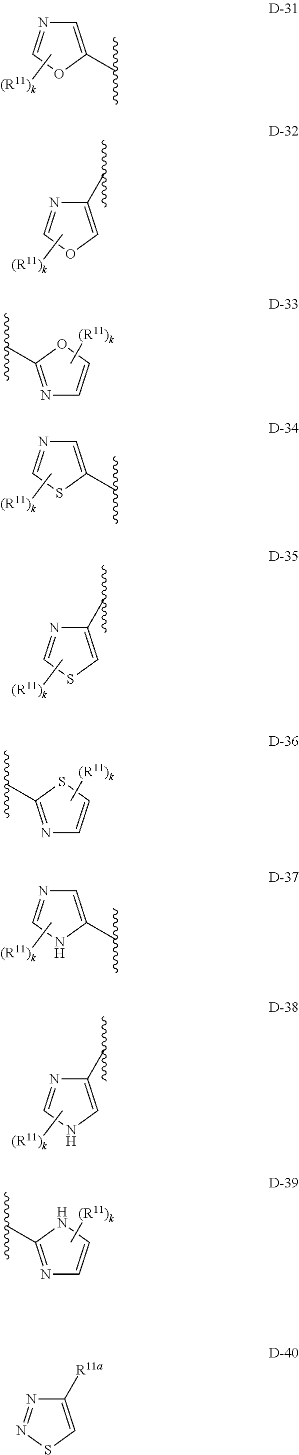 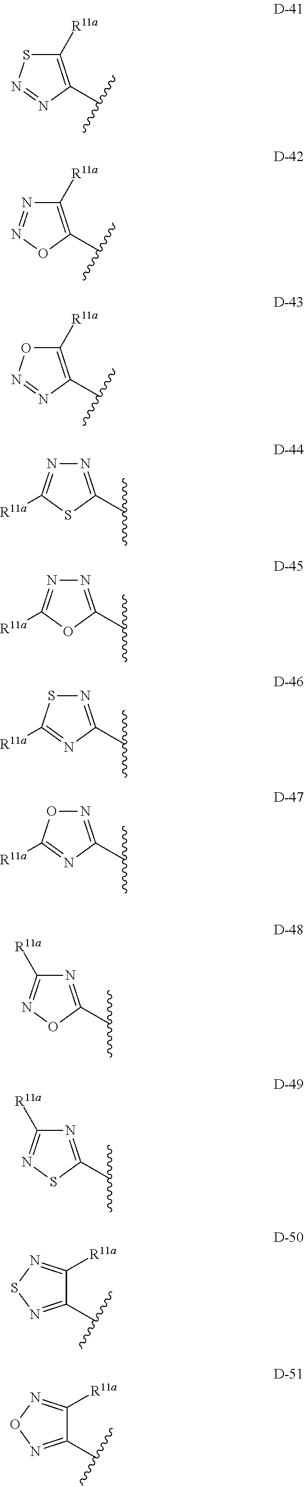 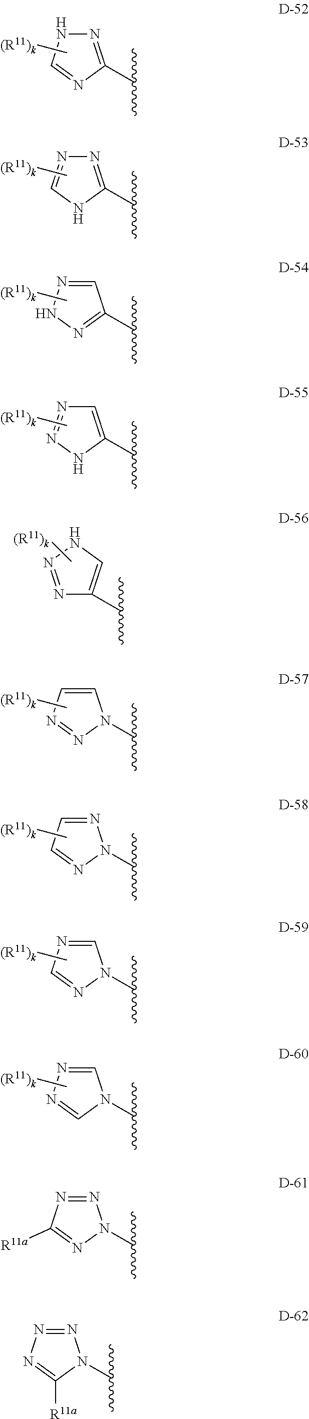 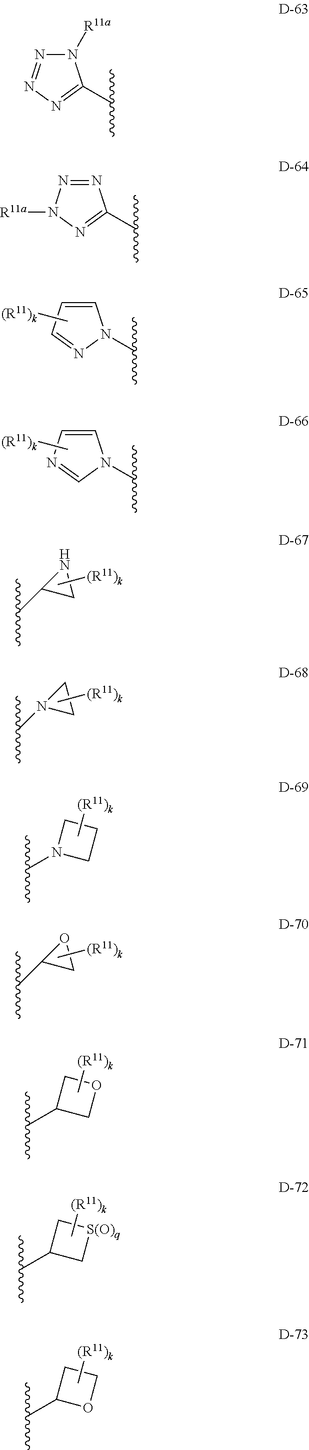 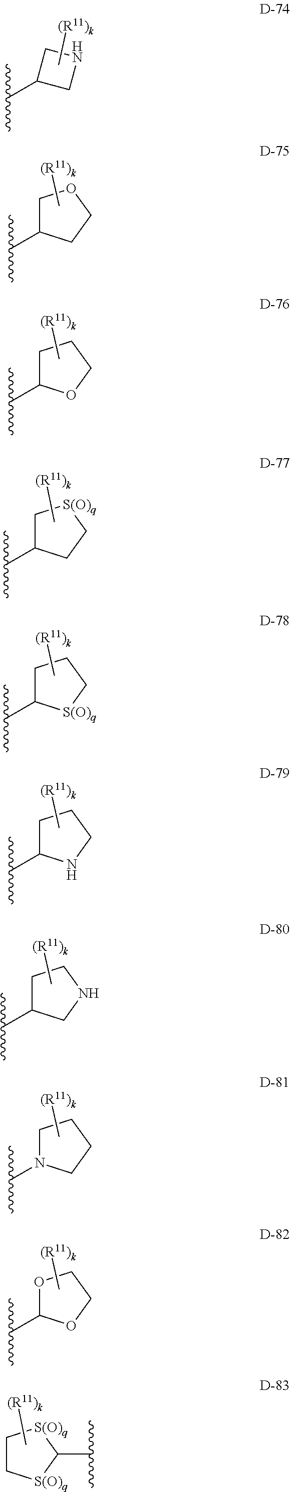 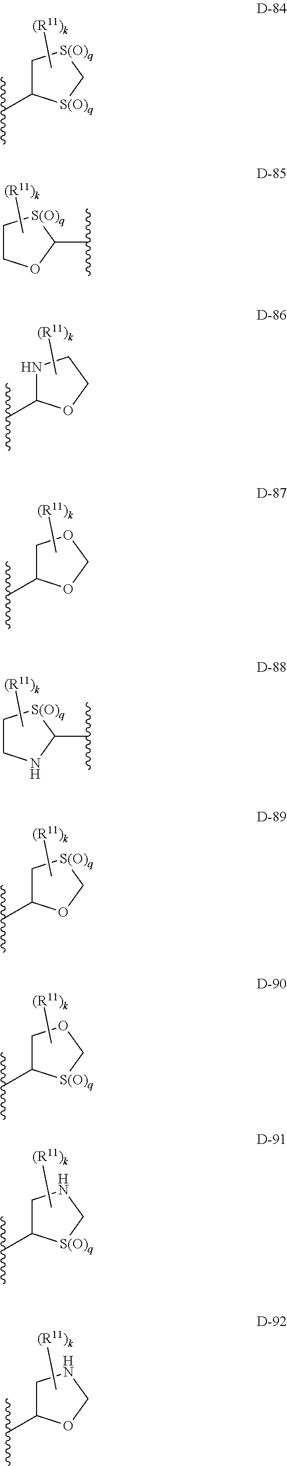 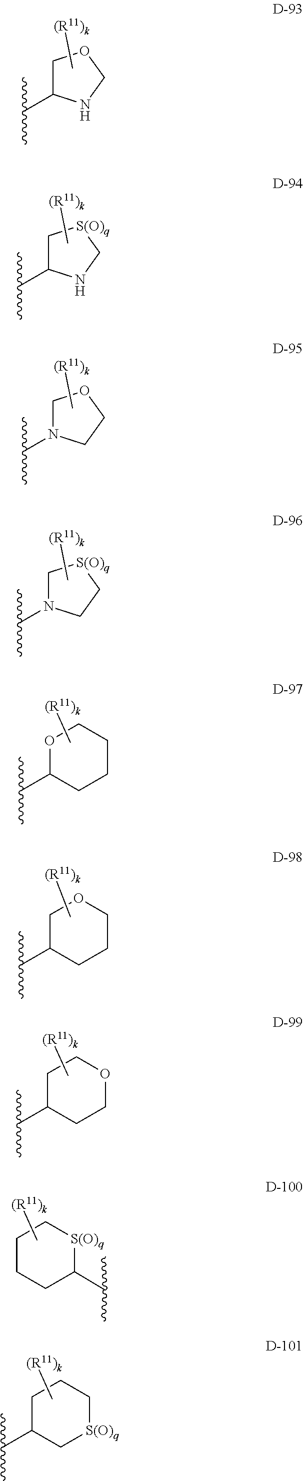 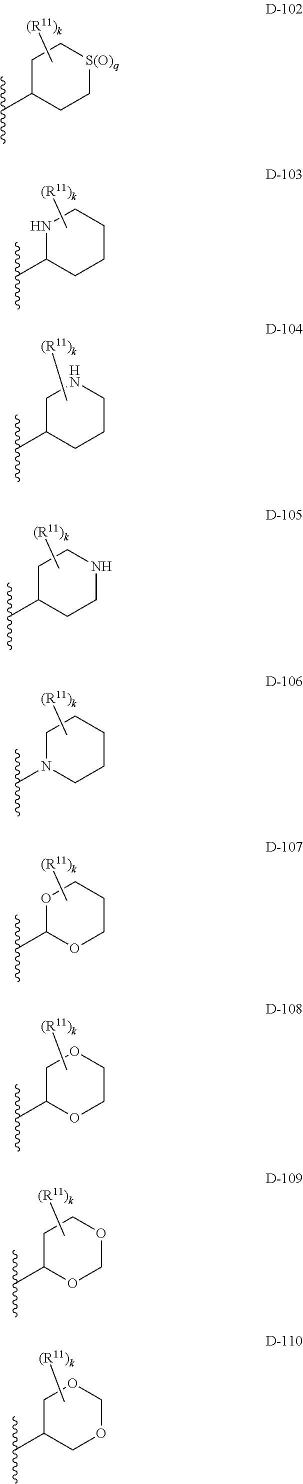 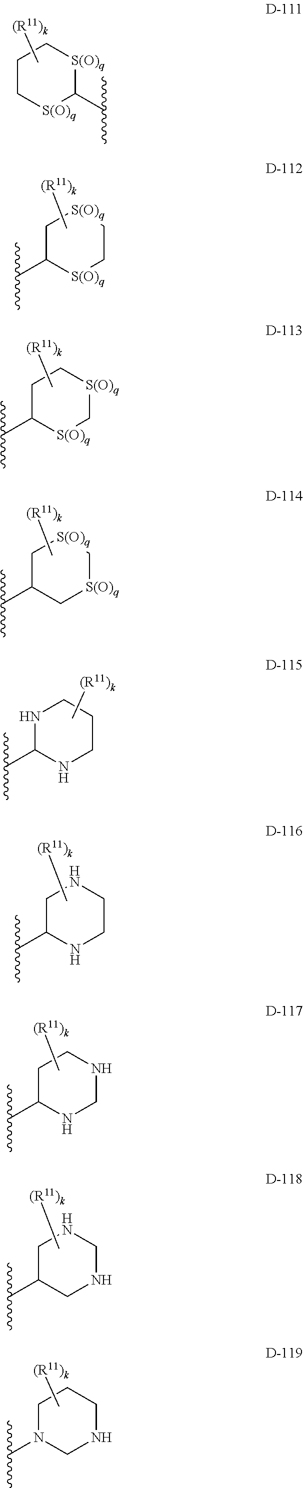 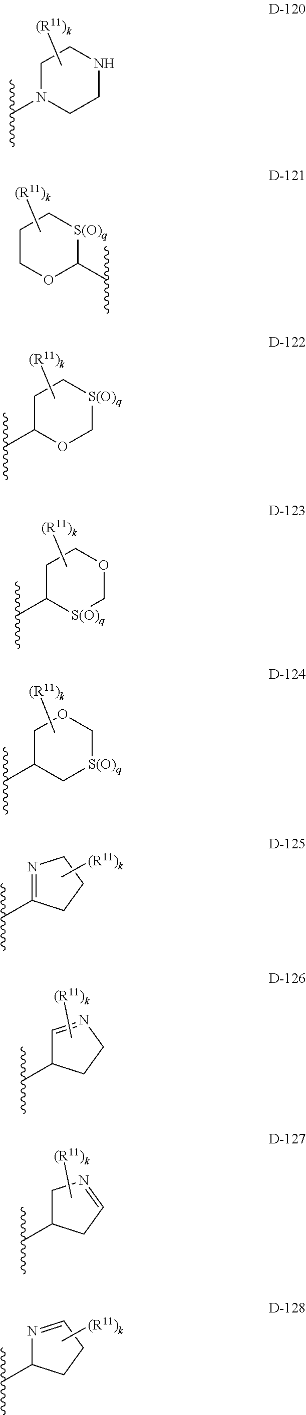 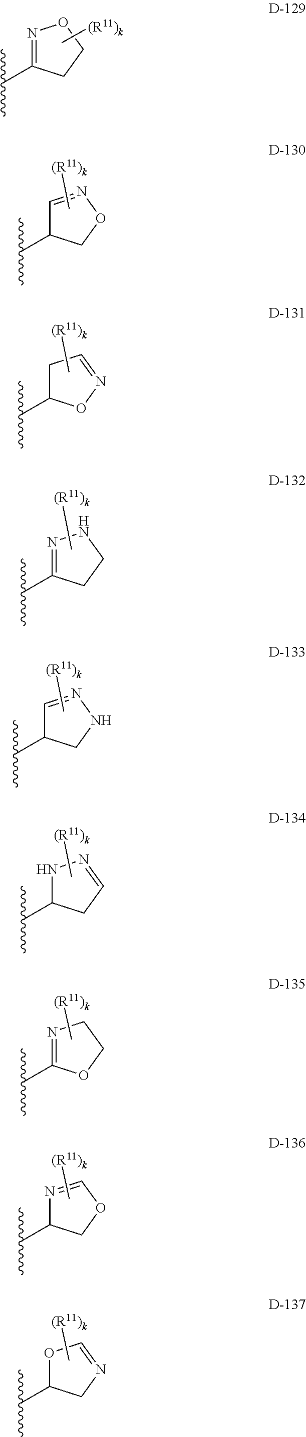 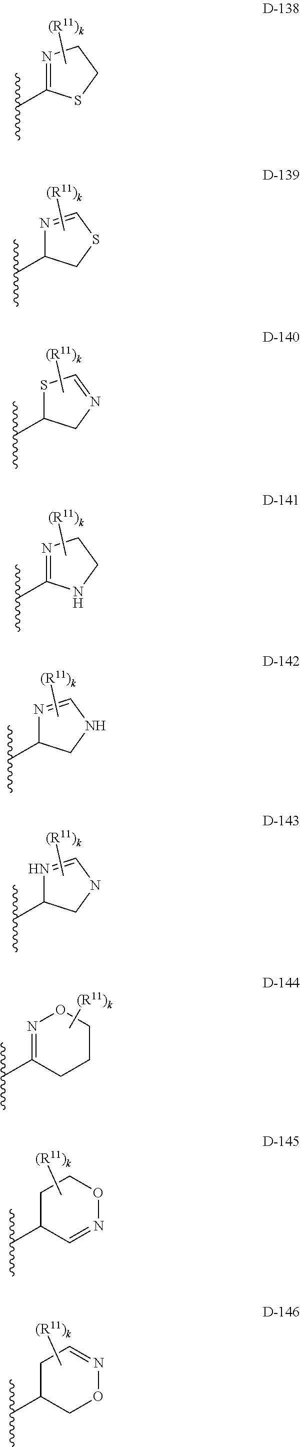 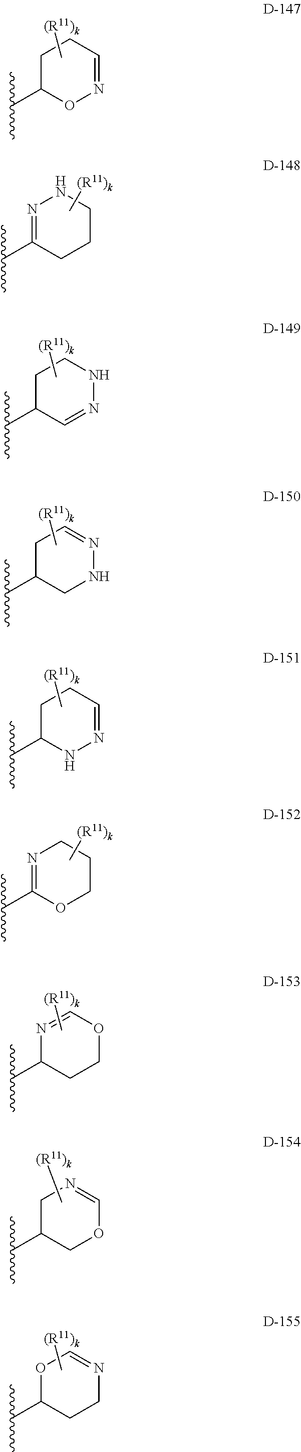 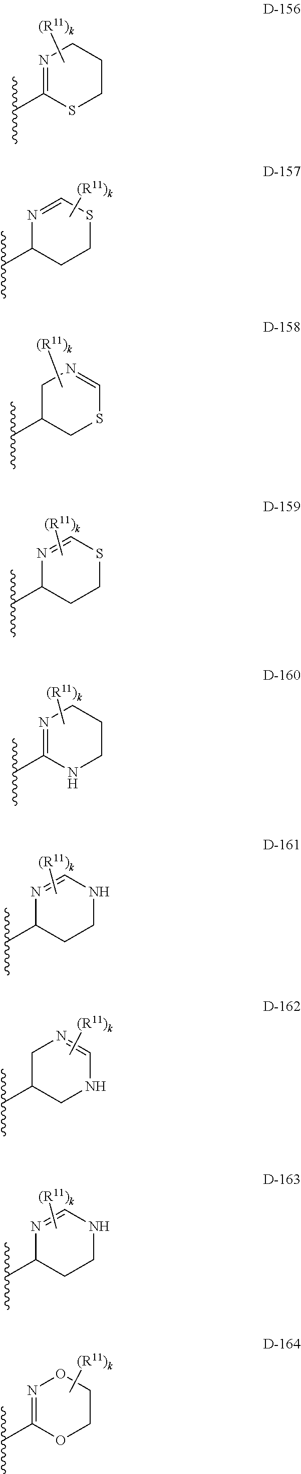 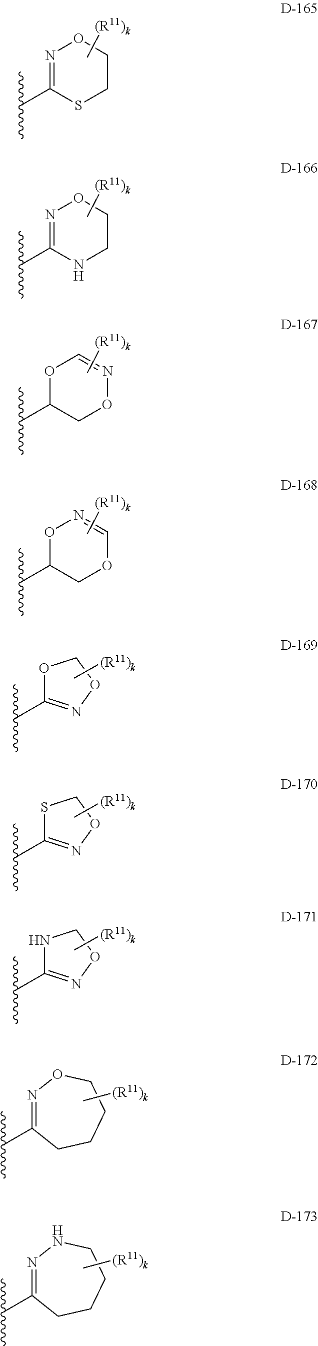
wherein [symbol]
denotes the bonding point to the remainder of the molecule,
k is 0, 1, 2 or 3;
q is 0, 1 or 2;
each R^{11a} is independently hydrogen or has one of the meanings given in claim 54 for R¹¹; and
each R¹¹ is independently as defined in claim 54.

84. The compound as claimed in claim 83, wherein
each R¹¹ is independently selected from the group consisting of halogen, cyano, nitro, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₃-C₆-cycloalkyl, C₃-C₆-halocycloalkyl, C₂-C₄-alkenyl, C₂-C₄-haloalkenyl, C₂-C₄-alkynyl and C₂-C₄-haloalkynyl; or
two R¹¹ present on the same carbon atom of a saturated or partially unsaturated ring may form together ═O or ═S.

85. The compound as claimed in claim 54, where B¹ and B⁵ are CH and B², B³ and B⁴ are CR², where R² has one of the meanings given in claim 54.

86. The compound as claimed in claim 85, where B² is CR², where R² is not hydrogen, and B³ and B⁴ are CR², where R² has one of the meanings given in claim 54.

87. The compound as claimed in claim 54, where R² is selected from hydrogen, halogen, cyano, azido, nitro, —SCN, —SF₅, C₁-C₆-alkyl, C₃-C₈-cycloalkyl, C₂-C₆-alkenyl, C₂-C₆-alkynyl, wherein the four last-mentioned aliphatic and cycloaliphatic radicals may be partially or fully halogenated and/or may be substituted by one or more radicals R⁸, —OR⁹, —S(O)ₙR⁹ and —N(R^{10a})R^{10b},
wherein R⁸, R⁹, R^{10a} and R^{10b} are as defined in claim 54.

88. The compound as claimed in claim 87, where R² is selected from hydrogen, halogen and C₁-C₂-haloalkyl.

89. The compound as claimed in claim 54, where R⁴ is selected from halogen, cyano, C₁-C₄-alkyl, C₁-C₄-haloalkyl, C₃-C₅-cycloalkyl, C₃-C₅-halocycloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₆-alkoxy-C₁-C₆-alkyl-, C₁-C₄-alkylthio and C₁-C₄-haloalkylthio,
and R^{4b} and R^{4c}, independently of each other, are selected from hydrogen, halogen, cyano, C₁-C₄-alkyl, C₁-C₄-haloalkyl, C₃-C₅-cycloalkyl, C₃-C₅-halocycloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₆-alkoxy-C₁-C₆-alkyl-, C₁-C₄-alkylthio and C₁-C₄-haloalkylthio.

90. The compound as claimed in claim 89, where R^{4b} is hydrogen, and R^{4c} is selected from hydrogen, halogen, cyano, C₁-C₄-alkyl, C₁-C₄-haloalkyl, C₃-C₅-cycloalkyl, C₃-C₅-halocycloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₆-alkoxy-C₁-C₆-alkyl-, C₁-C₄-alkylthio and C₁-C₄-haloalkylthio.

91. The compound as claimed in claim 54, where R¹ is selected from C₁-C₄ haloalkyl and C₁-C₄-alkoxycarbonyl.

92. The compound as claimed in claim 54, where L¹ is selected from hydrogen and C₁-C₄-alkyl.

93. The compound as claimed in claim 54, where E is selected from hydrogen, halogen and C₁-C₄-alkyl.

94. The compound as claimed in claim 54, where X is selected from hydrogen, halogen and C₁-C₄-alkyl.

95. The compound as claimed in claim 54, where R³ is selected from hydrogen, C₁-C₄-alkyl, C₂-C₃-alkynyl, —CH₂—CN and C₁-C₆-alkoxy-methyl.

96. The compound as claimed in claim 54, where Z is O.

97. The compound as claimed in claim 54, of formula I.1 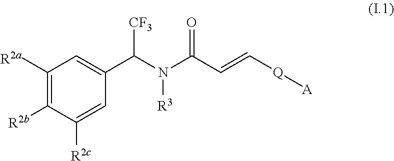
wherein
R³, Q and A are as defined in claim 54 and R^{2a}, R^{2b} and R^{2c}, independently of each other, have one of the meanings given in claim 54 for R².

98. An agricultural composition comprising at least one compound of the formula I, as defined in claim 54, a stereoisomer thereof and/or at least one agriculturally acceptable salt thereof, and at least one inert liquid and/or solid agriculturally acceptable carrier.

99. A veterinary composition comprising at least one compound of the formula I, as defined in claim 54, a stereoisomer thereof and/or at least one veterinarily acceptable salt thereof, and at least one inert liquid and/or solid veterinarily acceptable carrier.

100. A method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one imine compound of the formula I as defined in claim 54, a stereoisomer thereof and/or at least one agriculturally acceptable salt thereof.

101. The method as claimed in claim 102, for protecting plants from attack or infestation by invertebrate pests, which method comprises treating the plants with a pesticidally effective amount of at least one compound of the formula I as defined in claim 54, a stereoisomer thereof and/or at least one agriculturally acceptable salt thereof.

102. The method as claimed in claim 102, for protecting plant propagation material and/or the plants which grow therefrom from attack or infestation by invertebrate pests, which method comprises treating the plant propagation material with a pesticidally effective amount of at least one compound of the formula I as defined in claim 54, a stereoisomer thereof and/or at least one agriculturally acceptable salt thereof.

103. Plant propagation material, comprising at least one compound of the formula I as defined in claim 54, a stereoisomer thereof and/or at least one agriculturally acceptable salt thereof.

104. A method for treating or protecting an animal from infestation or infection by invertebrate pests which comprises bringing the animal in contact with a pesticidally effective amount of at least one compound of the formula I as defined in claim 54, a stereoisomer thereof and/or at least one veterinarily acceptable salt thereof.
